(19) United States
(12) Patent Application Publication
(10) Pub. No.: US 2022/0404360 A1
(43) Pub. Date: Dec. 22, 2022

(54) ASSAYS FOR VIRAL STRAIN DETERMINATION

(71) Applicant: Meso Scale Technologies, LLC., Rockville, MD (US)

(72) Inventors: George SIGAL, Rockville, MD (US); Seth B. HARKINS, Bethesda, MD (US); Timothy J. BREAK, Clarksburg, MD (US)

(21) Appl. No.: 17/749,716

(22) Filed: May 20, 2022

Related U.S. Application Data
(60) Provisional application No. 63/191,540, filed on May 21, 2021.
(60) Provisional application No. 63/209,783, filed on Jun. 11, 2021.
(60) Provisional application No. 63/214,291, filed on Jun. 24, 2021.
(60) Provisional application No. 63/215,231, filed on Jun. 25, 2021.
(60) Provisional application No. 63/225,229, filed on Jul. 23, 2021.
(60) Provisional application No. 63/260,310, filed on Aug. 16, 2021.
(60) Provisional application No. 63/247,892, filed on Sep. 24, 2021.
(60) Provisional application No. 63/252,653, filed on Oct. 6, 2021.
(60) Provisional application No. 63/271,830, filed on Oct. 26, 2021.
(60) Provisional application No. 63/285,739, filed on Dec. 3, 2021.
(60) Provisional application No. 63/288,695, filed on Dec. 13, 2021.
(60) Provisional application No. 63/296,723, filed on Jan. 5, 2022.
(60) Provisional application No. 63/300,878, filed on Jan. 19, 2022.
(60) Provisional application No. 63/308,680, filed on Feb. 10, 2022.
(60) Provisional application No. 63/332,102, filed on Apr. 18, 2022.

(51) Int. Cl. G01N 33/569; C12Q 1/6818; G16B 25/10; G16B 30/00

(52) U.S. Cl. CPC G01N 33/56983; C12Q 1/6818; G16B 25/10; G16B 30/00; G01N 2333/165; G01N 2469/20

(57) ABSTRACT

The invention relates to methods and kits for determining a SARS-CoV-2 strain in a sample. The invention also provides methods and kits for detecting a single nucleotide polymorphism (SNP) in a target nucleic acid, wherein the target nucleic acid is a SARS-CoV-2 nucleic acid. The invention further provides methods and kits for detecting one or more antibody biomarkers in a sample.

### INCORPORATION BY REFERENCE

Reference is made to U.S. Publication No. 2022/0003766; U.S. Publication No. 2021/0349104; PCT Publication No. WO 2021/222827; PCT Publication No. WO 2021/222830; and PCT Publication No. WO 2021/222832, the contents of each of which is incorporated by reference in its entirety.

### SEQUENCE LISTING

The instant application contains a Sequence Listing which has been submitted electronically in ASCII format and is hereby incorporated by reference in its entirety. Said ASCII copy, created on May 16, 2022, is named 0076-0031W04_ST25.txt and is 159,609 bytes in size.

### FIELD OF THE INVENTION

The invention relates to methods and kits for determining a SARS-CoV-2 strain in a sample. The invention also provides methods and kits for detecting a single nucleotide polymorphism (SNP) in a target nucleic acid, wherein the target nucleic acid is a SARS-CoV-2 nucleic acid. The invention further provides methods and kits for detecting one or more antibody biomarkers in a sample.

### BACKGROUND

Respiratory viruses, including coronaviruses, can cause outbreaks of severe respiratory illnesses that place great burden on communities and healthcare systems. During an outbreak, large-scale tests are needed to identify infected but asymptomatic or mildly ill individuals, which can mitigate widespread disease transmission.

The COVID-19 pandemic created an urgent need for assays for multiple reasons, for example: to detect infection, to determine the stage of infection, e.g., viral load, to determine transmissibility of the virus, to determine presence or absence of virus, e.g., on surfaces, to aid in the development of vaccines, for epidemiological studies, to follow the immune status and past viral exposure of individuals, for research into factors contributing to morbidity and mortality of viral infection. Although some assays were developed early in the pandemic, they were slow or low throughput, lacked sensitivity, were inaccurate, were expensive, or otherwise inadequate. For example, current PCR-based tests, e.g., for SARS-CoV-2, are analytically sensitive but require a lengthy, complex, and expensive sample processing procedure, and may be difficult to run at the scale needed to screen large populations. Moreover, accurate and sensitive serology tests can be useful for epidemiological studies and to identify individuals who are immune or at low risk of infection. Thus, high-quality assays are desperately needed to address the pandemic.

### SUMMARY OF THE INVENTION

In embodiments, the invention provides a method for determining a SARS-CoV-2 strain in a sample, comprising: (a) detecting at least a first antibody biomarker in the sample that binds to an antigen, e.g., an S protein, N protein, and/or S-RBD, from a first SARS-CoV-2 strain and at least a second antibody biomarker in the sample that binds to an antigen, e.g., an S protein, N protein, and/or S-RBD, from a second SARS-CoV-2 strain, wherein the detecting comprises contacting the sample with a surface comprising one or more binding domains, wherein the S protein from the first SARS-CoV-2 strain is immobilized on a first binding domain, and the S protein from the second SARS-CoV-2 strain is immobilized on a second binding domain; and (b) determining a ratio of the first antibody biomarker to the second antibody biomarker, thereby determining the SARS-CoV-2 strain. In embodiments, the detecting comprises forming a binding complex in each binding domain that comprises an antibody biomarker and the antigen, e.g., the S protein, N protein, or S-RBD; contacting the binding complex in each binding domain with a detection reagent; and measuring concentration of the antibody biomarker in each binding complex.

In embodiments, the invention provides method for detecting a single nucleotide polymorphism (SNP) in a target nucleic acid, wherein the target nucleic acid is a SARS-CoV-2 nucleic acid, comprising: (a) contacting a sample comprising the target nucleic acid with (i) a targeting probe, wherein the targeting probe comprises a first region complementary to a polymorphic site of the target nucleic acid that comprises the SNP, and wherein the targeting probe comprises an oligonucleotide tag; and (ii) a detection probe, wherein the detection probe comprises a second region complementary to an adjacent region of the target nucleic acid comprising the polymorphic site, and wherein the detection probe comprises a detectable label, wherein the targeting probe and the detection probe each independently comprises a sequence as shown in Table 10 or Table 14; (b) hybridizing the targeting and detection probes to the target nucleic acid; (c) ligating the targeting and detection probes that hybridize with perfect complementarity at the polymorphic site to form a ligated target complement comprising the oligonucleotide tag and the detectable label; (d) contacting the product of (c) with a surface comprising an immobilized binding reagent, wherein the binding reagent comprises an oligonucleotide complementary to the oligonucleotide tag; (e) forming a binding complex on the surface, wherein the binding complex comprises the binding reagent and the ligated target complement; and (f) detecting the binding complex, thereby detecting the SNP at the polymorphic site.

In embodiments, the invention provides a kit for detecting one or more antibody biomarkers of interest in a sample, the kit comprising, in one or more vials, containers, or compartments: (a) a surface comprising one or more binding domains, wherein each binding domain comprises an antigen immobilized thereon; and (b) one or more detection reagents, wherein each detection reagent comprises a detection antibody, a detection antigen, or an ACE detection reagent.

In embodiments, the invention provides a method of detecting one or more antibody biomarkers of interest in a sample, comprising: (a) contacting the sample with a surface comprising one or more binding domains, wherein each binding domain comprises an antigen immobilized thereon; (b) forming a binding complex in each binding domain, wherein the binding complex comprises the antigen and an antibody biomarker that binds to the antigen; (c) contacting the binding complex in each binding domain with a detection reagent; and (d) detecting the binding complexes on the surface, thereby detecting the one or more antibody biomarkers in the sample.

### BRIEF DESCRIPTION OF THE DRAWINGS

The following drawings form part of the present specification and are included to further demonstrate exemplary embodiments of certain aspects of the present invention.

FIG. 1 relates to Example 1. FIG. 1 shows the results of an embodiment of a bridging serology assay described herein. SARS-CoV-2 S-RBD was immobilized as binding reagent, and labeled S-RBD was used as detection reagent. The bridging serology assay was tested on serum samples from COVID-19 positive (red circles) and normal (non-COVID-19) (blue circles) patients, diluted 10-fold or 100-fold. Higher signal indicates increased number of antibodies bound to the immobilized antigen.

FIG. 2 relates to Example 2. FIG. 2 shows the results of an embodiment of a neutralization serology assay described herein. SARS-CoV-2 S protein was immobilized as binding reagent, and labeled ACE2 was added as a competitor to SARS-CoV-2 antibodies that may be present. The neutralization serology assay was tested on serum samples from COVID-19 positive (red circles) and normal (non-COVID-19) (blue circles) patients, diluted 10-fold or 100-fold. Lower signal (generated by competitor) indicates increased number of antibodies bound to the immobilized antigen.

FIGS. 3A-3D illustrate an embodiment of the methods described herein for detecting a single nucleotide polymorphism (SNP) in a viral nucleic acid. In FIGS. 9A-9C, a target nucleic acid (1) that comprises an SNP (2) is contacted with: a targeting probe (3) that comprises an oligonucleotide tag (4) and a sequence that is complementary to the SNP, and a detection probe (5) that comprises detectable label (6). The targeting and detection probes (3, 5) hybridize to the target nucleic acid, and the targeting and detection probes that hybridize with perfect complementarity at the SNP are ligated to form a ligated target complement (11) comprising the oligonucleotide tag and detectable label. The reaction mixture containing the ligated target complement is contacted with a surface comprising one or more binding reagents (7) immobilized in one or more binding domains (9). A signal (10) is detected if the ligated target complement is immobilized on the surface via hybridization of the complementary oligonucleotides in the oligonucleotide tag and the binding reagent. In FIG. 3D, the targeting probe has a mismatch with the SNP in the target nucleic acid, and thus, hybridization and ligation do not occur.

FIG. 4 illustrates an embodiment of the methods described herein for detecting a viral nucleic acid. RNA is extracted from a sample containing an RNA virus (e.g., SARS-CoV-2), and the extracted RNA is converted to cDNA. A “Master Mix” is prepared by combining a forward primer comprising a 5′ binding reagent complement sequence and a cDNA complement sequence, a reverse primer comprising a cDNA reverse complement sequence and a 3′ binding partner of a detectable label, and other PCR components such as dNTPs and DNA polymerase. The cDNA and Master Mix are combined, and PCR is performed for 30 to 40 cycles to form a plurality of PCR products, each PCR product comprising the 5′ binding reagent complement sequence and 3′ binding partner of a detectable label. Each PCR product hybridizes to a binding reagent on a surface. The surface is then contacted with a detectable label, which binds to the PCR product. The PCR product bound to the detectable label is then subjected to detection as described herein.

FIGS. 5, 6A, and 6B relate to Example 4. FIG. 5 shows the correlation between embodiments of serology assays described herein.

FIG. 6A shows the correlation results of the indirect serology assays for IgG against SARS-CoV-2 S with four other serology assays: IgG against SARS-CoV-2 N, IgG against SARS-CoV-2 S-RBD, IgM against SARS-CoV-2 S, and ACE2 competitor assay. FIG. 6B shows the assay performance (sensitivity and specificity) for the assay pairings of FIG. 6A.

FIG. 7 relates to Example 5. FIG. 7 shows the assay performance (sensitivity at early and late infections and specificity) of IgG indirect serology assay and IgM indirect serology assay and ACE2 competitor assay.

FIGS. 8-10 relate to Example 6. FIG. 8 shows the results from an exemplary oligonucleotide ligation assay (OLA) for detection of SARS-CoV-2 single nucleotide polymorphisms (SNPs) at genome locations 8782, 11083, 23403, and 28144, with a synthetic template oligonucleotide.

FIG. 9 shows the results of an exemplary singleplex OLA assay for detecting SARS-CoV-2 SNPs at genome locations 8782, 11083, 23403, and 28144, with samples obtained from SARS-CoV-2 positive patients.

FIG. 10 shows the results of an exemplary multiplex OLA assay for detecting SARS-CoV-2 SNPs at genome locations 8782, 11083, 23403, and 28144, with samples obtained from SARS-CoV-2 positive patients.

FIGS. 11-13 relate to Example 7. FIG. 11 shows the results of an exemplary assay for measuring the concentration (fg/mL) of SARS-CoV-2 nucleocapsid (N) protein from the following samples: nasopharyngeal swabs from 12 patients who tested positive for COVID-19, nasopharyngeal swabs from 6 patients who tested negative for COVID-19, and normal (COVID-19 negative) human saliva, serum, and EDTA plasma.

FIG. 12 shows the percent recovery results of an exemplary test to assess dilution linearity of the SARS-CoV-2 N protein detection assay. The normal human serum, EDTA plasma, saliva, and COVID-19 negative human nasopharyngeal swab samples were spiked with calibrator and tested at different dilutions.

FIG. 13 shows the percent recovery results of an exemplary test to assess spike recovery of the SARS-CoV-2 N protein detection assay. The normal human serum, EDTA plasma, saliva, and COVID-19 negative human nasopharyngeal swab samples were spiked with calibrator at three levels.

FIG. 14 shows the results of an exemplary serology assay performed on samples obtained from SARS-CoV-2-infected individuals in the United States during early 2020 (known to be infected with wild-type SARS-CoV-2 (“Wuhan”)); SARS-CoV-2-infected individuals in the United Kingdom (dominating strain: SARS-CoV-2 strain B.1.1.7); or SARS-CoV-2-infected individuals in South Africa (dominating strain: SARS-CoV-2 strain 501Y.V2, also known as B.1.351). The measured ratios of antibodies against wild-type SARS-CoV-2 versus SARS-CoV-2 strain B.1.1.7 were plotted on the x-axis, and the measured ratios of antibodies against wild-type SARS-CoV-2 versus SARS-CoV-2 strain 501Y.V2 were plotted on the y-axis.

FIG. 15 shows the results of an exemplary serology assay to determine antibody concentrations for endemic coronaviruses in finger-stick blood, saliva, and serum in samples from subjects as described in Table 17.

FIG. 16 shows the results of an exemplary serology assay to determine reactivity to SARS-CoV-2 antigens in finger-stick blood and saliva samples from subjects described in Table 17. Dashed lines indicate assay sensitivity and quantitation (LLOD=lower limit of detection; LLOQ=lower limit of quantitation; ULOQ=upper limit of quantitation). The dotted line labeled “98%” is drawn at the threshold set at the 98^{th} percentile for the presumed naïve (PN) donors. Filled circles indicates donors whose IgG levels in finger-stick blood exceeded the threshold for SARS-CoV-2 spike.

FIG. 17 shows the results of an exemplary serology assay to determine total immunoglobulin concentrations in finger-stick blood samples from subjects described in Table 17.

FIG. 18 shows the results of an exemplary serology to determine total immunoglobulin concentrations in saliva samples from subjects described in Table 17.

FIG. 19 shows the results of an exemplary serology assay to determine correlation in reactivity to SARS-CoV-2 antigens measured in self-collected saliva versus finger-stick blood from subjects described in Table 17.

FIG. 20 shows the results of an exemplary serology assay to determine correlation in salivary IgG levels for CoV-2 spike, RBD, and N antigens in samples from subjects described in Table 17. Dashed lines indicate the selected classification thresholds set at the 98^{th} percentile of saliva from subjects who reported no COVID-19 diagnosis, recent symptoms, or household exposure to COVID-19.

FIG. 21 shows the relative reactivity to the SARS-CoV-2 nucleocapsid (N) and spike (S) antigens measured in finger-stick blood and saliva from subjects whose levels of anti-spike IgG exceeded the threshold as shown in FIG. 16. Spearman coefficient=0.95, p=0.001. The dashed line has slope of 1 and represents the expected correlation.

FIG. 22 shows the results of exemplary indirect IgG serology and ACE2 competition assays using a 10-spot SARS-CoV-2 S-RBD antigen panel. The graph shows the signals for each of the antigens in the S-RBD antigen panel (identified in the inset table) after normalization to the signal from the wild-type SARS-CoV-2 S-RBD antigen spot.

FIGS. 23 and 24 show heat map results of the data in FIG. 22. Each lower row shows the signal for one of the 10 S-RBD antigen spots after normalization across the column. Each column is one individual sample (˜200 samples infected with wild-type SARS-CoV-2 and 32 samples infected with strain B.1.351). FIG. 23 shows results from the ACE2 competition assay, and FIG. 24 shows results from the IgG indirect serology assay.

FIG. 25 shows a subset of the data in FIGS. 22-24, with signals from two spots in the 10-spot S-RBD antigen panel. Each dot represents one individual.

FIGS. 26A and 66B show the results of exemplary indirect IgG serology (FIG. 26A) and ACE2 competition (FIG. 26B) assays to detect anti-CoV-2 spike antibodies. Both assays were tested against a set of 214 serum samples collected from individuals at different time points after confirmed SARS-CoV-2 infection (diagnosis by PCR; 0-14 days, 15-28 days, 29-56 days, and 57+ days) and 200 control samples collected prior to the emergence of SARS-CoV-2 in 2020. Horizontal line A shows the optimal threshold for classification accuracy.

FIG. 27 shows the results of exemplary multiplexed oligonucleotide ligation assay (OLA) panel for detection of SARS-CoV-2 single nucleotide polymorphisms (SNPs) in the S protein: 69-70del, D215G, D253G, K417N, K417T, L452R, E484K, N501Y, D614G, and P681H. The top panel shows the results from a known SARS-CoV-2 wild-type or B.1.1.7 strain. The bottom panel shows the results from 23 nasal swab samples from March or August 2020.

FIG. 28 depicts an embodiment of a sample collection device in accordance with certain aspects of the disclosure.

FIGS. 29A and 29B depict an embodiment of a sample collection device in accordance with certain aspects of the disclosure.

FIGS. 30A and 30B depict an embodiment of a sample collection device having a first opening and a second opening, in accordance with certain aspects of the disclosure.

FIGS. 31A and 31B depict an embodiment of a sample collection device having a retention material, in accordance with certain aspects of the disclosure.

FIG. 32 depicts a sample collection device that has a container sealing component which includes a compartment for storing a stabilizer fluid, in accordance with certain aspects of the disclosure.

FIG. 33 depicts a sample collection device that has a container sealing component which includes a compartment for storing a solid phase binding material, in accordance with certain aspects of the disclosure.

FIG. 34 depicts a sample collection device that has a container sealing component which includes compartments for storing a stabilizer fluid and a solid phase binding material, in accordance with certain aspects of the disclosure.

FIGS. 35A-35B and 36A-36B depict sample collection devices having a funnel which facilitates delivery of samples into the sample collection devices, in accordance with certain aspects of the disclosure.

FIG. 37 depicts a sample collection device adapted to equalize air pressure between a first portion of a sample container and a second portion of the sample container.

FIG. 38 show the results of an exemplary biomarker assay to assess levels of IL-6, IL-10, IL-12p70, IL-4, TNF-α, IL-2, IL-1β, IFN-γ, and IL-17A, performed on cerebrospinal fluid (CSF) and serum samples from acute COVID-19 patients and non-COVID-19 control subjects.

FIGS. 39A and 39B illustrate exemplary assay surfaces described in embodiments herein. FIG. 39A shows a well of an exemplary 384-well assay plate, comprising four distinct binding domains (“spots”). FIG. 39B shows a well of an exemplary 96-well assay plate, comprising ten distinct binding domains (“spots”).

### DETAILED DESCRIPTION OF THE INVENTION

Certain inventions disclosed herein were made jointly under Research Collaboration Agreement 2020-0351 between the National Institute of Allergy and Infectious Diseases (NIAID), which is a component of the National Institutes of Health (NIH), which is an agency of the U.S. Department of Health and Human Services, and Meso Scale Diagnostics, LLC., which is an affiliate of Meso Scale Technologies, LLC.

The disclosed embodiments fulfill the urgent need for high-quality viral assays and methods useful for the COVID-19 pandemic. Disclosed embodiments have been widely adopted for COVID-19 research, epidemiology, and vaccine development and have had a significant impact on the COVID-19 public health response. For example, serology embodiments are widely used (e.g., Johnson M et al. J Clin Virol 2020; 130:104572; Corbett K S et al. N Engl J Med 2020; 383:1544-55; Folegatti P M et al. The Lancet 2020; 396:467-78; Ramasamy M N et al. The Lancet 2020; 396:1979-93; Goldblatt D et al. J Hosp Infect 2021; 110:60-6; Majdoubi A et al. JCI Insight 2021, doi.org/10.1172/jci.insight.146316; Amjadi M F et al. MedRxiv 2021:2021.01.05.21249240, doi.org/10.1101/2021.01.05.21249240; Grandjean L et al. MedRxiv 2020:2020.07.16.20155663, doi.org/10.1101/2020.07.16.20155663; Majdoubi A et al. MedRxiv 2020:2020.10.05.20206664, doi.org/10.1101/2020.10.05.20206664). Certain embodiments disclosed herein were chosen by the United States government initiative, Operation Warp Speed, as the basis of its standard binding assay for immunogenicity assessments in all funded Phase III clinical trials of vaccines. Serology assay embodiments (e.g., assays to detect immunoglobulin(s) conducted on non-bodily samples or bodily samples (e.g., serum, plasma, saliva)) disclosed herein aid in assessing human immune responses to COVID-19 infection and vaccination and in understanding the interplay between COVID-19 and immunity to other coronaviruses and respiratory pathogens. The disclosed nucleic acid detection embodiments have advantages over PCR methods, e.g., in their speed, simplicity, cost, and high throughput. The disclosed intact virus detection embodiments provide improved accuracy and specificity of an active infection diagnosis as compared to detection of an individual viral component. Serology assays, nucleic acid detection assays, and other embodiments related to mutations and variants of SARS-CoV-2 are proving important as new mutations and variants arise. Other biomarker detection embodiments disclosed herein, e.g., detection of inflammatory and/or tissue damage response biomarkers and/or extracellular vesicles, e.g., from virus-infected cells, have wide applicability, regardless of viral mutation status, to studies on morbidity and mortality to understand factors underlying severe illness, death, and persistent symptoms following acute infection and may lead to better interventions. Data showing the high-quality nature of the disclosed embodiments are described in the Examples and elsewhere herein.

Immunoassays described herein for the detection of respiratory viruses, including coronaviruses, provide numerous advantages compared with nucleic acid amplification (e.g., PCR) based detection methods. For example, immunoassays are conducted in a simple and streamlined format with improved sensitivity. Improved sensitivity with immunoassays occurs because these assays not only detect viral particles, but also individual viral proteins in damaged tissue being cleared by the body at the site of infections. Moreover, immunoassays for biomarkers produced by the body in response to infection (e.g., antibodies against the virus or inflammatory factors associated with the host response to infection) take advantage of the natural amplification associated with the immune response.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the invention that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

### Respiratory Virus Detection

In embodiments, the invention provides an immunoassay method for detecting at least one respiratory virus, including a coronavirus, in a biological sample. As used herein, a “respiratory virus” refers to a virus that can cause a respiratory tract infection, e.g., in a human. Exemplary respiratory viruses include, but are not limited to, coronavirus, influenza virus, respiratory syncytial virus (RSV), paramyxovirus, adenovirus, parainfluenza virus (PIV), bocavirus, metapneumovirus, orthopneumovirus, enterovirus, rhinovirus, and the like. Respiratory virus infections can be difficult to diagnose because different viruses can often cause similar symptoms in a patient. For example, coughing and low-grade fever are typical symptoms of early disease progression or mild cases of a coronavirus infection (e.g., COVID-19), as well as influenza or a respiratory syncytial virus (RSV) infection. An assay that can simultaneously test for several potential causes of infection would advantageously allow a respiratory virus infection to be correctly and efficiently diagnosed in a single assay run and utilizing a single patient sample. In embodiments, the methods herein distinguish between and among different types of a given virus (e.g., distinguishing PIV-1, PIV-2, PIV-3, and PIV-4 from each other or influenza A from influenza B from each other), as well as between and among different subtypes or strains (e.g., distinguishing influenza A (H1N1) from influenza A (H3N2)).

In embodiments, the invention provides an immunoassay method for detecting at least one respiratory virus in a biological sample, comprising: (a) contacting the biological sample with a binding reagent that specifically binds a component of at least one respiratory virus in the biological sample; (b) forming a binding complex comprising the binding reagent and the respiratory virus component; and (c) detecting the binding complex, thereby detecting the at least one respiratory virus in the biological sample.

In embodiments, the at least one respiratory virus comprises a coronavirus, an influenza virus, a paramyxovirus, an adenovirus, a bocavirus, a pneumovirus, an enterovirus, a rhinovirus, or a combination thereof. Exemplary coronaviruses and methods for their detection are described herein and include, but are not limited to, SARS-CoV (also known as SARS-CoV-1), MERS-CoV, SARS-CoV-2, HCoV-OC43, HcoV-229E, HcoV-NL63, HcoV-HKU1. In embodiments, the method detects a coronavirus by detecting a coronavirus nonstructural protein, e.g., nsp1, nsp2, nsp3, nsp4, nsp5, nsp6, nsp7, nsp8, nsp9, nsp10, nsp11, nsp12, nsp13, nsp14, nsp15, or nsp16. In embodiments, the method detects a coronavirus by detecting a coronavirus structural protein, e.g., the E, S (including S1, S2, S-NTD, S-ECD, and S-RBD), M, HE, or N proteins. Coronaviruses and their proteins are further described herein.

Exemplary influenza viruses include, but are not limited to, influenza A (FluA), influenza B (FluB), and influenza C (FluC). Typically, the seasonal flu is caused by FluA and/or FluB. FluA viruses can be further characterized into various subtypes based on the hemagglutinin (HA) and neuraminidase (N) proteins present on the surface of the viral particle, e.g., H1N1, H1N2, H2N2, H3N2, H5N1, H7N2, H7N3, H7N7, H9N2, and H10N7. FluA strains include, e.g., H1/Michigan strain, H3/Hong Kong strain, H7/Shanghai strain, and the like. FluB viruses can be further characterized into genetic lineages, e.g., the FluB (Victoria) or FluB (Yamagata) viruses. In embodiments, the immunoassay detects an influenza virus component, e.g., an influenza virus-specific protein. In embodiments, the immunoassay detects an influenza structural protein. In embodiments, the immunoassay detects an influenza nonstructural protein. In embodiments, the immunoassay detects an influenza virus by detecting the influenza HA protein. In embodiments, the immunoassay detects an influenza virus by detecting the influenza N protein. In embodiments, the immunoassay detects an influenza virus by detecting an influenza nucleoprotein (NP). In embodiments, the immunoassay detects a FluA virus and is further capable of determining the subtype of the FluA virus. In embodiments, the immunoassay detects a FluB virus and is further capable of determining the lineage of the FluB virus.

Exemplary paramyxoviruses include, but are not limited to, parainfluenza virus type 1, parainfluenza virus type 2, parainfluenza virus type 3, and parainfluenza virus type 4. In embodiments, the immunoassay detects a paramyxovirus component, e.g., a paramyxovirus-specific protein. In embodiments, the immunoassay detects a paramyxovirus structural protein. In embodiments, the immunoassay detects a paramyxovirus nonstructural protein. Non-limiting examples of paramyxovirus proteins that can be detected by the immunoassay include a nucleocapsid (N) protein, transcriptase (L), phosphoprotein (P), fusion protein (F), hemagglutinin-neuraminidase (HN) or hemagglutinin (H), and non-glycosylated membrane protein (M).

Adenoviruses that can cause respiratory infections include, but are not limited to, adenovirus type 3, type 4, and type 7. In embodiments, the immunoassay detects an adenovirus component, e.g., an adenovirus-specific protein. In embodiments, the immunoassay detects an adenovirus structural protein. In embodiments, the immunoassay detects an adenovirus nonstructural protein. Non-limiting examples of adenovirus proteins that can be detected by the immunoassay include a capsid protein, encapsidation protein, L3 protease, E1A, E1B, E2A, E2B, E3, and E4.

Exemplary bocaviruses include, but are not limited to, HBoV1, HBoV2, HBoV3, and HBoV4. In embodiments, the immunoassay detects a bocavirus component, e.g., a bocavirus-specific protein. In embodiments, the immunoassay detects a bocavirus structural protein. In embodiments, the immunoassay detects a bocavirus nonstructural protein. Non-limiting examples of bocavirus proteins that can be detected by the immunoassay include NS1, NS2, NS3, NS4, VP1, VP2, and VP3.

Exemplary pneumoviruses include, but are not limited to, respiratory syncytial virus (RSV), including human respiratory syncytial virus B1 (HRSV-B1) and human respiratory syncytial virus A2 (HRSV-A2). In embodiments, the immunoassay detects a pneumovirus component, e.g., a pneumovirus-specific protein. In embodiments, the immunoassay detects a pneumovirus structural protein. In embodiments, the immunoassay detects a pneumovirus nonstructural protein. Non-limiting examples of pneumovirus proteins that can be detected by the immunoassay include fusion (F), attachment (G), lipoprotein (SH), nucleoprotein (N), phosphoprotein (P), membrane protein (M), and large protein (L).

Exemplary enterovirus include, but are not limited to, EV-A, EV-B, EV-C, including EV-C104, EV-C105, EV-C109, EV-C117, EV-C118, and EV-D, including EV-D68. In embodiments, the immunoassay detects an enterovirus component, e.g., an enterovirus-specific protein. In embodiments, the immunoassay detects an enterovirus structural protein. In embodiments, the immunoassay detects an enterovirus nonstructural protein. Non-limiting examples of enterovirus proteins that can be detected by the immunoassay include the capsid proteins VP1, VP2, VP3, and VP4, nonstructural proteins 2A, 2B, 2C, 3A, 3B, 3C, and 3D, and VPg.

Exemplary rhinoviruses include, but are not limited to, RV-A, RV-B, and RV-C. In embodiments, the immunoassay detects a rhinovirus component, e.g., a rhinovirus-specific protein. In embodiments, the immunoassay detects a rhinovirus structural protein. In embodiments, the immunoassay detects a rhinovirus nonstructural protein. Non-limiting examples of rhinovirus proteins that can be detected by the immunoassay include the capsid proteins VP1, VP2, VP3, and VP4, nonstructural proteins 2A, 2B, 2C, 3A, 3B, 3C, and 3D, and VPg.

In embodiments, the method detects SARS-CoV, MERS-CoV, SARS-CoV-2, HcoV-OC43, HcoV-229E, HcoV-NL63, HcoV-HKU1, influenza A, influenza B, RSV, or a combination thereof. In embodiments, the method is a multiplexed method capable of simultaneously detecting one or more of SARS-CoV, MERS-CoV, SARS-CoV-2, HcoV-OC43, HcoV-229E, HcoV-NL63, HcoV-HKU1, influenza A, influenza B, and RSV. In embodiments, the method further comprises repeating one or more of the method steps described herein to detect one or more respiratory viruses in the sample. In embodiments, the method further comprises repeating steps (a)-(c) of the method described herein, wherein each detected respiratory virus comprises a component that binds to a different binding reagent, thereby detecting the at least one respiratory virus. In embodiments, each of steps (a)-(c) is performed for each respiratory virus in parallel.

As used herein, the term “simultaneous” in reference to one or more events (e.g., detection of one or more viruses, viral components, or biomarkers as described herein) means that the events occur at exactly the same time or at substantially the same time, e.g., simultaneous events described herein can occur less than or about 30 minutes apart, less than or about 20 minutes apart, less than or about 15 minutes apart, less than or about 10 minutes apart, less than or about 5 minutes apart, less than or about 2 minutes apart, less than or about 1 minute apart, or less than or about 30 seconds apart. In the context of embodiments of multiplexed immunoassays provided herein, “simultaneous” refers to detecting a on single surface (e.g., a particle, an assay plate, an assay cartridge, or a well of a multi-well assay plate) the presence of one or more viruses, viral components or biomarkers described herein. In embodiments, a multiplexed assay is performed on a single assay plate. In embodiments, a multiplexed assay is performed in a single well of an assay plate. In embodiments, a multiplexed assay is performed in a single assay cartridge. In embodiments, a multiplexed immunoassay is performed on more than one assay plates. In embodiments, more than one multiplexed immunoassay (e.g., wherein each multiplexed immunoassay detects a combination of biomarkers and/or viral components as described herein) is performed on a single surface, e.g., a single well of an assay plate or a single assay cartridge. The number of assay wells and/or assay plates that may be required to perform a multiplexed assay can be determined, e.g., based on the number of substances of interest to be detected in one or more samples (e.g., a multiplex of about 2 to about 100, or about 2 to about 90, or about 2 to about 80, or about 2 to about 70, or about 2 to about 60, or about 2 to about 50, or about 2 to about 40, or about 2 to about 35, about 2 to about 30, or 2, 3, 4, 5, 6, 7, 8, 9, 10, 11, 12, 13, 14, 15, 16, 17, 18, 19, 20, 21, 22, 23, 24, 25, 26, 27, 28, 29, 30, or more viruses, viral components, and/or biomarkers described herein); the number of samples being assayed (e.g., from one or more subjects); the number of calibration reagents being measured to generate a calibration curve (e.g., 3, 4, 5, 6, 7, 8, 9, 10, 11, 12, or more); the number of control reagents being measured (e.g., 0, 1, 2, 3, or more); the number of replicates for each sample, calibration reagent, and/or control reagent being measured (e.g., singlicate, duplicate, triplicate, or more); and the number of wells per assay plate (e.g., 6, 12, 48, 96, 384, or 1536 wells per assay plate). When multiplexed immunoassay is conducted on multiple assay plates, the assay plates can be read simultaneously or at different times. The timing of reading the assay plates can be determined, e.g., based on the capacity of the assay reader instrument (e.g., capable of reading 1, 2, 3, 4, or more plates at once); the read-time of the assay reader instrument (e.g., about 1 s to about 600 s, about 10 s to about 500 s, about 20 s to about 300 s, about 30 s to about 180 s, about 60 s to about 120 s, about 70 s, or about 90 s per assay plate); the time required to prepare the assay components (e.g., about 10 s, 20 s, 30 s, 1 min, 2 min, 5 min, 10 min, 15 min, 30 min, 1 hr, or more per plate); and the equipment for performing the assay (e.g., a single-channel pipettor may require a longer time for pipetting the assay components as compared to a multi-channel pipettor; handling liquids from different containers, e.g., tubes, vials, or plates, may require different lengths of time). In embodiments, “simultaneous” refers to events occurring with respect to a single sample (e.g., a biological sample in a single vial or container from a single subject) or replicates or dilutions of a single sample. Factors affecting the timing of simultaneous events include the following: the number of multiplexed assays being performed at the same time on a single sample (e.g., a multiplex of or about 2 to about 100, or about 2 to about 90, or about 2 to about 80, or about 2 to about 70, or about 2 to about 60, or about 2 to about 50, or about 2 to about 40, or about 2 to about 35, about 2 to about 30, or 2, 3, 4, 5, 6, 7, 8, 9, 10, 11, 12, 13, 14, 15, 16, 17, 18, 19, 20, 21, 22, 23, 24, 25, 26, 27, 28, 29, 30, or more assays in a single well or cartridge); the number of assay modules in a panel (e.g., 1, 2, 3, or more plates or cartridges in a panel); the number of samples being assayed at the same time (e.g., a number of samples capable of being assayed in one kit or more than one kit); the number of points on a calibration curve (e.g., 5, 6, 7, 8, 9, 10, 12, or more); the presence and number of controls (e.g., 0, 1, 2, 3, or more controls); the read-time of the instrument (e.g., about 1 s to about 600 s, about 10 s to about 500 s, about 20 s to about 300 s, about 30 s to about 180 s, about 60 s to about 120 s, about 70 s, or about 90 s); the number of replicates of each calibrator, control, or sample (e.g., singlicate, duplicate, triplicate, or more); the number of wells per plate (e.g., 6, 12, 48, 96, 384, or 1536 wells per plate); and/or the type of equipment for performing the assay (e.g., a single channel or a multi channel pipettor, tubes or plates for dilution).

In embodiments, the binding reagent that specifically binds to the respiratory virus component described herein is an antibody, antigen, ligand, receptor, oligonucleotide, hapten, epitope, mimotope, or aptamer. In embodiments, the binding reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent comprises at least one heavy or light chain complementarity determining region (CDR) of an antibody. In embodiments, the binding reagent comprises at least two CDRs from one or more antibodies. In embodiments, the binding reagent is an antibody or antigen-binding fragment thereof. In embodiments, the binding reagent is a receptor for the respiratory virus component. In embodiments, the binding reagent is a binding partner of the respiratory virus component. In embodiments, the binding reagent is angiotensin-converting enzyme 2 (ACE2). In embodiments, the binding reagent is a neuropilin (NRP) receptor. In embodiments, the binding reagent is NRP1. In embodiments, the binding reagent is NRP2.

### Coronavirus Detection

Coronaviruses, which belong to the Coronaviridae family of viruses, are enveloped viruses with a positive-sense single-stranded RNA genome and a nucleocapsid of helical geometry. A characteristic feature of coronaviruses is the club-shaped spikes that project from the virus surface. In general, a coronavirus particle is assembled from its structural proteins, including an envelope (E), a spike glycoprotein (S), which includes S1 and S2 subunits that form the ectodomain (S-ECD), a viral membrane protein (M), a hemagglutinin-esterase dimer (HE), nucleocapsid (N), and RNA. The S protein comprises a N-terminal domain (N-Term or NTD). The S1 subunit comprises a receptor binding domain (S-RBD), which binds a host receptor (e.g., ACE2) during infection. The S1 subunit can also bind to the cell surface neuropilin-1 (NRP1) receptor. See, e.g., Daly et al., bioRxiv 2020.06.05. 134114 (2020) doi:10.1101/2020.06.05.134114. In embodiments, coronavirus S proteins, including recombinantly expressed S proteins and variants thereof, are further described, e.g., in WO 2018/081318. For example, two variants of SARS-CoV-2 each has a single polynucleotide morphism (SNP) at genome location 23403, which is in the gene encoding the S protein, resulting in a different amino acid at position 614 of the S protein: D614 and G614 (denoted as S: 23403A>G, D614G; see, e.g., Korber et al., bioRxiv 2020.04.29. 069054 (2020) doi:10.1101/2020.04.29.069054; also published as Korber et al., Cell 182(4):P812-827 (2020)), referred to herein respectively as S-D614 and S-D614G. Further mutations of the SARS-CoV-2 S protein are described in Tables 1A and 1B. Sequence alignments between the genetic material of various coronavirus species have also revealed additional conserved open reading frames for Coronaviruses also encode a number of nonstructural proteins (NSPs), which are expressed in infected cells but are generally not incorporated into the viral particle itself. Exemplary coronavirus NSPs include, but are not limited to, nsp1, nsp2, nsp3, nsp4, nsp5, nsp6, nsp7, nsp8, nsp9 (replicase), nsp10, nsp1 1, nsp12 (multi-domain RNA polymerase), nsp13 (helicase, RNA 5′ triphosphatase), nsp14 (N7-methyl transferase, exonuclease), nsp15 (endoribonuclease), nsp16 (2′-O-methyl transferase), and the like. See, e.g., Snijder et al., Adv Virus Res 96:59-126 (2016); Fehr et al., Coronaviruses 1281:1-23 (2015). Sequence alignments between the genetic material of various coronavirus species have revealed conserved open reading frames for several structural and nonstructural proteins, e.g., N, M, S, nsp1, nsp3, nsp6, nsp7, and nsp8. See, e.g., Grifoni et al., bioRxiv 2020.02.12.946087 (2020) doi:10.1101/2020.02.12.

While assays for a specific coronavirus species can identify infection by that particular coronavirus, such assays may have limited usefulness when new strains of infectious coronaviruses emerge. In embodiments, the invention provides a method for detecting a coronavirus in a sample by detecting a conserved coronavirus component, e.g., a protein that is generally conserved across all coronavirus species. Such a method would enable detection of novel coronaviruses of interest.

In embodiments, the invention provides an immunoassay method for detecting a coronavirus in a biological sample, comprising: a) contacting the biological sample with a binding reagent that specifically binds a component of the coronavirus; b) forming a binding complex comprising the binding reagent and the coronavirus component; and c) detecting the binding complex, thereby detecting the coronavirus in the biological sample. In embodiments, the method detects SARS-CoV, MERS-CoV, SARS-CoV-2, HcoV-OC43, HcoV-229E, HcoV-NL63, HcoV-HKU1, or a combination thereof. In embodiments, the biological sample is saliva.

In embodiments, the coronavirus component is on the outer surface of the viral particle. In embodiments, the coronavirus component is integrated in the membrane of the viral particle. In embodiments, the coronavirus component is a protein. In embodiments, the coronavirus component comprises a sugar, e.g., a glycoprotein. In embodiments, the coronavirus component is a structural protein. In embodiments, the coronavirus component is an envelope (E) protein. In embodiments, the coronavirus component is a spike glycoprotein (S) or a variant or subunit thereof, e.g., S-D614, S-D614G, or any of the S protein variants in Tables 1A and 1B, subunit 1 (S1), subunit 2 (S2), ectodomain (S-ECD), N-terminal domain (S-NTD or S—N-Term), or receptor binding domain (S-RBD). In embodiments, the S protein subunit (e.g., S1, S2, S-ECD, S-NTD, or S-RBD) comprises a mutation as described in Tables 1A and 1B. In embodiments, the coronavirus component is a viral membrane (M) protein. In embodiments, the coronavirus component is a hemagglutinin-esterase dimer (HE). In embodiments, the coronavirus component is a nucleocapsid (N) protein. In embodiments, the coronavirus component comprises a mutation as described in Table 1A.

In embodiments, the coronavirus component is a non-structural protein. In embodiments, the coronavirus component is nsp1, nsp2, nsp3, nsp4, nsp5, nsp6, nsp7, nsp8, nsp9, nsp10, nsp11, nsp12, nsp13, nsp14, nsp15, or nsp16. In embodiments, the coronavirus component is a protein substantially conserved across coronaviruses. It will be understood by one of ordinary skill in the art that a protein that is “substantially conserved” across a viral family, e.g., the coronavirus family, means that at least 50%, at least 60%, at least 70%, at least 80%, at least 90%, or at least 95% of species in the viral family contains a protein with at least 50%, at least 60%, at least 70%, at least 80%, at least 90%, or at least 95% sequence similarity, structural similarity, or both. Methods and tools for determining sequence and/or structural similarity are known in the field and include, e.g., algorithms such as Align, BLAST, and CLUSTAL for sequence similarity, and TM-align, DALI, STRUCTAL, and MINRMS.

In embodiments, the immunoassay method detects a coronavirus by detecting the coronavirus E protein. In embodiments, the immunoassay method detects a coronavirus by detecting the coronavirus S protein. In embodiments, the immunoassay method detects a coronavirus by detecting the coronavirus S1 protein subunit. In embodiments, the immunoassay method detects a coronavirus by detecting the coronavirus S2 protein subunit. In embodiments, the immunoassay method detects a coronavirus by detecting the coronavirus S-ECD. In embodiments, the immunoassay method detects a coronavirus by detecting the coronavirus S-RBD. In embodiments, the immunoassay method detects a coronavirus by detecting the coronavirus S-NTD. In embodiments, the immunoassay method detects a coronavirus by detecting the coronavirus M protein. In embodiments, the immunoassay method detects a coronavirus by detecting the coronavirus HE protein. In embodiments, the immunoassay method detects a coronavirus by detecting the coronavirus N protein. In embodiments, the immunoassay method detects a coronavirus by detecting one or more of the coronavirus nsp1, nsp2, nsp3, nsp4, nsp5, nsp6, nsp7, nsp8, nsp9, nsp10, nsp11, nsp12, nsp13, nsp14, nsp15, or nsp16. In embodiments, the immunoassay detects a coronavirus by detecting a combination of the coronavirus proteins described herein. In embodiments, the coronavirus is SARS-CoV-2. In embodiments, the immunoassay method detects SARS-CoV-2 by detecting SARS-CoV-2 N protein. In embodiments, the immunoassay method detects SARS-CoV-2 by detecting SARS-CoV-2 S protein. In embodiments, the immunoassay method detects SARS-CoV-2 by detecting SARS-CoV-2 S-D614. In embodiments, the immunoassay method detects SARS-CoV-2 by detecting SARS-CoV-2 S-D614G. In embodiments, the immunoassay method detects SARS-CoV-2 by detecting any of the SARS-CoV-2 S protein variants in Tables 1A and 1B. In embodiments, the immunoassay method detects SARS-CoV-2 by detecting SARS-CoV-2 E protein. In embodiments, the immunoassay method detects SARS-CoV-2 by detecting SARS-CoV-2 M protein. In embodiments, the immunoassay detects SARS-CoV-2 by detecting SARS-CoV-2 N protein and S protein. In embodiments, the immunoassay method detects SARS-CoV-2 by detecting SARS-CoV-2 S protein, N protein, E protein, and M protein. SARS-CoV-2 nonstructural proteins include the Orf1a and Orf1ab replicase/transcriptase proteins; the Orf3a protein; the Orf6a protein; the Orf7a and Orf7b accessory proteins; the Orf8 protein monomer, which is known to form oligomers; and the Orf10 protein. SARS-CoV-2 nonstructural proteins are further described in, e.g., Khailany et al., Gene Rep 19:100682 (2020); and Flower et al., Proc Nat Acad Sci 118(2): e2021785118 (2021). In embodiments, the immunoassay detects SARS-CoV-2 by detecting any of SARS-CoV-2 Orf1a, Orf1ab, Orf3a, Orf6a, Orf7a, Orf7b, Orf8 monomer, Orf8 oligomer, Orf10, RNA-dependent RNA polymerase (RdRp), or a combination thereof. In embodiments, the immunoassay method detects SARS-CoV-2 by detecting any of the SARS-CoV-2 protein variants in Table 1A.

In embodiments, the immunoassay method for detecting SARS-CoV-2 comprises: a) contacting the biological sample with a binding reagent that specifically binds a SARS-CoV-2 S, N, E, or M protein; b) forming a binding complex comprising the binding reagent and the SARS-CoV-2 S, N, E, or M protein; and c) detecting the binding complex, thereby detecting SARS-CoV-2 in the biological sample. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises any of the mutations shown in Tables 1A and 1B. In embodiments, the SARS-CoV-2 N protein comprises any of the mutations shown in Table 1A. In embodiments, the SARS-CoV-2 E protein comprises any of the mutations shown in Table 1A. In embodiments, the binding complex further comprises a detection reagent that specifically binds to the SARS-CoV-2 S, N, E, or M protein. In embodiments, the detection reagent comprises a detectable label. In embodiments, the detection reagent comprises a nucleic acid probe. Detection reagents are further described herein. In embodiments, the biological sample is saliva.

In humans, coronaviruses can cause respiratory tract infections ranging from mild to lethal. Infection by the coronaviruses SARS-CoV, MERS-CoV, and SARS-CoV-2 can cause severe respiratory illness symptoms, i.e., severe acute respiratory syndrome (SARS), Middle East respiratory syndrome (MERS), or coronavirus disease 2019 (COVID-19), respectively. Infection by the coronaviruses HcoV-OC43, HcoV-229E, HcoV-NL63, or HcoV-HKU1 can lead to mild respiratory illness symptoms, e.g., the common cold. Coronaviruses can also cause disease in animals such as cats, birds, chickens, cows, and pigs. As used herein, “respiratory tract infection” or “respiratory infection” can refer to an upper respiratory tract infection (URI or URTI) or a lower respiratory tract infection (LRI or LRTI). URTIs include infection of the nose, sinuses, pharynx, and larynx, e.g., tonsillitis, pharyngitis, laryngitis, sinusitis, otitis media, and the common cold. LRTIs include infection of the trachea, bronchial tubes, bronchioles, and the lungs, e.g., bronchitis and pneumonia. Symptoms of illnesses caused by coronaviruses include, e.g., fever, cough, shortness of breath, fatigue, congestion, chills, muscle pain, headache, sore throat, loss of taste or smell, diarrhea, etc.

In embodiments, the coronavirus component is a fragment of any of the proteins described herein, e.g., a structural or non-structural coronavirus protein. In embodiments, the fragment comprises a domain of the full length protein. For example, the S protein includes an N-terminal domain (S-NTD) and an ectodomain (S-ECD), which includes the spike S1 and S2 subunits. The S1 subunit also includes a receptor binding domain (S-RBD), which is responsible for binding the host receptor (e.g., ACE2 and/or NRP1). In some embodiments, the immunoassay detects a coronavirus by detecting the coronavirus S1 subunit. In some embodiments, the immunoassay detects a coronavirus by detecting the coronavirus S2 subunit. In some embodiments, the immunoassay method detects a coronavirus by detecting the coronavirus S-NTD. In some embodiments, the immunoassay method detects a coronavirus by detecting the coronavirus S-ECD. In some embodiments, the immunoassay method detects a coronavirus by detecting the coronavirus S-RBD. In embodiments, the S protein subunit (e.g., S1, S2, S-ECD, S-NTD, or S-RBD) comprises a mutation as described in Tables 1A and 1B. In embodiments, the immunoassay detects a coronavirus by detecting a combination of the coronavirus proteins described herein. In embodiments, the coronavirus is SARS-CoV-2. In embodiments, the immunoassay method detects SARS-CoV-2 by detecting SARS-CoV-2 N protein. In embodiments, the immunoassay method detects SARS-CoV-2 by detecting SARS-CoV-2 S protein. In embodiments, the immunoassay method detects SARS-CoV-2 by detecting SARS-CoV-2 S-D614. In embodiments, the immunoassay method detects SARS-CoV-2 by detecting SARS-CoV-2 S-D614G. In embodiments, the immunoassay method detects SARS-CoV-2 by detecting any of the SARS-CoV-2 S protein variants in Tables 1A and 1B. In embodiments, the immunoassay detects SARS-CoV-2 by detecting SARS-CoV-2 N protein and S protein.

In embodiments, the coronavirus component is a nucleic acid. As used herein in the context of viral components, a viral nucleic acid refers to a viral genome or portion thereof. The viral nucleic acid can encode a viral protein, or the viral nucleic acid can be a non-coding sequence. In embodiments, detection of a viral nucleic acid comprises detecting a sequence that is present in the viral genome, but not in the host genome. In embodiments, the coronavirus component is DNA or RNA. In embodiments, the coronavirus component comprises a nucleic acid secondary structure, e.g., an RNA loop. In embodiments, the coronavirus component is a lipid, e.g., that forms part of the viral envelope.

In embodiments, the invention provides methods for distinguishing between strains of a coronavirus. In embodiments, the coronavirus is SARS-CoV-2. In embodiments, the invention provides methods for assessing the transmissibility of a COVID-19 infection outbreak by determining the SARS-CoV-2 strain. In embodiments, the invention provides methods for assessing the virulence of a SARS-CoV-2 strain by determining the SNPs in the strain. In embodiments, the invention provides methods for assessing effectiveness of a vaccine against a particular strain of SARS-CoV-2. The term “strain” is used interchangeably herein with “variant,” “lineage,” and “type.” In embodiments, a mutant strain or variant of a virus described herein, e.g., SARS-CoV-2, comprises one or more mutations relative to a reference or parent or wild-type strain of the virus. As referred to throughout this application, the SARS-CoV-2 NC_045512 strain is the “reference” or “wild-type” strain, and all SNPs described herein are attributed to one or more “mutant” strains or “variants.” In embodiments, the invention provides methods to trace the lineage of a coronavirus in a population. For example, two strains of SARS-CoV-2 have been identified, referred to as the “L” strain (also known as “lineage B”) and “S” strain (also known as “lineage A”). The L strain can be differentiated from the more ancestral S strain based on two different SNPs that show nearly complete linkage: one at location 8782 (orflab: T8517C, synonymous) and one at location 28144 (ORFS: C251T, S84L). See, e.g., Tang et al., Natl Sci Rev, nwaa036; doi:10.1093/nsr/nwaa036 (3 Mar. 2020). Moreover, as discussed herein, two SARS-CoV-2 strains have been identified to contain an SNP at genome location 23403, which encodes the S protein, and are referred to herein as the “S-D614” and “S-D614G” strains. A further SARS-CoV-2 SNP of interest is at location 11083, where the 11083G to T mutation (denoted as “11083G>T”) is associated with asymptomatic presentation. In embodiments, the SARS-CoV-2 reference strain comprises the “L strain” SNP at genome locations 8782 and 28144, the “S-D614” SNP at genome location 23403, and a G nucleotide at genome location 11083.

Mutations in the SARS-CoV-2 S protein can affect, e.g., binding to the ACE2 receptor, overall structure and antibody recognition, and/or protein conformation. Critical residues in the SARS-CoV-2 S-RBD for binding to the ACE2 receptor include, e.g., K417, N439, Y453, L452, S477, T478, E484, Q493, and N501. See, e.g., Lan et al., Nature 581:215-220 (2020). In embodiments, mutations in the SARS-CoV-2 S protein alter binding of the S protein to its host binding partner, e.g., ACE2. In embodiments, mutations in the SARS-CoV-2 S protein affect transmissibility of the virus. In embodiments, mutations in the SARS-CoV-2 S protein affect vaccine effectiveness against the virus. In embodiments, SARS-CoV-2 strains are characterized by SNPs in the coding sequence of the S protein. Such SARS-CoV-2 strains include, e.g., A.23.1 (also referred to as the “Uganda strain”); A.VOI.V2 (also referred to as the “Tanzania strain”); B.1; B.1.1.519 (also referred to as the “Mexico/Texas BV-2 strain”); B.1.1.529 (also referred to as the “Omicron variant” or “BA.1,” which comprises sub-lineages BA.2 and BA.3); B.1.1.7 (also referred to as the “UK strain” or “Alpha variant”); B.1.351 or 501Y.V2 (referred to as the “South Africa strain” or “Beta variant”); B.1.429 or Ca1.20C (referred to as the “California strain” or “Epsilon variant”); B.1.525 (also referred to as the “Nigeria strain” or “Eta variant”); B.1.526 (also referred to as the “New York strain” or “Iota variant”); B.1.617 (also referred to as the “India strain”); the B.1.617.1 strain (also referred to as the “Kappa strain”); B.1.617.2 (also referred to as the “Delta variant”), which has been further reclassified into sub-lineages designated as “AY”; B.1.617.3; Texas BV-1; B.1.621 (also referred to as the “Mu variant”); C.37 (also referred to as the “Chile/Peru strain” or “Lambda variant”); P.1 (also referred to as the “Brazil strain” or “Gamma variant”); P.2 (also referred to as the “Zeta variant”); P.3 (also referred to as the “Philippines strain”); and R.1 (also referred to as the Kentucky strain). The B.1.1.529 strain comprises the following mutations in the S protein: A67V, 469-70, T95I , G142D/Δ143-145, 4211/L2121, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, of which G339D, S371L, S373P, S375F, K417N, N440K, G446S, S477N, T478K, E484A, Q493R, G496S, Q498R, N501Y, and Y505H are in the S-RBD. The B.1.1.7 strain is characterized by the following mutations in the S protein: a deletion of amino acid residues 69-70, E484K, N501Y, D614G, and P681H. The 501Y.V2 strain is characterized by the following mutations in the S protein: D215G, K417N, E484K, N501Y, and D614G. The P.1 strain is characterized by the following mutations in the S protein: K417T, E484K, N501Y, and D614G. The Ca1.20C strain is characterized by a L452R mutation in the S protein. The B.1.526 strain comprises the following mutations in the S protein: LSF, T95I, D253G, D614G, A701V, and either E484K or S477N. The B.1.526 strain comprising E484K is referred to herein as “B.1.526” or “B.1.526/E484K” and the B.1.526 strain comprising S477N is referred to herein as “B.1.526.2” “B.1.526/S477N.”

In embodiments, mutations in SARS-CoV-2 proteins, e.g., S protein, result from genetic recombination between two or more SARS-CoV-2 variants. For example, a host subject may be simultaneously infected by two variants, e.g., the B.1.617.2/AY.4 (“Delta”) and B.1.1.529/BA.1 (“Omicron”) variants, which may recombine when replicating in the host to produce a recombinant variant. The recombinant variant may be designated as the cross between its parent variants. For example, the recombinant variant resulting from Delta (AY.4) and Omicron (BA.1) variants is designated as the BA.1×AY.4 recombinant.

As used herein, all strain designations include all of its sub-strains. For example, the B.1.526 strain includes the B.1.526, B.1.526.1, and the B.1.526.2 strains, and the B.1.617 strain includes the B.1.617, B.1.617.1, B.1.617.2, and B.1.617.3 strains. The B.1.617.2 strain (“Delta variant”) includes all “AY” sub-lineage designations, including AY.1, AY.2, AY3, AY.4, AY.5, AY.6, AY.7, AY.8, AY.9, AY.10, AY.11, AY.12, AY.13, AY.14, AY.15, AY.16, AY.17, AY.18, AY.19, AY.20, AY.21, AY22, AY.23, AY.24, AY.25, and all sub-lineages thereof (e.g., AY4.2). As used herein, strains “characterized” by particular mutations include at least those particular mutations and may include additional mutations. These strains and associated mutations are summarized in Table 1A. Additional variants of SARS-CoV-2 comprise mutations in the S protein as shown in Tables 1B and 1D and are further described, e.g., in Faria et al., “Genomic characterisation of an emergent SARS-CoV-2 lineage in Manaus: preliminary findings” (2020). Accessed at virological.org/t/586; Wu et al., bioRxiv doi:10.1101/2021.01.25.427948 (2021); Guruprasad, Proteins 2021:1-8 (2021); Zhou et al., bioRxiv doi:10.1101/2021.03.24.436620 (2021). Further strains and mutations of SARS-CoV-2 are provided in the PANGO lineages database (cov-lineages.org); the Nextstrain database (nextstrain.org); the Global Evaluation of SARS-CoV-2/hCoV-19 Sequences (GESS) database provided by Fang et al., Nucleic Acid Res 49(D1):D706-D714 (2021) (wan-bioinfo.shinyapps.io/GESS); and the SARS-CoV-2 Mutation Browser provided by Rakha et al., bioRxiv doi: 10.1101/2020.06.10.145292 (2020) (covid-19.dnageography.com). The mutations denoted as “del” or “Δ” indicate a deletion of the indicated amino acid residues present in the reference sequence. For example, a variant S protein comprising a “A69-70” mutation means that amino acid residues at positions 69 and 70 of the wild-type S protein are deleted. The mutations denoted as “ins” indicates an insertion of one or more amino acid residues at the indicated amino acid position. For example, a variant S protein comprising an “ins146N” mutation means the variant S protein comprises an asparagine residue at amino acid position 146 of the variant S protein. The mutations denoted as (X1-X2)→Y denotes that the amino acid residues X1-X2 indicated in the parentheses are mutated to a single amino acid Y. For example, a variant S protein comprising a “(L24-A27)→S” mutation means the variant S protein comprises a replacement of the amino acid residues at positions 24 to 27 with a serine residue.

Throughout this application, when referring to an S protein comprising a specific mutation, the mutation is relative to the SARS-CoV-2 reference strain NC_045512. The S protein from the SARS-CoV-2 reference strain is also known as the “wild-type” S protein. For example, the S-D614G protein from SARS-CoV-2 comprises D to G substitution at amino acid residue 614 relative to the wild-type S protein from SARS-CoV-2.

TABLE 1A
SARS-CoV-2 Strains and Associated Mutations

| | | | |
|---|---|---|---|
| Genome location | | | |
| (based on SARS- | | Amino Acid Change | |
| CoV-2 reference | Nucleotide | in Corresponding | Exemplary Associated SARS- |
| strain NC_045512) | Change | Protein | CoV-2 Strain(s) |

5′ UTR

| | | |
|---|---|---|
| 241 | C > T | (N/A) |

nsp3

| | | |
|---|---|---|
| 3037 | C > T | F924F |

ORF1ab

| | | | |
|---|---|---|---|
| 1059 (Orf1a) | C > T | T265I | |
| 3267 | C > T | T1001I | B.1.1.7 |
| 3828 | C > T | S1188L | P.1 |
| 5230 | G > T | K1655N | 501Y.V2 |
| 5388 | C > A | A1708D | B.1.1.7 |
| 5648 | A > C | K1795Q | P.1 |
| 6954 | T > C | 12230T | B.1.1.7 |
| 11288-11296 | deletion | SGF3675-3677 del | B.1.1.7 |
| 17259 | G > T | E5665D | P.1 |

RdRp

| | | |
|---|---|---|
| 14408 | C > T | P323L |

S Protein

| | | | |
|---|---|---|---|
| 21614 | C > T | L18F | P.1 |
| 21618 | C > G | T19R | B.1.617.2, B.1.617.3 |
| 21621 | C > A | T20N | P.1 |
| 21638 | C > T | P26S | P.1 |
| 21765-21770 | deletion | HV69-70 del | B.1.1.7, B.1.525, B.1.1.529 |
| 21801 | A > C | D80A | 501Y.V2 |
| 21846 | C > T | T95I | B.1.621, B.1.526, B.1.617.1, |
| | | | B.1.1.529 |
| 21974 | G > T | D138Y | P.1 |
| 21991-21993 | deletion | Y144 del | B.1.1.7 |
| 22132 | G > T | R190S | P.1 |
| 22206 | A > G | D215G | 501Y.V2 |
| 22227 | C > T | A222V | |
| 22320 | A > G | D253G | B.1.526 |
| 22578 | G > A | G339D | B.1.1.529 |
| 22812 | A > C | K417T | P.1 |
| 22813 | G > T | K417N | 507Y.V2 (B.1.351), B.1.617.2 |
| | | | (AY.1 & AY.2), B.1.1.529 |
| 22865 | G > T | A435S | |
| 22917 | T > G | L452R | Cal.20C, B.1.427, B.1.429, |
| | | | B.1.526.1, B.1.617 (and sub- |
| | | | lineages) |
| 22995 | C > A | T478K | B.1.1.519, B.1.617.2 |
| 23012 | G > A | E484K | 501Y.V2, P.1, P.2, B.1.525, |
| | | | B.1.526, B.1.620 |
| 23012 | G > C | E484Q | B.1.617, B.1.617.1, B.1.617.3 |
| 23063 | A > T | N501Y | B.1.1.7, 501Y.V2, P.1 |
| 23271 | C > A | A570D | B.1.1.7 |
| 23403 | A > G | D614G | B.1.1.7, 501Y.V2, P.1, B.1.429, |
| | | | B.1.526, All B.1 lineages |
| 23525 | C > T | H655Y | P.1, C.1.2, B.1. 1.529 |
| 23593 | G > T | Q677H | B.1.525 |
| 23593 | G > C | Q677H | B.1.525 |
| 23604 | C > A | P681H | B.1.1.7, B.1.1.519, B.1.620 |
| 23604 | C > G | P681R | B.1.617.1, B.1.617.2, B.l.617.3 |
| 23664 | C > T | A701V | 501Y.V2 |
| 23709 | C > T | T716I | B.1.1.7 |
| 24224 | T > C | F888L | |
| 24506 | T > G | S982A | B.1.1.7 |
| 24642 | C > T | T10271 | P.1 |
| 24775 | A > T | Q1071H | B.1.617.1 |
| 24914 | G > C | D1118H | B.1.1.7 |
| 25088 | G > T | V1167F | |

Orf3a

| | | |
|---|---|---|
| 25563 | G > T | Q57H |
| 26144 | G > T | G251V |

E Protein

| | | | |
|---|---|---|---|
| 26456 | C > T | P71L | 501Y.V2 |

ORF8

| | | | |
|---|---|---|---|
| 27972 | C > T | Q27stop | B.1.1.7 |
| 28048 | G > T | R52I | B.1.1.7 |
| 28111 | A > G | Y73C | B.1.1.7 |
| 28167 | G > A | E92K | P.1 |

N Protein

| | | | |
|---|---|---|---|
| 28280 | GAT > CTA | D3L | B.1.1.7 |
| 28512 | C > G | P80R | P.1 |
| 28887 | C > T | T205I | 501Y.V2 |
| 28932 | G > T | A220V | |
| 28977 | C > T | S235F | B.1.1.7 |
| 29095 | C > T | F274F | |

Orf10

| | | |
|---|---|---|
| 29645 | G > T | V30L |

TABLE 1B
Additional Mutations of the SARS-CoV-2 S and N Proteins and Associated Strains

| | Associated Strain/Lineage |
|---|---|
| S Protein Mutation(s) (relative to S | |
| Protein of reference strain NC_045512) | |
| S Protein (e.g., S-ECD Domain) | |
| D614G | B.1 |
| A222V | |
| N439K | B.1.466.2 |
| Y453F | |
| S477N | |
| E484K | P.2; B.1.1.28.2 |
| N501T | |
| N501Y | |
| Q677H | C.36.2 |
| Q677P | |
| D936Y | |
| A222V, D614G | |
| N439K, D614G | |
| S477N, D614G | |
| Y453F, D614G | |
| E484K, D614G | |
| N501T, D614G | |
| N501Y, D614G | |
| D936Y, D614G | |
| Q677H, D614G | |
| A67V, Δ69-70, T95I, G142D/A143-145, Δ211/L212I, | B.1.1.529 |
| ins214EPE, G339D, S371L, S373P, S375F, K417N, N440K, | |
| G446S, S477N, T478K, E484A, Q493R, G496S, Q498R, | |
| N501Y, Y505H, T547K, D614G, H655Y, N679K, P681H, | |
| N764K, D796Y, N856K, Q954H, N969K, L981F | |
| A67V, Δ69-70, T95I, G142D/Δ143-145, Δ211/L212I, | B.1.1.529 + R346K |
| ins214EPE, G339D, R346K, S371L, S373P, S375F, K417N, | |
| N440K, G446S, S477N, T478K, E484A, Q493R, G496S, | |
| Q498R, N501Y, Y505H, T547K, D614G, H655Y, N679K, | |
| P681H, N764K, D796Y, N856K, Q954H. N969K, L981F | |
| A67V, Δ69-70, T95I, G142D/Δ143-145, Δ211/L212I, | B.1.1.529 + L452R |
| ins214EPE, G339D, S371L, S373P, S375F, K417N, N440K, | |
| L452R, G446S, S477N, T478K, E484A, Q493R, G496S, | |
| Q498R, N501Y, Y505H, T547K, D614G, H655Y, N679K, | |
| P681H, N764K, D796Y, N856K, Q954H, N969K, L981F | |
| A67V, H69-V70del, T95I, G142D/V143-Y145del, (N211- | B.1.1.529 + F817P, A892P, A899P, |
| L212)→I, ins214EPE, G339D, S371L, S373P, S375F, K417N, | A942P |
| N440K, G446S, S477N, T478K, E484A, Q493R, G496S, | |
| Q498R, N501Y, Y505H, T547K, D614G, H655Y, N679K, | |
| P681H, N764K, D796Y, F817P, N856K, A892P, A899P, | |
| A942P, Q954H, N969K, L981F | |
| T19R, A27S, T95I, G142D, E156-F157-R158→G, N211- | BA.1 × AY.4 recombinant |
| L212→I, ins214EPE, G339D, S371L, S373P, S375F, K417N, | |
| N440K, G446S, S477N, T478K, E484A, Q493R, G496S, | |
| Q498R, N501Y, Y505H, T547K, D614G, H655Y, N679K, | |
| P681H, N764K, D796Y, N856K, Q954H, N969K, L981F | |
| T19I, (L24-A27)→S, G142D, V213G, G339D, S371F, S373P, | BA.2 |
| S375F, T376A, D405N, R408S, K417N, N440K, S477N, | |
| T478K, E484A, Q493R, Q498R, N501Y, Y505H, D614G, | |
| H655Y, N679K, P681H, N764K, D796Y, Q954H, N969K | |
| T19I, (L24-A27)→S, G142D, V213G, G339D, S371F, S373P, | BA.2 + L452R |
| S375F, T376A, D405N, R408S, K417N, N440K, L452R, | |
| S477N, T478K, E484A, Q493R, Q498R, N501Y, Y505H, | |
| D614G, H655Y, N679K, P681H, N764K, D796Y, Q954H, | |
| N969K | |
| T19I, (L24-A27)→S, G142D, V213G, G339D, S371F, S373P, | BA.2 + L452M |
| S375F, T376A, D405N, R408S, K417N, N440K, L452M, | |
| S477N, T478K, E484A, Q493R, Q498R, N501Y, Y505H, | |
| D614G, H655Y, N679K, P681H, N764K, D796Y, Q954H, | |
| N969K | |
| T19I, (L24-A27)→S, G142D, V213G, G339D, S371F, S373P, | BA.2.12.1 |
| S375F, T376A, D405N, R408S, K417N, N440K, L452Q, | |
| S477N, T478K, E484A, Q493R, Q498R, N501Y, Y505H, | |
| D614G, H655Y, N679K, P681H, S704L, N764K, D796Y, | |
| Q954H, N969K | |
| A67V, H69-V70del, T95I, G142D, V143-Y145del, (N211- | BA.3 |
| L212)→I, G339D, S371F, S373P, S375F, D405N, K417N, | |
| N440K, G446S, S477N, T478K, E484A, Q493R, Q498R, | |
| N501Y, Y505H, D614G, H655Y, N679K, P681H, N764K, | |
| D796Y, Q954H, N969K | |
| T19I, (L24-A27)→S, H69-V70del, G142D, V213G, G339D, | BA.4 |
| S371F, S373P, S375F, T376A, D405N, R408S, K417N, | |
| N440K, L452R, S477N, T478K, E484A, F486V, Q493R, | |
| Q498R, N501Y, Y505H, D614G, H655Y, N658S, N679K, | |
| P681H, N764K, D796Y, Q954H, N969K | |
| T19I, (L24-A27)→S, H69-V70del, G142D, V213G, G339D, | BA.5 |
| S371F, S373P, S375F, T376A, D405N, R408S, K417N, | |
| N440K, L452R, S477N, T478K, E484A, F486V, Q493R, | |
| Q498R, N501Y, Y505H, D614G, H655Y, N679K, P681H, | |
| N764K, D796Y, Q954H, N969K | |
| del69-70, del144, N501Y, A570D, P681H, T716I, S982A, | B.1.1.7 |
| D1118H, D614G | |
| L18F, D80A, D215G, L242H, R246I, K417N, E484K, N501Y, | B.1.351 + L18F, L242H, R246I |
| A701V, D614G | |
| L18F, D80A, D215G, del242-244, R246I, K417N, E484K, | B.1.351 + L18F, del242-244, R246I |
| N501Y, A701V, D614G | |
| del69-70, del144, N501Y, A570D, P681H, T716I, S982A, | B.1.1.7 + N439K, Y453F |
| D1118H, N439K, Y453F, D614G | |
| L18F, T20N, P26S, D138Y, R190S, K417T, E484K, N501Y, | P.1 + V1176F |
| D614G, H655Y, T1027I, V1176F | |
| S13I, W152C, L452R, D614G | B.1.429 |
| E484K, N501Y, D614G | B.1.429 − S13I |
| del69-70, del144, N501Y, A570D, P681H, T716I, S982A, | B.1.1.7 + E484K |
| D1118H, D614G, E484K | |
| D80A, D215G, K417N, E484K, N501Y, A701V, D614G | B.1.351 |
| L18F, T20N, P26S, D138Y, R190S, K417T, E484K, N501Y, | P.1; B.1.1.28.1 |
| D614G, H655Y, T1027I | |
| L5F, T95I, D253G, E484K, D614G, A701V | B.1.526/E484K |
| L5F, T95I, D253G, S477N, D614G, A701V | B.1.526/S477N |
| Q52R, E484K, Q677H, D614G, F888L | B.1.525 |
| Q52R, A67V, del69-70, E484K, Q677H, D614G, F888L | B.1.525 + A67V + del69-70 |
| Q52R, A67V, del69-70, del144, E484K, Q677H, D614G, | B.1.525 + A67V + del69-70, del144 |
| F888L | |
| E484K, D614G, V1176F | P.2 |
| del141-143, E484K, N501Y, D614G, P681H | P.3 |
| del141-143, E484K, N501Y, D614G, P681H, E1092K, | P.3 + 1092K, 1101Y, 1176F |
| H1101Y, V1176F | |
| L452R, E484Q, D614G | B.1.617 |
| E154K, L452R, E484Q, D614G, P681R | B.1.617 + E154K, 681R |
| E154K, L452R, E484Q, D614G, P681R, Q1071H | B.1.617.1(2) |
| G142D, E154K, L452R, E484Q, D614G, P681R, Q1071H | B.1.617.1(3) |
| T95I, G142D, E154K, L452R, E484Q, D614G, P681R, | B.1.617.1(4) |
| Q1071H | |
| T95I, D253G, D614G | B.1.526 reference |
| D80G, Y144del, F157S, L452R, D614G, T859N, D950H | B.1.526.1 |
| L5F, T95I, D253G, S477N, D614G, Q957R | B.1.526.2 |
| R102I, F157L, V367F, Q613H, P681R | A.23.1 |
| F157L, V367F, Q613H, P681R | A.23.1 |
| D80Y, Y144del, 1210del, D215G, RSY246-248 del, L249M, | A.VOI.V2 |
| W258L, R346K, T478R, E484K, H655Y, P681H, Q957H | |
| L5F, ins214TDR, Q414K, N450K, D614G, T716I, | B.1.214.2 |
| D614G, Q677H, L938F | C.36.2 + L938F |
| del69-70, del144, Q493R, N501Y, A570D, D614G, P681H, | BV-1 |
| T716I, S982A, D1118H | |
| T478K, D614G, P681H, T732A | BV-2; B.1.1.1; C37; B.1.1.519 |
| G75V, T76I, R246del, S247del, Y248del, L249del, T250del, | B.1.1.1; C37 |
| P251del, G252del, D253N, L452Q, F490S, D614G, T859N | |
| W152L, E484K, D614G, G769V | R.1 |
| T19R, del157-158, L452R, T478K, D614G, P681R, D950N | B.1.617.2 (1) |
| T19R, L452R, T478K, D614G, P681R, D950N | B.1.617.2 (2) |
| T19R, del156-157, R158G, L452R, T478K, D614G, P681R, | B.1.617.2 (3) (“Delta”) |
| D950N | |
| T19R, G142D, del156-157, R158G, K417N, N439K, L452R, | B.1.617.2 + K417N, N439K, E484K, |
| T478K, E484K, N501Y, D614G, P681R, D950N | N501Y |
| T19R, G142D, del156/157, R158G, K417N, L452R, T478K, | B.1.617.2 + K417N, E484K, N501Y |
| E484K, N501Y, D614G, P681R, D950N | |
| T19R, G142D, del156/157, R158G, L452R, T478K, E484K, | B.1.617.2 + E484K, N501Y |
| N501Y, D614G, P681R, D950N | |
| T19R, G142D, del156/157, R158G, L452R, T478K, E484K, | B.1.617.2 + E484K |
| D614G, P681R, D950N | |
| T19R, G142D, L452R, E484Q, D614G, P681R, D950N | B.1.617.3(1) |
| T19R, L452R, E484Q, D614G, P681R | B.1.617.3(2) |
| P26S, del69-70, V126A, del144, del242-244, H245Y, S477N, | B.1.620 |
| E484K, D614G, P681H, TI0271, D1118H | |
| del69-70, L189F, N439K, D614G, V772I | B.1.258.17 |
| W152R, N439K, D614G, P681R | B.1.466.2 |
| T19R, del144, del157-158, L452R, T478K, D614G, P681R, | B.1.617.2 + del144 |
| D950N | |
| delY145-146, E484K, D614G | B.1.618 |
| T19R, del157-158, K417N, L452R, T478K, D614G, P681R, | AY.1 |
| D950N | (B.1.617.2(1) + K417N) |
| T19R, T95I, G142D, E156G, Δ157/158, W258L, K417N, | AY.1 (Alt Seq 1) |
| L452R, T478K, D614G, P681R, D950N | |
| T19R, V70F, G142D, del157-158, A222V, K417N, L452R, | AY.2 |
| T478K, D614G, P681R, D950N | |
| T19R, V70F, G142D, E156G, Δ157/158, A222V, K417N, | AY.2 (Alt Seq 1) |
| L452R, T478K, D614G, P681R, D950N | |
| T19R, K417N, L452R, T478K, D614G, P681R, D950N | B.1.617.2(2) + K417N |
| T95I, del144, E484K, D614G, P681H, D796H | B.1.1.318 |
| T95I, del144-145, E484K, D614G, P681H, D796H | B.1.1.318 |
| S12F, del69-70, W152R, R346S, L452R, D614G, Q677H, | C.36.3 |
| A899S | |
| D80A, D215G, K417N, D614G, A701V | B.1.351.1 |
| L18F, D80A, D215G, del242-244, K417N, E484K, N501Y, | B.1.351.2 |
| D614G, A701V | |
| D80G, T95I, G142D, Y144del, N439K, E484K, D614G, | AV-1 |
| P681R, I1130V, D1139H | |
| I210T, N440K, E484K, D614G, D936F, S939N, T1027I | B.1.619 |
| T95I, Y144T, Y145S, ins146N, R346K, E484K, N501Y, | B.1.621 |
| D614G, P681H, D950N | |
| T95I, Y144S, Y145N, R346K, E484K, N501Y, D614G, P681H, | B.1.621 |
| D950N | |
| T19R, T95I, G142D, Δ156/157, R158G, L452R, T478K, | AY.3 and B.1.617.2; |
| D614G, P681R, D950N | AY.4 (Alt Seq 2) |
| T19R, Δ156/157, R158G, L452R, T478K, D614G, P681R, | AY.3 (outbreak); AY.12 |
| D950N | |
| ΔLGV141-143, L452R, D614G, | A.2.5 |
| S477N, D614G | B.1.160 |
| D614G, P681R | |
| L452R, D614G | |
| T478K, D614G | |
| T19R, T95I, Δ156/157, R158G, L452R, T478K, D614G, | AY.4 and AY.12 |
| P681R, D950N | |
| T19R, T95I, G142D, Y145H, Δ156/157, R158G, A222V | AY.4.2 |
| L452R, T478K, D614G, P681R, D950N | |
| T19R, G142D, E156-F157del, R158G, A222V | AY.9 |
| L452R, T478K, D614G, P681R, D950N | |
| T19R, S112L, G142D, E156-F157del, R158G, | AY.25 |
| L452R, T478K, D614G, P681R, D950N | |
| P9L, P25L, C136F, Y144del, R190S, D215G, A243del, | C.1.2 |
| L244del, Y449H, T478K, E484K, N501Y, L585F, D614G, | |
| H655Y, P681H, N679K, T716I, T859N | |
| P9L, E96Q, C136-Y144del, R190S, D215H, R346S, N394S, | B.1.640.2 |
| Y449N, E484K, F490S, N501Y, D614G, P681H, T859N, | |
| D1139H | |
| S-RBD Domain | |
| G339D, S371L, S373P, S375F, K417N, N440K, G446S, | B.1.1.529 |
| S477N, T478K, E484A, Q493R, G496S, Q498R, N501Y, | |
| Y505H | |
| G339D, R346K, S371L, S373P, S375F, K417N, N440K, | B.1.1.529 + R346K |
| G446S, S477N, T478K, E484A, Q493R, G496S, Q498R, | (BA.1.1) |
| N501Y, Y505H | |
| G339D, S371L, S373P, S375F, K417N, N440K, L452R, | B.1. 1.529 + L452R |
| G446S, S477N, T478K, E484A, Q493R, G496S, Q498R, | |
| N501Y, Y505H | |
| G339D, S371F, S373P, S375F, T376A, D405N, R408S, | BA.2 |
| K417N, N440K, S477N, T478K, E484A, Q493R, Q498R, | |
| N501Y, Y505H | |
| G339D, S371F, S373P, S375F, T376A, D405N, R408S, | BA.2 + L452R |
| K417N, N440K, L452R, S477N, T478K, E484A, Q493R, | |
| Q498R, N501Y, Y505H | |
| G339D, S371F, S373P, S375F, T376A, D405N, R408S, | BA.2 + L452M |
| K417N, N440K, L452M, S477N, T478K, E484A, Q493R, | |
| Q498R, N501Y, Y505H | |
| G339D, S371F, S373P, S375F, T376A, D405N, R408S, | BA.2.12.1 |
| K417N, N440K, L452Q, S477N, T478K, E484A, Q493R, | |
| Q498R, N501Y, Y505H | |
| G339D, S371F, S373P, S375F, D405N, K417N, N440K, | BA.3 |
| G446S, S477N, T478K, E484A, Q493R, Q498R, N501Y, | |
| Y505H | |
| G339D, S371F, S373P, S375F, T376A, D405N, R408S, | BA.4; BA.5 |
| K417N, N440K, L452R, S477N, T478K, E484A, F486V, | |
| Q498R, N501Y, Y505H | |
| N501Y | B.1.1.7 |
| K417N, E484K, N501Y | B.1.351 |
| K417T, E484K, N501Y | P.1 |
| E484K, N501Y | P.3 |
| L452R | B.1.427; B.1.429; B.1.526.1 |
| E484K | B.1.525; B.1.526; B.1.526.1; P.2; R.1 |
| N439K | |
| K417N | |
| I472V | |
| Y453F | |
| S477N | B.1.526.2 |
| S477N, E484K | B.1.620 |
| N501T | |
| L452R, E484Q | B.1.617; B.1.617.1; B.1.617.3 |
| K417N, N439K L452R, T478K, E484K, N501Y | B.1.617.2 (4) |
| K417N, L452R, T478K, E484K, N501Y | B.1.617.2 + K417N, E484K, N501Y |
| L452R, T478K, E484K, N501Y | B.1.617.2 + E484K, N501Y |
| L452R, T478K, E484K | B.1.617.2 + E484K |
| Q414K, N450K | B.1.214.2 |
| R346K, T478R, E484K | A.VOI.V2 |
| V367F | A.23.1 |
| K417N | |
| K417T | |
| Q493R, N501Y | BV-1 |
| T478K | BV-2; B.1.1.519 |
| L452Q, F490S | B.1.1.1; C37 |
| L452R, T478K | B.1.617.2 |
| K417N, L452R, T478K | B.1.617.2 + K417N |
| N440K, E484K | B.1.619 |
| R346K, E484K, N501Y | B.1.621 |
| S1 Subunit | |
| del69-70, del144, N501Y, A570D, D614G, P681H | B.1.1.7 |
| K417N, E484K, N501Y, D614G | |
| D614G | |
| Q677H | |
| Q677P | |
| N Protein Mutation(s) (relative to S | |
| Protein of reference strain NC_045512) | |
| P13L, E31-S33del, R203K, G204R | B.1.1.529 |

Further SARS-CoV-2 SNPs have been identified, for example, at the genome locations listed in Table 1C, e.g., locations 3036, 8782 18060, 11083, 1397, 2891, 14408, 17746, 17857, 23403, 26143, 28144, and 28881. See, e.g., Pachetti et al., J Transl Med 18:179 (2020); Banerjee et al., bioRxiv, doi.org/10.1101/2020.04.06.027854 (9 Apr. 2020); Alouane et al., bioRxiv doi.org/10.1101/2020.06.20.163188 (21 Jun. 2020); Brufsky, J Med Virol 2020:1-5 (2020); and Mishra et al., bioRxiv doi.org/10.1101/2020.05.07.082768 (12 May 2020). The ability to determine viral strain and/or trace viral lineage in a population provides valuable epidemiological insight into the spread and evolution of the virus. Determining the particular viral strain that has infected a patient also allows more comprehensive treatment. For example, the patient can be treated with a strain-specific drug. If a particular strain is more transmissible and/or more likely to cause severe illness, early interventions can be provided to the patient.

TABLE 1C
SARS-CoV-2 Single Nucleotide Polymorphisms

| Genome location (based on SARS- | Nucleotide | Amino Acid |
|---|---|---|
| CoV-2 reference strain NC_045512) | Change | Change |
| 241 | C > T | 5′UTR |
| 1059 | C > T | T > I |
| 1604 | AATG > A | delTGA |
| 3037 | C > T | Synonymous |
| 4402 | T > C | Synonymous |
| 5062 | G > T | L > F |
| 8782 | C > T | Synonymous |
| 11083 | G > T | L > F |
| 11916 | C > T | S > L |
| 14408 | C > T | P > L |
| 14805 | C > T | Synonymous |
| 15324 | C > T | T > I |
| 17247 | T > C | Synonymous |
| 17747 | C > T | P > L |
| 17858 | A > G | M > V |
| 18060 | C > T | S > F |
| 18877 | C > T | H > Y |
| 21618 | C > G | T > R |
| 21765-21770 | Deletion | del69-70 |
| 21846 | C > T | T > I |
| 21991-21993 | Deletion | del144 |
| 22206 | A > G | D > G |
| 22132 | G > T | R > S |
| 22227 | C > T | A > V |
| 22277 | C > A | Q > K |
| 22578 | G > A | G > D |
| 22661 | G > T | V > F |
| 22320 | A > G | D > G |
| 22812 | A > C | K > T |
| 22813 | G > T | K > N |
| 22865 | G > T | A > S |
| 22917 | T > G | L > R |
| 22995 | C > A | T > K |
| 23012 | G > A | E > K |
| 23012 | G > C | E > Q |
| 23063 | A > T | N > Y |
| 23271 | C > A | A > D |
| 23403 | A > G | D > G |
| 23525 | C > T | H > Y |
| 23593 | G > T or G > C | Q > H |
| 23604 | C > A | P > H |
| 23604 | C > G | P > R |
| 23664 | C > T | A > V |
| 23709 | C > T | T > I |
| 24138 | C > A | T > N |
| 24224 | T > C | F > L |
| 24506 | T > G | S > A |
| 24775 | A > T | Q > H |
| 24914 | G > C | D > H |
| 25088 | G > T | V > F |
| 25563 | G > T | Q > H |
| 26144 | G > T | G > V |
| 27046 | C > T | T > M |
| 27964 | C > T | S > L |
| 28144 | T > C | L > S |
| 28311 | C > T | P > L |
| 28881-28883 | GGG > AAC | R > K, G > R |
| 28932 | C > T | A > V |
| 29095 | C > T | Synonymous |
| 29540 | G > A | Upstream |
| 29553 | G > A | Intergenic |
| 29645 | G > T | V > L |
| 29711 | G > T | Downstream |

TABLE 1D
Selected SARS-CoV-2 Strains and S Protein Mutations

| S Protein Mutation(s) (relative to reference strain NC_045512) | Associated Strain |
|---|---|
| F157L, V367F, Q613H, P681R | A.23.1 |
| D80Y, ΔΥ144, ΔI210, D215G, Δ246-248, L249M, W258L, R346K, | A.VOI.V2 |
| T478R, E484K, H655Y, P681H, Q957H | |
| T19R, Δ157/158, K417N, L452R, T478K, D614G, P681R, D950N | AY.1 |
| T19R, T95I, G142D, E156G, Δ157/158, W258L, K417N, L452R, T478K, | AY.1 (Alt Seq 1) |
| D614G, P681R, D950N | |
| T19R, V70F, G142D, Δ157/158, A222V, K417N, L452R, T478K, | AY.2 |
| D614G, P681R, D950N | |
| T19R, V70F, G142D, E156G, Δ157/158, A222V, K417N, L452R, T478K, | AY.2 (Alt Seq 1) |
| D614G, P681R, D950N | |
| T19R, T95I, G142D, Y145H, Δ156/157, R158G, A222V L452R, T478K, | AY.4.2 |
| D614G, P681R, D950N | |
| T19R, Δ156/157, R158G, L452R, T478K, D614G, P681R, D950N | AY.12 |
| D614G | B.1 |
| T478K, D614G, P681H, T732A | B.1.1.519 |
| A67V, Δ69-70, T95I, G142D/Δ143-145, Δ211/L212I, ins214EPE, | B.1.1.529 |
| G339D, S371L, S373P, S375F, K417N, N440K, G446S, S477N, T478K, | |
| E484A, Q493R, G496S, Q498R, N501Y, Y505H, T547K, D614G, | |
| H655Y, N679K, P681H, N764K, D796Y, N856K, Q954H, N969K, | |
| L981F | |
| A67V, Δ69-70, T95I, G142D/Δ143-145, Δ211/L212I, ins214EPE, | B.1.1.529 + R346K |
| G339D, R346K, S371L, S373P, S375F, K417N, N440K, G446S, S477N, | |
| T478K, E484A, Q493R, G496S, Q498R, N501Y, Y505H, T547K, | |
| D614G, H655Y, N679K, P681H, N764K, D796Y, N856K, Q954H, | |
| N969K, L981F | |
| A67V, Δ69-70, T95I, G142D/Δ143-145, Δ211/L212I, ins214EPE, | B.1.1.529 + L452R |
| G339D, S371L, S373P, S375F, K417N, N440K, L452R, G446S, S477N, | |
| T478K, E484A, Q493R, G496S, Q498R, N501Y, Y505H, T547K, | |
| D614G, H655Y, N679K, P681H, N764K, D796Y, N856K, Q954H, | |
| N969K, L981F | |
| A67V, H69-V70del, T95I, G142D/V143-Y145del, (N211-L212)→I, | B.1.1.529 + F817P, A892P, |
| ins214EPE, G339D, S371L, S373P, S375F, K417N, N440K, G446S, | A899P, A942P |
| S477N, T478K, E484A, Q493R, G496S, Q498R, N501Y, Y505H, T547K, | |
| D614G, H655Y, N679K, P681H, N764K, D796Y, F817P, N856K, | |
| A892P, A899P, A942P, Q954H, N969K, L981F | |
| T19I, (L24-A27)→S, G142D, V213G, G339D, S371F, S373P, S375F, | BA.2 |
| T376A, D405N, R408S, K417N, N440K, S477N, T478K, E484A, Q493R, | |
| Q498R, N501Y, Y505H, D614G, H655Y, N679K, P681H, N764K, | |
| D796Y, Q954H, N969K | |
| A67V, H69-V70del, T95I, G142D, V143-Y145del, (N211-L212)→I, | BA.3 |
| G339D, S371F, S373P, S375F, D405N, K417N, N440K, G446S, S477N, | |
| T478K, E484A, Q493R, Q498R, N501Y, Y505H, D614G, H655Y, | |
| N679K, P681H, N764K, D796Y, Q954H, N969K | |
| ΔH69-V70, ΔΥ144, N501Y, A570D, D614G, P681H, T716I, S982A, | B.1.1.7 |
| D1118H | |
| ΔH69-V70, ΔΥ144, E484K, N501Y, A570D, D614G, P681H, T716I, | B.1.1.7 + E484K |
| S982A, D1118H | |
| ΔH69-V70, L189F, N439K, D614G, V772I | B.1.258.17 |
| L18F, D80A, D215G, Δ242-244, R246I, K417N, E484K, N501Y, D614G, | B.1.351 |
| A701V | |
| D80A, D215G, K417N, E484K, N501Y, D614G, A701V | B.1.351.1 |
| S13I, W152C, L452R, D614G | B.1.429 |
| W152R, N439K, D614G, P681R | B.1.466.2 |
| Q52R, A67V, ΔH69-V70, Δ144, E484K, Q677H, D614G, F888L | B.1.525 |
| L5F, T95I, D253G, E484K, D614G, A701V | B.1.526 |
| D80G, ΔΥ144, F157S, L452R, D614G, T859N, D950H | B.1.526.1 |
| L452R, E484Q, D614G | B.1.617 |
| T95I, G142D, E154K, L452R, E484Q, D614G, P681R, Q1071H | B.1.617.1 |
| T19R, Δ157-158, L452R, T478K, D614G, P681R, D950N | B.1.617.2 |
| T19R, G142D, Δ156-157, R158G, L452R, T478K, D614G, P681R, | B.1.617.2 (AY.3; AY.5; |
| D950N | AY.6; AY.7; AY.14) (Alt |
| | Seq 1, previously |
| | designated as “CDC”) |
| T19R, T95I, G142D, Δ156/157, R158G, L452R, T478K, D614G, P681R, | B.1.617.2 (AY.4) (Alt Seq |
| D950N | 2, previously designated as |
| | “Alternative”) |
| T19R, ΔΥ144, Δ157/158, L452R, T478K, D614G, P681R, D950N | B.1.617.2 + ΔΥ144 |
| T19R, G142D, L452R, E484Q, D614G, P681R, D950N | B.1.617.3 |
| ΔΥ145/146, E484K, D614G | B.1.618 |
| P26S, ΔH69-V70, V126A, ΔΥ144, Δ242-244, H245Y, S477N, E484K, | B.1.620 |
| D614G, P681H, T1027I, D1118H | |
| T95I, Y144T, Y145S, insl46N, R346K, E484K, N501Y, D614G, P681H, | B.1.621 |
| D950N | |
| P9L, E96Q, C136-Y144del, R190S, D215H, R346S, N394S, Y449N, | B.1.640.2 |
| E484K, F490S, N501Y, D614G, P681H, T859N, D1139H | |
| Δ69-70, ΔΥ144, Q493R, N501Y, A570D, D614G, P681H, T716I, S982A, | BV-1 |
| D1118H | |
| G75V, T76I, ΔR246, ΔS247, ΔΥ248, ΔL249, ΔT250, ΔP251, ΔG252, | C.37 |
| D253N, L452Q, F490S, D614G, T859N | |
| L18F, T20N, P26S, D138Y, R190S, K417T, E484K, N501Y, D614G, | P.1 |
| H655Y, T1027I, V1176F | |
| E484K, D614G, V1176F | P.2 |
| Δ141-143, E484K, N501Y, D614G, P681H, E1092K, H1101Y, V1176F | P.3 |
| W152L, E484K, D614G, G769V | R.1 |

Alternative mutations for the S protein of SARS-CoV-2 strains AY.1, AY.2, and B.1.617.2 are listed as “Alt Seq #.”

TABLE 1E
Selected SARS-CoV-2 Strains and S-RBD Mutations

| S-RBD Mutation(s) (relative to reference strain NC_045512) | Associated Strain |
|---|---|
| V367F | A.23.1 |
| R346K, T478R, E484K | A.VOI.V2 |
| N501YK417N, L452R, T478K | BAY.1.1.7 |
| K417N, L452R, T478K | AY.2 |
| L452R, T478K | AY.3 |
| L452R, T478K | AY.4 |
| L452R, T478K | AY.5 |
| L452R, T478K | AY.6 |
| L452R, T478K | AY.7 |
| L452R, T478K | AY.12 |
| L452R, T478K | AY.14 |
| T478K | B.1.1.519 |
| G339D, S371L, S373P, S375F, K417N, N440K, G446S, S477N, T478K, | B.1.1.529 |
| E484A, Q493R, G496S, Q498R, N501Y, Y505H | |
| G339D, R346K, S371L, S373P, S375F, K417N, N440K, G446S, S477N, | B.1.1.529 + R346K |
| T478K, E484A, Q493R, G496S, Q498R, N501Y, Y505H | (BA.1.1) |
| G339D, S371L, S373P, S375F, K417N, N440K, L452R, G446S, S477N, | B.1.1.529 + L452R |
| T478K, E484A, Q493R, G496S, Q498R, N501Y, Y505H | |
| G339D, S371F, S373P, S375F, T376A, D405N, R408S, K417N, N440K, | BA.2 |
| S477N, T478K, E484A, Q493R, Q498R, N501Y, Y505H | |
| G339D, S371F, S373P, S375F, D405N, K417N, N440K, G446S, S477N, | BA.3 |
| T478K, E484A, Q493R, Q498R, N501Y, Y505H | |
| N501Y | B.1.1.7 |
| E484K, N501Y | B.1.1.7 + E484K |
| Q414K, N450K | B.1.214.2 |
| N439K | B.1.258.17 |
| K417N, E484K, N501Y | B.1.351 |
| K417N, E484K, N501Y | B.1.351.1 |
| L452R | B.1.427 |
| L452R | B.1.429 |
| N439K | B.1.466.2 |
| E484K | B.1.525 |
| E484K | B.1.526 |
| L452R | B.1.526.1 |
| S477N | B.1.526.2 |
| L452R, E484Q | B.1.617 |
| L452R, E484Q | B.1.617.1 |
| L452R, T478K | B.1.617.2 |
| L452R, T478K | B.1.617.2 + ΔΥ144 |
| L452R, E484Q | B.1.617.3 |
| E484K | B.1.618 |
| S477N, E484K | B.1.620 |
| Q493R, N501Y | BV-1 |
| L452Q, F490S | C.37 |
| K417T, E484K, N501Y | P.1 |
| E484K | P.2 |
| E484K, N501Y | P.3 |
| E484K | R.1 |

In embodiments, the invention provides a method for detecting a coronavirus in a biological sample, comprising: a) contacting the biological sample with a binding reagent that specifically binds a nucleic acid of the coronavirus; b) forming a binding complex comprising the binding reagent and the coronavirus nucleic acid; and c) detecting the binding complex, thereby detecting the coronavirus in the biological sample. In embodiments, the coronavirus nucleic acid is RNA. In embodiments, the coronavirus is SARS-CoV-2. In embodiments, the binding reagent comprises an oligonucleotide comprising a sequence complementary to the coronavirus nucleic acid sequence. In embodiments, the binding reagent binds to a nucleic acid from a specific strain of the coronavirus, e.g., the L strain or S strain of SARS-CoV-2, or the S-D614 or S-D614G strain of SARS-CoV-2, or the B.1.1.7 strain, 501Y.V2 strain, P.1 strain, or Ca1.20C strain of SARS-CoV-2. In embodiments, the binding reagent binds to a SARS-CoV-2 nucleic acid encoding the N protein (i.e., the N gene). The SARS-CoV-2 N gene can be detected at three different regions: N1, N2, and N3. The N1 and N2 regions are specific to SARS-CoV-2, and the N3 region is universal to the coronaviruses in the same clade as SARS-CoV-2 (e.g., clade 2 and 3 viruses within the subgenus Sarbecovirus, including SARS-CoV-2, SARS-CoV, and bat- and civet-SARS-like CoVs. See, e.g., Lu et al., Emerg Infect Dis 26(8):1654-1665 (2020)). In embodiments, the binding reagent binds to SARS-CoV-2 N1 region, N2 region, N3 region, or a combination thereof. In embodiments, the biological sample is saliva, the coronavirus is SARS-CoV-2 and the nucleic acid is RNA.

In embodiments, the coronavirus is capable of infecting a human. In embodiments, the coronavirus causes a respiratory tract infection in a human. In embodiments, the coronavirus is SARS-CoV, MERS-CoV, SARS-CoV-2, HcoV-OC43, HcoV-229E, HcoV-NL63, HcoV-HKU1, or a combination thereof. In embodiments, the method detects a coronavirus component that is substantially conserved in SARS-CoV, MERS-CoV, SARS-CoV-2, HcoV-OC43, HcoV-229E, HcoV-NL63, and HcoV-HKU1. In embodiments, the method detects a protein or peptide fragment that is substantially conserved in SARS-CoV, MERS-CoV, SARS-CoV-2, HcoV-0C43, HcoV-229E, HcoV-NL63, and HcoV-HKU1.

In embodiments, the immunoassay described herein is a multiplexed immunoassay method. A multiplexed immunoassay can simultaneously detect multiple substances of interest, e.g., coronavirus components, in a sample. A multiplexed immunoassay can also use multiple binding reagents that specifically bind a substance of interest, e.g., a coronavirus component, in a sample. Multiplexed immunoassays can provide reliable results while reducing processing time and cost. In embodiments, a multiplexed immunoassay for detecting a coronavirus comprises multiple binding reagents, each of which binds to a different coronavirus component, e.g., a conserved coronavirus protein. In embodiments, a multiplexed immunoassay comprising binding reagents that each specifically binds a different coronavirus component provides improved detection accuracy, e.g., over a singleplex method utilizing a single binding reagent. In embodiments, the immunoassay method detects a coronavirus by detecting one or more of the coronavirus E protein, S protein, including S1 and S2 subunits, S-NTD, S-ECD, and S-RBD, M protein, HE protein, N protein, nsp1, nsp2, nsp3, nsp4, nsp5, nsp6, nsp7, nsp8, nsp9, nsp10, nsp11, nsp12, nsp13, nsp14, nsp15, and nsp16. In embodiments, the coronavirus is SARS-CoV-2. In embodiments, the coronavirus is SARS-CoV-2. In embodiments, the immunoassay method detects SARS-CoV-2 by detecting SARS-CoV-2 N protein. In embodiments, the immunoassay method detects SARS-CoV-2 by detecting SARS-CoV-2 S protein. In embodiments, the immunoassay method detects SARS-CoV-2 by detecting SARS-CoV-2 S-D614. In embodiments, the immunoassay method detects SARS-CoV-2 by detecting SARS-CoV-2 S-D614G. In embodiments, the immunoassay method detects SARS-CoV-2 by detecting any of the SARS-CoV-2 S protein variants in Tables 1A and 1B. In embodiments, the immunoassay detects SARS-CoV-2 by detecting SARS-CoV-2 N protein and S protein. In embodiments, the immunoassay detects SARS-CoV-2 by detecting any combination of the SARS-CoV-2 N protein, S protein, E protein, and M protein. In embodiments, the immunoassay detects SARS-CoV-2 by detecting SARS-CoV-2 N protein, S protein, E protein, and M protein. In embodiments, the immunoassay detects SARS-CoV-2 by detecting any of the SARS-CoV-2 protein variants in Table 1A.

In embodiments, the immunoassay method is a multiplexed method comprising: contacting the biological sample with a surface comprising a binding reagent in each binding domain on the surface, wherein the binding reagent in each binding domain independently binds to a viral protein selected from SARS-CoV-2 N protein, SARS-CoV-2 S protein, SARS-CoV-2 E protein, SARS-CoV-2 M protein, or a combination thereof; forming a binding complex in each binding domain comprising the viral protein and the binding reagent that binds to the viral protein; and measuring the concentration of the viral protein in each binding complex. In embodiments, the SARS-CoV-2 S protein is SARS-CoV-2 S-D614. In embodiments, the SARS-CoV-2 S protein is SARS-CoV-2 S-D614G. In embodiments, the SARS-CoV-2 S protein comprises any of the mutations shown in Tables 1A and 1B. In embodiments, each binding complex further comprises a detection reagent that specifically binds to the viral protein of the binding complex. Detection reagents are further described herein.

In embodiments, the immunoassay method is a multiplexed method capable of simultaneously detecting multiple coronaviruses in a biological sample. In embodiments, the multiplexed method is capable of simultaneously detecting one or more of SARS-CoV, MERS-CoV, SARS-CoV-2, HcoV-OC43, HcoV-229E, HcoV-NL63, and HcoV-HKU1.

In embodiments, the binding reagent and/or the detection reagent that specifically binds to the coronavirus component described herein is an antibody, antigen, ligand, receptor, oligonucleotide, hapten, epitope, mimotope, or aptamer. In embodiments, the binding reagent and/or the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent and/or the detection reagent comprises at least one heavy or light chain complementarity determining region (CDR) of an antibody. In embodiments, the binding reagent and/or the detection reagent comprises at least two CDRs from one or more antibodies. In embodiments, the binding reagent and/or the detection reagent is an antibody or antigen-binding fragment thereof. In embodiments, the binding reagent and/or the detection reagent is a receptor for the coronavirus component. In embodiments, the binding reagent and/or the detection reagent is a receptor for the coronavirus S protein. In embodiments, the binding reagent and/or the detection reagent is angiotensin-converting enzyme 2 (ACE2). In embodiments, the binding reagent and/or the detection reagent is neuropilin-1 (NRP1). In embodiments, the binding reagent and/or the detection reagent is CD147.

In embodiments where the method comprises detecting one or more variants of an SARS-CoV-2 protein (e.g., an S protein comprising a mutation shown in Tables 1A and 1B or an Orf1ab, E, Orf8, or N protein comprising a mutation shown in Table 1A), the binding reagent comprises an antibody or antigen-binding fragment thereof that is capable of specifically binding the wild-type, protein variant(s), or both the protein variant and the wild-type, and the detection reagent comprises an antibody or antigen-binding fragment thereof that is capable of binding the wild-type, protein variant(s), or both the wild-type and variant forms of the protein. In embodiments, the SARS-CoV-2 protein is an S protein, an N protein, an E protein, an Orf1ab protein, an Orf8 protein, or a combination thereof. In embodiments, the SARS-CoV-2 protein is an S protein.

In embodiments, the method is capable of detecting about 1 fg/mL to about 1 ng/mL, about 1 fg/mL to about 0.8 ng/mL, about 1 fg/mL to about 0.5 ng/mL, about 1 fg/mL to about 0.1 ng/mL, about 1 fg/mL to about 50 pg/mL, about 1 fg/mL to about 20 pg/mL, about 1 fg/mL to about 10 pg/mL, about 1 fg/mL to about 5 pg/mL, about 1 fg/mL to about 2 pg/mL, about 1 fg/mL to about 1 pg/mL, about 5 fg/mL to about 100 fg/mL, about 7 fg/mL to about 75 fg/mL, or about 10 fg/mL to about 50 fg/mL of a virus (e.g., a coronavirus such as SARS-CoV-2). In embodiments, the method is capable of detecting less than or about 5 pg/mL, less than or about 2 pg/mL, less than or about 1 pg/mL, less than or about 500 fg/mL, less than or about 100 fg/mL, less than or about 75 fg/mL, less than or about 50 fg/mL, or less than or about 10 fg/mL of a virus (e.g., a coronavirus such as SARS-CoV-2). In embodiments, the method is capable of detecting less than or about 10⁹ viral particles per mL, less than or about 10⁸ viral particles per mL, less than or about 10⁷ viral particles per mL, less than or about 10⁶ viral particles per mL, less than or about 100000 viral particles per mL, less than or about 10000 viral particles per mL, less than or about 1000 viral particles per mL, or less than or about 100 viral particles per mL. In embodiments where the method detects a viral nucleic acid, one viral particle is one viral genome equivalent. In embodiments, the method is capable of detecting less than or about 10⁹ viral genome equivalents per mL, less than or about 10⁸ viral genome equivalents per mL, less than or about 10⁷ viral genome equivalents per mL, less than or about 10⁶ viral genome equivalents per mL, less than or about 100000 viral genome equivalents per mL, less than or about 10000 viral genome equivalents per mL, less than or about 1000 viral genome equivalents per mL, or less than or about 100 viral genome equivalents per mL.

### Biomarkers

In embodiments, the invention provides a method for detecting a biomarker that is produced by a host (e.g., a human subject) in response to a viral infection, e.g., by a respiratory virus, including coronaviruses such as SARS-CoV-2. As used herein, “host” refers to a subject who has been infected with or suspected of being infected with a virus described herein, e.g., a coronavirus such as SARS-CoV-2. Unless otherwise specified, the biomarkers described herein are produced by a host, e.g., a human subject, in response to viral exposure and/or infection as described herein. In embodiments, the biomarker is an immune response biomarker. In embodiments, the biomarker is an antibody. The terms “antibody biomarker” and “antibody” are used interchangeably throughout the present disclosure. In embodiments, the biomarker is an inflammation response biomarker. In embodiments, the biomarker is a damage response biomarker. In embodiments, the method is used to assess the severity and/or prognosis of a viral infection in a subject. In embodiments, the method is used to determine whether a subject has been previously exposed to a virus. In embodiments, the method is used to estimate the time of virus exposure and/or infection. In embodiments, the method is used to determine whether a subject has immunity to a virus. In embodiments, the virus is a coronavirus. In embodiments, the virus is SARS-CoV-2.

As used herein, the term “biomarker” refers to a biological substance that is indicative of a normal or abnormal process, e.g., disease, infection, or environmental exposure. Biomarkers can be small molecules such as ligands, signaling molecules, or peptides, or macromolecules such as antibodies, receptors, or proteins and protein complexes. A change in the levels of a biomarker can correlate with the risk or progression of a disease or abnormality or with the susceptibility or responsiveness of the disease or abnormality to a given treatment. A biomarker can be useful in the diagnosis of disease risk or the presence of disease in an individual, or to tailor treatments for the disease in an individual (e.g., choices of drug treatment or administration regimes). In evaluating potential drug therapies, a biomarker can be used as a surrogate for a natural endpoint such as survival or irreversible morbidity. If a treatment alters a biomarker that has a direct connection to improved health, the biomarker serves as a “surrogate endpoint” for evaluating clinical benefit. Biomarkers are further described in, e.g., Mayeux, NeuroRx 1(2): 182-188 (2004); Strimbu et al., Curr Opin HIV AIDS 5(6): 463-466 (2010); and Bansal et al., Statist Med 32: 1877-1892 (2013). The term “biomarker,” when used in the context of a specific organism (e.g., human, nonhuman primate or another animal), refers to the biomarker native to that specific organism. Unless specified otherwise, the biomarkers referred to herein encompass human biomarkers.

As used herein, the term “level” in the context of a biomarker refers to the amount, concentration, or activity of a biomarker. The term “level” can also refer to the rate of change of the amount, concentration, or activity of a biomarker. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a biomarker accumulated in a cell, including, for example, the amount of particular post-synthetic modifications of a biomarker such as a polypeptide (e.g., an antibody), nucleic acid, or small molecule. “Level” can also refer to an absolute amount of a biomarker in a sample or to a relative amount of the biomarker, including amount or concentration determined under steady-state or non-steady-state conditions. “Level” can further refer to an assay signal that correlates with the amount, concentration, activity or rate of change of a biomarker. The level of a biomarker can be determined relative to a control marker in a sample.

Measurement of biomarker values and levels before and after a particular event, e.g., cellular or environmental event, may be used to gain information regarding an individual's response to the event. For example, samples or model organisms can be subjected to stress- or disease-inducing conditions, or a treatment or prevention regimen, and a particular biomarker can then be detected and quantitated in order to determine its changes in response to the condition or regimen. However, the opposite, i.e., measuring biomarker values and levels to determine whether an organism has been subjected to stress- or disease-inducing condition, tends to be much more complicated, as changes in the levels of a single biomarker are sometimes not definitively associated with a particular condition.

In embodiments, the measured levels of the one or more biomarkers described herein provides information regarding infection and immune response to infection, e.g., the course or maturity of infection, the etiology of severe illness, and the potential severity of illness. In embodiments, the measured levels of the one or more biomarkers described herein provides information regarding a subject's antibody response, cytokine response, neutrophil, macrophage, and/or monocyte production, complement activation, B cell and/or T cell activation, or a combination thereof.

In embodiments, detection and/or measurement of a single biomarker is sufficient to provide a prediction and/or diagnosis of a disease or condition. In embodiments, combinations of biomarkers are used to provide a strong prediction and/or diagnosis. Although a linear combination of biomarkers (i.e., the combination comprises biomarkers that individually provide a relatively strong correlation) can be utilized, linear combinations may not be available in many situations, for example, when there are not enough biomarkers available and/or with strong correlation. In alternative approaches, a biomarker combination is selected such that the combination is capable of achieving improved performance (i.e., prediction or diagnosis) compared with any of the individual biomarkers, each of which may not be a strong correlator on its own. Biomarkers for inclusion in a biomarker combination can be selected for based on their performance in different individuals, e.g., patients, wherein the same biomarker may not have the same performance in different individuals, but when combined with the remaining biomarkers, provide an unexpectedly strong correlation for prediction or diagnosis in a population. For example, Bansal et al., Statist Med 32: 1877-1892 (2013) describe methods of determining biomarkers to include in such a combination, noting in particular that optimal combinations may not be obvious to one of skill in the art, especially when subgroups are present or when individual biomarker correlations are different between cases and controls. Thus, selecting a combination of biomarkers for providing a consistent and accurate prediction and/or diagnosis can be particularly challenging and unpredictable.

Even when a suitable combination of biomarkers is determined, utilizing the combination of biomarkers in an assay poses its own set of difficulties. For example, detecting and/or quantitating each biomarker in the combination in its own separate assay may not be feasible with small samples, and using a separate assay to measure each biomarker in a sample may not provide consistent and comparable results. Furthermore, running an individual assay for each biomarker in a combination can be a cumbersome and complex process that can be inefficient and costly.

A multiplexed assay that can simultaneously measure the concentrations of multiple biomarkers can provide reliable results while reducing processing time and cost. Challenges of developing a multi-biomarker assay (such as, e.g., a multiplexed assay described in embodiments herein) include, for example, determining compatible reagents for all of the biomarkers (e.g., capture and detection reagents described herein should be highly specific and not be cross-reactive; all assays should perform well in the same diluents); determining concentration ranges of the reagents for consistent assay (e.g., comparable capture and detection efficiency for the assays described herein); having similar levels in the condition and sample type of choice such that the levels of all of the biomarkers fall within the dynamic range of the assays at the same dilution; minimizing non-specific binding between the biomarkers and binding reagents thereof or other interferents; and accurately and precisely detecting a multiplexed output measurement.

In embodiments, the invention provides methods of assessing an individual's immune response to a viral infection. In embodiments, the invention provides methods of assessing a group of individuals immune response to a viral infection. In embodiments, assessing an immune response comprises determining the type and/or strength of the immune response, e.g., detecting the molecular components produced in response to a viral infection (e.g., acute phase reactants, antibodies, cytokines, etc.) and measuring the amounts of each component produced. In embodiments, the invention provides methods of assessing the differences in immune responses by age, race, ethnicity, socioeconomic backgrounds, and/or underlying conditions, e.g., lung disease, diabetes, cancer, etc., which may be associated with poor clinical outcomes. In embodiments, the invention provides methods of determining the epidemiology of diseases caused by the viruses described herein, e.g., COVID-19. In embodiments, the virus is a coronavirus. In embodiments, the virus is SARS-CoV-2.

In embodiments, the invention provides methods of assessing cross-reactivity of an individual's immune response between different coronaviruses (e.g., SARS-CoV, MERS-CoV, SARS-CoV-2, HcoV-OC43, HcoV-229E, HcoV-NL63, and HcoV-HKU1). In embodiments, the invention provides methods of mapping the epitopes recognized by an individual's immune response, e.g., epitopes on a coronavirus S protein. In embodiments, the invention provides methods of assessing the individual's clinical outcome based on the mapped epitopes of immune responses. In embodiments, the invention provides methods of assessing an individual's immune response by detecting different IgG classes and/or subclasses. In embodiments, the invention provides methods of assessing the individual's clinical outcome based on the IgG classes and/or subclasses. In embodiments, the invention provides methods of assessing the affinity and/or avidity of an individual's immune response to different viral antigens. In embodiments, the invention provides methods of assessing the strength of an immune response, e.g., measuring the total antibody concentration or the concentration of different classes or subclasses of antibodies in an individual. In embodiments, the invention provides methods of determining the natural interacting partner(s) of the virus, e.g., a coronavirus such as SARS-CoV-2. As used in the context of viral infections, a “natural interacting partner” refers to a substance in the host cell (e.g., proteins or carbohydrate moieties on a host cell surface) that interacts with a viral component described herein. Natural interacting partners of viruses are further described in, e.g., Brito et al., Front Microbiol 8:1557 (2017). Natural interacting partners of SARS-CoV-2 include, e.g., ACE2, NRP1, and CD147, and are further described in Gordon et al., bioRxiv 2020.03.22.002386v1 (2020) doi:10.1101/2020.03.22.002386v1, Daly et al., bioRxiv 2020.06.05. 134114 (2020) doi:10.1101/2020.06.05.134114, and Bojkova et al., Nature Research (Pre-Print 11 Mar. 2020) doi:10.21203/rs.3.rs-17218/v1. In embodiments, the invention provides a competitive assay for SARS-CoV-2 utilizes ACE2, NRP1, CD147, or different sialic acid-containing substances to determine the interacting partner(s) of the SARS-CoV-2 S protein.

In embodiments, the invention provides methods of assessing changes in the immune response over time. In embodiments, the invention provides methods of assessing an individual's immune response at different time points after infection and/or after the first onset of a symptom. In embodiments, the invention provides methods of assessing the cytokines present in an individual at different time points after infection and/or after the first onset of a symptom. Symptoms of viral infections are described herein. In embodiments, the invention provides methods of assessing the long-term effects of an infection on an individual. For example, the coronavirus SARS-CoV-2 can cause post-acute COVID-19 syndrome (also known as post-COVID syndrome or “long COVID”), in which symptoms of the infection, including fatigue, headaches, shortness of breath, anosmia, muscle weakness, low fever, and cognitive dysfunction, persist for weeks or months after the typical convalescence period of COVID-19. In embodiments, the invention provides methods of assessing an individual's immune response at different time points after vaccination. In embodiments, the invention provides methods of determining the immune response components that provide immunity to a viral infection. In embodiments, the invention provides methods of assessing an individual's immune response at different time points after receiving a treatment for the viral infection. In embodiments, the invention provides methods of assessing the effect of convalescent serum treatment in an individual, e.g., comprising measuring the individual's immune response after administration of the convalescent serum. In embodiments, the invention provides methods of assessing the immune response components (e.g., antibodies) present in a convalescent serum sample, e.g., comprising determining its effectiveness, half life, and/or functional window of treatment in an individual. In embodiments, the invention provides methods of assessing the effectiveness, half life, and/or functional window of protection of a therapeutic antibody treatment. In embodiments, the virus is a coronavirus. In embodiments, the virus is SARS-CoV-2.

In embodiments, the invention provides methods of assessing an individual's immune response, e.g., an antibody, to a coronavirus (e.g., an endemic coronavirus such as HcoV-OC43, HcoV-229E, HcoV-NL63, and HcoV-HKU) to determine a clinical outcome of infection by a different coronavirus, e.g., SARS-CoV-2. In embodiments, the invention provides methods of assessing an individual's immune response, e.g., an antibody, to a respiratory virus (e.g., influenza or RSV) to determine a clinical outcome of infection by a different respiratory virus, e.g., SARS-CoV-2. In embodiments, the invention provides methods of assessing an individual's immune response and/or clinical outcome in a SARS-CoV-2 infection by determining a ratio of the individual's antibody level against the SARS-CoV-2 N protein to the individual's antibody level against the SARS-CoV-2 S protein. In embodiments, the antibody levels are measured in a blood sample. In embodiments, the antibody levels are measured in a saliva sample.

In embodiments, the invention provides a serology assay for determining the SARS-CoV-2 strain that has infected an individual. Currently, the only available methods for determining SARS-CoV-2 strain are nucleic acid-based methods such as PCR or sequencing, which typically require a nasopharyngeal or oropharyngeal sample from a subject. Assessment of the subject's antibody or immune response, as described herein, would require a further serology sample. Thus, a serology assay that determines SARS-CoV-2 strain reduces the amount and type of sample required from the subject, thereby reducing sample collection and processing time, and stress on the subject. It was surprisingly discovered that antibody biomarkers from an individual infected with a particular SARS-CoV-2 strain had highly specific activity against the S protein and/or S-RBD of that particular strain as compared to other strains. This unexpected result demonstrates a population-wide immune response (e.g., antibody response) bias for strain-specific epitopes on the SARS-CoV-2 S protein and/or S-RBD, which was not observed with other infectious diseases, e.g., other viruses, which typically have highly variable immune responses (e.g., antibody responses) between infected individuals that do not correlate with the viral strain. In embodiments, the invention provides methods of assessing an individual's immune response to different strains or variants of a coronavirus, e.g., SARS-CoV-2. In embodiments, the invention provides methods of mapping SARS-CoV-2 strain-specific epitopes on the SARS-CoV-2 S protein and/or S-RBD. Such methods are also useful for epidemiological studies to determine circulating variants in a population or geographical region.

In embodiments, the invention provides a method of determining the SARS-CoV-2 strain that has infected one or more individuals, comprising: performing a multiplexed serology assay on a sample obtained from the one or more individuals to detect one or more antibody biomarkers against S proteins and/or S-RBD from multiple SARS-CoV-2 strains; and differentiating the detected antibody biomarker(s) based on binding of the antibody biomarker(s) to the S protein and/or S-RBD from each SARS-CoV-2 strain. In embodiments, the differentiating comprises determining a ratio of: a first antibody biomarker that binds an S protein and/or S-RBD from a first SARS-CoV-2 strain (e.g., wild-type SARS-CoV-2), to a second antibody biomarker that binds an S protein and/or S-RBD from a second SARS-CoV-2 strain (e.g., SARS-CoV-2 strain B.1.1.7). SARS-CoV-2 strains are further described herein, e.g., in Table 1A. Multiplexed serology assays are further described herein. In embodiments, the multiplexed serology assay detects one or more antibody biomarkers that binds to one or more of: an S protein from wild-type SARS-CoV-2, an S protein from SARS-CoV-2 strain P.1, an S protein from SARS-CoV-2 strain B.1.1.7, and an S protein from SARS-CoV-2 strain 501Y.V2. In embodiments, the multiplexed serology assay detects one or more antibody biomarkers that binds to one or more of: an S protein from wild-type SARS-CoV-2, an S-D614G from SARS-CoV-2, an S protein from SARS-CoV-2 strain P.1, an S protein from SARS-CoV-2 strain B.1.1.7, an S protein from SARS-CoV-2 strain 501Y.V2, and an S-RBD from wild-type SARS-CoV-2. In embodiments, the multiplexed serology assay detects one or more antibody biomarkers that binds to one or more of: an S protein from wild-type SARS-CoV-2, an S-RBD from wild-type SARS-CoV-2, an S protein from SARS-CoV-2 strain B.1.1.7, an S-RBD from SARS-CoV-2 strain B.1.1.7, an S protein from SARS-CoV-2 strain 501Y.V2, an S-RBD from SARS-CoV-2 strain 501Y.V2, an S protein from SARS-CoV-2 strain P.1, and an S-RBD from SARS-CoV-2 strain P.1. In embodiments, the multiplexed serology assay detects one or more antibody biomarkers that binds to one or more of: an S protein from wild-type SARS-CoV-2, an S-RBD from wild-type SARS-CoV-2, an S protein from SARS-CoV-2 strain B.1.429, an S-RBD from SARS-CoV-2 strain B.1.429, an S protein from SARS-CoV-2 strain B.1.526/E484K, an S-RBD from SARS-CoV-2 strain B.1.526/E484K, an S protein from SARS-CoV-2 strain B.1.526/S477N, and an S-RBD from SARS-CoV-2 strain B.1.526/S477N. In embodiments, the multiplexed serology assay detects one or more antibody biomarkers that binds to one or more S proteins or subunit or fragment thereof that comprises any of the mutations shown in Tables 1A and 1B. In embodiments, the multiplexed serology assay is a classical serology assay, bridging serology assay, or competitive serology assay as described herein.

In embodiments, the invention provides a method of determining one or more SARS-CoV-2 strains in a sample. The method described herein is useful for tracking spread of one or more SARS-CoV-2 strains. The method provided herein is further useful for tracking the spread of one or more SARS-CoV-2 strains in one or more geographical regions and/or for tracking the spread of one or more SARS-CoV-2 strains over time. In embodiments, the invention provides a method for determining a SARS-CoV-2 strain in a sample, comprising: detecting at least a first antibody biomarker in the sample that binds to an antigen from a first SARS-CoV-2 strain and at least a second antibody biomarker in the sample that binds to an antigen from a second SARS-CoV-2 strain, wherein the detecting comprises contacting the sample with a surface comprising at least two binding domains, wherein the antigen from the first SARS-CoV-2 strain is immobilized on a first binding domain, and the antigen from the second SARS-CoV-2 strain is immobilized on a second binding domain; and determining a ratio of the first antibody biomarker to the second antibody biomarker, thereby determining the SARS-CoV-2 strain.

In some embodiments, the sample is from one or more individuals, wherein the one or more individuals are currently infected with SARS-CoV-2. In some embodiments, the sample is from one or more individuals, wherein the one or more individuals were previously infected with SARS-CoV-2. In some embodiments, the sample is from at least two individuals, wherein at least one individual is currently infected with SARS-CoV-2 and at least one individual was previously infected with SARS-CoV-2. In some embodiments, the sample is from at least one individual, wherein the individual is currently infected and was previously infected with SARS-CoV-2. In embodiments, the sample is from one or more individuals, wherein the one or more individuals are located in one or more geographical regions. In embodiments, the sample is from one or more individuals obtained at different time points. In embodiments, the sample comprises a pooled sample from at least two individuals. Pooled samples are further described herein.

In embodiments, the method further comprises determining the SARS-CoV-2 from one or more samples. In embodiments, the one or more samples are from one or more individuals as described herein. In embodiments, the method further comprises comparing the SARS-CoV-2 in one or more samples from one or more individuals located in one or more geographical regions, thereby tracking spread of the SARS-CoV-2 strain in the one or more geographical regions.

In embodiments, the SARS-CoV-2 strain is determined by inputting the ratio of the first antibody biomarker to the second antibody biomarker into a classification algorithm. Classification algorithms are further described herein. In embodiments, the method further comprises training a classification algorithm. In embodiments, the training comprises: measuring the amount of antibody biomarkers in a sample from a subject infected with a known SARS-CoV-2 strain that bind to an antigen from one or more SARS-CoV-2 strains, wherein the one or more SARS-CoV-2 strains comprise the known SARS-CoV-2 strain; normalizing the amount of measured antibody biomarker that bind to an antigen from the known SARS-CoV-2 strain against the amount of measured antibody biomarker that bind to an antigen from a further SARS-CoV-2 strain; and providing the normalized antibody biomarker amount to the classification algorithm.

In embodiments, the invention provides a method for differentiating infection associated with different SARS-CoV-2 strains. In embodiments, the method comprises training a classification algorithm. In embodiments, the method comprises obtaining a sample from a subject infected with a known SARS-CoV-2 strain; and measuring the amount of antibody biomarkers in the sample that bind to the S protein and/or S-RBD from multiple SARS-CoV-2 strains. In embodiments, the measuring comprises performing a multiplexed serology assay, e.g., a classical, bridging, or competitive multiplexed serology assay as described herein. In embodiments, the measured antibody biomarker amount for a particular strain is normalized against the measured antibody biomarker amount for a different strain. In embodiments, the normalized antibody biomarker amount is used to train the classification algorithm. In embodiments, normalized antibody biomarker amounts from multiple subjects, each infected with a known SARS-CoV-2 strain, are used to train the classification algorithm. Classification algorithms are known in the field and include but are not limited to, e.g., linear regression, logistic regression, random forest, support vector machine, and neural network. In embodiments, the invention provides a method of determining the SARS-CoV-2 strain that has infected one or more individuals, comprising: performing a multiplexed serology assay on a sample obtained from the one or more individuals to detect one or more antibody biomarkers against S proteins and/or S-RBD from multiple SARS-CoV-2 strains as described herein; and applying the classification algorithm described herein to determine the SARS-CoV-2 strain.

Serology tests that assess the presence of an antibody biomarker against a SARS-CoV-2 antigen have received U.S. FDA Emergency Use Authorization (EUA) with specificity of 95%. In embodiments, the invention provides improved sensitivity and/or specificity in determining whether a subject is currently infected or has previously been infected with a virus, e.g., a coronavirus such as SARS-CoV-2. In embodiments, the invention provides improved sensitivity and/or specificity in determining whether a subject has immunity to a virus, e.g., a coronavirus such as SARS-CoV-2. In embodiments, the methods herein have a sensitivity of greater than 90%, greater than 95%, greater than 96%, greater than 97%, greater than 98%, greater than 99%, greater than 99.5%, or greater than 99.9%. In embodiments, the methods herein have a specificity of greater than 90%, greater than 95%, greater than 96%, greater than 97%, greater than 98%, greater than 99%, greater than 99.5%, or greater than 99.9%. Assays with high sensitivity and specificity are important to correctly diagnose active infections and to correctly determine whether an individual has been previously exposed and/or immune to a virus, e.g., a coronavirus such as SARS-CoV-2. In particular, assays with high specificity are useful for conducting epidemiological studies in populations with low disease prevalence. Moreover, assays with high specificity are important for individual assessment due to the high risk of a false positive to the individual and the individual's community; individuals who received a false positive serology test result for SARS-CoV-2 may believe themselves to be immune and therefore erroneously engage in activity that can increase the likelihood of infection and spread of the virus.

Antibody Biomarkers

In embodiments, the invention provides a method for detecting a respiratory virus, e.g., a coronavirus such as SARS-CoV-2, in a biological sample, by detecting a biomarker produced in response to an infection by the virus. In embodiments, the biomarker produced in response to a viral infection is an antibody.

In embodiments, the invention provides a method for detecting a biomarker that is capable of binding to a viral antigen in a biological sample. As used herein, a virus or viral antigen is any component or secretion of a virus that prompts an immune response in a host (e.g., a human). In embodiments, the viral antigen is a viral protein or fragment thereof. In embodiments, the viral antigen is a virus structural protein. In embodiments, the viral antigen is a virus nonstructural protein. Structural and nonstructural proteins of viruses, e.g., respiratory viruses such as coronaviruses, are described herein. In embodiments, the method is capable of determining whether a subject has been exposed to a particular virus, e.g., a coronavirus such as SARS-CoV-2. In embodiments, the method is capable of determining whether a subject is at risk of being infected by a particular virus, e.g., a coronavirus such as SARS-CoV-2. In embodiments, the method is capable of determining whether a subject has immunity to a particular virus, e.g., a coronavirus such as SARS-CoV-2.

In embodiments, the invention provides an immunoassay method comprising: quantifying the amounts of one or more biomarkers capable of binding to a respiratory virus antigen in a biological sample, wherein the respiratory virus is a coronavirus, an influenza virus, a paramyxovirus, an adenovirus, a bocavirus, a pneumovirus, an enterovirus, a rhinovirus, or a combination thereof, wherein the quantifying comprises measuring the concentrations of each of the one or more biomarkers in an immunoassay.

In embodiments, the immunoassay method comprises: contacting the biological sample with a surface comprising a viral antigen in a binding domain on the surface; forming a binding complex in the binding domain comprising the viral antigen and a biomarker that binds to the viral antigen; and measuring the concentration of the biomarker in the binding complex. In embodiments, the biomarker is IgG, IgA, IgM, or combination thereof. In embodiments, the concentration of the biomarker is measured by contacting the binding complex with a detection reagent that specifically binds IgG, IgA, or IgM. In embodiments, the biomarker is a human biomarker, a mouse biomarker, a rat biomarker, a ferret biomarker, a minx biomarker, a bat biomarker, or a combination thereof. In embodiments, the biomarker is human IgG, IgA, or IgM. In embodiments, the biomarker is mouse IgG, IgA, or IgM. In embodiments, the biomarker is rat IgG, IgA, or IgM. In embodiments, the biomarker is ferret IgG, IgA, or IgM. In embodiments, the biomarker is minx IgG, IgA, or IgM. In embodiments, the biomarker is bat IgG, IgA, or IgM. Detection reagents are further described herein.

Respiratory viruses and proteins thereof are further described herein. In embodiments, the immunoassay method is capable of detecting a coronavirus, an influenza virus, a respiratory syncytial virus (RSV), or a combination thereof. In embodiments, the immunoassay method detects a biomarker that binds to a viral antigen from SARS-CoV-2, SARS-CoV, MERS-CoV, HcoV-OC43, HcoV-229E, HcoV-NL63, HcoV-HKU1, influenza A, influenza B, RSV, or a combination thereof. In embodiments, the viral antigen comprises nucleocapsid protein (N) from SARS-CoV-2, N protein from SARS-CoV, N protein from MERS-CoV, N protein from HcoV-229E, N protein from HcoV-NL63, N protein from HcoV-HKU1, N protein from HcoV-OC43, spike protein (S) from SARS-CoV-2, S protein from SARS-CoV, S protein from MERS-CoV, S protein from HcoV-229E, S protein from HcoV-NL63, S protein from HcoV-HKU1, S protein from HcoV-OC43, hemagglutinin (HA) from influenza B strain, influenza A H1 strain (e.g., H1/Michigan strain), influenza A H3 strain (e.g., H3/Hong Kong strain), influenza A H7 strain (e.g., H7/Shanghai strain), fusion protein (F), including, e.g., pre-fusion and post-fusion variants, from respiratory syncytial virus (RSV), or a combination thereof. In some embodiments, the S protein is a subunit, domain, or fragment thereof, e.g., S1, S2, S-NTD, S-ECD, or S-RBD as described herein. In some embodiments, the S protein is SARS-CoV-2 S-D614. In some embodiments, the S protein is SARS-CoV-2 S-D614G. In embodiments, the S protein is a SARS-CoV-2 S protein or subunit or fragment thereof that comprises any of the mutations shown in Tables 1A and 1B. In embodiments, the N protein is a SARS-CoV-2 N protein that comprises any of the mutations shown in Table 1A.

In embodiments, the immunoassay method detects a biomarker that binds to an N protein from SARS-CoV-2. In embodiments, the immunoassay method detects a biomarker that binds to a S protein from SARS-CoV-2. In embodiments, the immunoassay method detects a biomarker that binds to S1, S2, S-ECD, S-NTD, or S-RBD from SARS-CoV-2. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A. In embodiments, the immunoassay method comprises: contacting the biological sample with a surface comprising a viral antigen in a binding domain on the surface; forming a binding complex in the binding domain comprising the viral antigen and a biomarker that binds to the viral antigen; and measuring the concentration of the biomarker in the binding complex. In embodiments, the biomarker is IgG, IgA, IgM, or combination thereof. In embodiments, the biomarker is an IgG, IgA, and/or IgM from a human, mouse, rat, ferret, minx, bat, or combination thereof. In embodiments, the concentration of the biomarker is measured by contacting the binding complex with a detection reagent that specifically binds IgG, IgA, or IgM as described herein. In embodiments, the immunoassay method is a classical serology assay. In embodiments, the immunoassay method is a bridging serology assay. In embodiments, the immunoassay method is a competitive serology assay. In embodiments, the detection reagent comprises a labeled competitor of the biomarker. In embodiments, the competitor is ACE2. Classical, bridging, and competitive serology assays are described herein.

In embodiments, the method is a multiplexed method capable of simultaneously detecting and/or quantifying the amounts of the one or more biomarkers that bind to a respiratory virus antigen. As discussed herein, a method that is capable of simultaneously testing for several potential causes of infection (e.g., multiple different viruses) can advantageously allow a respiratory virus infection to be correctly and efficiently diagnosed in a single assay run and utilizing a single patient sample. Such as method can also be useful for assessing a patient's immune response to different respiratory virus infections.

In embodiments, the multiplexed method simultaneously detects and/or quantifies one or more biomarkers that binds to S proteins from different strains SARS-CoV-2. In embodiments, the multiplexed method is capable of determining the SARS-CoV-2 strain that has infected an individual and/or the SARS-CoV-2 strain that is circulating in a population or geographical region, as described herein. For example, as described herein, the S protein of SARS-CoV-2 strain B.1.1.7 (“UK”) comprises a deletion of residues 69-70, and the substitutions N501Y, D614G, and P681H. The S protein of SARS-CoV-2 strain 501Y.V2 (“South Africa”) strain comprises the substitutions D215G, K417N, E484K, N501Y, D614G, and A701V. The S protein of SARS-CoV-2 strain P.1 (“Brazil”) comprises the substitutions R190S, K417T, E484K, N501Y, and D614G. The S protein of SARS-CoV-2 strain Ca1.20C (“California”) strain comprises the substitution L452R. As discussed herein, when referring to an S protein comprising a specific mutation, the mutation is relative to the SARS-CoV-2 reference strain NC_045512, and the S protein from the SARS-CoV-2 reference strain is also known as the “wild-type” S protein. Moreover, an S protein (or subunit thereof) referred to herein as being from a specific SARS-CoV-2 strain includes all of the S protein mutations of that strain as described herein. Unless otherwise specified, the SARS-CoV-2 strains B.1.1.7, 501Y.V2, P.1, and Ca1.20C do not comprise mutations in the N protein, envelope protein, membrane protein, or other nonstructural proteins (e.g., Orf7a, Orf8) relative to the reference strain.

In embodiments, the invention provides a method for determining a SARS-CoV-2 strain in a sample, comprising detecting at least a first antibody biomarker in the sample that binds to an antigen, e.g., an S protein, N protein, and/or S-RBD, from a first SARS-CoV-2 strain and at least a second antibody biomarker in the sample that binds to an antigen, e.g., an S protein, N protein, and/or S-RBD, from a second SARS-CoV-2 strain, wherein the detecting comprises contacting the sample with a surface comprising one or more binding domains, wherein the antigen, e.g., the S protein, N protein, or S-RBD from the first SARS-CoV-2 strain is immobilized on a first binding domain, and the antigen, e.g., the S protein, N protein, or S-RBD from the second SARS-CoV-2 strain is immobilized on a second binding domain; and determining a ratio of the first antibody biomarker to the second antibody biomarker, thereby determining the SARS-CoV-2 strain. In embodiments, the detecting comprises performing a multiplexed method described herein. In embodiments, the multiplexed method simultaneously detects and/or quantifies one or more biomarkers that bind to an antigen, e.g., an S-protein, N protein, and/or an S-RBD from two or more SARS-CoV-2 strains as shown in Table 1A, Table 1D, and/or Table 1E. In embodiments, the sample is a biological sample. In embodiments, the sample is from one or more individuals as described herein. In embodiments, the sample is a saliva sample.

In embodiments, each antigen is immobilized on a distinct binding domain on the surface, wherein the antigens comprise an S protein, an N protein, and/or an S-RBD from a SARS-CoV-2 strain described herein. In embodiments, the antigens comprise an S protein, an N protein, and/or an S-RBD from a SARS-CoV-2 strain selected from: an S protein, an S-RBD, and/or an N protein from a SARS-CoV-2 strain selected from: wild-type; P.1; P.2; P.3; B.1.1.519; B.1.1.529; B.1.1.529 (+R346K); B.1.1.529 (+L452R); BA.1; BA.1.1; BA.2; BA.3; B.1.1.7; B.1.1.7 (+E484K); B.1.258.17; B.1.351; B.1.351.1; B.1.429; B.1.466.2; B.1.525; B.1.526/E484K; B.1.526/S477N; B.1.526.1; B.1.617; B.1.617.1; B.1.617.2; B.1.617.2 (+ΔY144); B.1.617.2 (+E484K); B.1.617.2 (+E484K/N501Y); B.1.617.2 (+K417N/N439K/E484K/N501Y); B.1.617.2 (+K417N/E484K/N501Y); AY.1; AY.2; AY.3, AY.4; AY.5, AY.6, AY.7, AY.4.2; AY.12; AY.14; B.1.617.3; B.1.618; B.1.620; B.1.621; B.1.640.2; BV-1; A.23.1; A.VOI.V2; C.37; and R.1; and/or an S protein and/or an S-RBD from SARS-CoV-2 comprising one or more mutations selected from: R346K, V367F; Q414K, K417N, K417T, N439K, N450K, L452R, L452Q, S477N, T478K, T478R, E484K, E484Q, F490S, Q493R, N501Y.

In embodiments, the multiplexed method simultaneously detects and/or quantifies one or more biomarkers that binds to: a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the multiplexed method simultaneously detects and/or quantifies one or more biomarkers that binds to: a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the multiplexed method simultaneously detects and/or quantifies one or more biomarkers that binds to: a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the multiplexed method simultaneously detects and/or quantifies one or more biomarkers that binds to: a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the multiplexed method simultaneously detects and/or quantifies one or more biomarkers that binds to: a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2. In embodiments, the S protein mutations from these SARS-CoV-2 strains are described in Table 1D. In embodiments, the S-RBD mutations from these SARS-CoV-2 strains are described in Table 1E. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: a wild-type S protein from SARS-CoV-2, an S-RBD from SARS-CoV-2 strain B.1.429, an N protein from SARS-CoV-2, an S-RBD from SARS-CoV-2 strain B.1.526, an S-RBD from SARS-CoV-2 strain B.1.526.2, an S protein from SARS-CoV-2 strain B.1.526, an S protein from SARS-CoV-2 strain B.1.429, and a wild-type S-RBD from SARS-CoV-2. In embodiments, the multiplexed method simultaneously detects and/or quantifies one or more biomarkers that binds to: a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises an N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD, wherein all mutations are relative to wild-type S-RBD from SARS-CoV-2. In embodiments, the multiplexed method simultaneously detects and/or quantifies one or more biomarkers that binds to: a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises N501Y and A570D mutations; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD, wherein all mutations are relative to wild-type S-RBD from SARS-CoV-2. In embodiments, the S protein mutations from these SARS-CoV-2 strains are described in Table 1D. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2. In embodiments, the multiplexed method simultaneously detects and/or quantifies one or more biomarkers that binds to: a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the multiplexed method simultaneously detects and/or quantifies one or more biomarkers that binds to a Spike protein from the following SARS-CoV-2 strains: wild type; A.23.1; A.VOI.V2; B.1.617.2; C.37; R.1; P.3; B.1.525; B.1.1.519; and BV-1. In embodiments, the multiplexed method simultaneously detects and/or quantifies one or more biomarkers that bind to a Spike protein from the following SARS-CoV-2 strains: wild type; AY.1, AY.2, B.1.617.2 plus deletion of Y144; B.1.620; B.1.258.17; B.1.466.2; B.1.1.7 plus the E484K mutation; B.1.351.1; and B.1.618. In embodiments, the Spike protein mutations from these SARS-CoV-2 strains are described in Table 1D. In embodiments, the Spike protein from SARS-CoV-2 strain B.1.617.2 comprises the mutations T19R, Δ157/158, L452R, T478K, D614G, P681R, and D950N. In embodiments, the Spike protein from SARS-CoV-2 strain B.1.617.2 comprises the mutations T19R, G142D, Δ156/157, R158G, L452R, T478K, D614G, P681R, and D950N. In embodiments, the Spike protein from SARS-CoV-2 strain B.1.617.2 comprises the mutations T19R, T95I, G142D, Δ156/157, R158G, L452R, T478K, D614G, P681R, and D950N. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD, wherein all mutations are relative to wild-type S-RBD from SARS-CoV-2. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: a wild-type S protein from SARS-CoV-2; an S-D614G from SARS-CoV-2; an N protein from SARS-CoV-2; an S protein from SARS-CoV-2 strain B.1.617.2; an S protein from SARS-CoV-2 strain P.1; an S protein from SARS-CoV-2 strain B.1.1.7; an S protein from SARS-CoV-2 strain B.1.351; and a wild-type S-RBD from SARS-CoV-2. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to a Spike protein from the following SARS-CoV-2 strains: wild type; P.2; B.1.617.1; B.1.617.3; B.1.617; P.1; and B.1.1.7. In embodiments, the Spike protein mutations from these SARS-CoV-2 strains are described in Table 1D. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: a Spike protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14). In embodiments, the Spike protein mutations from these SARS-CoV-2 strains are described in Table 1D. In embodiments, the Spike protein from SARS-CoV-2 strain AY.2 comprises the mutations T19R, V70F, G142D, E156G, Δ157/158, A222V, K417N, L452R, T478K, D614G, P681R, and D950N. In embodiments, the Spike protein from SARS-CoV-2 strain B.1.617.2 (AY.4) comprises the mutations T19R, T95I, G142D, Δ156/157, R158G, L452R, T478K, D614G, P681R, and D950N. In embodiments, the Spike protein from SARS-CoV-2 strain AY.1 comprises the mutations T19R, T95I, G142D, E156G, Δ157/158, W258L, K417N, L452R, T478K, D614G, P681R, and D950N. In embodiments, the Spike protein from SARS-CoV-2 strain B.1.617.2 (AY.3, AY.5, AY.6, AY.7, AY.14) comprises the mutations T19R, G142D, Δ156/157, R158G, L452R, T478K, D614G, P681R, and D950N. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: a Spike protein from the following SARS-CoV-2 strains: wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2. In embodiments, the Spike protein mutations from these SARS-CoV-2 strains are described in Table 1D. In embodiments, the Spike protein from SARS-CoV-2 strain B.1.617.2 (+K417N/N439K/E484K/N501Y) comprises the mutations T19R, G142D, Δ156/157, R158G, K417N, N439K, L452R, T478K, E484K, N501Y, D614G, P681R, and D950N. In embodiments, the Spike protein from SARS-CoV-2 strain B.1.617.2 (+K417N/E484K/N501Y) comprises the mutations T19R, G142D, Δ156/157, R158G, K417N, L452R, T478K, E484K, N501Y, D614G, P681R, and D950N. In embodiments, the Spike protein from SARS-CoV-2 strain B.1.617.2 (+E484K/N501Y) comprises the mutations T19R, G142D, Δ156/157, R158G, L452R, T478K, E484K, N501Y, D614G, P681R, and D950N. In embodiments, the Spike protein from SARS-CoV-2 strain B.1.617.2 (+E484K) comprises the mutations T19R, G142D, del156/157, R158G, L452R, T478K, E484K, D614G, P681R, and D950N. In embodiments, the Spike protein from SARS-CoV-2 strain B.1.617.2 comprises the mutations T19R, G142D, Δ156/157, R158G, L452R, T478K, D614G, P681R, and D950N. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: a Spike protein from the following SARS-CoV-2 strains: wild-type; B.1.1.529; AY.4.2; AY.4; P.1; B.1.1.7; B.1.351; and B.1.617.2. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: an S-RBD from the following SARS-CoV-2 strains: B.1.1.529; B.1.351; P.1; B.1.1.7; B.1.617.2; and wild-type. In embodiments, the S-RBD mutations from these SARS-CoV-2 strains are described in Table 1E. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: an S protein from the following SARS-CoV-2 strains: wild-type, B.1.1.529, AY.4, P.1, B.1.1.7, B.1.351; an N protein from wild-type SARS-CoV-2; and an S-RBD from wild-type SARS-CoV-2. In embodiments, the S protein mutations from these SARS-CoV-2 strains are described in Table 1D. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: an S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2. In embodiments, the S protein mutations from these SARS-CoV-2 strains are described in Table 1D. In embodiments, the one or more biomarkers is IgG, IgA, IgM, or combination thereof. In embodiments, the IgG, IgA, and/or IgM is from a human, mouse, rat, ferret, minx, bat, or combination thereof.

In embodiments, the multiplexed method simultaneously detects and/or quantifies one or more biomarkers that binds to: an S-RBD from the following SARS-CoV-2 strains: B.1.1.529 (BA.1); B.1.351; BA.2; P.1; B.1.1.7; BA.1.1; B.1.617.2; and wild-type. In embodiments, the S-RBD mutations from these SARS-CoV-2 strains are described in Table 1E. In embodiments, the one or more biomarkers is IgG, IgA, IgM, or combination thereof. In embodiments, the IgG, IgA, and/or IgM is from a human, mouse, rat, ferret, minx, bat, or combination thereof.

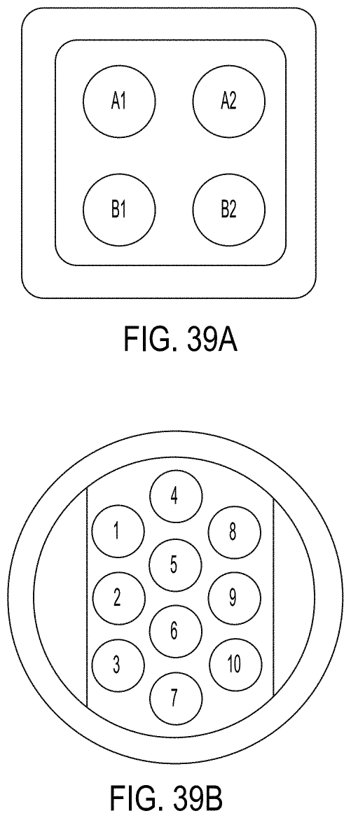

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an N protein from SARS-CoV-2, an S-RBD from SARS-CoV-2 strain 501Y.V2, and an S protein from SARS-CoV-2 strain 501Y.V2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises four distinct binding domains. An embodiment of a well in a 384-well assay plate, comprising four binding domains (“spots”), is shown in FIG. 39A. In embodiments, Spot A1 of FIG. 39A comprises an immobilized wild-type S protein from SARS-CoV-2, Spot A2 of FIG. 39A comprises an immobilized N protein from SARS-CoV-2, Spot B1 of FIG. 39A comprises an immobilized S-RBD from SARS-CoV-2 strain 501Y.V2, and Spot B2 of FIG. 39A comprises an immobilized S protein from SARS-CoV-2 strain 501Y.V2. In embodiments, the S protein mutations from these SARS-CoV-2 strains are described in Table 1D. In embodiments, the S-RBD mutations from these SARS-CoV-2 strains are described in Table 1E.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, and Spots 2, 4, 5, 6, and 10 of FIG. 39B each comprises an immobilized BSA. In embodiments, the S protein mutations from these SARS-CoV-2 strains are described in Table 1D.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-D614G from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, Spot 10 of FIG. 39B comprises an immobilized wild-type S-RBD from SARS-CoV-2, and Spots 4, 5, and 6 of FIG. 39B each comprises an immobilized BSA. In embodiments, the S protein mutations from these SARS-CoV-2 strains are described in Table 1D.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-D614G from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, and Spots 4, 5, 6, and 10 of FIG. 39B each comprises an immobilized BSA. In embodiments, the S protein mutations from these SARS-CoV-2 strains are described in Table 1D.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-RBD from SARS-CoV-2 strain 501Y.V2, Spot 3 of FIG. 39B comprises an immobilized N protein from SARS-CoV-2, Spot 4 of FIG. 39B comprises an immobilized S-RBD from SARS-CoV-2 strain P.1, Spot 6 of FIG. 39B comprises an immobilized S-RBD from SARS-CoV-2 strain B.1.1.7, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, Spot 10 of FIG. 39B comprises an immobilized wild-type S-RBD from SARS-CoV-2, and Spot 5 of FIG. 39B comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2, Spot 4 of FIG. 39B comprises an immobilized S-RBD from SARS-CoV-2 strain B.1.526/E484K, Spot 6 of FIG. 39B comprises an immobilized S-RBD from SARS-CoV-2 strain B.1.526/S477N, Spot 7 of FIG. 39B comprises an immobilized S protein from SARS-CoV-2 strain B.1.526/E484K, Spot 8 of FIG. 39B comprises an immobilized S protein from SARS-CoV-2 strain B.1.526/S477N, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spot 5 of FIG. 39B comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2, an S-RBD from SARS-CoV-2 strain B.1.429, an N protein from SARS-CoV-2, an S-RBD from SARS-CoV-2 strain B.1.526, an S-RBD from SARS-CoV-2 strain B.1.526.2, an S protein from SARS-CoV-2 strain B.1.526, an S protein from SARS-CoV-2 strain B.1.429, and a wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2, Spot 4 of FIG. 39B comprises an immobilized S-RBD from SARS-CoV-2 strain B.1.526, Spot 6 of FIG. 39B comprises an immobilized S-RBD from SARS-CoV-2 strain B.1.526.2, Spot 8 of FIG. 39B comprises an immobilized S protein from SARS-CoV-2 strain B.1.526, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spots 5 and 7 of FIG. 39B each comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, the multiplexed immunoassay method comprises: contacting the biological sample with a surface comprising a viral antigen in each binding domain on the surface, wherein the viral antigen in each binding domain is independently: a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises an N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD, wherein all mutations are relative to wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S-RBD that comprises an L452R mutation, Spot 2 of FIG. 39B comprises an immobilized SARS-CoV-2 S-RBD that comprises K417N, E484K, and N501Y mutations, Spot 3 of FIG. 39B comprises an immobilized SARS-CoV-2 S-RBD that comprises an E484K mutation, Spot 4 of FIG. 39B comprises an immobilized SARS-CoV-2 S-RBD that comprises K417T, E484K, and N501Y mutations, Spot 5 of FIG. 39B comprises an immobilized SARS-CoV-2 S-RBD that comprises an S477N mutation, Spot 6 of FIG. 39B comprises an immobilized SARS-CoV-2 S-RBD that comprises an N501Y mutation, Spot 7 of FIG. 39B comprises an immobilized SARS-CoV-2 S-RBD that comprises E484K and N501Y mutations, Spot 8 of FIG. 39B comprises an immobilized SARS-CoV-2 S-RBD that comprises L452R and E484Q mutations, Spot 9 of FIG. 39B comprises an immobilized SARS-CoV-2 S-RBD that comprises Q414K and N450K mutations, and Spot 10 of FIG. 39B comprises an immobilized wild-type SARS-CoV-2 S-RBD, wherein all mutations are relative to wild-type S-RBD from SARS-CoV-2.

In embodiments, the multiplexed immunoassay method comprises: contacting the biological sample with a surface comprising a viral antigen in each binding domain on the surface, wherein the viral antigen in each binding domain is independently: a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises N501Y and A570D mutations; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD, wherein all mutations are relative to wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S-RBD that comprises an L452R mutation, Spot 2 of FIG. 39B comprises an immobilized SARS-CoV-2 S-RBD that comprises K417N, E484K, and N501Y mutations, Spot 3 of FIG. 39B comprises an immobilized SARS-CoV-2 S-RBD that comprises an E484K mutation, Spot 4 of FIG. 39B comprises an immobilized SARS-CoV-2 S-RBD that comprises K417T, E484K, and N501Y mutations, Spot 5 of FIG. 39B comprises an immobilized SARS-CoV-2 S-RBD that comprises an S477N mutation, Spot 6 of FIG. 39B comprises an immobilized SARS-CoV-2 S-RBD that comprises N501Y and A570D mutations, Spot 7 of FIG. 39B comprises an immobilized SARS-CoV-2 S-RBD that comprises E484K and N501Y mutations, Spot 8 of FIG. 39B comprises an immobilized SARS-CoV-2 S-RBD that comprises L452R and E484Q mutations, Spot 9 of FIG. 39B comprises an immobilized SARS-CoV-2 S-RBD that comprises Q414K and N450K mutations, and Spot 10 of FIG. 39B comprises an immobilized wild-type SARS-CoV-2 S-RBD, wherein all mutations are relative to wild-type S-RBD from SARS-CoV-2.

In embodiments, the multiplexed immunoassay method comprises: contacting the biological sample with a surface comprising a viral antigen in each binding domain on the surface, wherein the viral antigen in each binding domain is independently: a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, the following immobilized antigens: a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2.

In embodiments, the multiplexed immunoassay method comprises: contacting the biological sample with a surface comprising a viral antigen in each binding domain on the surface, wherein the viral antigen in each binding domain is independently: a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, the following immobilized antigens: a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2.

In embodiments, the multiplexed immunoassay method comprises: contacting the biological sample with a surface comprising a viral antigen in each binding domain on the surface, wherein the viral antigen in each binding domain is independently: a Spike protein from the following SARS-CoV-2 strains: wild type; P.2; B.1.617.1; B.1.617.2; B.1.617.3; B.1.617; P.1; B.1.1.7; B.1.351; and B.1.526.1; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, the following immobilized antigens: a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the Spike protein mutations from these SARS-CoV-2 strains are described in Table 1D. In embodiments, the Spike protein from SARS-CoV-2 strain B.1.617.2 comprises the mutations T19R, Δ157/158, L452R, T478K, D614G, P681R, and D950N.

In embodiments, the multiplexed immunoassay method comprises: contacting the biological sample with a surface comprising a viral antigen in each binding domain on the surface, wherein the viral antigen in each binding domain is independently: a Spike protein from the following SARS-CoV-2 strains: wild type; A.23.1; A.VOI.V2; B.1.617.2; C.37; R.1; P.3; B.1.525; B.1.1.519; and BV-1; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, the following immobilized antigens: a Spike protein from the following SARS-CoV-2 strains: wild type; A.23.1; A.VOI.V2; B.1.617.2; C.37; R.1; P.3; B.1.525; B.1.1.519; and BV-1. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, the multiplexed immunoassay method comprises: contacting the biological sample with a surface comprising a viral antigen in each binding domain on the surface, wherein the viral antigen in each binding domain is independently: a Spike protein from the following SARS-CoV-2 strains: wild type; AY.1, AY.2, B.1.617.2 plus deletion of Y144; B.1.620; B.1.258.17; B.1.466.2; B.1.1.7 plus the E484K mutation; B.1.351.1; and B.1.618; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, the following immobilized antigens: a Spike protein from the following SARS-CoV-2 strains: wild type; AY.1, AY.2, B.1.617.2 plus deletion of Y144; B.1.620; B.1.258.17; B.1.466.2; B.1.1.7 plus the E484K mutation; B.1.351.1; and B.1.618. In embodiments, the Spike protein mutations from these SARS-CoV-2 strains are described in Table 1D. In embodiments, the Spike protein from SARS-CoV-2 strain B.1.617.2 plus deletion of Y144 comprises the mutations T19R, ΔY144, Δ157/158, L452R, T478K, D614G, P681R, and D950N.

In embodiments, the multiplexed immunoassay method comprises: contacting the biological sample with a surface comprising a viral antigen in each binding domain on the surface, wherein the viral antigen in each binding domain is independently: a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD, wherein all mutations are relative to wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-4 of FIG. 39B comprise, respectively, the following immobilized antigens: a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; Spots 7-10 of FIG. 39B comprise, respectively, the following immobilized antigens: a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD, wherein all mutations are relative to wild-type S-RBD from SARS-CoV-2; and Spots 5-6 of FIG. 39B each comprises BSA.

In embodiments, the multiplexed immunoassay method comprises: contacting the biological sample with a surface comprising a viral antigen in each binding domain on the surface, wherein the viral antigen in each binding domain is independently: a wild-type S protein from SARS-CoV-2; an S-D614G from SARS-CoV-2; an N protein from SARS-CoV-2; an S protein from SARS-CoV-2 strain B.1.617.2; an S protein from SARS-CoV-2 strain P.1; an S protein from SARS-CoV-2 strain B.1.1.7; an S protein from SARS-CoV-2 strain B.1.351; and a wild-type S-RBD from SARS-CoV-2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises a wild-type S protein from SARS-CoV-2; Spot 2 of FIG. 39B comprises an S-D614G from SARS-CoV-2; Spot 3 of FIG. 39B comprises an N protein from SARS-CoV-2; Spot 4 of FIG. 39B comprises an S protein from SARS-CoV-2 strain B.1.617.2; Spot 7 of FIG. 39B comprises an S protein from SARS-CoV-2 strain P.1; Spot 8 of FIG. 39B comprises an S protein from SARS-CoV-2 strain B.1.1.7; Spot 9 of FIG. 39B comprises an S protein from SARS-CoV-2 strain B.1.351; Spot 10 of FIG. 39B comprises a wild-type S-RBD from SARS-CoV-2; and Spots 5 and 6 of FIG. 39B comprise BSA. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, the multiplexed immunoassay method comprises: contacting the biological sample with a surface comprising a viral antigen in each binding domain on the surface, wherein the viral antigen in each binding domain is independently: a Spike protein from the following SARS-CoV-2 strains: wild type; P.2; B.1.617.1; B.1.617.2; B.1.617.3; B.1.617; P.1; B.1.1.7; B.1.351; and B.1.526.1; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, the following immobilized antigens: a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the Spike protein mutations from these SARS-CoV-2 strains are described in Table 1D. In embodiments, the Spike protein from SARS-CoV-2 strain B.1.617.2 comprises the mutations T19R, T95I, G142D, Δ156/157, R158G, L452R, T478K, D614G, P681R, and D950N.

In embodiments, the multiplexed immunoassay method comprises: contacting the biological sample with a surface comprising a viral antigen in each binding domain on the surface, wherein the viral antigen in each binding domain is independently: a Spike protein from the following SARS-CoV-2 strains: wild type; P.2; B.1.617.1; B.1.617.3; B.1.617; P.1; and B.1.1.7; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-3 of FIG. 39B comprise, respectively, an immobilized Spike protein from the following SARS-CoV-2 strains: wild type; P.2; and B.1.617.1; Spot 4 of FIG. 39B comprises BSA; and Spots 5-10 of FIG. 39B comprise, respectively, an immobilized Spike protein from the following SARS-CoV-2 strains: B.1.617.3; B.1.617; P.1; and B.1.1.7. In embodiments, the Spike protein mutations from these SARS-CoV-2 strains are described in Table 1D.

In embodiments, the multiplexed immunoassay method comprises: contacting the biological sample with a surface comprising a viral antigen in each binding domain on the surface, wherein the viral antigen in each binding domain is independently: a Spike protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14); forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, an immobilized Spike protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14). In embodiments, the Spike protein mutations from these SARS-CoV-2 strains are described in Table 1D. In embodiments, the Spike protein from SARS-CoV-2 strain AY.2 comprises the mutations T19R, V70F, G142D, E156G, Δ157/158, A222V, K417N, L452R, T478K, D614G, P681R, and D950N. In embodiments, the Spike protein from SARS-CoV-2 strain B.1.617.2 (AY.4) comprises the mutations T19R, T95I, G142D, Δ156/157, R158G, L452R, T478K, D614G, P681R, and D950N. In embodiments, the Spike protein from SARS-CoV-2 strain AY.1 comprises the mutations T19R, T95I, G142D, E156G, Δ157/158, W258L, K417N, L452R, T478K, D614G, P681R, and D950N. In embodiments, the Spike protein from SARS-CoV-2 strain B.1.617.2 (AY.3, AY.5, AY.6, AY.7, AY.14) comprises the mutations T19R, G142D, Δ156/157, R158G, L452R, T478K, D614G, P681R, and D950N.

In embodiments, the multiplexed immunoassay method comprises: contacting the biological sample with a surface comprising a viral antigen in each binding domain on the surface, wherein the viral antigen in each binding domain is independently: a Spike protein from the following SARS-CoV-2 strains: wild-type; wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, an immobilized Spike protein from the following SARS-CoV-2 strains: wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2. In embodiments, the Spike protein mutations from these SARS-CoV-2 strains are described in Table 1D. In embodiments, the Spike protein from SARS-CoV-2 strain B.1.617.2 (+K417N/N439K/E484K/N501Y) comprises the mutations T19R, G142D, Δ156/157, R158G, K417N, N439K, L452R, T478K, E484K, N501Y, D614G, P681R, and D950N. In embodiments, the Spike protein from SARS-CoV-2 strain B.1.617.2 (+K417N/E484K/N501Y) comprises the mutations T19R, G142D, Δ156/157, R158G, K417N, L452R, T478K, E484K, N501Y, D614G, P681R, and D950N. In embodiments, the Spike protein from SARS-CoV-2 strain B.1.617.2 (+E484K/N501Y) comprises the mutations T19R, G142D, Δ156/157, R158G, L452R, T478K, E484K, N501Y, D614G, P681R, and D950N. In embodiments, the Spike protein from SARS-CoV-2 strain B.1.617.2 (+E484K) comprises the mutations T19R, G142D, del156/157, R158G, L452R, T478K, E484K, D614G, P681R, and D950N. In embodiments, the Spike protein from SARS-CoV-2 strain B.1.617.2 comprises the mutations T19R, G142D, Δ156/157, R158G, L452R, T478K, D614G, P681R, and D950N.

In embodiments, the multiplexed immunoassay method comprises: contacting the biological sample with a surface comprising a viral antigen in each binding domain on the surface, wherein the viral antigen in each binding domain is independently: a Spike protein from the following SARS-CoV-2 strains: wild-type; B.1.1.529; AY.4.2; AY.4; P.1; B.1.1.7; B.1.351; and B.1.617.2; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-4 of FIG. 39B comprise, respectively, an immobilized Spike protein from the following SARS-CoV-2 strains: wild-type; B.1.1.529; AY.4.2; AY.4; Spots 5-6 each comprises BSA; and Spots 7-10 comprise, respectively, an immobilized Spike protein from the following SARS-CoV-2 strains: P.1; B.1.1.7; B.1.351; and B.1.617.2. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, the multiplexed immunoassay method comprises: contacting the biological sample with a surface comprising a viral antigen in each binding domain on the surface, wherein the viral antigen in each binding domain is independently: an S-RBD from the following SARS-CoV-2 strains: B.1.1.529; B.1.351; P.1; B.1.1.7; B.1.617.2; and wild-type. In embodiments, the one or more biomarkers is IgG, IgA, IgM, or combination thereof; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1 and 2 of FIG. 39B comprise, respectively, an immobilized S-RBD from the following SARS-CoV-2 strains: B.1.1.529 and B.1.351; Spot 3 comprises BSA; Spot 4 comprises an immobilized S-RBD from SARS-CoV-2 strain P.1; Spot 5 comprises BSA; Spot 6 comprises an immobilized S-RBD from SARS—Co-V-2 strain B.1.1.7; Spots 7 and 8 each comprise BSA; Spot 9 comprises an immobilized S-RBD from SARS-CoV-2 strain B.1.617.2; and Spot 10 comprises an immobilized S-RBD from wild type SARS-CoV-2. In embodiments, the S-RBD mutations from these SARS-CoV-2 strains are described in Table 1E.

In embodiments, the multiplexed immunoassay method comprises: contacting the biological sample with a surface comprising a viral antigen in each binding domain on the surface, wherein the viral antigen in each binding domain is independently: an S protein from the following SARS-CoV-2 strains: wild-type, B.1.1.529, AY.4, P.1, B.1.1.7, B.1.351; an N protein from wild-type SARS-CoV-2; and an S-RBD from wild-type SARS-CoV-2. In embodiments, the one or more biomarkers is IgG, IgA, IgM, or combination thereof; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1 and 2 of FIG. 39B comprise, respectively, an immobilized S protein from the following SARS-CoV-2 strains: wild-type and B.1.1.529; Spot 3 comprises an immobilized N protein from wild-type SARS-CoV-2; Spot 4 comprises an immobilized S protein from SARS-CoV-2 strain AY.4; Spots 5 and 6 each comprises BSA; Spots 7-9 comprise, respectively, an immobilized S protein from the following SARS-CoV-2 strains: P.1; B.1.1.7; and B.1.351; and Spot 10 comprises an immobilized S-RBD from wild type SARS-CoV-2. In embodiments, the Spike protein mutations from these SARS-CoV-2 strains are described in Table 1D.

In embodiments, the multiplexed immunoassay method comprises: contacting the biological sample with a surface comprising a viral antigen in each binding domain on the surface, wherein the viral antigen in each binding domain is independently: an S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2. In embodiments, the one or more biomarkers is IgG, IgA, IgM, or combination thereof; forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, an immobilized S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2. In embodiments, the S protein mutations from these SARS-CoV-2 strains are described in Table 1D.

In embodiments, the multiplexed immunoassay method comprises: contacting the biological sample with a surface comprising a viral antigen in each binding domain on the surface, wherein the viral antigen in each binding domain is independently: an S-RBD from the following SARS-CoV-2 strains: B.1.1.529 (BA.1); B.1.351; BA.2; P.1; B.1.1.7; BA.1.1; B.1.617.2; and wild-type. In embodiments, the one or more biomarkers is IgG, IgA, IgM, or combination thereof forming a binding complex in each binding domain comprising the viral antigen and a biomarker that binds to the viral antigen; and measuring the concentration of the biomarker in each binding complex. In embodiments, the biomarker is IgG, IgA, IgM, or combination thereof. In embodiments, the IgG, IgA, and/or IgM is from a human, mouse, rat, ferret, minx, bat, or combination thereof. In embodiments, the concentration of the biomarker is measured by contacting the binding complex with a detection reagent that specifically binds IgG, IgA, or IgM. Detection reagents are further described herein. In embodiments, the detection reagent is an antibody or antigen-binding fragment thereof. In embodiments, the detection reagent is a detectably labeled viral antigen. In embodiments, the immunoassay method is a classical serology assay. In embodiments, the immunoassay method is a bridging serology assay. In embodiments, the immunoassay is a competitive serology assay. Classical, bridging, and competitive serology assays are provided herein. In embodiments, the competitor is ACE2. In embodiments, the competitor is NRP1. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-4 of FIG. 39B comprise, respectively, an immobilized S-RBD from the following SARS-CoV-2 strains: B.1.1.529 (BA.1); B.1.351; BA.2; and P.1; Spot 6 comprises an immobilized S-RBD from SARS-CoV-2 strain B.1.1.7; Spots 8-10 comprise, respectively, an immobilized S-RBD from the following SARS-CoV-2 strains: BA.1.1; B.1.617.2; and wild-type; and Spots 5 and 7 each comprises an immobilized BSA. In embodiments, the S-RBD mutations from these SARS-CoV-2 strains are described in Table 1E.

In embodiments, the immunoassay method comprises detecting one or more viral antigens that are specific to SARS-CoV-2. As discussed herein, SARS-CoV-2 causes the respiratory illness COVID-19, which can cause mild to severe symptoms in patients. Sensitive and specific detection of SARS-CoV-2 is important for providing an accurate diagnosis, identifying asymptomatic infected individuals, and tracking spread of the disease. A method that detects biomarkers produced by an individual in response to a SARS-CoV-2 infection (e.g., antibodies) is also useful for identifying those who may be immune to the virus and therefore may be at lower risk when interacting with the general public or infected patients, and also may be potential candidates for plasma transfusions. In embodiments, the one or more biomarkers is capable of binding to a SARS-CoV-2 S-D614 protein, S-D614G, S1 subunit, S2 subunit, S-NTD, S-RBD, M protein, E protein, N protein, or a combination thereof. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A.

In embodiments, the method is a multiplexed method capable of simultaneously quantifying the one or more biomarkers that bind to a SARS-CoV-2 antigen. In embodiments, the multiplexed immunoassay method comprises: contacting the biological sample with a surface comprising a viral antigen in each binding domain on the surface, wherein the viral antigen in each binding domain is independently the SARS-CoV-2 S-D614, S-D614G, the SARS-CoV-2 S1 subunit, the SARS-CoV-2 S2 subunit, the SARS-CoV-2 S-RBD, the SARS-CoV-2 S-ECD, the SARS-CoV-2 S-NTD, the SARS-CoV-2 M protein, the SARS-CoV-2 E protein, or the SARS-CoV-2 N protein. In embodiments, the method is capable of simultaneously detecting a biomarker that binds to at least one of the SARS-CoV-2 S-D614, S-D614G, the SARS-CoV-2 S1 subunit, the SARS-CoV-2 S2 subunit, the SARS-CoV-2 S-RBD, the SARS-CoV-2 S-ECD, the SARS-CoV-2 S-NTD, the SARS-CoV-2 M protein, the SARS-CoV-2 E protein, and the SARS-CoV-2 N protein. In embodiments, the SARS-CoV-2 S protein or subunit or fragment thereof comprises a mutation as shown in Tables 1A and 1B. In embodiments, the SARS-CoV-2 N protein comprises a mutation as shown in Table 1A.

In embodiments, the immunoassay comprises: (a) contacting the biological sample with the viral antigen that specifically binds to a first biomarker of the one or more biomarkers; (b) forming a binding complex comprising the viral antigen and the first biomarker; and (c) measuring the concentration of the first biomarker in the binding complex.

In embodiments, the method further comprises repeating one or more of the method steps described herein to quantify the amounts of one or more biomarkers in the sample. In embodiments, the method further comprises repeating steps (a)-(c), wherein each biomarker specifically binds to a different viral antigen, thereby quantifying one or more biomarkers. In embodiments, each of steps (a)-(c) is performed for each biomarker in parallel.

In embodiments, the method is a multiplexed method. In embodiments, the multiplexed method is capable of simultaneously quantifying at least two biomarkers in the biological sample, wherein each of the at least two biomarkers is independently capable of binding to a viral antigen, e.g., any of HA, F, S, S1, S2, S-NTD, S-ECD, S-RBD, M, E, or N as described herein. In embodiments, the multiplexed method is capable of simultaneously quantifying two, three, four, five, or more than five biomarkers in the biological sample, wherein each biomarker is independently capable of binding to a viral antigen, e.g., any of HA, F, S, S1, S2, S-NTD, S-ECD, S-RBD, M, E, or N as described herein. In embodiments, the multiplexed method comprising quantifying a combination of the biomarkers provided herein has improved sensitivity and/or dynamic range, compared to a method in which only a single biomarker is quantified. For example, a multiplexed method can provide earlier and more sensitive detection compared to a method that detects a single biomarker, since responses to each viral antigen may vary between individuals. Moreover, the ability to simultaneously measure antibody responses against multiple similar viruses, e.g., a newly-emerged coronavirus such as SARS-CoV-2 and similar coronaviruses viruses such as hCoV-OC43, hCoV-HKU1, and hCoV-NL63, which have been circulating in the general population, improves understanding of how an individual's prior exposure to similar circulating viruses affects the individual's response to the newly-emerged virus of interest.

In embodiments, the method is used to diagnose whether a subject is infected with a virus, e.g., SARS-CoV-2. In embodiments, the method is used to assess the severity and/or prognosis of a viral infection in a subject. In embodiments, the method is used to determine whether a subject has been previously exposed to a virus. In embodiments, the method is used to estimate the time of virus exposure and/or infection. In embodiments, the method is used to determine whether a subject has immunity to a virus. In embodiments, the virus is a coronavirus. In embodiments, the virus is SARS-CoV-2.

In embodiments, the method is used to identify individuals with previous virus exposure for epidemiological studies (e.g., to understand true disease prevalence and evaluate the efficacy of infection control measures). In embodiments, the method is used to identify individuals at lower risk of future infection. Moreover, the method can be an important tool in the research, development, and validation of a vaccine for the virus. In embodiments, the method is used to assess differences in immune responses (e.g., antibody response) between individuals whose immunity is achieved by natural infection or vaccination. For example, a multiplexed method differentiates an individual's response to vaccination with different constructs of a viral antigen (e.g., different fragments of the S protein), compared with the individual's response to natural infection by the virus. Such a method can advantageously distinguish between individuals with biomarkers produced in response an active infection and are potentially contagious and individuals with biomarkers produced in response to the vaccine. In embodiments, the virus is a coronavirus. In embodiments, the virus is SARS-CoV-2.

In embodiments, the biomarker capable of binding to a viral antigen is an immune biomarker. In embodiments, the biomarker is an antibody or antigen-binding fragment thereof. In embodiments, the biomarker is an immunoglobulin A (IgA), immunoglobulin G (IgG; including IgG subclasses IgG1, IgG2, IgG3, and IgG4), immunoglobulin M (IgM), immunoglobulin E (IgE), or immunoglobulin D (IgD), or antigen-binding fragments thereof capable of binding to S, S1, S2, S-NTD, S-ECD, S-RBD, M, E, and/or N. In embodiments, the IgG, IgA, IgM, IgD, and/or IgE is from a human, mouse, rat, ferret, minx, bat, or combination thereof. In embodiments, the biomarker is an IgA or antigen-binding fragment thereof capable of binding to S, S1, S2, S-NTD, S-ECD, S-RBD, M, E, and/or N. In embodiments, the biomarker is an IgG or antigen-binding fragment thereof capable of binding to S, S1, S2, S-NTD, S-ECD, S-RBD, M, E, and/or N. In embodiments, the biomarker is an IgG1 or antigen-binding fragment thereof capable of binding to S, S1, S2, S-NTD, S-ECD, S-RBD, M, E, and/or N. In embodiments, the biomarker is an IgG2 or antigen-binding fragment thereof capable of binding to S, S1, S2, S-NTD, S-ECD, S-RBD, M, E, and/or N. In embodiments, the biomarker is an IgG3 or antigen-binding fragment thereof capable of binding to S, S1, S2, S-NTD, S-ECD, S-RBD, M, E, and/or N. In embodiments, the biomarker is an IgG4 or antigen-binding fragment thereof capable of binding to S, S1, S2, S-NTD, S-ECD, S-RBD, M, E, and/or N. In embodiments, the biomarker is an IgM or antigen-binding fragment thereof capable of binding to S, S1, S2, S-NTD, S-ECD, S-RBD, M, E, and/or N. In embodiments, the biomarker is an IgE or antigen-binding fragment thereof capable of binding to S, S1, S2, S-NTD, S-ECD, S-RBD, M, E, and/or N. In embodiments, the biomarker is an IgD or antigen-binding fragment thereof capable of binding to S, S1, S2, S-NTD, S-ECD, S-RBD, M, E, and/or N. In embodiments, the viral antigen is a coronavirus antigen. In embodiments, the coronavirus is SARS-CoV-2. In embodiments, the biomarker binds to SARS-CoV-2 S-D614. In embodiments, the biomarker binds to SARS-CoV-2 S-D614G. In embodiments, the biomarker binds to a SARS-CoV-2 S protein or subunit or fragment thereof that comprises a mutation as shown in Tables 1A and 1B. In embodiments, the biomarker binds to a SARS-CoV-2 N protein that comprises a mutation as shown in Table 1A.

In embodiments, the biomarker to be detected is an antibody biomarker, and the binding reagent is a viral antigen that is bound by the antibody biomarker. In embodiments, the binding reagent is a viral protein described herein, e.g., HA, F, S, S1, S2, S-NTD, S-ECD, S-RBD, M, E, N.

In embodiments, the binding reagent is a peptide antigen. Peptide antigens are short peptides of a native, full-length protein that include the antibody binding epitope. Peptide antigens can be easier to produce and provide greater flexibility in performing an immunoassay to detect an antibody biomarker. Peptide antigens can also have higher specificity to the antibody biomarker compared with a full-length viral protein or domain described herein. In embodiments, an immunoassay utilizing a peptide antigen as the binding reagent has reduced cross-reactivity with antibody biomarkers for a different virus that are present in the biological sample. For example, an immunoassay utilizing a SARS-CoV-2 peptide antigen can have reduced cross-reactivity for antibodies that may be present in a subject for a circulating coronavirus.

In embodiments, the peptide antigen is a fragment of a viral protein, e.g., a coronavirus protein. In embodiments, the peptide antigen comprises about 10 to about 100 amino acids. In embodiments, the peptide antigen comprises about 20 to about 80 amino acids. In embodiments, the peptide antigen comprises about 30 to about 60 amino acids. In embodiments, the peptide antigen comprises about 40 to about 50 amino acids. In embodiments, the peptide antigen is a fragment of S, S1, S2, S-NTD, S-ECD, S-RBD, M, E, or N. In embodiments, the peptide antigen comprises an immunodominant region (IDR) of a viral protein. In embodiments, the peptide antigen comprises amino acids 1-49 of the N protein IDR. In embodiments, the peptide antigen comprises amino acids 340-390 of the N protein IDR. In embodiments, the peptide antigen comprises amino acids 192-220 of the of the N protein IDR. In embodiments, the peptide antigen comprises amino acids 182-216 of the M protein IDR.

IgA, IgG (and subclasses thereof), IgM, IgE, and IgD are different isotypes of antibodies that have different immunological properties and functional locations. For example, IgA is typically found in the mucosal areas, such as the respiratory and gastrointestinal tracts, saliva, and tears and can prevent colonization by pathogens. IgG, the most abundant antibody isotype, has four subclasses as described herein and is found in all bodily fluids and provides the majority of antibody-based immunity against pathogens. IgM is mainly found in the blood and lymph fluid and is typically the first antibody made by the body to fight a new infection. IgE is mainly associated with allergic reactions (e.g., as part of aberrant immune response) and is found in the lungs, skin, and mucous membranes. IgD mainly functions as an antigen receptor on B cells and may activate basophils and mast cells to produce antimicrobial factors. Based on the timing and/or type of infection, different amounts of each isotype are produced.

In embodiments, the method is a multiplexed immunoassay method capable of quantifying the amount of each isotype of antibodies, e.g., IgG, IgA, IgE, and IgM, present in the biological sample. In embodiments, the amounts of the different isotypes of antibodies measured in a biological sample, e.g., the amounts of each of IgG, IgA, IgE, and IgM, can be used to determine whether a subject has been previously exposed to a virus. In embodiments, the amounts of the different isotypes of antibodies measured in a biological sample, e.g., the amounts of each of IgG, IgA, IgE, and IgM, can be used to estimate the time of virus exposure and/or infection. In embodiments, the amounts of the different isotypes of antibodies measured in a biological sample, e.g., the amounts of each of IgG, IgA, IgE, and IgM, can be used to determine whether a subject has immunity to a virus, e.g., a coronavirus such as SARS-CoV-2.

In embodiments, the method comprises: (a) contacting the biological sample with: at least a first, second, third, and fourth viral antigens, wherein each viral antigen specifically binds to IgG, IgA, IgE, and IgM, respectively; (b) forming at least a first, second, third, and fourth binding complex comprising the viral antigens and IgG, IgA, IgE, or IgM; and (c) measuring the concentration of IgG, IgA, IgE, or IgM in each of the binding complexes. In embodiments, each viral antigen is independently S, S1, S2, S-NTD, S-ECD, S-RBD, M, E, N, or a peptide antigen described herein. In embodiments, the IgG, IgA, IgE, and/or IgM is from a human, mouse, rat, ferret, minx, bat, or combination thereof.

IgG is further divided into four subclasses, IgG1, IgG2, IgG3, and IgG4, based on properties such as ability to activate complement, bind to macrophages, and/or pass through the placenta. Each subclass also has a distinct biological function. For example, the response to protein antigens is primarily mediated by IgG1 and IgG3, while IgG2 primarily mediates the response to polysaccharide antigens. IgG4 plays a role in protection against certain hypersensitivity reactions and pathogenesis of some autoimmune diseases. IgG subclass screening is performed to monitor a subject's infection response and/or determine whether a subject has antibody deficiency, and/or assess a subject's risk of an adverse response to infection. In embodiments, the method comprises determining the amount of IgG1, IgG2, IgG3, and IgG4 in the biological sample. In embodiments, the IgG is from a human, mouse, rat, ferret, minx, bat, or combination thereof.

In embodiments, the method comprises: (a) contacting the biological sample with: at least a first, second, third, and fourth viral antigens, wherein each viral antigen specifically binds to IgG1, IgG2, IgG3, and IgG4 respectively; (b) forming at least a first, second, third, and fourth binding complex comprising the viral antigens and IgG1, IgG2, IgG3, or IgG4; and (c) measuring the concentration of IgG1, IgG2, IgG3, or IgG4 in each of the binding complexes. In embodiments, each viral antigen is independently S, S1, S2, S-NTD, S-ECD, S-RBD, M, E, N, or a peptide antigen described herein. In embodiments, the IgG is from a human, mouse, rat, ferret, minx, bat, or combination thereof.

In embodiments, the method comprises: (a) contacting the biological sample with: a plurality of viral antigens, wherein each viral antigen specifically binds to an immunoglobulin selected from IgG1, IgG2, IgG3, IgG4, IgA, IgE, and IgM; (b) forming a plurality of binding complexes comprising the viral antigens and immunoglobulins; and (c) measuring the concentration of the immunoglobulin in each of the binding complexes. In embodiments, each viral antigen is independently S, S1, S2, S-NTD, S-ECD, S-RBD, M, E, N, or a peptide antigen described herein. In embodiments, the IgG, IgA, IgE, and/or IgM is from a human, mouse, rat, ferret, minx, bat, or combination thereof.

Inflammatory/Tissue Damage Response Biomarkers

In embodiments, the invention provides a method for detecting a biomarker in a subject to detect a viral infection, e.g., by a respiratory virus, including coronaviruses such as SARS-CoV-2. In embodiments, the invention provides a method for detecting a biomarker in a subject to assess the severity and/or prognosis of a viral infection, e.g., by a respiratory virus, including coronaviruses such as SARS-CoV-2. In embodiments, the biomarker is produced in response to the viral infection. In embodiments, the biomarker is a stress response protein. In embodiments, the biomarker is an inflammatory response biomarker. In embodiments, the biomarker is a tissue damage response biomarker. In embodiments, the biomarker is a T cell activation biomarker. In embodiments, the biomarker is an extracellular vesicle.

In embodiments, the immunoassay method simultaneously detects and/or quantifies IL-6, IL-10, IL-12p70, IL-4, TNF-α, IL-2, IL-1β, IFN-γ, and IL-17A in a biological sample. In embodiments, the immunoassay detecting and/or quantifying IL-6, IL-10, IL-12p70, IL-4, TNF-α, IL-2, IL-1β, IFN-γ, and IL-17A is detected and/or quantified is an ultrasensitive assay. In embodiments, the biological sample is obtained from a human subject. In embodiments, the biological sample is cerebrospinal fluid (CSF) or serum from a human subject. In embodiments, a subject who has been infected with a virus described herein, e.g., SARS-CoV-2, has higher CSF and/or serum levels of IL-2, IL-6, IL-10, IFN-γ, and TNF-α as compared to a subject who has not been infected with the virus, e.g., SARS-CoV-2.

In embodiments, the binding reagent that specifically binds the biomarker described herein is an antibody, antigen, ligand, receptor, oligonucleotide, hapten, epitope, mimotope, or aptamer. In embodiments, the binding reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent comprises at least one heavy or light chain complementarity determining region (CDR) of an antibody. In embodiments, the binding reagent comprises at least two CDRs from one or more antibodies. In embodiments, the binding reagent is an antibody or antigen-binding fragment thereof.

Extracellular Vesicles

In embodiments, the biomarker is an extracellular vesicle. Extracellular vesicles, also known as EVs or exosomes, are small membrane vesicles released by most cell types. For example, virus-infected cells release EVs that can mediate further in vivo viral spread in a variety of ways and produce other pathogenic effects. For example, EVs have been shown to transfer membrane-associated viral proteins, viral cargo proteins or RNAs, indirectly assist pathogens in escaping the immune system, or inhibit an immune response. EVs can also transfer viral genes from SARS-CoV-2 infected to non-infected cells and can induce inflammation in the absence of direct viral infection.

In embodiments, detecting EVs from infected cells is used to identify reservoirs of infection. In embodiments, EV populations in a biological sample are analyzed to determine the mechanism of infection, disease prognosis, and adaptive immunity. In embodiments, an EV released from a particular cell, e.g., an immune cell, comprises one or more of the same surface marker as that cell. In embodiments, the biomarker is an EV comprising an inflammatory damage and/or a tissue damage protein as described herein, on the surface of the EV.

Viral Component and Biomarker Detection

In embodiments, the invention provides a method comprising simultaneously detecting a host biomarker (e.g., an antibody biomarker or inflammatory and/or tissue damage response biomarker) described herein and a viral component described herein. A method that simultaneously determines, from a single sample, whether a subject is infected by a virus (e.g., a coronavirus such as SARS-CoV-2) and assesses the subject's immune response is capable of determining the subject's disease prognosis, for example, determining whether the subject will likely have poor disease progression and increased likelihood of intensive care treatment. Thus, the method enables preparation of an early response to a potentially serious illness.

In embodiments, the method is a multiplexed immunoassay method. In embodiments, the multiplexed immunoassay method detects a viral nucleic acid, a host antibody biomarker, a host inflammatory and/or tissue damage response biomarker, or a combination thereof.

In embodiments, a subject's infection status, disease progression, prognosis, or combination thereof is assessed by simultaneously detecting (1) a viral component, (2) a host antibody biomarker, and (3) a host inflammatory and/or tissue damage response biomarker as described herein. For example, Table 2 provides exemplary outcomes and assessments based on the combined detection for diagnosis and prognosis of COVID-19, the disease caused by SARS-CoV-2 infection.

TABLE 2
Exemplary Scenarios and Predicted Outcomes

| | | | Inflammatory/ | |
|---|---|---|---|---|
| | | | Tissue Damage | COVID-19 |
| | SARS-COV- | Host | Response (e.g., | Diagnosis, |
| Scenario | 2 Viral | Antibody | cytokine/ | Disease |
| # | Component | (Serology) | chemokine) | Stage |
| 1 | + | − | Normal | Early stage; |
| | | | | asymptomatic |
| 2 | − | + | Normal | Convalescent; |
| | | | | recovered |
| 3 | + | + | Elevated | Late stage, |
| | | | | active |
| | | | | infection |
| 4 | − | − | Elevated | Non-COVID-19 |
| | | | | inflammation |
| 5 | + | +/− | Highly | Active acute |
| | | | Elevated | stage infection. |
| | | | | May require |
| | | | | hospital |
| | | | | admission |
| 6 | − | − | Normal | No exposure |
| | | | | to COVID-19 |

### Samples and Assay Devices

In embodiments, the viruses, viral components, and/or biomarkers described herein are measured in a biological sample. In embodiments, the biological sample comprises a mammalian fluid, secretion, or excretion. In embodiments, the sample is a purified mammalian fluid, secretion, or excretion. In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, a respiratory sample, bronchial/bronchoalveolar lavage, saliva, mucus, oropharyngeal swab, sputum, endotracheal aspirate, pharyngeal/nasal swab, throat swab, amniotic fluid, nasal secretions, nasopharyngeal wash or aspirate, nasal mid-turbinate swab, vaginal secretions, a surface biopsy, sperm, semen/seminal fluid, wound secretions and excretions, ear secretions or discharge, or an extraction, purification therefrom, or dilution thereof. In embodiments, the biological sample is diluted such that the assay signal is within the upper and lower detection limits of the assay. In embodiments, the biological sample is diluted to achieve a desired assay sensitivity. Further exemplary biological samples include but are not limited to physiological samples, samples containing suspensions of cells such as mucosal swabs, tissue aspirates, endotracheal aspirates, tissue homogenates, cell cultures, and cell culture supernatants. In embodiments, the biological sample is a respiratory sample obtained from the respiratory tract of a subject. Examples of respiratory samples include, but are not limited to, bronchial/bronchoalveolar lavage, saliva, mucus, endotracheal aspirate, sputum, nasopharyngeal/nasal swab, throat swab, oropharyngeal swab and the like. In embodiments, the biological sample is whole blood, serum, plasma, cerebrospinal fluid (CSF), urine, saliva, sputum, endotracheal aspirate, nasopharyngeal/nasal swab, bronchoalveolar lavage, or an extraction or purification therefrom, or dilution thereof. In embodiments, the biological sample is blood that has been dried and reconstituted. In embodiments, the biological sample is serum or plasma. In embodiments, the plasma is in EDTA, heparin, or citrate. In embodiments, the biological sample is saliva. In embodiments, the biological sample is endotracheal aspirate. In embodiments, the biological sample is a nasal swab. In embodiments, the virus, viral component, and/or biomarkers described herein have substantially levels in the saliva or endotracheal aspirate of a subject. In embodiments, the virus, viral components, and/or biomarkers described herein are present in higher amounts in certain bodily fluids (e.g., saliva) compared to others (e.g., throat swab). In embodiments, certain antibody biomarker levels, e.g., IgG (including subclasses thereof) and IgA, are substantially similar in blood and saliva of a subject. In embodiments, the ratio of antibody levels to different components from a virus (e.g., SARS-CoV-2 S and N proteins) are highly correlated in blood and saliva of a subject. In embodiments, the ratio of antibody levels to different components from a virus, e.g., the ratio of the antibody levels against the SARS-CoV-2 S protein and the SARS-CoV-2 N protein is used to assess the immune response and/or clinical outcome of a subject infected with SARS-CoV-2.

In embodiments, the biological sample is from an animal. In embodiments, the biological sample from an animal is useful for animal model studies, e.g., for vaccine and/or drug research and development, and/or to better understand disease progression and infection lethality. Exemplary animals that are useful for animal model studies include, but are not limited to, mouse, rat, rabbit, pig, primate such as monkey, and the like.

In embodiments, the biological sample is from a human or an animal subject. In embodiments, the subject is susceptible or suspected to be susceptible to infection by the viruses described herein. In embodiments, the subject is known or suspected to transmit the viruses described herein. Virus transmission may occur among the same species (e.g., human-to-human) or inter-species (e.g., bat-to-human). Non-limiting examples of animal subjects include domestic animals, such as dog, cat, horse, goat, sheep, donkey, pig, cow, chicken, duck, rabbit, gerbil, hamster, guinea pig, and the like; non-human primates (NHP) such as macaque, baboon, marmoset, gorilla, orangutan, chimpanzee, monkey, and the like; big cats such as tiger, lion, puma, leopard, snow leopard, and the like; and other mammals such as bats and pangolins. In embodiments, the biological sample is from a human, a mouse, a rat, a ferret, a minx, or a bat. In embodiments, the subject is a host that has been exposed to and/or infected by a virus as described herein. In embodiments, the biological ample comprises a plasma (e.g., in EDTA, heparin, or citrate) sample from a subject. In embodiments, the biological sample comprises a serum sample from a subject. In embodiments, the biological sample is from a healthy subject. In embodiments, the biological sample is from a subject known to never have been exposed to a virus described herein. In embodiments, the biological sample is from a subject known to be immune to a virus described herein. In embodiments, the biological sample is from a subject known to be infected with a virus described herein. In embodiments, the biological sample is from a subject suspected of having been exposed to a virus described herein. In embodiments, the biological sample is from a subject at risk of being exposed to a virus described herein. In embodiments, the virus is a coronavirus. In embodiments, the virus is SARS-CoV-2.

In embodiments, the sample is an environmental sample. In embodiments, the environmental sample is aqueous, including but not limited to, fresh water, drinking water, marine water, reclaimed water, treated water, desalinated water, sewage, wastewater, surface water, ground water, runoff, aquifers, lakes, rivers, streams, oceans, and other natural or non-natural bodies of water. In embodiments, the aqueous sample contains bodily solids or fluids (e.g., feces or urine) from subjects who have been exposed to or infected with a virus herein (e.g., a coronavirus such as SARS-CoV-2). In embodiments, the environmental sample is from a air filtration device, e.g., air filters in a healthcare or long-term care facility or other communal places of gathering. Detection of a virus described herein (e.g., a coronavirus such as SARS-CoV-2) in an environmental sample can provide early identification and/or tracing of an outbreak or potential outbreak, thereby allowing a more prompt and robust response. Moreover, detection of a biomarker, e.g., one or more antibody biomarkers that specifically binds a viral antigen (e.g., from a coronavirus such as SARS-CoV-2) in an environmental sample can provide an estimation of the percentage of a population with detectable antibodies against the virus (i.e., seroconversion), which is useful for epidemiology studies. In some embodiments, the sample comprises wastewater. Detection of SARS-CoV-2 in wastewater is described, e.g., in U.S. Publication No. 2022/0003766 and U.S. Publication No. 2021/0349104.

Wastewater samples are also useful for determining the viral strain, i.e., the genotype, of SARS-CoV-2 in a population. SARS-CoV-2 strains are further described herein and include, e.g., the L strain and the S strain, which differ at genome locations 8782 and 28144; and the S-D614 strain and the S-D614G strain, which differ by a single polynucleotide at genome location 23403, and the strains described in Table 1A, e.g., strains B.1.1.7, 501Y.V2, P.1, and Cal.20C. In embodiments, the invention provides a method for detecting SARS-CoV-2 nucleic acid in a wastewater sample, comprising: a) contacting the wastewater sample with a binding reagent that specifically binds a SARS-CoV-2 nucleic acid; b) forming a binding complex comprising the binding reagent and the SARS-CoV-2 nucleic acid; and c) detecting the binding complex, thereby detecting the SARS-CoV-2 nucleic acid in the wastewater sample. In embodiments, the SARS-CoV-2 nucleic acid comprises a SARS-CoV-2 single nucleotide polymorphism (SNPs) or mutation as described herein, e.g., in Tables 1A and 1C. In embodiments, the method is a multiplexed method that simultaneously detects one or more, two or more, three or more, four or more, five or more, six or more, seven or more, eight or more, nine or more, or ten or more SARS-CoV-2 SNPs. Methods of detecting SNPs in viral nucleic acids, e.g., SARS-CoV-2 RNA, are provided herein. In embodiments, levels of IgA, IgG, and/or IgM in wastewater samples are used as controls for normalizing the detected amount of viral protein and/or genetic material (e.g., RNA) in the wastewater sample.

In embodiments where the sample comprises a liquid (e.g., endotracheal aspirate, saliva, blood, serum, plasma and the like), the sample is about 0.05 mL to about 50 mL, about 0.1 mL to about 10 mL, about 0.2 mL to about 5 mL, or about 0.3 mL to about 3 mL. In embodiments where the sample is solid or semi-solid (e.g., a swab such as a nasopharyngeal swab or oropharyngeal swab, mucus, sputum and the like), the sample is provided into a storage liquid of about 0.05 mL to about 50 mL, about 0.1 mL to about 10 mL, about 0.2 mL to about 5 mL, or about 0.3 mL to about 3 mL. In embodiments, the storage liquid is Viral Transport Medium (VTM), Amies transport medium, or sterile saline. In embodiments, the storage liquid comprises a substance for stabilizing nucleic acids, e.g., EDTA. In embodiments, the storage liquid comprises a reagent for inactivating live virus as described herein.

In embodiments, the sample comprises saliva. In embodiments, the invention provides a method of identifying a saliva sample in which the viral component and/or biomarker of interest has degraded, i.e., a low quality saliva sample. In embodiments, a low quality saliva sample is not suitable for the assays described herein. In embodiments, a low quality saliva sample comprises low levels of total antibodies as compared to a freshly obtained sample and/or as compared to a threshold total antibody level. In embodiments, a low quality saliva sample comprises low levels of IgA as compared to a freshly obtained sample and/or as compared to a threshold antibody level. In embodiments, the threshold antibody level is determined based on the average of an aggregate of samples. In embodiments, a low quality saliva sample comprises low levels of antibodies against circulating coronaviruses (e.g., hCoV-NL63, hCoV-HKU1, hCoV-229E, and/or hCoV-OC43) as compared to a freshly obtained sample and/or a threshold antibody level. In embodiments, identifying the low quality saliva sample comprises determining the total antibody level in a sample and, if the sample has low antibody levels as compared to a freshly isolated control sample and/or as compared to a threshold total antibody level, identifying the sample as a low quality saliva sample. In embodiments, identifying the low quality saliva sample comprises determining the IgA level in a sample and, if the sample has low IgA levels as compared to a freshly isolated control sample and/or as compared to a threshold antibody level, identifying the sample as a low quality saliva sample. In embodiments, identifying the low quality saliva sample comprises determining the levels of antibodies against one or more circulating coronaviruses in a sample and, if the sample has low antibody levels against the one or more circulating coronaviruses as compared to a freshly isolated control sample and/or a threshold antibody level, identifying the sample as a low quality saliva sample.

In embodiments, the sample comprises an extracellular vesicle. As described herein, extracellular vesicles (also known as EVs or exosomes) are small membrane vesicles released by most cell types, including immune cells and infected cells (e.g., by a respiratory virus described herein such as SARS-CoV-2). Detection and analysis of EVs are further described, e.g., in US 2022/0003766; US 2021/0349104; WO 2019/222708; and WO 2020/086751.

In embodiments, the sample is pretreated prior to being subjected to the methods provided herein. In embodiments, the sample is pretreated prior to being handled by, processed by, or in contact with laboratory and/or clinical personnel. In embodiments, pretreating the sample comprises subjecting the sample to conditions sufficient to inactivate live virus in the sample. Inactivation of live virus that may be present in the sample reduces the risk of infection of the laboratory and/or clinical personnel handling and/or processing the sample, e.g., by performing the methods described herein on the sample. In embodiments, pretreating the sample comprises heating the sample to at least 55° C., at least 56° C., at least 57° C., at least 58° C., at least 59° C., at least 60° C., at least 65° C., at least 70° C., at least 75° C., at least 80° C., at least 85° C., at least 90° C., at least 95° C., or at least 100° C. In embodiments, the sample is heated for about 10 minutes to about 4 hours, about 20 minutes to about 2 hours, or about 30 minutes to about 1 hour. In embodiments, the sample is heated to about 65° C. for at least 10 minutes. In embodiments, the sample is heated to about 65° C. for at least 30 minutes. In embodiments, the sample is heated to about 58° C. for at least 1 hour.

In embodiments, pretreating the sample comprises contacting the sample with an inactivation reagent. In embodiments, the inactivation reagent comprises a detergent, a chaotropic agent, a fixative, or a combination thereof. Non-limiting examples of detergents include sodium dodecyl sulfate and TRITON™ X-100. Non-limiting examples of chaotropic agents include guanidium thiocyanate, guanidium isothiocyanate, and guanidium hydrochloride. Non-limiting examples of fixatives include formaldehyde, formalin, paraformaldehyde, and glutaraldehyde. In embodiments, pretreating the sample comprises subjecting the sample to UV or gamma irradiation. In embodiments, pretreating the sample comprises subjecting the sample to a highly alkaline (e.g., above pH 10, above pH 11, or above pH 12) condition. In embodiments, pretreating the sample comprises subjecting the sample to a highly acidic (e.g., below pH 4, below pH 3, below pH 2) condition. Additional methods of pretreating samples, e.g., containing the viruses described herein, is further discussed in Bain et al., Curr Protoc Cytometry 93:e77 (2020).

In embodiments, the sample comprises a viral nucleic acid. In embodiments, the sample comprising the viral nucleic acid is pretreated with a reagent that stabilizes and/or prevents degradation of the viral nucleic acid. In embodiments, the pretreating comprises removing and/or inhibiting activity of a nuclease, e.g., an rNase, in the sample. In embodiments, the viral nucleic acid is SARS-CoV-2 RNA.

In embodiments, the sample comprises an RT-PCR product. In embodiments, the RT-PCR product comprises a cDNA that is generated from a viral RNA. In embodiments, the sample comprising the RT-PCR product is pretreated to remove the viral RNA and/or a reagent used in the RT-PCR. In embodiments, the pretreating comprises contacting the sample with rNase. In embodiments, the pretreating comprises heating the sample, e.g., as described herein. In embodiments, the viral RNA is SARS-CoV-2 RNA.

In embodiments, the sample is pretreated immediately after being collected, e.g., from a subject described herein. Sample collection methods are provided herein. In embodiments, the sample is pretreated while being transported to a facility, e.g., a laboratory, for processing and analyzing the sample, e.g. using the methods described herein. In embodiments, the sample is pretreated after arrival at a facility, e.g., a laboratory, for processing and analyzing the sample, e.g. using the methods described herein. In embodiments, the sample is pretreated prior to being stored. In embodiments, the sample is stored prior to processing and analysis, e.g. using the methods described herein. In embodiments, the sample is stored at about −80° C. to about 30° C., about −70° C. to about 25° C., about −60° C. to about 20° C., about −20° C. to about 15° C., about 0° C. to about 10° C., about 2° C. to about 8° C., or about 4° C. to about 12° C. Methods and conditions for storing the samples described herein are known to one of ordinary skill in the art.

As used herein, the term “exposure,” in the context of a subject being exposed to a virus, refers to the introduction of a virus into the subject's body. “Exposure” does not imply any particular amount of virus; introduction of a single viral particle into the subject's body can be referred to herein as an “exposure” to the virus. As used herein, the term “infection,” in the context of a subject being infected with a virus, means that the virus has penetrated a host cell and has begun to replicate, assemble, and release new viruses from the host cell. The term “infection” can also be used to refer to an illness or condition caused by a virus, e.g., respiratory tract infection as described herein.

In embodiments, the virus, viral component, and/or biomarker are detectable in a subject immediately (e.g., within seconds) after the subject is exposed to the virus and/or infected with the virus. In embodiments, the virus, viral component, and/or biomarker are detectable in a subject within about 5 minutes to about 1 year, about 1 hour to about 9 months, about 6 hours to about 6 months, about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject is exposed to the virus and/or infected with the virus. In embodiments, the virus, viral component, and/or biomarker are detectable in a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject is exposed to the virus and/or infected with the virus. Different biomarkers, e.g., antibody biomarkers or inflammatory or tissue damage response biomarkers, in the same subject may have a varying magnitude of change in response to virus exposure and/or infection, for example, depending on whether the biomarker is an acute response biomarker or a biomarker related to a long-term effect. For some viral infections, the antibody biomarker IgG typically plateaus after 10 days of disease onset and persist (e.g., potentially signifying longer-term immunity); the antibody biomarkers IgA and IgM are detectable within 6 days of disease onset, peak around 10 days, and diminish after approximately 14 days (e.g., as part of the initial infection response). Different viruses can trigger biomarker responses at different times. For example, antibodies to SARS-CoV-2 may not be consistently detected in a subject until about three weeks after infection, which is longer than the typical timing for other types of viral infections. The timing of producing the same biomarker type, e.g., IgM or IgG antibody, can also vary widely among different subjects. Thus, in embodiments, the methods for multiplexed assays for a combination of biomarkers disclosed herein includes a determination or consideration of the response timing of each of the biomarkers.

In embodiments, the biological sample is obtained from a subject who has not been exposed to the virus. In embodiments, the biological sample is obtained from a subject immediately (e.g., within seconds) after the subject is known or suspected to be exposed to the virus. In embodiments, the biological sample is obtained from a subject within about 5 minutes to about 1 year, about 1 hour to about 9 months, about 6 hours to about 6 months, about 12 hours to about 90 days, 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject is known or suspected to be exposed to the virus. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject is known or suspected to be exposed to the virus.

In embodiments, the biological sample is obtained from a subject prior to the subject showing any symptoms of a viral infection. In embodiments, the biological sample is obtained from a subject immediately (e.g., within seconds) after the subject begins to show symptoms of a viral infection. In embodiments, the biological sample is obtained from a subject within about 5 minutes to about 1 year, about 1 hour to about 9 months, about 6 hours to about 6 months, about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after the subject begins to show symptoms of a viral infection. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after the subject begins to show symptoms of a viral infection. Symptoms of a viral infection are described herein and include, e.g., cough, shortness of breath, fever, and fatigue.

In embodiments, the biological sample is obtained from a subject after the subject is diagnosed with a viral infection. As described herein, the SARS-CoV-2 virus can cause post-acute COVID-19 syndrome, with certain symptoms persisting weeks or months after the initial illness period. In embodiments, the biological sample is obtained from a subject after about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 9 months, about 1 year, about 2 years, about 3 years, about 4 years, about 5 years, about 6 years, about 7 years, about 8 years, about 9 years, about 10 years, or more than 10 years after the subject is diagnosed with the viral infection.

In embodiments, the biological sample is obtained from a subject prior to the subject being administered with a vaccine or a treatment for the virus described herein. In embodiments, the biological sample is obtained from a subject immediately (e.g., within seconds) after a vaccine or a treatment is administered to the subject. In embodiments, the biological sample is obtained from a subject within about 12 hours to about 90 days, about 1 day to about 60 days, about 2 days to about 50 days, about 3 days to about 40 days, about 4 days to about 30 days, about 5 days to about 28 days, about 6 days to about 25 days, about 7 days to about 22 days, or about 8 days to about 20 days after a vaccine or a treatment is administered to the subject. In embodiments, the biological sample is obtained from a subject within about 5 minutes, about 1 hour, about 3 hours, about 6 hours, about 12 hours, about 1 day, about 2 days, about 3 days, about 4 days, about 5 days, about 6 days, about 7 days, about 10 days, about 14 days, about 21 days, about 1 month, about 2 months, about 3 months, about 6 months, about 1 year, or more than 1 year after a vaccine or a treatment is administered to the subject.

Sample Pooling

Samples may be obtained from a single source described herein, or may contain a mixture from two or more sources, e.g., pooled from one or more individuals who may have been exposed to or infected by a particular virus in a similar manner. Sample pooling strategies are further described, e.g., in U.S. Publication No. 2022/0003766 and U.S. Publication No. 2021/0349104. For example, the individuals may live or have lived in the same household, visited the same location(s), and/or associated with the same people. In embodiments, samples are pooled from two or more, three or more, four or more, five or more, six or more, seven or more, eight or more, nine or more, ten or more, 15 or more, 20 or more, 25 or more, 30 or more, 40 or more, 50 or more, 100 or more, 150 or more, 200 or more, 300 or more, 400 or more, 500 or more, 1000 or more, 5000 or more, or 10000 or more individuals. For example, a “negative” result for an active viral infection from a pooled sample indicates that none of the individuals from the pooled sample have an active infection, which can significantly reduce the number of tests needed to test every individual in a population. In embodiments, the sample comprises a respiratory sample, e.g., bronchial/bronchoalveolar lavage, saliva, mucus, oropharyngeal swab, sputum, endotracheal aspirate, pharyngeal/nasal swab, throat swab, nasal secretion, or combination thereof. In embodiments, the sample comprises saliva. In embodiments, the sample comprises blood. In embodiments, the sample comprises serum or plasma. In embodiments, the virus is a coronavirus. In embodiments, the virus is SARS-CoV-2. In embodiments, a “positive” result for an active viral infection in the pooled sample prompts or indicates a need for further testing using the methods and/or kits provided by the invention of individual samples comprised in the pool of samples.

In embodiments, the pooled sample is subjected to a single layer pooling strategy. A “single layer pooling strategy,” as used herein, refers to testing a pooled sample, and if the result of the pooled sample is “positive” for an active viral infection, each individual sample comprised in the pooled sample is then individually tested, e.g., using the methods and/or kits provided in the invention. In embodiments, the pooled sample is subjected to a multi-layer pooling strategy, e.g., a two-layer pooling strategy. In a “multi-layer pooling strategy,” a pooled sample containing n number of individual sample is tested in a first round, and if the result of the first round is “positive” for an active viral infection, then the pooled sample is divided into smaller pools, e.g., wherein each smaller pool comprises a number of individual samples equal to the square root of n, and re-tested in a second round. The smaller pool(s) with the “positive” results can be further divided into even smaller pools for one or more additional rounds of testing until the positive individual samples are identified. In an exemplary two-layer pooling strategy, a pooled sample containing 100 individual samples is tested in a first round, and if the pooled sample is tested to be “positive” for an active viral infection, then the pooled sample is divided into pools containing 10 individual samples. Each individual sample comprised in any 10-sample pools that tested “positive” are then tested.

In embodiments, the invention provides a method for determining the number of individual samples to be included in a pooled sample. In embodiments, the number of individual samples included in a pooled sample is based on disease prevalence in a population. For example, if disease prevalence is high, the likelihood of a pooled sample, containing a large number of individual samples, testing “positive” is also high, which reduces the benefits of testing pooled samples because additional tests are required to determine the positive individual samples.

In embodiments, each individual sample is about 0.1 mL to about 10 mL, about 0.2 mL to about 5 mL, or about 0.3 mL to about 3 mL. In embodiments, about 1%, about 2%, about 3%, about 4%, about 5%, about 10%, or about 20% of the total volume of each individual sample is added to the pooled sample. In embodiments, about 1 μL to about 100 μL, about 5 μL to about 50 μL, or about 10 μL to about 20 μL of each individual sample is added to the pooled sample. In embodiments, the amount of each individual sample not added to the pooled sample is sufficient for one or more additional rounds of testing (e.g., in a multi-layered pooling strategy as described herein).

Collection and Assay Devices

In embodiments, the biological sample is a liquid sample. In embodiments, the biological sample is in contact with a sample collection device. In embodiments, the sample collection device is an applicator stick. In embodiments, the sample collection device comprises an elongated handle (e.g., a rod or a rectangular prism) and a sample collection head configured to collect sample from a biological tissue (e.g., from a subject's nasal or oral cavity) or a surface. In embodiments, the sample collection head comprises an absorbent material (e.g., cotton) or a scraping blade. In embodiments, the sample collection device is a swab. In embodiments, the sample collection device is a tissue scraper.

In embodiments, the disclosure provides a sample collection device for collecting a biological sample or any other sample described herein, which may contain analytes at a concentration too low to support an accurate or reliable analysis result. The sample collection device may be used to store the sample and/or to transport the sample to a laboratory or other site at which the sample may be analyzed via, e.g., an assay described herein, e.g., a classical, bridging, or competitive serology assay or an assay for detecting a virus, viral component, or biomarker. The sample collection device may be used to increase a concentration and/or purity of an analyte of interest (e.g., virus, viral component, or biomarker as described herein) in the sample, so as to facilitate an ability of the assay to detect or measure the analyte. In embodiments, the virus or viral component is SARS-CoV-2 or component thereof. In embodiments, the biomarker is an antibody that binds a SARS-CoV-2 protein or fragment thereof.

In various embodiments, the sample collection device may include a sample container for storing the sample, a solid phase binding material disposed within the sample container, and a container sealing component, such as a cap, for creating a seal around the sample container to prevent leakage of the container's content. The solid phase binding material may be used to increase a concentration of the analyte before the assay is performed. In some instances, the solid phase binding material may exhibit a relatively high level of affinity to the analyte. The affinity may cause the analyte to bind to the solid phase binding material in a higher concentration relative to the analyte's concentration in the sample. The higher concentration may lead to a more accurate and reliable assay result. In some instances, the solid phase binding material may increase a purity of the sample. The higher purity may be achieved, e.g., by binding the analyte of interest to the solid phase binding material, while washing out or otherwise removing components in the sample which may interfere with the accuracy of the assay and lead to a sample matrix effect. In embodiments, the analyte comprises a virus, viral component, or biomarker described herein. In embodiments, the virus or viral component is SARS-CoV-2 or component thereof. In embodiments, the biomarker is an antibody that binds a SARS-CoV-2 protein or fragment thereof.

In a further embodiment, the sample collection device may include a container sealing component which forms a compartment that holds a fluid, e.g., adapted to stabilize a property of the biological sample or adapted to resuspend the analyte bound to the solid phase binding material. The sample collection device may be adapted to cause the fluid to be released from the fluid compartment into the sample container when the container sealing component is attached to or is being attached to the sample container.

In a further embodiment, the container sealing component may be a cap that includes a membrane which forms at least part of the fluid compartment. The sample container may be adapted, when being attached to the container cap, to pierce the membrane to cause the fluid within the fluid compartment to be released into the space enclosed by the housing of the sample container.

In a further embodiment, the container sealing component may form a compartment which holds the solid phase binding material. The sample collection device may be adapted to cause the solid phase binding material to be released from the compartment into the sample container when the container sealing component is attached to or is being attached to the sample container.

In a further embodiment, the sample collection device may have a first opening located at a first end of the sample collection device, and have a second opening located at a second end of the sample collection device. The sample collection device in this embodiment may include a first container sealing component which is removably attached to the first end of the sample container, and include a second container sealing component which is removably attached to the second end of the sample container. The sample container is adapted, when an eluent is delivered into the sample container, to allow the eluent to flow from the first opening toward the second opening of the sample container.

In a further embodiment, the solid phase binding material may be disposed between the first opening and the second opening of the sample container. The sample collection device further may comprise a retention material, such as a porous frit, disposed between the solid phase binding material and the second opening of the sample container. The retention material may be adapted to retain the solid phase binding material within the sample container when the eluent is moving through the sample container.

In a further embodiment, the sample collection device may include a funnel for aiding collection of the biological sample, wherein the funnel is adapted to fit around the opening of the sample container, and to increase in width or diameter as the funnel extends in a direction away from the opening.

In a further embodiment, solid phase binding material forms a first pad disposed in the sample container. The sample collection device includes a second pad formed from a nonspecifically absorbent material, and disposed below the solid phase binding material.

In a further embodiment, the sample collection device includes a component to equalize pressure between opposite sides of a sample as liquid content of the sample flows through a sample container of the sample collection device.

The solid phase binding material may include a variety of materials, such as a ligand, antibodies, proteins adapted to bind specifically to antibodies, a matrix of beads, resin, or any other material.

The sample container may have a variety of shapes and volumes, such as the shape of a tube or column, and may have a volume in a variety of ranges, such as the range of 1 mL to 20 mL, 50 μL to 1 mL, or a volume greater than 20 mL.

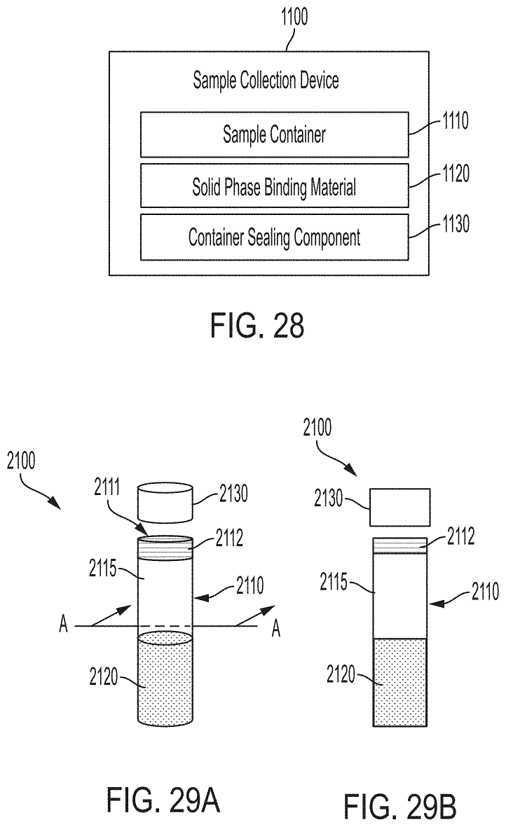

In embodiments, the sample collection device collects and stores a sample, such as a biological sample, and improves a concentration and/or purity of an analyte of interest in the collected sample, so as to facilitate detection and measurement of the analyte. FIG. 28 provides an example of a sample collection device 1100, which may include a sample container 1110, solid phase binding material 1120, and a container sealing component 1130. The sample container 1110 may be used to receive the sample, and may hold or otherwise store the sample, so as to facilitate transport of the sample from a sample collection site to an analysis site, such as a laboratory in which an assay (e.g., an assay described herein such as a classical, bridging, or competitive serology assay or an assay for detecting a virus, viral component, or biomarker) is performed on the sample. The sample may be, e.g., a biological sample (e.g., saliva, urine, sweat, tears, blood), an environmental sample, a food sample, or any other sample as described herein, e.g., that comprises a virus, viral component, or biomarker described herein. In embodiments, the virus or viral component is SARS-CoV-2 or component thereof. In embodiments, the biomarker is an antibody that binds a SARS-CoV-2 protein or fragment thereof. As illustrated below in more detail, the sample container 1110 may include or otherwise provide a housing for storing the sample. The housing of the sample container 1110 may form an opening for receiving the sample, and may enclose a space for holding the sample. In other words, the housing may form a compartment 2115 (see, e.g., FIG. 29A) for holding the sample. The sample container 1110 may be sealed via the container sealing component 1130, such as a cap, which may be removably attachable to the sample container. That is, the container sealing component 1130 may be removeable from the sample container 1110 so as to allow a sample to be deposited into or extracted from the opening of the container 1110, and may be attachable to the sample container 1110 so as to form a seal that prevents the sample or other content from leaking out of the sample container 1110.

In various embodiments, the solid phase binding material 1120 may be adapted to bind specifically to an analyte of interest (e.g., a protein, antibody, antigen, cell, or cell component). More particularly, the solid phase binding material 1120 may exhibit a high level of affinity to the analyte, relative to a level of affinity between the analyte and other materials. Thus, the solid phase binding material 1120 may be adapted to bind specifically to the analyte (if any) in the biological sample. The specific binding may also be referred to as selective binding, because the solid phase binding material may exhibit a relatively high level of affinity to the analyte, while exhibiting relatively low affinity or no affinity towards other materials. As a result, the solid phase binding material 1120 may bind specifically to the analyte, and may have a much lower level of binding (e.g., ten times lower) or no binding with other materials. In embodiments, the analyte comprises a virus, viral component, or biomarker described herein. In embodiments, the virus or viral component is SARS-CoV-2 or component thereof. In embodiments, the biomarker is an antibody that binds a SARS-CoV-2 protein or fragment thereof.

As stated above, the solid phase binding material 1120 may improve a concentration and/or purity of an analyte in a collected sample. In various embodiments, the collected sample may have a relatively low concentration of the analyte. For instance, a liquid biological sample such as urine or saliva may have a relatively high volume of liquid content, but a relatively low concentration of various analytes of interest, such as specific types of antibodies. The low concentration of the analyte may limit an accuracy or reliability of various assays, such as a lateral flow immunoassay performed directly using the sample. By exhibiting a relatively high level of affinity toward the analyte of interest, the solid phase binding material 1120 may draw the analyte from the liquid content by binding to the analyte, and thus may tend to concentrate the analyte in the solid phase binding material 1120. As discussed below in more detail, elution may be performed to release the analyte from the solid phase binding material 1120 in some embodiments. The elution may cause the analyte to be released by or into an eluent that flows through the solid phase binding material 1120, wherein the eluent may flow into, e.g., a multi-well plate on which an assay is performed. The concentration of the analyte in the eluent or other solution in the multi-well plate may be relatively high, because the solid phase binding material 1120 may have a relatively high concentration of the analyte, and because the elution may be performed in a manner that limits a total volume of the eluent. As further discussed below, the specific binding of the solid phase binding material 1120 to the analyte may help retain the analyte with the material 1120 during one or more washing steps, in which other material is removed, which may increase a purity of the analyte and remove components that may lead to a sample matrix effect or other inaccuracies in an assay. In embodiments, the analyte comprises a virus, viral component, or biomarker described herein. In embodiments, the virus or viral component is SARS-CoV-2 or component thereof. In embodiments, the biomarker is an antibody that binds a SARS-CoV-2 protein or fragment thereof.

FIG. 29A illustrates a perspective view of a sample collection device 2100, which may be an embodiment of the sample collection device 1100, while FIG. 29B provides a cross-sectional view of the sample collection device as viewed along the line A-A in FIG. 29A. The sample collection device 2100 in FIGS. 29A and 29B may include a sample container 2110, solid phase binding material 2120, and a container sealing component 2130 (which may be embodiments of the sample container 1110, solid phase binding material 1120, and container sealing component 1130, respectively, of FIG. 28).

In various embodiments, the sample container 2110 may form a tube, column, bottle, vial, or other structure which encloses a space or compartment for storing or otherwise holding a sample, and may have an opening 2111 for receiving the sample. The sample may be collected from a sample collection site, and stored in the sample container 2110 so that the sample can be transported to a laboratory or other analysis site. As stated above, the sample may be, e.g., a biological sample, an environmental sample, a food sample, or any other sample described herein, e.g., that comprises a virus, viral component, or biomarker described herein. In embodiments, the virus or viral component is SARS-CoV-2 or component thereof. In embodiments, the biomarker is an antibody that binds a SARS-CoV-2 protein or fragment thereof. For instance, the biological sample may include, e.g., saliva, blood, serum, plasma urine, wound exudate, a nasal swab, a nasopharyngeal mucosal swab, bronchial/bronchoalveolar lavage, mucus, oropharyngeal swab, sputum, endotracheal aspirate, a throat swab, nasal secretions, nasopharyngeal wash or aspirate, nasal mid-turbinate swab, or an extraction or purification therefrom, or dilution thereof. The environmental sample may include, e.g., drinking water, wastewater, soil extract, and/or a plant extract, such as a leaf swab. In some instances, the environmental sample may include air collected from a particular environment, so that air quality can be assessed. The sample container 2110 may have a volume that is large enough to hold a desired amount of the sample. For instance, the sample container 2110 may have a volume that is in a range from, e.g., 50 μL, to 1 mL (e.g., to collect a sample on which an assay can be directly performed), 1 mL to 20 mL (e.g., to collect a sample for which an analyte may be concentrated before an assay is performed), or a volume greater than 20 mL (e.g., to collect a wastewater sample). Further, the sample container 2110 may have a cylindrical shape, as illustrated in FIG. 29A, or may have any other shape (e.g., a rectangular shape).

In various embodiments, the container sealing component 2130 may be a cap which is removably attachable to the sample container 2110 at the opening 2111 of the container 2110, so as to form a seal around the opening 2111 when the container sealing component 2130 is attached to the container 2110. In this example, the sample container 2110 may form a threaded portion 2112 that is located at a first end (e.g., top end) of the sample container 2110. The container sealing component 2130 in such an example may have a portion adapted to mate with the threaded portion of the sample container 2110, so as to allow the cap to be screwed onto the threaded portion 2112, which forms the seal around the opening 2111. In some implementations, the container sealing component 2130 may include an O-ring, which may fit around the threaded portion 2112, so as to enhance the seal.

In various embodiments, the solid phase binding material 2120 of FIGS. 29A and 29B is disposed within the space enclosed by the housing of the sample container 2110. As stated above, the solid phase binding material 2120 may be adapted to specifically bind to an analyte of interest, e.g., a virus, viral component, or biomarker described herein. For instance, the solid phase binding material 2120 may be a binding reagent or affinity medium that is adapted to form a binding complex with the analyte. If a biological sample or other sample containing the analyte is deposited in the sample container 2110, the solid phase binding material may be adapted to bind specifically to the analyte in the biological sample. The solid phase binding material 2120 may thus form a purification module or purification matrix that is adapted to increase a level of concentration or purity of the analyte.

The solid phase binding material 2120 includes material which is in a solid phase. For instance, the solid phase binding material 2120 may form a powder, resin (e.g., cross-linked agarose resin), or matrix of beads (e.g., polystyrene-divinylbenzene beads) which provide an absorbent for binding to an analyte. For instance, the solid phase binding material may include a powder having particles which have a particle size that is in a range from 1 μm (micron) to 400 μm. In various embodiments, because the solid phase binding material 2120 has a solid phase, it may absorb liquid content in a sample. The absorption of the liquid may also enhance a concentration of an analyte of interest. For instance, if the analyte of interest is a type of antibodies (e.g., antibodies that bind to a SARS-CoV-2 protein or fragment thereof), such antibodies may be collected via saliva or other biological sample. The biological sample may have a relatively low concentration of such antibodies. Thus, while an assay may be performed directly on the collected saliva or other biological sample to attempt to detect or measure the antibodies, the low concentration of the antibodies in the biological sample may limit an accuracy of a detection result or measurement. The accuracy may further be limited by other components in the sample that may interfere with the detection or measurement of the analyte, which may lead to a sample matrix effect that can limit accuracy. In various embodiments, the solid phase binding material 2120 may address such limitations by absorbing liquid content in the sample and by binding specifically to the analyte of interest. The absorption may reduce a volume of liquid content in the sample, while the specific binding may tend to increase concentration of the analyte in the solid phase binding material. The solid phase binding material 2120 may be washed with an eluent at an analysis site, in preparation for performing an assay or other analysis. As an eluent flows through the solid phase binding material 2120, the eluent may cause the antibodies or other analyte to be released or otherwise disassociated from the solid phase binding material 2120 and be carried with the eluent into, e.g., a multi-well plate. In some implementations, the elution may be performed in a manner which controls a total volume of eluent that is used. For instance, an amount of eluent used in the elution may be less than an amount of liquid content originally present in the biological sample, which may further improve a concentration of the analyte. In embodiments, the analyte comprises a virus, viral component, or biomarker described herein. In embodiments, the virus or viral component is SARS-CoV-2 or component thereof. In embodiments, the biomarker is an antibody that binds a SARS-CoV-2 protein or fragment thereof.

As stated above, the solid phase binding material 2120 may exhibit affinity to an analyte of interest, such as a type of antibodies. Further, the level of affinity between the solid phase binding material and the analyte may be relatively high, compared to a level of affinity between the analyte and other materials. In some instances, the analyte of interest in a biological sample may be a type of antigen, such as a fragment of a virus. For example, the biological sample may be collected from a person to evaluate whether that person has been infected with the virus. In such instances, the solid phase binding material 2120 may include antibodies which are adapted to bind specifically to the antigen. In embodiments, the analyte comprises a virus or viral component described herein. In embodiments, the virus is a coronavirus. In embodiments, the virus is SARS-CoV-2.

In some instances, the analyte of interest may be a particular type of antibody, such as immunoglobulin G (IgG), IgA, and/or IgM antibodies, in a biological sample. For example, the biological sample may be collected from a person to evaluate an effectiveness of a vaccine in triggering that person to produce such antibodies. In such instances, the solid phase binding material 2120 may include, e.g., a protein (e.g., protein A, protein A/G, protein G, protein L) adapted to bind specifically to such antibodies. In embodiments, the analyte comprises an antibody biomarker that specifically binds a SARS-CoV-2 protein or fragment thereof.

Other examples of the solid phase binding material 2120 or its components include enzymes, ligands, receptors, nanomolecules, and resins which are adapted to bind specifically to an analyte of interest. For example, the nanomolecules may include nanotrap particles or dye molecules which have a relatively high level of binding to the analyte of interest. As another example, the resins may be, e.g., an ion-exchange resin or reverse-phase resin adapted to bind specifically to the analyte.

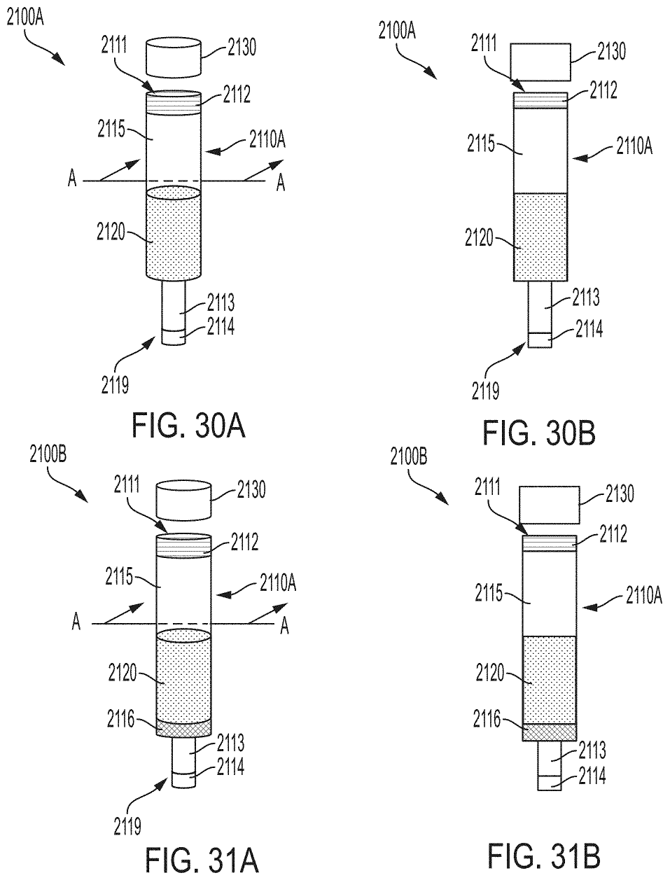

As discussed above, the analyte which is bound to the solid phase binding material 2120 may be eluted from the material 2120, so as to release the analyte for detection, measurement, or other analysis. FIGS. 30A and 30B illustrate a sample collection device 2100A which has openings 2111, 2119 at opposite ends of the device 2100A to facilitate an elution process that releases the analyte from the solid phase binding material 2120. The sample collection device 2100A may include the solid phase binding material 2120, a container 2110A, and container sealing components 2130, 2114. The sample container 2110A may have an opening 2111 at a first end (e.g., top end) of the container 2110A, and an opening 2119 at a second, opposite end (e.g., bottom end) of the container 2110A. The opening 2111 may lead to a compartment 2115 for holding or otherwise storing the solid phase binding material 2120 and a sample, while the sample container 2110A may include a passage 2113 that leads from the compartment 2115 to the opening 2119. In the example of FIGS. 30A and 30B, the passage 2113 may be formed from a tube, pipe, or other structure, and this structure may be narrower relative to the compartment 2115.

In various embodiments, container sealing component 2114 may be removably attachable to the second end of the container 2110A, so as to provide an ability to form a seal around the opening 2119 at the second end of the container 2110A. Further, as discussed above, the container sealing component 2130 may be removably attachable to the first end of the container 2110A, so as to provide an ability to form a seal around the opening 2111 at the first end of the container 2110A. When a sample is being transported and/or stored in the sample collection device 2100A, both container sealing components 2130, 2114 may be attached to the sample container 2110A, so as to prevent any content of the container 2110A from leaking out of the container 2110A. When an assay is to be performed on the biological sample, both of the container sealing components 2130, 2114 may be removed from the container 2110A, so as to allow for an elution process that causes the analyte to the released from the solid phase binding material 2120. The elution may be performed by causing an eluent to flow from the opening 2111 of the container 2110A toward the opening 2119 thereof, so that the eluent passes through the solid phase binding material 2120 to cause an analyte to be released from the solid phase binding material into the eluent.

In some instances, one or more washing steps may be performed on the solid phase binding material 2120 in a manner which filters out interfering components, such as contaminants which may interfere with the performance of an assay, so as to purify a sample before an eluent is passed through the sample. During the one or more wash steps, a wash fluid may be passed through the solid phase binding material 2120. The wash fluid may have relatively weak affinity or no affinity to the analyte, and thus may wash out interfering components while leaving the analyte bound to the solid phase binding material 2120. The analyte may later be released from the solid phase binding material 2120 during the elution step.

In various embodiments, a retention material may be used to retain the solid phase binding material 2120 within the sample container 2110A during elution. For example, FIGS. 31A-31B depict a sample collection device 2100B which includes the solid phase binding material 2120, sample container 2110A, and container sealing components 2130, 2114 discussed above. The sample collection device 2100B further includes a retention material 2116 disposed between the solid phase binding material 2120 and the opening 2119 at the second end of the container 2110A. If an analyte of interest is disassociated from the solid phase binding material 2120 during elution, the retention material 2116 may be adapted to an allow the analyte to be carried by an eluent out of the opening 2119 during elution, but may keep the solid phase binding material 2120 from also being carried out by the eluent. Thus, the retention material may provide a filter that prevents particles of the solid phase binding material 2120, or any other material which is not the analyte, from flowing out of the opening 2119. In some implementations, the retention material 2116 may include a porous frit which retains the solid phase binding material 2120 within the container 2110A. In one example, the porous frit may include particles with particle sizes that are, e.g., in a range of 0.1 μm to 400 μm, or more specifically in a range of 1 μm to 50 μm.

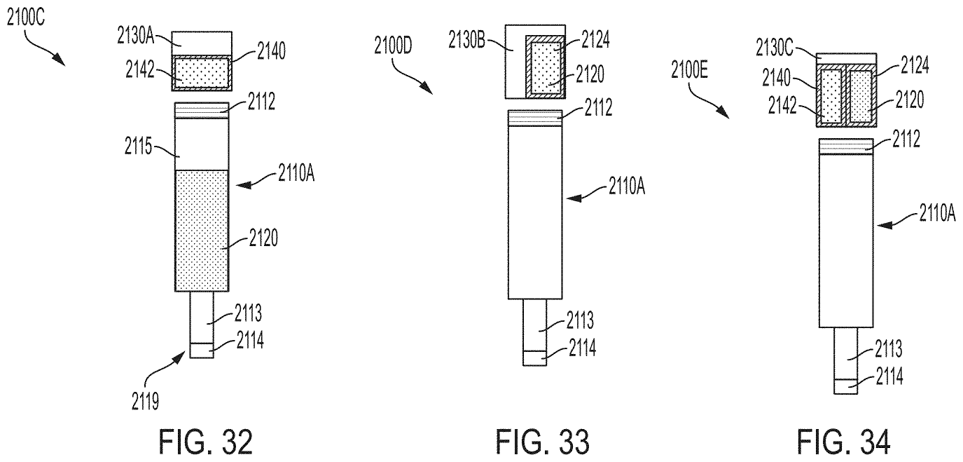

In various embodiments, a sample collection device may have a container sealing component that forms a compartment(s) for various material(s), wherein the compartment(s) may be disposed within or attached to the container sealing component. FIG. 32 depicts a sample collection device 2100C that includes the sample container 2110A and the solid phase binding material 2120 of the previous figures, and further includes a container sealing component 2130A which may form a compartment 2140 for holding or otherwise storing a stabilizer fluid 2142, which may be a fluid adapted to stabilize a property of a sample. For instance, stabilizer fluid 2142, also referred to as a storage solution, stabilizer solution or stabilizer buffer, may prevent or slow a rate of deterioration of the sample, wherein the deterioration may occur as a result of, e.g., denaturation, aggregation, and/or precipitation of an analyte or other components in the sample. In some implementations, the fluid 2142 may inhibit bacterial growth in the sample, maintain a pH of the sample at a specified level or within a specified range, and/or stabilize some other property in the sample, such as a level of folding and/or solubility of an analyte of interest in the sample. If the sample collection device 2100C is being transported to from a sample collection site to an analysis site (e.g., a laboratory), the stabilizer fluid 2142 may stabilize the sample while the device 2100C is in transit.

In various embodiments, the sample collection device 2100C may be adapted to cause the stabilizer fluid to be released from the compartment 2140 into the sample container 2110A when the container sealing component 2130A (e.g., cap) is attached to or is being attached to the sample container 2110A. For example, the container sealing component 2130A may be a cap that includes a membrane, such as a plastic film, that seals off a space within the cap from an external environment. The space may provide the compartment 2140 for the stabilizer fluid 2142, while the membrane may define at least part of a boundary of the compartment 2140. In this example, the sample container 2110A may be adapted, when being attached to the container sealing component 2130A, to pierce the membrane to cause the stabilizer fluid 2142 to be released from within the compartment 2140 into the space/compartment 2115 enclosed by the housing of the sample container 2110A. For instance, the sample collection device 2100A may have a threaded portion 2112 which is adapted to make contact with the membrane when the sample container 2110A is attached or being attached to the container sealing component 2130A. The threaded portion 2112 may be sufficiently sharp to pierce the membrane when the threaded portion 2112 makes contact with the membrane, so as to cause the stabilizer fluid 2142 to leak out of the compartment 2140 and into the compartment 2115 formed by the sample container 2110A.

In various embodiments, a container sealing component may include a compartment for holding or otherwise storing the solid phase binding material 2120. For example, FIG. 33 depicts a sample collection device 2100D which includes the sample container 2110A discussed above, and further includes a container sealing component 2130B (e.g., cap) that forms a compartment 2124 for holding the solid phase binding material 2120. In some instances, the compartment 2124 may include a solution for keeping the solid phase binding material 2120 hydrated. Like the discussion regarding FIG. 32, the compartment 2124 may similarly be formed by a membrane, which may define at least part of a boundary of the compartment 2124. The sample container 2110A in this embodiment may also be adapted to pierce the membrane when the container 2110A is attached or being attached to the container sealing component 2130B. As a result, the solid phase binding material 2120 may be released from the compartment 2124 into the sample container 2110A.

In various embodiments, both the solid phase binding material 2120 and the stabilizer fluid 2142 may be stored in a container sealing component. More specifically, FIG. 34 depicts a container sealing component 2130C which has both the compartment 2140 storing the stabilizer fluid 2142 and the compartment 2124 storing the solid phase binding material 2120. Like in the discussion of FIGS. 32 and 33, the sample container 2110A may be configured to pierce the compartments 2140, 2124 when the container 2110A is attached or is being attached to the container sealing component 2130C, so as to cause the stabilizer fluid 2142 and the solid phase binding material 2120 to be released into the container 2110A.

In some instances, a sample container of the embodiments herein (e.g., any one of 2100 through 2100G) may include the solid phase binding material 2120, which has affinity specifically to an analyte of interest (e.g., a virus, viral component, or biomarker described herein), and further include a pad (e.g., cotton pad) or other layer formed from a nonspecifically absorbent material adapted to absorb liquid content in a sample. For example, a sample collection device in such instances may include at least a first pad and a second pad that are disposed within a sample container. The first pad, such as a top pad, may be formed by the solid phase binding material, which may bind specifically to the analyte as a sample is deposited into the sample container and liquid content of the sample (if any) flows downward through the sample container. The second pad may be placed above or below the solid phase binding material of the first pad, and may include the nonspecifically absorbent material. The nonspecifically absorbent material may have general affinity to the liquid content and many different types of material in the liquid content, in a non-selective manner. The nonspecifically absorbent material may thus be adapted to draw liquid content in the sample toward itself. By drawing the liquid content toward itself, the second pad may promote flow or other movement of the liquid content in the sample through the sample container, which may in turn promote flow of the liquid content of the sample through the solid phase binding material. For example, the second pad may be placed below the solid phase binding material, which would cause the solid phase binding material to be sandwiched between the second pad and the opening 2111 of FIGS. 29A through 34. When a sample having liquid content, such as a saliva sample, is deposited into the sample container via the opening 2111, the second pad may absorb excess liquid content from the sample, wherein the excess liquid may include liquid content not absorbed by the solid phase binding material. Because the second pad is placed under the solid phase binding material, the absorption of the liquid content by the second pad in this example may promote flow of the liquid content through solid phase binding material. If the liquid content of the sample contains the analyte of interest, then promoting this flow of the liquid content may promote binding of an analyte to the solid phase binding material. In some implementations, the second pad may be considerably thicker (e.g., twice as thick) as the first pad, so as to promote absorption of the liquid content in the sample.

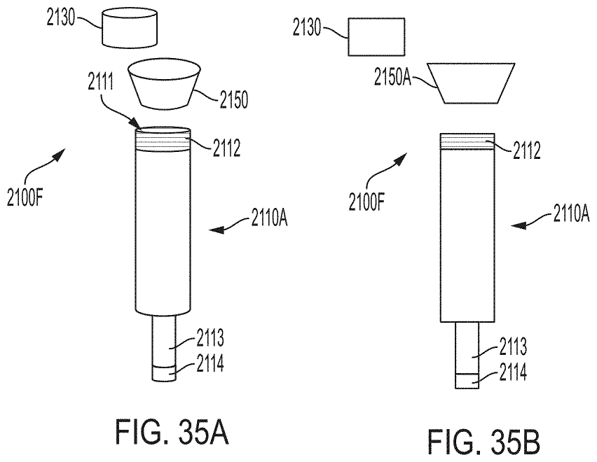

In various embodiments, depositing a sample (e.g., saliva sample) into a sample collection device may require a person or other host at a sample collection site to aim the sample toward an opening of the device's sample container. A sample collection aid may be included as part of the sample collection device to increase an ease by which the sample can be deposited into the container. The sample collection aid may include, e.g., a straw or a funnel which may be positioned by the person into alignment with the opening of the sample container. For example, FIGS. 35A and 35B depict a sample collection device 2100F that includes the sample container 2110A and the container sealing component 2130 discussed above with respect to FIGS. 30A and 30B, and further includes a funnel 2150. The funnel may have a relatively wide opening at one end of the funnel, so as to increase an ability of the funnel to collect a sample. Further, the funnel may narrow toward an opposite end thereof, so as to guide the sample into the sample container 2110A via the opening 2111 of the container 2110A.

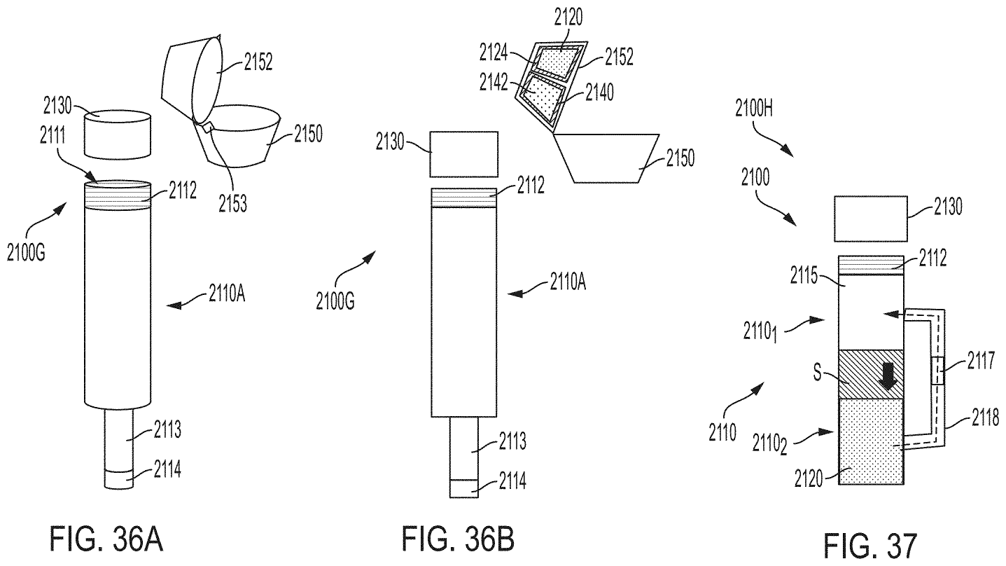

FIGS. 36A and 36B depict a sample collection device 2100G in which the stabilizer fluid 2142 and the solid phase binding material 2120 are disposed in compartments which are within or attached to a storage component 2152. In this example, the sample collection device 2100G may include the sample container 2110A, the container sealing component 2130, and the funnel 2150 of FIGS. 35A and 35B. The funnel 2150 may be part of a funnel assembly which includes the funnel 2150 and the storage component 2152. The storage component 2152 may provide a housing that forms the compartment 2140 for the stabilizer fluid 2142, and forms the compartment 2124 for the solid phase binding material 2120. The storage component 2152 may be attached to the funnel 2150 via a hinge or other attachment mechanism. The hinge may allow the storage component 2152 to be folded toward the funnel 2150 and folded away from the funnel 2150. In some implementations, the compartments 2140 and 2124 may be formed with membranes, and the funnel assembly in these implementations may include a component or structure 2153 (e.g., a sharp structure) which is adapted to pierce the membranes when the storage component 2152 is folded toward the funnel 2150. In this embodiment, a person may use the funnel 2150 of the funnel assembly as a sample delivery aid to deposit a sample into the sample container 2110A. Before or after the person delivers the sample into the sample container 2110A, the person may cause the membranes to be pierced, such as by folding the storage component 2152 toward the funnel 2150, so that the stabilizer fluid 2142 and the solid phase binding material 2120 are released into the sample container 2110A. After the sample, the stabilizer fluid 2142, and the solid phase binding material 2120 are deposited into the sample container 2110A, the container 2110A may be sealed via the container sealing component 2130.

In various embodiments, a sample collection device may have a mechanism to equalize pressure as liquid content of a sample flows or otherwise moves through a sample container, especially when the sample container is sealed. For instance, if the sample container has a vertical orientation, the liquid content may flow downward while passing through the solid phase binding material. As the liquid content flows downward, the air or other gas beneath the liquid content may become compressed and build up in pressure, while the air or other gas above the liquid content may expand and decrease in pressure. This pressure difference between opposite sides of the liquid content may stop or slow movement of the liquid content of the sample through the sample container, and more specifically through the solid phase binding material, which may impair an ability of the solid phase binding material to bind to the analyte.

In some implementations, the sample container may include a one-way air valve to compensate for the pressure difference on opposite sides of the sample. For instance, FIG. 37 depicts a sample collection device 2100H which has the sample container 2110 and the container sealing component 2130, and further includes a tube 2118 and a one-way air valve 2117. As liquid content of a sample S flows downward from a top portion 21101 of the sample container 2110 toward a bottom portion 21102 of the container 2110, the tube 2118 and/or air valve 2117 may aid in equalizing air pressure between the top portion 21101 and the bottom portion 21102. More particularly, the one-way air valve 2117 in this example may allow air or other gas to travel from one portion of the sample container 2110, such as the bottom portion 21102 that is below the liquid content of the sample S, to another portion, such as the top portion 21101 of the sample container which is above the liquid content of the sample S. Further, the one-way air valve 2117 may limit or prevent passage of air in the other direction. In such an example, as liquid content from a sample S flows downward, the downward flow may cause air pressure below the liquid content to rise above air pressure above the liquid content. This may create a pressure difference that opposes the downward flow of the sample S, which may interfere with an ability of the solid phase binding material 2120 to bind to an analyte in the sample S. The one-way air valve 2117 in this example may allow the higher air pressure to push air from a bottom portion 21102 of the sample container into a top portion 21101 of the sample container, which may cause top portion 21101 and the bottom portion 21102 of the sample container 2120 to equalize in terms of air pressure. In one implementation, the sample collection device may include a tube 2118. The tube 2118 may be provided in addition to the air valve 2117, as illustrated in FIG. 37, or may be provided instead of the one-way air valve. The tube 2118 may permit flow of air from the bottom portion 21102 of the sample container to the top portion 21101 of the sample container 2110, so as to equalize air pressure between the top portion 21101 of the container and the bottom portion 21102 of the container 2110.

In embodiments, the sample collection device or the liquid sample is contacted with an assay cartridge. Assay cartridges are further described in, e.g., U.S. Publication No. 2022/0003766 and U.S. Publication No. 2021/0349104. Assay cartridges may be used with assay cartridge readers known in the art. An exemplary assay cartridge reader is the MSD® Cartridge Reader instrument. Further exemplary assay cartridges and assay cartridge readers are described, e.g., in U.S. Pat. Nos. 9,921,166; 10,184,884; 9,731,297; 8,343,526; 10,281,678; 10,272,436; US 2018/0074082; and US 2019/0391170.

In embodiments, the method is performed in an assay plate. Assay plates are known in the art and described, e.g., in U.S. Publication No. 2022/0003766 and U.S. Publication No. 2021/0349104. Further exemplary assay plates are disclosed in, e.g., U.S. Pat. Nos. 7,842,246; 8,790,578; and 8,808,627. In embodiments, the assay plate result is read in a plate reader, e.g., the MESO® QUICKPLEX® or MESO® SECTOR® instruments.

In embodiments, the method is performed on a particle. Particles known in the art, e.g., as described in U.S. Publication No. 2022/0003766 and U.S. Publication No. 2021/0349104, can be used in conjunction with the methods and kits described herein. In embodiments, the particle comprises a microsphere.

Further exemplary devices for performing the methods herein include, but are not limited to, cassettes, measurement cells, dipsticks, reaction vessels, and assay modules described in, e.g., U.S. Pat. Nos. 8,298,934 and 9,878,323.

Assay Methods and Components

The viruses, viral components, and/or biomarkers described herein can be measured using a number of techniques available to a person of ordinary skill in the art, e.g., direct physical measurements (e.g., mass spectrometry) or binding assays (e.g., immunoassays, agglutination assays and immunochromatographic assays). Exemplary methods are described in, e.g., U.S. Publication No. 2022/0003766 and U.S. Publication No. 2021/0349104.

Exemplary binding assay methods include sandwich or competitive binding assays. Examples of sandwich immunoassays are described in U.S. Pat. Nos. 4,168,146 and 4,366,241. Examples of competitive immunoassays include those described in U.S. Pat. Nos. 4,235,601; 4,442,204; and 5,208,535.

Multiple viruses, viral components, and/or biomarkers can be measured using a multiplexed assay format, e.g., as described in US 2022/0003766; US 2021/0349104; US 2003/0113713; US 2003/0207290; US 2004/0022677; US 2004/0189311; US 2005/0052646; US 2005/0142033; US 2006/0069872; U.S. Pat. Nos. 5,807,522; 6,110,426; 6,977,722; 7,842,246; 10,189,023; and 10,201,812.

The methods herein can be conducted in a single assay chamber, such as a single well of an assay plate. The methods herein can also be conducted in an assay chamber of an assay cartridge as described herein. The assay modules, e.g., assay plates or assay cartridges, methods and apparatuses for conducting assay measurements suitable for the present invention, are described, e.g., in U.S. Pat. Nos. 8,343,526; 9,731,297; 9,921,166; 10,184,884; 10,281,678; 10,272,436; US 2004/0022677; US 2004/0189311; US 2005/0052646; US 2005/0142033; US 2018/0074082; and US 2019/0391170.

Binding

Binding reagents that specifically bind to viruses, viral components, and/or biomarkers are described herein and, e.g., in U.S. Publication No. 2022/0003766 and U.S. Publication No. 2021/0349104. In embodiments where the method comprises quantifying the amounts of one or more biomarkers capable of binding to a viral antigen (e.g., an antibody biomarker), the binding complex comprises the binding reagent and the antibody biomarker. In embodiments, the binding reagent is immobilized on a binding domain. In embodiments, the binding complex is formed on the binding domain.

In embodiments where the method is a multiplexed immunoassay method, more than one binding complex is formed, and each binding complex comprises a different binding reagent and its binding partner (e.g., a biomarker described herein). Multiplexed immunoassay methods are described herein and, e.g., in U.S. Publication No. 2022/0003766 and U.S. Publication No. 2021/0349104. In embodiments, each of the binding reagents are immobilized on separate binding domains. In embodiments, each binding domain comprises a targeting agent capable of binding to a targeting agent complement, wherein the targeting agent complement is connected to a linking agent, and each binding reagent comprises a supplemental linking agent capable of binding to the linking agent.

In embodiments, an optional bridging agent, which is a binding partner of both the linking agent and the supplemental linking agent, bridges the linking agent and supplemental linking agent, such that the binding reagents, each bound to its respective targeting agent complement, are contacted with the binding domains and bind to their respective targeting agents via the bridging agent, the targeting agent complement on each of the binding reagents, and the targeting agent on each of the binding domains.

In embodiments, the targeting agent and targeting agent complement, and the linking agent and supplemental linking agent, are each two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and targeting agent complement are cross-reactive moieties, e.g., thiol and maleimide or iodoacetamide; aldehyde and hydrazide; or azide and alkyne or cycloalkyne. In embodiments, the targeting agent is biotin, and the targeting agent complement is avidin or streptavidin. In embodiments, the linking agent is avidin or streptavidin, and the supplemental linking agent is biotin. In embodiments, the targeting agent and targeting agent complement are complementary oligonucleotides. In embodiments, the targeting agent complement is streptavidin, the targeting agent is biotin, and the linking agent and the supplemental linking agent are complementary oligonucleotides.

In embodiments, each binding domain is an element of an array of binding elements. In embodiments, the binding domains are on a surface. In embodiments, the surface is a plate. In embodiments, the surface is a well in a multi-well plate. In embodiments, the array of binding elements is located within a well of a multi-well plate. Non-limiting examples of plates include the MSD® SECTOR™ and MSD QUICKPLEX® assay plates, e.g., MSD® GOLD™ 96-well Small Spot Streptavidin plate. In embodiments, the surface is a particle. In embodiments, the particle comprises a microsphere. In embodiments, the particle comprises a paramagnetic bead. In embodiments, each binding domain is positioned on one or more particles. In embodiments, the particles are in a particle array. In embodiments, the particles are coded to allow for identification of specific particles and distinguish between each binding domain. In embodiments, the surface is an assay cartridge surface. In embodiments, each binding domain is positioned in a distinct location on the assay cartridge surface.

Detection

In embodiments, the method further comprises detecting the binding complex described herein. In embodiments, the binding complex comprising a binding reagent and its binding partner (e.g., a biomarker described herein) further comprises a detection reagent. In embodiments, the detection reagent specifically binds to the biomarker described herein. Detection methods are known in the art and further described, e.g., in U.S. Publication No. 2022/0003766 and U.S. Publication No. 2021/0349104.

In embodiments, the method comprises contacting the binding reagent with its binding partner and the detection reagent simultaneously or substantially simultaneously to form a binding complex. In embodiments, the method comprises contacting the binding reagent with its binding partner and the detection reagent sequentially to form a binding complex. In embodiments, the method comprises contacting the detection reagent with its binding partner and the binding reagent sequentially. In embodiments, the binding partner comprises a biomarker, e.g., antibody biomarker described herein.

In embodiments, the detection reagent is an antibody, antigen, ligand, receptor, oligonucleotide, hapten, epitope, mimotope, or aptamer. In embodiments, the detection reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, detection reagent comprises at least one heavy or light chain complementarity determining region (CDR) of an antibody. In embodiments, the detection reagent comprises at least two CDRs from one or more antibodies. In embodiments, the detection reagent is an antibody or antigen-binding fragment thereof. In embodiments where the method comprises detecting and/or quantifying the amounts of one or more biomarkers capable of binding to a viral antigen (e.g., an antibody biomarker), the detection reagent comprises an antigen (e.g., a viral protein described herein).

In embodiments, the detection reagent comprises a detectable label. In embodiments, measuring the concentration of the biomarkers in each of the binding complexes comprises measuring the presence and/or amount of the detectable label. In embodiments, the detectable label is measured by light scattering, optical absorbance, fluorescence, luminescence, chemiluminescence, electrochemiluminescence (ECL), bioluminescence, phosphorescence, radioactivity, magnetic field, or combination thereof. In embodiments, the detectable label comprises an electrochemiluminescence label. In embodiments, the detectable label comprises ruthenium. In embodiments, measuring the concentration of the biomarkers comprises measuring the presence and/or amount of the detectable label by electrochemiluminescence. In embodiments, the measuring of the detectable label comprises measuring an electrochemiluminescence signal.

In embodiments, detection reagent comprises a nucleic acid probe. In embodiments, the immunoassay further comprises binding the nucleic acid probe to a template oligonucleotide and extending the nucleic acid probe to form an extended sequence. In embodiments, the extended sequence binds to an anchoring reagent immobilized on the surface comprising the binding reagent. In embodiments, the virus, viral component, and/or biomarker is detected and/or quantified by detecting or quantifying the amount of extended sequence bound to the surface. In embodiments, the surface is contacted with a labeled probe that binds to the extended sequence, wherein the labeled probe comprises a detectable label.

In embodiments, the binding complex comprising the binding reagent and its binding partner (e.g., a biomarker described herein) further comprises a first detection reagent and a second detection reagent. In embodiments, the first detection reagent comprises a first nucleic acid probe, and the second detection reagent comprises a second nucleic acid probe. In embodiments, the immunoassay method further comprises binding the first and second nucleic acid probes to a template oligonucleotide and extending the second nucleic acid probe to form an extended sequence. In embodiments, the extended sequence binds to an anchoring reagent immobilized on the surface comprising the binding reagent. In embodiments, the biomarker is detected and/or quantified by detecting or quantifying the amount of extended sequence bound to the surface. In embodiments, the surface is contacted with a labeled probe that binds to the extended sequence, wherein the labeled probe comprises a detectable label. Detection methods are further described, e.g., in WO2014/165061; WO2014/160192; WO2015/175856; WO2020/180645; U.S. Pat. No. 9,618,510; U.S. Ser. No. 10/908,157; and U.S. Ser. No. 10/114,015.

In embodiments, the immunoassay is described in U.S. Publication No. 2022/0003766 and U.S. Publication No. 2021/0349104 and comprises:

(a) contacting a biotinylated binding reagent and a biotinylated anchoring reagent with a surface comprising streptavidin or avidin, e.g., for about 30 minutes to about 2 hours at room temperature, or about 6 hours to about 12 hours at 4° C.; and optionally washing the surface to remove unbound binding reagent and/or anchoring reagent;

(b) contacting a sample comprising the analyte of interest (e.g., biomarker described herein) with the surface, e.g., for about 1 hour to about 2 hours at about 20° C. to about 35° C. (e.g., about 27° C.); and optionally washing the surface to remove unbound analyte;

(c) contacting a detection reagent comprising a nucleic acid probe with the surface, e.g., for about 30 minutes to about 1 hour at about 20° C. to about 35° C. (e.g., about 27° C.), thereby forming a binding complex comprising the binding reagent, the analyte, and the detection reagent; and optionally washing the surface to remove unbound detection reagent;

(d) contacting a template oligonucleotide with the surface and ligating the template oligonucleotide to form a circular template, e.g., for about 10 minutes to about 30 minutes at about 20° C. to about 35° C. (e.g., about 27° C.), thereby hybridizing the nucleic acid probe to the circular template; and optionally washing the surface to remove excess template oligonucleotide;

(e) incubating the surface under conditions sufficient to perform rolling circle amplification, e.g., for about 5 minutes to about 30 minutes, thereby forming an extended sequence that binds to the anchoring reagent;

(f) contacting a labeled probe comprising a detectable label with the surface, e.g., for about 1 hour to about 2 hours at about 20° C. to about 35° C. (e.g., about 27° C.), thereby binding the labeled probe to the extended sequence; and optionally washing the surface to remove excess labeled probe; and

(g) measuring the amount of extended sequence by quantifying the amount of detectable label, thereby detecting and/or measuring the amount of analyte (e.g., biomarker described herein) in the sample.

In embodiments, the surface comprising the binding domains described herein comprises an electrode. In embodiments, the electrode is a carbon ink electrode. In embodiments, the measuring of the detectable label comprises applying a potential to the electrode and measuring electrochemiluminescence. In embodiments, applying a potential to the electrode generates an electrochemiluminescence signal. In embodiments, the strength of the electrochemiluminescence signal is based on the amount of detected analyte, e.g., biomarker described herein, in the binding complex.

In embodiments, the immunoassay described herein further comprises measuring the concentration of one or more calibration reagents. In embodiments, a calibration reagent comprises a known concentration of a biomarker described herein. In embodiments, the calibration reagent comprises a mixture of known concentrations of multiple biomarkers. Measurement of calibration reagents is known in the art and further described, e.g., in U.S. Publication No. 2022/0003766 and U.S. Publication No. 2021/0349104.

Competitive Assays

In embodiments, the methods provided herein are in a competitive assay format. In general terms, a competitive assay, e.g., a competitive immunoassay or a competitive inhibition assay, an analyte (e.g., a biomarker described herein) and a competitor compete for binding to a binding reagent (e.g., a viral antigen described herein). In such assays, the analyte is typically indirectly measured by directly measuring the competitor. As used herein, “competitor” refers to a compound capable of binding to the same binding reagent as an analyte, such that the binding reagent can only bind either the analyte or the competitor, but not both. In embodiments, competitive assays are used to detect and measure analytes that are not capable of binding more than one binding reagents, e.g., small molecule analytes or analytes that do not have more than one distinct binding sites. Examples of competitive immunoassays include those described in U.S. Pat. Nos. 4,235,601; 4,442,204; and 5,028,535.

Assays for Antibody Biomarkers

In embodiments where the biomarker detected is an antibody, e.g., an antibody capable of binding to a viral antigen such as S, S1, S2, S-NTD, S-ECD, S-RBD, M, E, or N, the binding reagent is an antigen that is bound by the antibody biomarker. In embodiments, antibody biomarkers are detected using a bridging serology assay. In a bridging serology assay, the binding complex further comprises a detection reagent described herein, and both the binding reagent and the detection reagent are an antigen that that is bound by the antibody biomarker. Since antibodies are typically bivalent, the antibody biomarker can bind both the binding reagent antigen and the detection reagent antigen.

In embodiments, antibody biomarkers are detected using a regular bridging serology assay. In a regular bridging serology assay, the antibody biomarker, binding reagent antigen, and detection reagent antigen are incubated together to form a complex where the antibody biomarker bivalently binds both the binding reagent antigen and the detection reagent antigen, e.g., a bridged complex. The incubation can be performed in any appropriate container, for example, in the well of a polypropylene plate, or in a chamber of an assay cartridge. In embodiments, the binding reagent antigen is conjugated to a biotin, and the bridged complex solution can be transferred to contact a surface comprising streptavidin, e.g., a streptavidin plate. In this embodiment, the biotin conjugated to the binding reagent antigen binds to the streptavidin plate, causing the entire bridged complex to be immobilized on the streptavidin plate.

In embodiments, antibody biomarkers are detected using a stepwise bridging serology assay. In a first step of a stepwise bridging serology assay, the binding reagent antigen is first immobilized on a surface. In embodiments where the binding reagent antigen is conjugated to biotin, the binding reagent antigen can be immobilized on a streptavidin plate. In a second step, after the binding reagent antigen is immobilized on the surface, a solution containing the antibody biomarker is contacted with the surface, allowing the first bivalent position on the antibody biomarker to bind the binding reagent antibody. In a third step, the detection reagent antigen is then contacted with the surface, allowing the second bivalent position on the antibody to bind the detection reagent antibody. In this stepwise method, the bridging complex is formed stepwise on the surface, rather than forming the entire bridging complex before immobilization, as is done in the regular bridging assay described above. In the stepwise bridging assay, the surface may optionally be rinsed or washed between any of the steps.

In either of the regular bridging serology assay or stepwise bridging serology assay, a method may be used where the detectable label is not directly conjugated to the detection reagent antigen but is instead attached to the detection antigen reagent using a binding complex such as streptavidin/biotin or other binding pair. The advantage of using this method is that it is not necessary to prepare separately conjugated binding reagent antigen and detection reagent antigen. In a non-limiting example of this method, a biotin conjugated antigen is prepared. Some of this biotin conjugated antigen is then incubated with a detectable label conjugated with streptavidin. The binding of biotin to streptavidin causes the detectable label to become attached to the biotin conjugated antigen, creating a detection reagent antigen comprising a detectable label as follows:

Antigen—biotin—streptavidin—detectable label

In embodiments, additional free biotin is added to the antigen—detectable label reagent to fully occupy the streptavidin binding sites and prevent other biotin conjugates from binding to the antigen—detectable label reagent. An additional amount of the biotin conjugated antigen, which is not attached to a detectable label, is then used as the binding reagent antigen. Binding reagent antigen and detection reagent antigen prepared in this way may be used in any of the assay methods described herein.

In embodiments, the antibody biomarker is detected using a classical serology assay. In embodiments of a classical serology assay, the binding reagent is an antigen that is bound by the antibody biomarker. After the antibody biomarker is bound by the binding reagent antigen, the binding complex is detected using a detection reagent antibody that binds the antibody biomarker. In embodiments, the detection reagent antibody is an anti-human antibody that binds human antibody biomarkers. In embodiments, the detection reagent antibody is an anti-human IgG, an anti-human IgM or an anti-human IgA isotype antibody. In embodiments, the detection reagent antibody is an anti-mouse antibody that binds mouse antibody biomarkers, or an anti-rat antibody that binds rat antibody biomarkers, or an anti-ferret antibody that binds ferret antibody biomarkers, or an anti-minx antibody that binds minx antibody biomarkers, or an anti-bat antibody that binds bat antibody biomarkers. In embodiments, the detection reagent antibody is an anti-mouse IgG, IgM, or IgA antibody, an anti-rat IgG, IgM, or IgA antibody, an anti-ferret IgG, IgM, or IgA antibody, an anti-minx IgG, IgM, or IgA antibody, or an anti-bat IgG, IgM, or IgA antibody.

In embodiments, the antibody biomarker is detected using a competitive serology assay (also termed a neutralization serology assay). Competitive immunoassays are described herein. In embodiments of a competitive serology assay, the binding reagent is an antigen that is bound by the antibody biomarker and by a competitor. In embodiments, the competitor is a substance that binds a specific region of the viral antigen. In embodiments, the competitor is a recombinant antibody or antigen-binding fragment thereof that binds specifically to an epitope of the viral antigen, e.g., a neutralizing epitope. In embodiments, the competitor is a monoclonal antibody against an epitope of the viral antigen, e.g., a neutralizing epitope. In embodiments, the competitor comprises a detectable label described herein. For example, the biomarker can be an antibody that binds specifically to a coronavirus spike protein, and the competitor can be the ACE2 receptor, NRP1 receptor, or CD147, i.e., natural interaction partners of the spike protein. In embodiments, the competitor is the ACE2 receptor. In embodiments, the receptor is the NRP1 receptor. In embodiments, the competitor is CD147. In embodiments, the competitor comprises a sialic acid. In embodiments, the binding reagent is a substance that binds a viral antigen (e.g., ACE2, NRP1, or CD147), and the competitor is the viral antigen (e.g., spike protein or a variant thereof described herein, such as, e.g., S1, S2, S-NTD, S-ECD, or S-RBD). In embodiments, the coronavirus is SARS-CoV-2. In embodiments, a competitive serology assay as described herein is used to assess a potential protective serological response, e.g., the ability of the immune response to block binding of a viral antigen to its host cell receptor such as ACE2, NRP1, or CD147.

In embodiments, the antibody biomarker serology assay (either bridging, classical, or competitive) described herein comprises measuring the concentration of one or more calibration reagents. In embodiments, the calibration reagent is a positive control. In embodiments, the positive control comprises an antigen for which an antibody is known or expected to be present in the biological sample. In embodiments, the positive control comprises an antigen from a prevalent influenza strain, to which most subjects are expected to have antibodies. In embodiments, the positive control is an antigen from the H1 Michigan influenza virus. In embodiments, the positive control is immobilized in a binding domain of a surface that further comprises one or more viral antigens immobilized thereon in one or more additional binding domains, as described herein. In embodiments, antibody biomarker serology assay further comprises measuring the total levels of a particular antibody, e.g., total IgG, IgA, or IgM.

In embodiments, the calibration reagent is a negative control. In embodiments, the negative control comprises an antigen for which no antibodies are expected to be present in the biological sample. In embodiments, the negative control comprises a substance obtained from a non-human subject, and the biological sample is obtained from a human subject. In embodiments, the negative control comprises bovine serum albumin (BSA). In embodiments, the negative control, e.g., BSA, is immobilized in a binding domain of a surface that further comprises one or more viral antigens immobilized thereon in one or more additional binding domains, as described herein.

In embodiments, the calibration reagent comprises a combination of biological samples from subjects known to be infected or exposed to a virus described herein. In embodiments, the calibration reagent comprises a pooled sample of serum and/or plasma from subjects known to be infected or exposed to a virus described herein. In embodiments, the calibration reagent is the same biological material as the sample to be assayed. For example, if the biological sample for the antibody biomarker serology assay is a serum sample, then the calibration reagent is a pooled serum sample. Similarly, if the biological sample for the antibody biomarker serology assay is a plasma sample, then the calibration reagent is a pooled plasma sample. In embodiments, the pooled sample comprises a known amount of IgG, IgA, and/or IgM that specifically bind to one or more viral antigens of interest. Methods of measuring IgG, IgA, and/or IgM concentration in a serum or plasma sample is known in the art, e.g., as described in Quataert et al., Clinical and Diagnostic Laboratory Immunology 2(5):590-597 (1995). In embodiments, the antibody biomarker serology assay comprises measuring the concentration of viral antigen-specific IgG, IgA, and/or IgM in multiple pooled samples to provide a calibration curve. In embodiments, the antibody biomarker serology assay comprises measuring the concentration of viral antigen-specific IgG, IgA, and/or IgM in multiple pooled samples, wherein the multiple pooled samples correspond to high, medium, and low levels of viral antigen-specific IgG, IgA, and/or IgM (referred to herein as “high pooled sample,” “medium pooled sample,” and “low pooled sample,” respectively). In embodiments, the pooled sample comprises serum and/or plasma from subjects known to never have been exposed to a virus described herein, i.e., a negative pooled sample. Pooled samples as calibration reagents allow baseline immune response thresholds to be defined and provides a better understanding of the levels of antibody response to a viral infection. In embodiments, the virus is a coronavirus. In embodiments, the virus is SARS-CoV-2.

In embodiments, the biological sample for the antibody biomarker serology assay is a saliva sample, and the calibration reagent comprises a calibration saliva sample. In embodiments, the calibration saliva sample contains a known amount of viral antigen-specific IgG, IgA, and/or IgM. In embodiments, the calibration saliva sample comprises serum from a subject known to be infected or exposed to a virus described herein. In embodiments, the calibration saliva sample comprises about 0.1% to about 1% of high pooled serum sample described herein. In embodiments, the calibration saliva sample comprises about 0.1%, about 0.2%, about 0.3%, about 0.4%, or about 0.5% of high pooled serum sample described herein. In embodiments, the calibration saliva sample comprises levels of viral antigen-specific IgG, IgA, and/or IgM equivalent to a 1:500 dilution of the high pooled serum sample as described herein. In embodiments, the calibration saliva sample is obtained from a subject known to never have been exposed to a virus described herein, i.e., a negative saliva sample. In embodiments, the calibration saliva sample provides a consistent threshold for comparing viral antigen-specific IgG, IgA, and/or IgM levels in saliva samples. In embodiments, the virus is a coronavirus. In embodiments, the virus is SARS-CoV-2.

In embodiments, the calibration reagents, e.g., the pooled sample and/or the calibration saliva sample described herein, is subjected to an antibody biomarker serology assay, e.g., the classical, bridging, and/or competitive serology assays described herein. In embodiments, the assay comprises measuring the total amount of IgG, IgA, and/or IgM in a dilution series of the calibration reagent. In embodiments, the assay further comprises generating a standard curve based on the measured amounts of IgG, IgA, and/or IgM in the calibration reagent dilution series. In embodiments, the assay comprises determining the amount of IgG, IgA, and/or IgM in a biological sample based on the standard curve. In embodiments, the IgG, IgA, and/or IgM is from a human, a mouse, a rat, a ferret, a minx, a bat, or a combination thereof.

An exemplary multiplexed serology assay detecting human IgG and/or IgM against SARS-CoV-2 antigens, as described in embodiments herein, comprises:

1A. Preparation of assay plate. In embodiments, the assay plate is a 384-well assay plate. In embodiments, the assay plate is a 96-well assay plate. In embodiments, each well comprises four distinct binding domains. In embodiments, the first binding domain comprises an immobilized SARS-CoV-2 S protein, the second binding domain comprises an immobilized SARS-CoV-2 N protein, and the third binding domain comprises an immobilized SARS-CoV-2 S-RBD. In embodiments, the fourth binding domain comprises a control protein that does not bind to human IgG or IgM. In embodiments, the fourth binding domain comprises immobilized BSA. An embodiment of a well in a 384-well assay plate, comprising four binding domains (“spots”), is shown in FIG. 39A. In embodiments, Spot A1 of FIG. 39A comprises an immobilized SARS-CoV-2 S protein, Spot A2 of FIG. 39A comprises an immobilized SARS-CoV-2 N protein, Spot B1 of FIG. 39A comprises an immobilized SARS-CoV-2 S-RBD, and Spot B2 of FIG. 39A comprises an immobilized BSA. In embodiments, Spot A1 of FIG. 39A comprises an immobilized S protein from SARS-CoV-2, Spot A2 of FIG. 39A comprises an immobilized N protein from SARS-CoV-2, Spot B1 of FIG. 39A comprises an immobilized S-RBD from SARS-CoV-2 strain 501Y.V2, and Spot B2 of FIG. 39A comprises an immobilized S protein from SARS-CoV-2 strain 501Y.V2. In embodiments, the S protein mutations from these SARS-CoV-2 strains are described in Table 1D.

In embodiments, each well comprises ten distinct binding domains. An embodiment of a well in a 96-well assay plate, comprising ten binding domains (“spots”), is shown in FIG. 39B.

In embodiments, Spot 1 of FIG. 39B comprises an immobilized wild-type S protein from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, and Spots 2, 4, 5, 6, and 10 of FIG. 39B each comprises an immobilized BSA. In embodiments, the S protein mutations from these SARS-CoV-2 strains are described in Table 1D.

In embodiments, Spot 1 of FIG. 39B comprises an immobilized wild-type S protein from SARS-CoV-2, Spot 2 of FIG. 39B comprises an immobilized S-D614G from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, Spot 10 of FIG. 39B comprises an immobilized wild-type S-RBD from SARS-CoV-2, and Spots 4, 5, and 6 of FIG. 39B each comprises an immobilized BSA. In embodiments, the S protein mutations from these SARS-CoV-2 strains are described in Table 1D.

In embodiments, Spot 1 of FIG. 39B comprises an immobilized wild-type S protein from SARS-CoV-2, Spot 2 of FIG. 39B comprises an immobilized S-D614G from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, and Spots 4, 5, 6, and 10 of FIG. 39B each comprises an immobilized BSA. In embodiments, the S protein mutations from these SARS-CoV-2 strains are described in Table 1D.

In embodiments, Spot 1 of FIG. 39B comprises an immobilized wild-type S protein from SARS-CoV-2, Spot 2 of FIG. 39B comprises an immobilized S-RBD from SARS-CoV-2 strain 501Y.V2, Spot 3 of FIG. 39B comprises an immobilized N protein from SARS-CoV-2, Spot 4 of FIG. 39B comprises an immobilized S-RBD from SARS-CoV-2 strain P.1, Spot 6 of FIG. 39B comprises an immobilized S-RBD from SARS-CoV-2 strain B.1.1.7, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, Spot 10 of FIG. 39B comprises an immobilized wild-type S-RBD from SARS-CoV-2, and Spot 5 of FIG. 39B comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2, Spot 4 of FIG. 39B comprises an immobilized S-RBD from SARS-CoV-2 strain B.1.526/E484K, Spot 6 of FIG. 39B comprises an immobilized S-RBD from SARS-CoV-2 strain B.1.526/S477N, Spot 7 of FIG. 39B comprises an immobilized S protein from SARS-CoV-2 strain B.1.526/E484K, Spot 8 of FIG. 39B comprises an immobilized S protein from SARS-CoV-2 strain B.1.526/S477N, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spot 5 of FIG. 39B comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2, Spot 4 of FIG. 39B comprises an immobilized S-RBD from SARS-CoV-2 strain B.1.526, Spot 6 of FIG. 39B comprises an immobilized S-RBD from SARS-CoV-2 strain B.1.526.2, Spot 8 of FIG. 39B comprises an immobilized S protein from SARS-CoV-2 strain B.1.526, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spots 5 and 7 of FIG. 39B each comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, Spot 1 of FIG. 39B comprises an immobilized SARS-CoV-2 S-RBD that comprises an L452R mutation, Spot 2 of FIG. 39B comprises an immobilized SARS-CoV-2 S-RBD that comprises K417N, E484K, and N501Y mutations, Spot 3 of FIG. 39B comprises an immobilized SARS-CoV-2 S-RBD that comprises an E484K mutation, Spot 4 of FIG. 39B comprises an immobilized SARS-CoV-2 S-RBD that comprises K417T, E484K, and N501Y mutations, Spot 5 of FIG. 39B comprises an immobilized SARS-CoV-2 S-RBD that comprises an S477N mutation, Spot 6 of FIG. 39B comprises an immobilized SARS-CoV-2 S-RBD that comprises an N501Y mutation, Spot 7 of FIG. 39B comprises an immobilized SARS-CoV-2 S-RBD that comprises E484K and N501Y mutations, Spot 8 of FIG. 39B comprises an immobilized SARS-CoV-2 S-RBD that comprises L452R and E484Q mutations, Spot 9 of FIG. 39B comprises an immobilized SARS-CoV-2 S-RBD that comprises Q414K and N450K mutations, and Spot 10 of FIG. 39B comprises an immobilized wild-type SARS-CoV-2 S-RBD, wherein all mutations are relative to wild-type S-RBD from SARS-CoV-2.

In embodiments, Spot 1 of FIG. 39B comprises an immobilized SARS-CoV-2 S-RBD that comprises an L452R mutation, Spot 2 of FIG. 39B comprises an immobilized SARS-CoV-2 S-RBD that comprises K417N, E484K, and N501Y mutations, Spot 3 of FIG. 39B comprises an immobilized SARS-CoV-2 S-RBD that comprises an E484K mutation, Spot 4 of FIG. 39B comprises an immobilized SARS-CoV-2 S-RBD that comprises K417T, E484K, and N501Y mutations, Spot 5 of FIG. 39B comprises an immobilized SARS-CoV-2 S-RBD that comprises an S477N mutation, Spot 6 of FIG. 39B comprises an immobilized SARS-CoV-2 S-RBD that comprises N501Y and A570D mutations, Spot 7 of FIG. 39B comprises an immobilized SARS-CoV-2 S-RBD that comprises E484K and N501Y mutations, Spot 8 of FIG. 39B comprises an immobilized SARS-CoV-2 S-RBD that comprises L452R and E484Q mutations, Spot 9 of FIG. 39B comprises an immobilized SARS-CoV-2 S-RBD that comprises Q414K and N450K mutations, and Spot 10 of FIG. 39B comprises an immobilized wild-type SARS-CoV-2 S-RBD, wherein all mutations are relative to wild-type S-RBD from SARS-CoV-2.

In embodiments, Spots 1-10 of FIG. 39B comprise, respectively, the following immobilized antigens: a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2.

In embodiments, Spots 1-10 of FIG. 39B comprise, respectively, the following immobilized antigens: a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2.

In embodiments, Spots 1-10 of FIG. 39B comprise, respectively, the following immobilized antigens: a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the Spike protein mutations from these SARS-CoV-2 strains are described in Table 1D. In embodiments, the Spike protein from SARS-CoV-2 strain B.1.617.2 comprises the mutations T19R, Δ157/158, L452R, T478K, D614G, P681R, and D950N.

In embodiments, Spots 1-10 of FIG. 39B comprise, respectively, the following immobilized antigens: a Spike protein from the following SARS-CoV-2 strains: wild type; A.23.1; A.VOI.V2; B.1.617.2; C.37; R.1; P.3; B.1.525; B.1.1.519; and BV-1. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, Spots 1-10 of FIG. 39B comprise, respectively, the following immobilized antigens: a Spike protein from the following SARS-CoV-2 strains: wild type; AY.1, AY.2, B.1.617.2 plus deletion of Y144; B.1.620; B.1.258.17; B.1.466.2; B.1.1.7 plus the E484K mutation; B.1.351.1; and B.1.618. In embodiments, the Spike protein mutations from these SARS-CoV-2 strains are described in Table 1D. In embodiments, the Spike protein from SARS-CoV-2 strain B.1.617.2 plus deletion of Y144 comprises the mutations T19R, AY144, Δ157/158, L452R, T478K, D614G, P681R, and D950N.

In embodiments, Spots 1-4 of FIG. 39B comprise, respectively, the following immobilized antigens: a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; Spots 7-10 of FIG. 39B comprise, respectively, the following immobilized antigens: a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD, wherein all mutations are relative to wild-type S-RBD from SARS-CoV-2; and Spots 5-6 of FIG. 39B each comprises BSA.

In embodiments, Spot 1 of FIG. 39B comprises a wild-type S protein from SARS-CoV-2; Spot 2 of FIG. 39B comprises an S-D614G from SARS-CoV-2; Spot 3 of FIG. 39B comprises an N protein from SARS-CoV-2; Spot 4 of FIG. 39B comprises an S protein from SARS-CoV-2 strain B.1.617.2; Spot 7 of FIG. 39B comprises an S protein from SARS-CoV-2 strain P.1; Spot 8 of FIG. 39B comprises an S protein from SARS-CoV-2 strain B.1.1.7; Spot 9 of FIG. 39B comprises an S protein from SARS-CoV-2 strain B.1.351; Spot 10 of FIG. 39B comprises a wild-type S-RBD from SARS-CoV-2; and Spots 5 and 6 of FIG. 39B comprise BSA. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, Spots 1-10 of FIG. 39B comprise, respectively, the following immobilized antigens: a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the Spike protein mutations from these SARS-CoV-2 strains are described in Table 1D. In embodiments, the Spike protein from SARS-CoV-2 strain B.1.617.2 comprises the mutations T19R, T95I, G142D, Δ156/157, R158G, L452R, T478K, D614G, P681R, and D950N.

In embodiments, Spots 1-3 of FIG. 39B comprise, respectively, an immobilized Spike protein from the following SARS-CoV-2 strains: wild type; P.2; and B.1.617.1; Spot 4 of FIG. 39B comprises BSA; and Spots 5-10 of FIG. 39B comprise, respectively, an immobilized Spike protein from the following SARS-CoV-2 strains: B.1.617.3; B.1.617; P.1; and B.1.1.7. In embodiments, the Spike protein mutations from these SARS-CoV-2 strains are described in Table 1D.

In embodiments, Spots 1-10 of FIG. 39B comprise, respectively, an immobilized Spike protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14). In embodiments, the Spike protein mutations from these SARS-CoV-2 strains are described in Table 1D. In embodiments, the Spike protein from SARS-CoV-2 strain AY.2 comprises the mutations T19R, V70F, G142D, E156G, Δ157/158, A222V, K417N, L452R, T478K, D614G, P681R, and D950N. In embodiments, the Spike protein from SARS-CoV-2 strain B.1.617.2 (AY.4) comprises the mutations T19R, T95I, G142D, Δ156/157, R158G, L452R, T478K, D614G, P681R, and D950N. In embodiments, the Spike protein from SARS-CoV-2 strain AY.1 comprises the mutations T19R, T95I, G142D, E156G, Δ157/158, W258L, K417N, L452R, T478K, D614G, P681R, and D950N. In embodiments, the Spike protein from SARS-CoV-2 strain B.1.617.2 (AY.3, AY.5, AY.6, AY.7, AY.14) comprises the mutations T19R, G142D, Δ156/157, R158G, L452R, T478K, D614G, P681R, and D950N.

In embodiments, Spots 1-10 of FIG. 39B comprise, respectively, an immobilized Spike protein from the following SARS-CoV-2 strains: wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2. In embodiments, the Spike protein mutations from these SARS-CoV-2 strains are described in Table 1D. In embodiments, the Spike protein from SARS-CoV-2 strain B.1.617.2 (+K417N/N439K/E484K/N501Y) comprises the mutations T19R, G142D, Δ156/157, R158G, K417N, N439K, L452R, T478K, E484K, N501Y, D614G, P681R, and D950N. In embodiments, the Spike protein from SARS-CoV-2 strain B.1.617.2 (+K417N/E484K/N501Y) comprises the mutations T19R, G142D, Δ156/157, R158G, K417N, L452R, T478K, E484K, N501Y, D614G, P681R, and D950N. In embodiments, the Spike protein from SARS-CoV-2 strain B.1.617.2 (+E484K/N501Y) comprises the mutations T19R, G142D, Δ156/157, R158G, L452R, T478K, E484K, N501Y, D614G, P681R, and D950N. In embodiments, the Spike protein from SARS-CoV-2 strain B.1.617.2 (+E484K) comprises the mutations T19R, G142D, del156/157, R158G, L452R, T478K, E484K, D614G, P681R, and D950N. In embodiments, the Spike protein from SARS-CoV-2 strain B.1.617.2 comprises the mutations T19R, G142D, Δ156/157, R158G, L452R, T478K, D614G, P681R, and D950N.

In embodiments, Spots 1-4 of FIG. 39B comprise, respectively, an immobilized Spike protein from the following SARS-CoV-2 strains: wild-type; B.1.1.529; AY.4.2; AY.4; Spots 5-6 each comprises BSA; and Spots 7-10 comprise, respectively, an immobilized Spike protein from the following SARS-CoV-2 strains: P.1; B.1.1.7; B.1.351; and B.1.617.2. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, Spots 1 and 2 of FIG. 39B comprise, respectively, an immobilized S-RBD from the following SARS-CoV-2 strains: B.1.1.529 and B.1.351; Spot 3 comprises BSA; Spot 4 comprises an immobilized S-RBD from SARS-CoV-2 strain P.1; Spot 5 comprises BSA; Spot 6 comprises an immobilized S-RBD from SARS—Co-V-2 strain B.1.1.7; Spots 7 and 8 comprise BSA; Spot 9 comprises an immobilized S-RBD from SARS-CoV-2 strain B.1.617.2; and Spot 10 comprises an immobilized S-RBD from wild type SARS-CoV-2. In embodiments, the S-RBD mutations from these SARS-CoV-2 strains are described in Table 1E.

In embodiments, Spots 1 and 2 of FIG. 39B comprise, respectively, an immobilized S protein from the following SARS-CoV-2 strains: wild-type and B.1.1.529; Spot 3 comprises an immobilized N protein from wild-type SARS-CoV-2; Spot 4 comprises an immobilized S protein from SARS-CoV-2 strain AY.4; Spots 5 and 6 each comprises BSA; Spots 7-9 comprise, respectively, an immobilized S protein from the following SARS-CoV-2 strains: P.1; B.1.1.7; and B.1.351; and Spot 10 comprises an immobilized S-RBD from wild type SARS-CoV-2. In embodiments, the S protein mutations from these SARS-CoV-2 strains are described in Table 1D.

In embodiments, Spots 1-10 of FIG. 39B comprise, respectively, an immobilized S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2. In embodiments, the S protein mutations from these SARS-CoV-2 strains are described in Table 1D.

In embodiments, Spots 1-4 of FIG. 39B comprise, respectively, an immobilized S-RBD from the following SARS-CoV-2 strains: B.1.1.529 (BA.1); B.1.351; BA.2; and P.1; Spot 6 comprises an immobilized S-RBD from SARS-CoV-2 strain B.1.1.7; Spots 8-10 comprise, respectively, an immobilized S-RBD from the following SARS-CoV-2 strains: BA.1.1; B.1.617.2; and wild-type; and Spots 5 and 7 each comprises an immobilized BSA. In embodiments, the S-RBD mutations from these SARS-CoV-2 strains are described in Table 1E.

In embodiments, about 1 μL to about 200 μL, about 3 μL to about 150 μL, about 5 μL to about 100 μL, about 10 μL to about 90 μL, about 15 μL to about 80 μL, about 20 μL to about 70 μL, about 30 μL to about 60 μL, about 50 μL, or about 150 μL of a blocking solution to each well of the plate. In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

1B. Preparation of reagents. In embodiments, the assay comprises measuring the amount of one or more calibration reagents. In embodiments, the calibration reagent comprises a known quantity of IgG and/or IgM. In embodiments, the calibration reagent comprises a blank solution containing no IgG or IgM. In embodiments, the assay comprises measuring the amount of multiple calibration reagents, e.g., at least 3, at least 4, at least 5, at least 6, at least 7, at least 8, at least 9, or at least 10 calibration reagents. In embodiments, the assay comprises generating a standard curve from the multiple calibration reagents. In embodiments, the multiple calibration reagents comprise a range of concentrations of IgG and/or IgM. In embodiments, the assay comprises diluting a concentration reagent to provide multiple calibration reagents comprising a range of concentrations. In embodiments, the calibration reagent is diluted 1:10, 1:20, 1:30, 1:40, 1:50, 1:60, 1:70, 1:80, 1:90, 1:100, 1:140, 1:160, 1:200, 1:300, 1:400, 1:500, 1:600, 1:700, 1:800, 1:900, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10000, 1:20000, 1:30000, 1:40000, or 1:50000 to provide multiple concentrations of the calibration reagent. Calibration reagents are further described herein.

In embodiments, the assay comprises measuring the amount of one or more control reagents. In embodiments, the control reagent comprises a known quantity of IgG and/or IgM against the specific viral antigens in the assay, e.g., SARS-CoV-2 S, SARS-CoV-2 N, and/or SARS-CoV-2 S-RBD. In embodiments, the one or more control reagents comprises a first control reagent obtained from a subject known to never have been exposed to SARS-CoV-2, a second control reagent obtained from a subject during an early stage of infection by SARS-CoV-2, a third control reagent obtained from a subject during a late stage infection by SARS-CoV-2, a fourth control reagent obtained from a subject who has recovered from an infection by SARS-CoV-2, or a combination thereof. Control reagents are further described herein.

Examples of samples, e.g., biological samples, are provided herein. In embodiments, the sample is diluted about 2-fold, about 5-fold, about 10-fold, about 20-fold, about 30-fold, about 40-fold, about 50-fold, about 60-fold, about 70-fold, about 80-fold, about 90-fold, about 100-fold, about 250-fold, about 500-fold, about 750-fold, about 1000-fold, about 1500-fold, about 2000-fold, about 2500-fold, about 3000-fold, about 3500-fold, about 4000-fold, about 4500-fold, or about 5000-fold for use in the assay.

2. Addition of samples and reagents. In embodiments, the assay plate is washed at least once, at least twice, at least three times, at least four times, or at least five times with a wash buffer after incubation with the blocking solution. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the sample, one or more calibration reagents, and one or more control reagents are added to their respectively designated wells of the plate. In embodiments, about 5 μL to about 50 μL, about 10 μL to about 40 μL, about 20 μL to about 30 μL, about 15 μL, about 25 μL, or about 50 μL of the sample, calibration reagent, or control reagent is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, about 500 rpm to about 1000 rpm, or about 1200 rpm to about 1600 rpm. In embodiments, the plate is incubated for about 10 minutes to about 12 hours, or about 30 minutes to about 8 hours, or about 45 minutes to about 6 hours, or about 1 hour, or about 4 hours. In embodiments, the plate is incubated at about room temperature (e.g., about 22° C. to about 28° C.) while shaken at about 1500 rpm for about 4 hours. In embodiments, the plate is incubated at about room temperature (e.g., about 22° C. to about 28° C.) while shaken at about 700 rpm for about 1 hour.

3. Addition of detection reagent. In embodiments, the detection reagent is diluted from a stock solution of detection reagent to obtain a solution comprising a working concentration of detection reagent. Detection reagents are further described herein.

In embodiments, the assay plate is washed at least once, at least twice, at least three times, at least four times, or at least five times with a wash buffer after incubation with the sample, calibration reagent, or control reagent. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the detection reagent solution is added to each well of the plate. In embodiments, about 5 μL to about 50 μL, about 10 μL to about 40 μL, about 10 μL to about 20 μL, about 20 μL to about 30 μL, about 15 μL, about 25 μL, or about 50 μL of the detection reagent solution is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, about 500 rpm to about 1000 rpm, or about 1200 rpm to about 1600 rpm. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) while shaken at about 1500 rpm for about 1 hour. In embodiments, the plate is incubated at about room temperature (e.g., about 22° C. to about 28° C.) while shaken at about 700 rpm for about 1 hour.

4. Addition of read buffer. In embodiments, the assay plate is washed at least once, at least twice, at least three times, at least four times, or at least five times with a wash buffer after incubation with the detection reagent. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, the read buffer is added to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, about 40 μL, about 50 μL, about 100 μL, or about 150 μL of the read buffer is added to each well.

In embodiments, the assay comprises reading the plate, e.g., on a plate reader as described herein. In embodiments, the assay comprises reading the plate immediately following addition of the read buffer.

A further exemplary serology assay for detecting an antibody biomarker that binds to a SARS-CoV-2 antigen comprises:

(a) mixing (i) a coating solution comprising a binding reagent bound to a linking agent and (ii) a detection reagent, wherein each of the binding reagent and the detection reagent comprises a SARS-CoV-2 antigen, and wherein the detection reagent comprises a detectable label;

(b) contacting a surface with: (i) a sample comprising the antibody biomarker, (ii) a calibration reagent, or (iii) a control reagent, wherein the surface comprising one or more binding domains, wherein each binding domain comprises a targeting agent;

(c) contacting the surface with the mixture of (a);

(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

A further exemplary serology assay for detecting an antibody biomarker that binds to a SARS-CoV-2 antigen comprises:

(a) mixing (i) a biotinylated binding reagent and (ii) a detection reagent, wherein each of the binding reagent and the detection reagent comprises a SARS-CoV-2 antigen, and wherein the detection reagent comprises a detectable label;

(b) contacting a surface with: (i) a sample comprising the antibody biomarker, (ii) a calibration reagent, or (iii) a control reagent, wherein the surface comprising one or more binding domains, wherein each binding domain comprises avidin or streptavidin;

(c) contacting the surface with the mixture of (a);

(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

In embodiments, the surface is a multi-well plate. In embodiments, the assay further comprises a wash step prior to one or more of the assay steps. In embodiments, the wash step comprises washing the assay plate at least once, at least twice, at least three times, at least four times, or at least five times with a wash buffer. In embodiments, the assay plate is washed with at least about 10 μL, at least about 15 μL, at least about 20 μL, at least about 25 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, prior to step (a), a blocking solution is added to the plate to reduce non-specific binding of the coating solution or the biotinylated binding reagent to the surface. In embodiments, about 50 μL to about 250 μL, about 100 μL to about 200 μL, or about 150 μL of blocking solution is added per well of the plate. In embodiments, the plate is incubated at about 15° C. to about 30° C., about 18° C. to about 28° C., about 20° C. to about 26° C., or about 22° C. to about 24° C. In embodiments, the plate is incubated while shaken at about at about 500 rpm to about 2000 rpm, about 600 rpm to about 1500 rpm, or about 700 rpm to about 1000 rpm. In embodiments, the method comprises incubating the blocking solution on the plate for about 10 minutes to about 4 hours, about 20 minutes to about 3 hours, or about 30 minutes to about 2 minutes. In embodiments, the plate is incubated at about room temperature (e.g., about 22° C. to about 28° C.) while shaken at about 700 rpm for about 30 minutes to about 2 hours.

In embodiments comprising a coating solution, the assay further comprises, prior to step (a), mixing a linking agent connected to a targeting agent complement with a binding reagent comprising a supplemental linking agent, thereby forming the coating solution comprising the binding reagent bound to the linking agent. In embodiments, the method comprises forming about 200 μL to about 1000 μL, or about 300 μL to about 800 μL, or about 400 μL to about 600 μL of the coating solution. In embodiments, step (a) comprises incubating the linking agent and the binding reagent at about 15° C. to about 30° C., about 18° C. to about 28° C., about 20° C. to about 26° C., or about 22° C. to about 24° C. In embodiments, the method comprises forming about 500 μL of the coating solution by incubating about 300 μL of a solution comprising the linking agent and about 200 μL of a solution comprising the binding reagent, at about room temperature (e.g., about 22° C. to about 28° C.) for about 30 minutes. In embodiments, the incubating is performed without shaking. In embodiments, the assay further comprises contacting the coating solution with a stop solution (e.g., about 100 μL to about 500 μL, or about 150 μL to about 300 μL, or about 200 μL of a stop solution) to stop the binding reaction between the linking agent and supplemental linking agent. In embodiments, the coating solution and the stop solution are incubated for about 10 minutes to about 1 hour, about 20 minutes to about 40 minutes, or about 30 minutes. In embodiments, the coating solution and the stop solution are incubated at about 15° C. to about 30° C., about 18° C. to about 28° C., about 20° C. to about 26° C., or about 22° C. to about 24° C. In embodiments, the method further comprises, following incubation of the coating solution with the stop solution, diluting the coating solution using the stop solution, e.g., by 2-fold, 5-fold, 10-fold, or 20-fold, to a working concentration as described herein. In embodiments, the targeting agent and targeting agent complement comprise complementary oligonucleotides. In embodiments, the linking agent comprises avidin or streptavidin, and the supplemental linking agent comprises biotin.

In embodiments, step (b) comprises adding about 5 μL to about 50 μL, about 10 μL to about 40 μL, about 20 μL to about 30 μL, about 15 μL, about 25 μL, about 35 μL, or about 50 μL of the sample, calibration reagent, or control reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (c) comprises adding about 5 μL to about 50 μL, about 10 μL to about 40 μL, about 10 μL to about 20 μL, about 20 μL to about 30 μL, about 15 μL, about 25 μL, about 35 μL, or about 50 μL of the mixture of (a) comprising the binding reagent and the detection reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (d) comprises adding a read buffer to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, about 40 μL, about 50 μL, about 100 μL, or about 150 μL of the read buffer is added to each well. In embodiments, the measuring comprises reading the plate, e.g., on a plate reader as described herein. In embodiments, the assay comprises reading the plate immediately following addition of the read buffer.

An exemplary multiplexed competitive serology assay detecting human neutralizing antibodies (also known as blocking antibodies) against SARS-CoV-2 antigens, as described in embodiments herein, comprises:

1A. Preparation of assay plate. In embodiments, the assay plate is a 384-well assay plate. In embodiments, the assay plate is a 96-well assay plate. In embodiments, each well comprises four distinct binding domains. In embodiments, the first binding domain comprises an immobilized SARS-CoV-2 S protein, the second binding domain comprises an immobilized SARS-CoV-2 N protein, and the third binding domain comprises an immobilized SARS-CoV-2 S-RBD. In embodiments, the fourth binding domain comprises a control protein that does not bind to human antibodies. In embodiments, the fourth binding domain comprises immobilized BSA. An embodiment of a well in a 384-well assay plate, comprising four binding domains (“spots”), is shown in FIG. 39A. In embodiments, Spot A1 of FIG. 39A comprises an immobilized SARS-CoV-2 S protein, Spot A2 of FIG. 39A comprises an immobilized SARS-CoV-2 N protein, Spot B1 of FIG. 39A comprises an immobilized SARS-CoV-2 S-RBD, and Spot B2 of FIG. 39A comprises an immobilized BSA. In embodiments, Spot A1 of FIG. 39A comprises an immobilized SARS-CoV-2 S protein, Spot A2 of FIG. 39A comprises an immobilized SARS-CoV-2 N protein, Spot B1 of FIG. 39A comprises an immobilized S-RBD from SARS-CoV-2 strain 501Y.V2, and Spot B2 of FIG. 39A comprises S protein from SARS-CoV-2 strain 501Y.V2. In embodiments, the S protein mutations from these SARS-CoV-2 strains are described in Table 1D.

In embodiments, each well comprises ten distinct binding domains. An embodiment of a well in a 96-well assay plate, comprising ten binding domains (“spots”), is shown in FIG. 39B.

In embodiments, Spot 1 of FIG. 39B comprises an immobilized wild-type S protein from SARS-CoV-2, Spot 3 of FIG. 39B comprises an N protein from immobilized SARS-CoV-2, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, and Spots 2, 4, 5, 6, and 10 of FIG. 39B each comprises an immobilized BSA. In embodiments, the S protein mutations from these SARS-CoV-2 strains are described in Table 1D.

In embodiments, Spot 1 of FIG. 39B comprises an immobilized wild-type S protein from SARS-CoV-2, Spot 2 of FIG. 39B comprises an S-D614G from SARS-CoV-2, Spot 3 of FIG. 39B comprises an N protein from immobilized SARS-CoV-2, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, Spot 10 of FIG. 39B comprises a wild-type S-RBD from SARS-CoV-2, and Spots 4, 5, and 6 of FIG. 39B each comprises an immobilized BSA. In embodiments, the S protein mutations from these SARS-CoV-2 strains are described in Table 1D.

In embodiments, Spot 1 of FIG. 39B comprises an immobilized wild-type S protein from SARS-CoV-2, Spot 2 of FIG. 39B comprises an immobilized S-D614G from SARS-CoV-2, Spot 3 of FIG. 39B comprises an immobilized N protein from SARS-CoV-2, Spot 7 of FIG. 39B comprises an immobilized S protein from SARS-CoV-2 strain P.1, Spot 8 of FIG. 39B comprises an immobilized S protein from SARS-CoV-2 strain B.1.1.7, Spot 9 of FIG. 39B comprises an immobilized S protein from SARS-CoV-2 strain 501Y.V2, and Spots 4, 5, 6, and 10 of FIG. 39B each comprises an immobilized BSA. In embodiments, the S protein mutations from these SARS-CoV-2 strains are described in Table 1D.

In embodiments, Spot 1 of FIG. 39B comprises an immobilized wild-type S protein from SARS-CoV-2, Spot 2 of FIG. 39B comprises an S-RBD from SARS-CoV-2 strain 501Y.V2, Spot 3 of FIG. 39B comprises an N protein from immobilized SARS-CoV-2, Spot 4 of FIG. 39B comprises an S-RBD from SARS-CoV-2 strain P.1, Spot 6 of FIG. 39B comprises an S-RBD from SARS-CoV-2 strain B.1.1.7, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, Spot 10 of FIG. 39B comprises a wild-type S-RBD from SARS-CoV-2, and Spot 5 of FIG. 39B each comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2, Spot 4 of FIG. 39B comprises an immobilized S-RBD from SARS-CoV-2 strain B.1.526/E484K, Spot 6 of FIG. 39B comprises an immobilized S-RBD from SARS-CoV-2 strain B.1.526/S477N, Spot 7 of FIG. 39B comprises an immobilized S protein from SARS-CoV-2 strain B.1.526/E484K, Spot 8 of FIG. 39B comprises an immobilized S protein from SARS-CoV-2 strain B.1.526/S477N, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spot 5 of FIG. 39B comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2, Spot 4 of FIG. 39B comprises an immobilized S-RBD from SARS-CoV-2 strain B.1.526, Spot 6 of FIG. 39B comprises an immobilized S-RBD from SARS-CoV-2 strain B.1.526.2, Spot 8 of FIG. 39B comprises an immobilized S protein from SARS-CoV-2 strain B.1.526, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spots 5 and 7 of FIG. 39B each comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, Spot 1 of FIG. 39B comprises an immobilized SARS-CoV-2 S-RBD that comprises an L452R mutation, Spot 2 of FIG. 39B comprises an immobilized SARS-CoV-2 S-RBD that comprises K417N, E484K, and N501Y mutations, Spot 3 of FIG. 39B comprises an immobilized SARS-CoV-2 S-RBD that comprises an E484K mutation, Spot 4 of FIG. 39B comprises an immobilized SARS-CoV-2 S-RBD that comprises K417T, E484K, and N501Y mutations, Spot 5 of FIG. 39B comprises an immobilized SARS-CoV-2 S-RBD that comprises an S477N mutation, Spot 6 of FIG. 39B comprises an immobilized SARS-CoV-2 S-RBD that comprises an N501Y mutation, Spot 7 of FIG. 39B comprises an immobilized SARS-CoV-2 S-RBD that comprises E484K and N501Y mutations, Spot 8 of FIG. 39B comprises an immobilized SARS-CoV-2 S-RBD that comprises L452R and E484Q mutations, Spot 9 of FIG. 39B comprises an immobilized SARS-CoV-2 S-RBD that comprises Q414K and N450K mutations, and Spot 10 of FIG. 39B comprises an immobilized wild-type SARS-CoV-2 S-RBD, wherein all mutations are relative to wild-type S-RBD from SARS-CoV-2.

In embodiments, Spot 1 of FIG. 39B comprises an immobilized SARS-CoV-2 S-RBD that comprises an L452R mutation, Spot 2 of FIG. 39B comprises an immobilized SARS-CoV-2 S-RBD that comprises K417N, E484K, and N501Y mutations, Spot 3 of FIG. 39B comprises an immobilized SARS-CoV-2 S-RBD that comprises an E484K mutation, Spot 4 of FIG. 39B comprises an immobilized SARS-CoV-2 S-RBD that comprises K417T, E484K, and N501Y mutations, Spot 5 of FIG. 39B comprises an immobilized SARS-CoV-2 S-RBD that comprises an S477N mutation, Spot 6 of FIG. 39B comprises an immobilized SARS-CoV-2 S-RBD that comprises N501Y and A570D mutations, Spot 7 of FIG. 39B comprises an immobilized SARS-CoV-2 S-RBD that comprises E484K and N501Y mutations, Spot 8 of FIG. 39B comprises an immobilized SARS-CoV-2 S-RBD that comprises L452R and E484Q mutations, Spot 9 of FIG. 39B comprises an immobilized SARS-CoV-2 S-RBD that comprises Q414K and N450K mutations, and Spot 10 of FIG. 39B comprises an immobilized wild-type SARS-CoV-2 S-RBD, wherein all mutations are relative to wild-type S-RBD from SARS-CoV-2.

In embodiments, Spots 1-10 of FIG. 39B comprise, respectively, the following immobilized antigens: a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2.

In embodiments, Spots 1-10 of FIG. 39B comprise, respectively, the following immobilized antigens: a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2.

In embodiments, Spots 1-10 of FIG. 39B comprise, respectively, the following immobilized antigens: a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the Spike protein mutations from these SARS-CoV-2 strains are described in Table 1D. In embodiments, the Spike protein from SARS-CoV-2 strain B.1.617.2 comprises the mutations T19R, Δ157/158, L452R, T478K, D614G, P681R, and D950N.

In embodiments, Spots 1-10 of FIG. 39B comprise, respectively, the following immobilized antigens: a Spike protein from the following SARS-CoV-2 strains: wild type; A.23.1; A.VOI.V2; B.1.617.2; C.37; R.1; P.3; B.1.525; B.1.1.519; and BV-1. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, Spots 1-10 of FIG. 39B comprise, respectively, the following immobilized antigens: a Spike protein from the following SARS-CoV-2 strains: wild type; AY.1, AY.2, B.1.617.2 plus deletion of Y144; B.1.620; B.1.258.17; B.1.466.2; B.1.1.7 plus the E484K mutation; B.1.351.1; and B.1.618. In embodiments, the Spike protein mutations from these SARS-CoV-2 strains are described in Table 1D. In embodiments, the Spike protein from SARS-CoV-2 strain B.1.617.2 plus deletion of Y144 comprises the mutations T19R, AY144, Δ157/158, L452R, T478K, D614G, P681R, and D950N.

In embodiments, Spots 1-4 of FIG. 39B comprise, respectively, the following immobilized antigens: a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; Spots 7-10 of FIG. 39B comprise, respectively, the following immobilized antigens: a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD, wherein all mutations are relative to wild-type S-RBD from SARS-CoV-2; and Spots 5-6 of FIG. 39B each comprises BSA.

In embodiments, Spot 1 of FIG. 39B comprises a wild-type S protein from SARS-CoV-2; Spot 2 of FIG. 39B comprises an S-D614G from SARS-CoV-2; Spot 3 of FIG. 39B comprises an N protein from SARS-CoV-2; Spot 4 of FIG. 39B comprises an S protein from SARS-CoV-2 strain B.1.617.2; Spot 7 of FIG. 39B comprises an S protein from SARS-CoV-2 strain P.1; Spot 8 of FIG. 39B comprises an S protein from SARS-CoV-2 strain B.1.1.7; Spot 9 of FIG. 39B comprises an S protein from SARS-CoV-2 strain B.1.351; Spot 10 of FIG. 39B comprises a wild-type S-RBD from SARS-CoV-2; and Spots 5 and 6 of FIG. 39B comprise BSA. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, Spots 1-10 of FIG. 39B comprise, respectively, the following immobilized antigens: a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the Spike protein mutations from these SARS-CoV-2 strains are described in Table 1D. In embodiments, the Spike protein from SARS-CoV-2 strain B.1.617.2 comprises the mutations T19R, T95I, G142D, Δ156/157, R158G, L452R, T478K, D614G, P681R, and D950N.

In embodiments, Spots 1-3 of FIG. 39B comprise, respectively, an immobilized Spike protein from the following SARS-CoV-2 strains: wild type; P.2; and B.1.617.1; Spot 4 of FIG. 39B comprises BSA; and Spots 5-10 of FIG. 39B comprise, respectively, an immobilized Spike protein from the following SARS-CoV-2 strains: B.1.617.3; B.1.617; P.1; and B.1.1.7. In embodiments, the Spike protein mutations from these SARS-CoV-2 strains are described in Table 1D.

In embodiments, Spots 1-10 of FIG. 39B comprise, respectively, an immobilized Spike protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14). In embodiments, the Spike protein mutations from these SARS-CoV-2 strains are described in Table 1D. In embodiments, the Spike protein from SARS-CoV-2 strain AY.2 comprises the mutations T19R, V70F, G142D, E156G, Δ157/158, A222V, K417N, L452R, T478K, D614G, P681R, and D950N. In embodiments, the Spike protein from SARS-CoV-2 strain B.1.617.2 (AY.4) comprises the mutations T19R, T95I, G142D, Δ156/157, R158G, L452R, T478K, D614G, P681R, and D950N. In embodiments, the Spike protein from SARS-CoV-2 strain AY.1 comprises the mutations T19R, T95I, G142D, E156G, Δ157/158, W258L, K417N, L452R, T478K, D614G, P681R, and D950N. In embodiments, the Spike protein from SARS-CoV-2 strain B.1.617.2 (AY.3, AY.5, AY.6, AY.7, AY.14) comprises the mutations T19R, G142D, Δ156/157, R158G, L452R, T478K, D614G, P681R, and D950N.

In embodiments, Spots 1-10 of FIG. 39B comprise, respectively, an immobilized Spike protein from the following SARS-CoV-2 strains: wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2. In embodiments, the Spike protein mutations from these SARS-CoV-2 strains are described in Table 1D. In embodiments, the Spike protein from SARS-CoV-2 strain B.1.617.2 (+K417N/N439K/E484K/N501Y) comprises the mutations T19R, G142D, Δ156/157, R158G, K417N, N439K, L452R, T478K, E484K, N501Y, D614G, P681R, and D950N. In embodiments, the Spike protein from SARS-CoV-2 strain B.1.617.2 (+K417N/E484K/N501Y) comprises the mutations T19R, G142D, Δ156/157, R158G, K417N, L452R, T478K, E484K, N501Y, D614G, P681R, and D950N. In embodiments, the Spike protein from SARS-CoV-2 strain B.1.617.2 (+E484K/N501Y) comprises the mutations T19R, G142D, Δ156/157, R158G, L452R, T478K, E484K, N501Y, D614G, P681R, and D950N. In embodiments, the Spike protein from SARS-CoV-2 strain B.1.617.2 (+E484K) comprises the mutations T19R, G142D, del156/157, R158G, L452R, T478K, E484K, D614G, P681R, and D950N. In embodiments, the Spike protein from SARS-CoV-2 strain B.1.617.2 comprises the mutations T19R, G142D, Δ156/157, R158G, L452R, T478K, D614G, P681R, and D950N.

In embodiments, Spots 1-4 of FIG. 39B comprise, respectively, an immobilized Spike protein from the following SARS-CoV-2 strains: wild-type; B.1.1.529; AY.4.2; AY.4; Spots 5-6 each comprises BSA; and Spots 7-10 comprise, respectively, an immobilized Spike protein from the following SARS-CoV-2 strains: P.1; B.1.1.7; B.1.351; and B.1.617.2. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, Spots 1 and 2 of FIG. 39B comprise, respectively, an immobilized S-RBD from the following SARS-CoV-2 strains: B.1.1.529 and B.1.351; Spot 3 comprises BSA; Spot 4 comprises an immobilized S-RBD from SARS-CoV-2 strain P.1; Spot 5 comprises BSA; Spot 6 comprises an immobilized S-RBD from SARS-CoV-2 strain B.1.1.7; Spots 7 and 8 comprise BSA; Spot 9 comprises an immobilized S-RBD from SARS-CoV-2 strain B.1.617.2; and Spot 10 comprises an immobilized S-RBD from wild type SARS-CoV-2. In embodiments, the S-RBD mutations from these SARS-CoV-2 strains are described in Table 1E.

In embodiments, Spots 1 and 2 of FIG. 39B comprise, respectively, an immobilized S protein from the following SARS-CoV-2 strains: wild-type and B.1.1.529; Spot 3 comprises an immobilized N protein from wild-type SARS-CoV-2; Spot 4 comprises an immobilized S protein from SARS-CoV-2 strain AY.4; Spots 5 and 6 each comprises BSA; Spots 7-9 comprise, respectively, an immobilized S protein from the following SARS-CoV-2 strains: P.1; B.1.1.7; and B.1.351; and Spot 10 comprises an immobilized S-RBD from wild type SARS-CoV-2. In embodiments, the S protein mutations from these SARS-CoV-2 strains are described in Table 1D.

In embodiments, Spots 1-10 of FIG. 39B comprise, respectively, an immobilized S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2. In embodiments, the S protein mutations from these SARS-CoV-2 strains are described in Table 1D.

In embodiments, Spots 1-4 of FIG. 39B comprise, respectively, an immobilized S-RBD from the following SARS-CoV-2 strains: B.1.1.529 (BA.1); B.1.351; BA.2; and P.1; Spot 6 comprises an immobilized S-RBD from SARS-CoV-2 strain B.1.1.7; Spots 8-10 comprise, respectively, an immobilized S-RBD from the following SARS-CoV-2 strains: BA.1.1; B.1.617.2; and wild-type; and Spots 5 and 7 each comprises an immobilized BSA. In embodiments, the S-RBD mutations from these SARS-CoV-2 strains are described in Table 1E.

In embodiments, about 1 μL to about 200 μL, about 3 μL to about 150 μL, about 5 μL to about 100 μL, about 10 μL to about 90 μL, about 15 μL to about 80 μL, about 20 μL to about 70 μL, about 30 μL to about 60 μL, about 50 μL, or about 150 μL of a blocking solution to each well of the plate. In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

1B. Preparation of reagents. In embodiments, the assay comprises measuring the amount of one or more calibration reagents. In embodiments, the calibration reagent comprises a known quantity of IgG and/or IgM. In embodiments, the calibration reagent comprises a blank solution containing no IgG or IgM. In embodiments, the assay comprises measuring the amount of multiple calibration reagents, e.g., at least 3, at least 4, at least 5, at least 6, at least 7, at least 8, at least 9, or at least 10 calibration reagents. In embodiments, the assay comprises generating a standard curve from the multiple calibration reagents. In embodiments, the multiple calibration reagents comprise a range of concentrations of IgG and/or IgM. In embodiments, the assay comprises diluting a concentration reagent to provide multiple calibration reagents comprising a range of concentrations. In embodiments, the calibration reagent is diluted 1:10, 1:20, 1:30, 1:40, 1:50, 1:60, 1:70, 1:80, 1:90, 1:100, 1:140, 1:160, 1:200, 1:300, 1:400, 1:500, 1:600, 1:700, 1:800, 1:900, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10000, 1:20000, 1:30000, 1:40000, or 1:50000 to provide multiple concentrations of the calibration reagent. Calibration reagents are further described herein.

Examples of samples, e.g., biological samples, are provided herein. In embodiments, the sample is diluted about 2-fold, about 5-fold, about 10-fold, about 20-fold, about 30-fold, about 40-fold, about 50-fold, about 60-fold, about 70-fold, about 80-fold, about 90-fold, about 100-fold, about 250-fold, about 500-fold, about 750-fold, about 1000-fold, about 1500-fold, about 2000-fold, about 2500-fold, about 3000-fold, about 3500-fold, about 4000-fold, about 4500-fold, or about 5000-fold for use in the assay.

2. Addition of samples and reagents. In embodiments, the assay plate is washed at least once, at least twice, at least three times, at least four times, or at least five times with a wash buffer after incubation with the blocking solution. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the sample and one or more calibration reagents are added to their respectively designated wells of the plate. In embodiments, about 5 μL to about 50 μL, about 10 μL to about 40 μL, about 10 μL to about 20 μL, about 20 μL to about 30 μL, about 15 μL, about 25 μL, or about 50 μL of the sample or calibration reagent is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, or about 1200 rpm to about 1600 rpm. In embodiments, the plate is incubated for about 10 minutes to about 12 hours, or about 30 minutes to about 8 hours, or about 45 minutes to about 6 hours, or about 1 hour, or about 4 hours. In embodiments, the plate is incubated at about room temperature (e.g., about 22° C. to about 28° C.) while shaken at about 1500 rpm for about 4 hours. In embodiments, the plate is incubated at about room temperature (e.g., about 22° C. to about 28° C.) while shaken at about 700 rpm for about 1 hour.

3. Addition of ACE2 detection reagent. In embodiments, the ACE2 detection reagent is diluted from a stock solution of detection reagent to obtain a solution comprising a working concentration of ACE2 detection reagent. ACE2 is further described herein.

In embodiments, the assay plate is washed at least once, at least twice, at least three times, at least four times, or at least five times with a wash buffer after incubation with the sample or calibration reagent. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, after the washing, the ACE2 detection solution is added to each well of the plate. In embodiments, about 5 μL to about 50 μL, about 10 μL to about 40 μL, about 10 μL to about 20 μL, about 20 μL to about 30 μL, about 25 μL, or about 50 μL of the ACE2 detection solution is added to each well.

In embodiments, the plate is sealed or covered, e.g., with an adhesive seal or a plate cover. In embodiments, the plate is incubated at about 15° C. to about 30° C., about 18° C. to about 28° C., about 20° C. to about 26° C., or about 22° C. to about 24° C. In embodiments, the plate is incubated while shaken at about 500 rpm to about 3000 rpm, about 800 rpm to about 2000 rpm, about 1000 rpm to about 1800 rpm, about 500 rpm to about 1000 rpm, or about 1200 rpm to about 1600 rpm. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) while shaken at about 1500 rpm for about 1 hour. In embodiments, the plate is incubated at about room temperature (e.g., about 22° C. to about 28° C.) while shaken at about 700 rpm for about 1 hour.

4. Addition of read buffer. In embodiments, the assay plate is washed at least once, at least twice, at least three times, at least four times, or at least five times with a wash buffer after incubation with the ACE2 detection solution. In embodiments, the assay plate is washed with at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer.

In embodiments, the read buffer is added to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, or about 40 μL of the read buffer is added to each well.

In embodiments, the assay comprises reading the plate, e.g., on a plate reader as described herein. In embodiments, the assay comprises reading the plate immediately following addition of the read buffer.

A further exemplary competitive serology assay for detecting an antibody biomarker that binds to a SARS-CoV-2 antigen comprises:

(a) contacting a coating solution comprising a binding reagent bound to a linking reagent with a surface comprising one or more binding domains, wherein each binding domain comprises a targeting agent, and wherein the binding reagent is a SARS-CoV-2 antigen;

(b) contacting each binding domain with: (i) a sample comprising the antibody biomarker, (ii) a calibration reagent, or (iii) a control reagent;

(c) contacting each binding domain with an ACE2 detection reagent comprising a detectable label;

(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

A further exemplary competitive serology assay for detecting an antibody biomarker that binds to a SARS-CoV-2 antigen comprises:

(a) contacting a biotinylated binding reagent with a surface comprising one or more binding domains, wherein each binding domain comprises avidin or streptavidin, and wherein the binding reagent is a SARS-CoV-2 antigen;

(b) contacting each binding domain with: (i) a sample comprising the antibody biomarker, (ii) a calibration reagent, or (iii) a control reagent;

(c) contacting each binding domain with an ACE2 detection reagent comprising a detectable label;

(d) measuring the amount of detectable label on the surface, thereby detecting and/or measuring the amount of the antibody biomarker. In embodiments, the SARS-CoV-2 antigen is SARS-CoV-2 N protein. In embodiments, the SARS-CoV-2 antigen is SARS-CoV-2 S protein. In embodiments, the SARS-CoV-2 antigen is SARS-CoV-2 S-RBD. In embodiments, the SARS-CoV-2 antigen comprises the SARS-CoV-2 N protein and SARS-CoV-2 S-RBD, and the assay is a multiplexed assay that detects antibody biomarkers that bind to the SARS-CoV-2 N protein and the SARS-CoV-2 S-RBD.

In embodiments, the surface is a multi-well plate. In embodiments, the assay further comprises a wash step prior to one or more of the assay steps. In embodiments, the wash step comprises washing the assay plate at least once, at least twice, at least three times, at least four times, or at least five times with a wash buffer. In embodiments, the assay plate is washed with at least about 10 μL, at least about 15 μL, at least about 20 μL, at least about 25 μL, at least about 30 μL, at least about 40 μL, at least about 50 μL, at least about 60 μL, at least about 70 μL, at least about 80 μL, at least about 90 μL, at least about 100 μL, at least about 150 μL, or at least about 200 μL of wash buffer. In embodiments, the assay step does not comprise a wash step prior to any of steps (a), (b), or (c).

In embodiments comprising a coating solution, the assay further comprises, prior to step (a), mixing a linking agent connected to a targeting agent complement with a binding reagent comprising a supplemental linking agent, thereby forming the coating solution comprising the binding reagent bound to the linking agent. In embodiments, the method comprises forming about 200 μL to about 1000 μL, or about 300 μL to about 800 μL, or about 400 μL to about 600 μL of the coating solution. In embodiments, step (a) comprises incubating the linking agent and the binding reagent at about 15° C. to about 30° C., about 18° C. to about 28° C., about 20° C. to about 26° C., or about 22° C. to about 24° C. In embodiments, the method comprises forming about 500 μL of the coating solution by incubating about 300 μL of a solution comprising the linking agent and about 200 μL of a solution comprising the binding reagent, at about room temperature (e.g., about 22° C. to about 28° C.) for about 30 minutes. In embodiments, the incubating is performed without shaking. In embodiments, the assay further comprises contacting the coating solution with a stop solution (e.g., about 100 μL to about 500 μL, or about 150 μL to about 300 μL, or about 200 μL of a stop solution) to stop the binding reaction between the linking agent and supplemental linking agent. In embodiments, the coating solution and the stop solution are incubated for about 10 minutes to about 1 hour, about 20 minutes to about 40 minutes, or about 30 minutes. In embodiments, the coating solution and the stop solution are incubated at about 15° C. to about 30° C., about 18° C. to about 28° C., about 20° C. to about 26° C., or about 22° C. to about 24° C. In embodiments, the method further comprises, following incubation of the coating solution with the stop solution, diluting the coating solution using the stop solution, e.g., by 2-fold, 5-fold, 10-fold, or 20-fold, to a working concentration as described herein. In embodiments, the targeting agent and targeting agent complement comprise complementary oligonucleotides. In embodiments, the linking agent comprises avidin or streptavidin, and the supplemental linking agent comprises biotin.

In embodiments, step (a) comprises adding about 10 μL to about 200 μL, about 5 μL to about 100 μL, about 10 μL to about 90 μL, about 15 μL to about 80 μL, about 20 μL to about 70 μL, about 30 μL to about 60 μL, or about 50 μL of the coating solution or a solution containing the biotinylated binding reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (b) comprises adding about 5 μL to about 50 μL, about 10 μL to about 40 μL, about 20 μL to about 30 μL, about 15 μL, about 25 μL, about 35 μL, or about 50 μL of the sample, calibration reagent, or control reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (c) comprises adding about 5 μL to about 50 μL, about 10 μL to about 40 μL, about 10 μL to about 20 μL, about 20 μL to about 30 μL, about 15 μL, about 25 μL, about 35 μL, or about 50 μL of a solution comprising the ACE2 detection reagent comprising the binding reagent and the detection reagent to each well of the plate. In embodiments, the plate is incubated at about 15° C. to about 30° C., about 18° C. to about 28° C., about 20° C. to about 26° C., or about 22° C. to about 24° C. In embodiments, the plate is incubated for about 10 minutes to about 6 hours, or about 30 minutes to about 4 hours, or about 45 minutes to about 2 hours, or about 1 hour. In embodiments, the plate is incubated at about room temperature (e.g., about 22° C. to about 28° C.) for at least 30 minutes. In embodiments, the plate is incubated at about room temperature (e.g., about 22° C. to about 28° C.) for about 1 hour. In embodiments, the plate is incubated without shaking. In embodiments, the plate is incubated with shaking, e.g., at about 500 to 1000 rpm. In embodiments, the plate is incubated with shaking at about 700 rpm.

In embodiments, step (d) comprises adding a read buffer to each well of the plate. Read buffers are further described herein. In embodiments, about 5 μL to about 200 μL, about 5 μL to about 150 μL, about 5 μL to about 100 μL, about 10 μL to about 80 μL, about 20 μL to about 60 μL, about 40 μL, about 50 μL, about 100 μL, or about 150 μL of the read buffer is added to each well. In embodiments, the measuring comprises reading the plate, e.g., on a plate reader as described herein. In embodiments, the assay comprises reading the plate immediately following addition of the read buffer.

### Quantitation Embodiments

In embodiments, the invention further provides a method of determining viral exposure in a subject, (a) comprising conducting an immunoassay method described herein on a biological sample of the subject; (b) detecting the virus, viral component, and/or biomarker (e.g., antibody biomarker or inflammatory or tissue damage biomarker) as described herein; (c) determining if the amount of detected virus, viral component, and/or biomarker is higher or lower relative to a control; and (d) determining the viral exposure of the subject based on the determination of (c). In embodiments, the method comprises normalizing the detected amount of biomarker (e.g., antibody biomarker) to a control and determining whether the subject is exposed to, infected by, and/or immune to the virus. In embodiments, the control is a biological sample containing a known amount of biomarkers (e.g., antibody biomarkers or inflammatory or tissue damage biomarkers). In embodiments, the control is a biological sample obtained from a subject known to have never been exposed to the virus. In embodiments, the control is a biological sample obtained from a subject known to have recovered from an infection by the virus. In embodiments, the virus is a coronavirus. In embodiments, the virus is SARS-CoV-2.

In embodiments comprising a multiplexed immunoassay for quantifying the amount of a biomarker (e.g., an antibody biomarker or an inflammatory or tissue damage biomarker), the method further comprises determining a threshold value of the biomarker in a healthy subject. In embodiments, the threshold value is determined from the aggregate results of measured biomarker amounts in multiple healthy subjects. For example, the aggregate results from a certain number of samples can determine the percentile, e.g., 99^{th} percentile or greater, of biomarker levels in a healthy subject. Various statistical models and algorithms can be utilized to calculate the extent of viral infection and/or degree of immunity in a subject by comparing and/or normalizing the subject's measured biomarker amount to the threshold value of that biomarker.

In embodiments, the multiplexed immunoassay for quantifying the amount of an antibody biomarker (e.g., a serology assay described herein) that specifically binds to a viral antigen is performed substantially with an assay that measures inhibition of binding between a viral protein and its associated host receptor, e.g., the binding of the coronavirus spike protein to the ACE2 receptor or the NRP1 receptor. In embodiments, the antibody biomarker inhibits binding between the viral protein and its associated host receptor. In embodiments, the inhibition assay indirectly detects the antibody biomarker. In embodiments, simultaneous direct detection (e.g., utilizing a viral antigen as a binding reagent) and indirect detection (e.g., measuring inhibition between a viral protein and its receptor) of the antibody biomarker improves specificity of the antibody biomarker detection.

In embodiments, the invention provides methods of assessing the affinity of an antibody biomarker to a viral antigen described herein, e.g., a SARS-CoV-2 antigen. As used in the context of antibodies, “affinity” refers to the strength of interaction between an epitope (e.g., on a viral antigen) and an antibody's antigen-binding site. In embodiments, the invention provides methods of assessing the binding kinetics between an antibody biomarker and viral antigen described herein. Methods of measuring antibody affinity and/or binding kinetics include, e.g., surface plasmon resonance (SPR) and bio-layer interference (BLI). Antibody affinity measurement is further described in, e.g., Underwood, Advances in Virus Research 34:283-309 (1988); Azimzadeh et al., J Mol Recognition 3(3):108-116 (1990); Hearty et al., Methods Mol Biol 907:411-442 (2012); and Singhal et al., Anal Chem 82(20):8671-8679 (2010).

In embodiments, the invention provides methods of assessing the affinity of a neutralizing antibody to a viral antigen described herein, e.g., a SARS-CoV-2 antigen. In embodiments, the affinity determination of a neutralizing antibody in a serum or plasma sample for SARS-CoV-2 comprises: a) titrating a labeled competitor to a surface comprising a known amount of SARS-CoV-2 S protein to determine the K_{dL}, between the labeled ACE2 competitor and S protein; b) titrating: (i) a plasma sample known to contain neutralizing antibody for SARS-CoV-2 S while maintaining a constant ACE2 concentration, as described by equation 1(a); and (ii) ACE2 while maintaining a constant sample concentration, as described by equation 1(b); and c) solving the system of equations 1(a) and 1(b) to determine the average antibody concentration in sample [A] and average affinity K_{dA}. As described in equation 1(a), if the binding of fixed labeled ACE2, [L], is plotted against the log concentration of the unlabeled competitor antibody, [A], the resulting inhibition curve will be shifted by a factor of log(1+[L]/K_{dL}).

IC₅₀=K_{dLapp}=K_{dL}(1+[A]/K_{dA}) 1(a)

EC₅₀=K_{dAapp}=K_{dA}(1+[L]/K_{dL}) 1(b)

Affinity measurements are further described, e.g., in Hulme et al., British Journal of Pharmacology 161: 1219-1237 (2010).

### Assays for Viral Nucleic Acids

Amplification and Detection of Viral Nucleic Acid

In embodiments, the invention provides a method for detecting a coronavirus in a biological sample, comprising: a) contacting the biological sample with a binding reagent that specifically binds a nucleic acid of the coronavirus; b) forming a binding complex comprising the binding reagent and the coronavirus nucleic acid; and c) detecting the binding complex, thereby detecting the coronavirus in the biological sample. In embodiments, the coronavirus is SARS-CoV-2. In embodiments, the coronavirus nucleic acid is RNA. In embodiments, the binding reagent comprises a single stranded oligonucleotide.

In embodiments, the sample comprises a coronavirus nucleic acid. In embodiments, the method further comprises amplifying the coronavirus nucleic acid to form one or more additional copies of the coronavirus nucleic acid sequence, forming a plurality of binding complexes, each binding complex comprising a copy of the coronavirus nucleic acid sequence, and detecting the plurality of binding complexes, thereby detecting the coronavirus in the biological sample. In embodiments, the coronavirus nucleic acid is RNA, and the amplifying comprises reverse transcribing the RNA to form a cDNA, and amplifying the cDNA using polymerase chain reaction (PCR) to form a PCR product comprising a copy of the coronavirus nucleic acid sequence. In embodiments, the reverse transcription to form a cDNA and the PCR to amplify the cDNA are performed in a single reaction mixture. In embodiments, the reaction mixture further comprises a glycosylase enzyme. In embodiments, the glycosylase removes non-specific products from the reaction mixture. In embodiments, the glycosylase is uracil-N-glycosylase. In embodiments, the sample comprises an RT-PCR product, e.g., cDNA. In embodiments, the cDNA is generated from a coronavirus nucleic acid. In embodiments, the method comprises amplifying the cDNA using PCR to form a PCR product comprising a copy of the coronavirus nucleic acid sequence. In embodiments, the PCR is performed for about 10 to about 60 cycles, about 20 to about 50 cycles, or about 30 to about 40 cycles. In embodiments, the cDNA is amplified with a first primer that comprises a binding partner of the binding reagent and a second primer that comprises a detectable label or binding partner thereof, to form the PCR product. In embodiments, the first primer is a PCR forward primer and comprises the binding partner of the binding reagent at a 5′ end. In embodiments, the second primer is a PCR reverse primer and comprises the detectable label or binding partner thereof at a 3′ end. In embodiments, the PCR product comprises, in 5′ to 3′ order: the binding partner of the binding reagent, a copy of the coronavirus nucleic acid sequence, and the detectable label or binding partner thereof.

In embodiments, the first and second primers amplify a coronavirus nucleic acid sequence that encodes a protein, e.g., any of the coronavirus proteins described herein such as S, E, M, N, or a nonstructural protein. In embodiments, the first and second primers amplify a non-coding coronavirus nucleic acid sequence, i.e., that does not encode a gene. In embodiments, the first and second primers amplify a coronavirus nucleic acid sequence capable of identifying a coronavirus species. In embodiments, the coronavirus nucleic acid is SARS-CoV-2 RNA.

In embodiments, the method is a multiplexed method. In embodiments, the cDNA is amplified using multiple sets of primers, wherein each set of primers comprises a PCR forward primer and a PCR reverse primer as described herein. In embodiments, the PCR forward primer in each set of primers comprises a binding partner of the same binding reagent. In embodiments, the PCR forward primer in each set of primers comprises a binding partner of different binding reagents. In embodiments, each set of primers amplifies a different region of the cDNA to generate a plurality of PCR products, each having a different coronavirus nucleic acid sequence. For example, a first set of primers amplifies a region that encodes for the S protein, a second set of primers amplifies a region that encodes for the N protein, a third set of primers amplifies a region for a noncoding region, etc. In embodiments, each coronavirus nucleic acid sequence corresponds to a different binding reagent. In embodiments, the coronavirus nucleic acid sequence of the PCR product is identified by determining the binding reagent that binds the PCR product. In embodiments, the coronavirus nucleic acid is SARS-CoV-2 RNA. In embodiments, the primers for amplifying a region that encodes for the S protein are described in Table 25. In embodiments, the primer comprises a modified nucleotide, e.g., a locked nucleic acid (LNA).

In embodiments, the binding reagent comprises a single-stranded oligonucleotide, and the binding partner of the binding reagent comprises a complementary oligonucleotide of the binding reagent. In embodiments, the binding reagent further comprises a targeting agent complement. In embodiments, the targeting agent complement comprises an oligonucleotide that is complementary to a targeting agent on a surface, as described herein. In embodiments, the binding reagent is immobilized to the surface via the targeting agent—targeting agent complement interaction. In embodiments, each PCR product binds to a binding reagent to form one or more binding complexes on the surface. In embodiments comprising different binding reagents corresponding to different coronavirus nucleic acid sequences, each binding reagent is located at a distinct binding domain on the surface, and the detected coronavirus nucleic acid sequence is identified by the location of the binding complex on the surface.

In embodiments, the method comprises detecting the binding complex(es). In embodiments, the PCR product comprises a detectable label. In embodiments, the PCR product comprises a binding partner of a detectable label. Detectable labels are described herein. In embodiments, the detectable label is an electrochemiluminescence (ECL) label. In embodiments, the PCR product comprises biotin, and the detectable label comprises an ECL label linked to avidin or streptavidin. In embodiments, the PCR product comprises avidin or streptavidin, and the detectable label comprises an ECL label linked to biotin. Additional non-limiting examples of binding partners that can be on the detection probe and detectable label are provided herein.

In embodiments, RNA is extracted from a sample containing an RNA virus (e.g., SARS-CoV-2), and the extracted RNA is converted to cDNA. A “Master Mix” is prepared by combining a forward primer comprising a 5′ binding reagent complement sequence and a cDNA complement sequence, a reverse primer comprising a cDNA reverse complement sequence and a 3′ binding partner of a detectable label, and other PCR components such as dNTPs and DNA polymerase (e.g., Taq polymerase). The cDNA and Master Mix are combined, and PCR is performed for 30 to 40 cycles to form a plurality of PCR products, each PCR product comprising the 5′ binding reagent complement sequence and 3′ binding partner of a detectable label. Each PCR product hybridizes to a binding reagent on a surface. The surface is then contacted with a detectable label, which binds to the PCR product. The PCR product bound to the detectable label is then subjected to detection as described herein.

In an alternative embodiment, the Master Mix comprises the components for performing the reverse transcription reaction and the PCR reaction, e.g., reverse transcriptase, DNA polymerase, forward and reverse primers, nucleotides, magnesium, ribonuclease inhibitor, and glycosylase, and the RNA extracted from the sample is added to the Master Mix, such that the reverse transcription reaction and the PCR reaction are performed with a single reaction mixture to form the PCR product. In embodiments, the single reaction mixture is: (1) incubated at a first temperature sufficient to activate the glycosylase; (2) incubated at a second temperature sufficient to perform the reverse transcription; and (3) incubated at temperature sufficient to perform PCR. In embodiments, the PCR product is bound to the surface and detected as described herein.

Detection of Viral Nucleic Acids and Single Nucleotide Polymorphisms (SNPs)

In embodiments, the invention provides a method for detecting a coronavirus nucleic acid in a biological sample. In embodiments, the invention provides a method of identifying the circulating strains of SARS-CoV-2 without sequencing a large number of SARS-CoV-2 isolates. Certain strains of SARS-CoV-2 are associated with increased transmissibility (e.g., the B.1.1.7, 501Y.V2, and P.1 strains) and diminished efficacy against currently available vaccines. In embodiments, the invention provides a method of real-time monitoring and assessing transmission patterns of SARS-CoV-2. In embodiments, the invention provides a method for determining a SARS-CoV-2 strain (e.g., the L strain or S strain, or the S-D614 or S-D614G strain, or the variants in Table 1A as described herein) in a biological sample.

In embodiments, the invention provides method for detecting a single nucleotide polymorphism (SNP) in a target nucleic acid, wherein the target nucleic acid is a SARS-CoV-2 nucleic acid, comprising: (a) contacting a sample comprising the target nucleic acid with (i) a targeting probe, wherein the targeting probe comprises a first region complementary to a polymorphic site of the target nucleic acid that comprises the SNP, and wherein the targeting probe comprises an oligonucleotide tag; and (ii) a detection probe, wherein the detection probe comprises a second region complementary to an adjacent region of the target nucleic acid comprising the polymorphic site, and wherein the detection probe comprises a detectable label; (b) hybridizing the targeting and detection probes to the target nucleic acid; (c) ligating the targeting and detection probes that hybridize with perfect complementarity at the polymorphic site to form a ligated target complement comprising the oligonucleotide tag and the detectable label; (d) contacting the product of (c) with a surface comprising an immobilized binding reagent, wherein the binding reagent comprises an oligonucleotide complementary to the oligonucleotide tag; (e) forming a binding complex on the surface, wherein the binding complex comprises the binding reagent and the ligated target complement; and (f) detecting the binding complex, thereby detecting the SNP at the polymorphic site. In embodiments, the targeting probe and the detection probe each independently comprises a sequence as shown in Table 10 or Table 14.

In embodiments, the method comprises an oligonucleotide ligation assay (OLA). OLA and other nucleic acid detection methods are described, e.g., in WO 2020/227016. In embodiments, the OLA method is used to detect, identify, and/or quantify a coronavirus nucleic acid (e.g., RNA). In embodiments, the coronavirus nucleic acid encodes the N gene. In embodiments, the coronavirus nucleic acid is the N1 region, N2 region, or N3 region of the N gene, as described herein. In embodiments, the OLA method is used to detect, identify, and/or quantify a single nucleotide polymorphism (SNP) at a polymorphic site in a coronavirus nucleic acid (e.g., RNA). In embodiments, the coronavirus is SARS-CoV-2. In embodiments, the method detects any of the SNPs as shown in Table 1A and Table 1C.

In embodiments, the OLA method for detecting a coronavirus nucleic acid comprises: (a) contacting the biological sample with: (i) a targeting probe, wherein the targeting probe is complementary to a first region of a target nucleic acid (e.g., the coronavirus nucleic acid or an RT-PCR product described herein), and wherein the targeting probe comprises an oligonucleotide tag; and (ii) a detection probe, wherein the detection probe is complementary to a second region that is adjacent to the first region of the target nucleic acid; (b) hybridizing the targeting and detection probes to the target nucleic acid; (c) ligating the targeting and detection probes that hybridize with perfect complementarity to the first and second regions of the target nucleic acid to form a ligated target complement comprising the oligonucleotide tag and the detectable label; (d) contacting the product of (c) with a surface comprising a binding reagent immobilized in one or more binding domains, wherein the binding reagent comprises an oligonucleotide complementary to the oligonucleotide tag; (e) forming a binding complex on the surface between the binding reagent and the ligated target complement; and (f) detecting the binding complex, thereby detecting, identifying, and/or quantifying the coronavirus nucleic acid. In embodiments, the coronavirus is SARS-CoV-2. In embodiments, the coronavirus nucleic acid is RNA. In embodiments, the sample comprises the coronavirus nucleic acid. In embodiments, the sample comprises an RT-PCR product, e.g., cDNA that is generated from the coronavirus nucleic acid.

In embodiments, the OLA method for detecting an SNP comprises: (a) contacting the biological sample with: (i) a targeting probe, wherein the targeting probe is complementary to a polymorphic site of target nucleic acid (e.g., the coronavirus nucleic acid or an RT-PCR product described herein), and wherein the targeting probe comprises an oligonucleotide tag; and (ii) a detection probe, wherein the detection probe is complementary to an adjacent region of the target nucleic acid containing the distinct SNP; (b) hybridizing the targeting and detection probes to the target nucleic acid; (c) ligating the targeting and detection probes that hybridize with perfect complementarity at the polymorphic site to form a ligated target complement comprising the oligonucleotide tag and the detectable label; (d) contacting the product of (c) with a surface comprising a binding reagent immobilized in one or more binding domains, wherein the binding reagent comprises an oligonucleotide complementary to the oligonucleotide tag; (e) forming a binding complex on the surface between the binding reagent and the ligated target complement; and (f) detecting the binding complex, thereby detecting, identifying, and/or quantifying the SNP at the polymorphic site of the coronavirus nucleic acid. In embodiments, the coronavirus is SARS-CoV-2. In embodiments, the coronavirus nucleic acid is RNA. In embodiments, the sample comprises the coronavirus nucleic acid. In embodiments, the sample comprises an RT-PCR product, e.g., cDNA that is generated from the coronavirus nucleic acid.

In embodiments, the ligating of the oligonucleotide probes is dependent on three events: (1) the targeting and detection probes must hybridize to complementary sequences within the target nucleic acid; (2) the targeting and detection probes must be adjacent to one another in a 5′- to 3′-orientation with no intervening nucleotides; and (3) the targeting and detection probes must have perfect base-pair complementarity with the target nucleic acid at the ligation site. A single nucleotide mismatch between the primers and target may inhibit ligation. In embodiments, the melting temperature (T_{M}) of the oligonucleotide probes is about 55° C. to about 70° C., about 58° C. to about 68° C., about 60° C. to about 67° C., or about 62° C. to about 66° C. In embodiments, the ligation is performed at about 60° C. to about 70° C., about 61° C. to about 69° C., or about 62° C. to about 68° C. In embodiments, the ligation is performed at about 60° C., about 61° C., about 62° C., about 63° C., about 64° C., about 65° C., about 66° C., about 67° C., about 68° C., about 69° C., or about 70° C.

In embodiments, the targeting probe comprises, in 5′- to 3′-order: the oligonucleotide tag, and a sequence that is complementary to a first region of the target nucleic acid. In embodiments where the method detects a polymorphic site (SNP), the first region of the target nucleic acid comprises the polymorphic site. In embodiments, the oligonucleotide tag comprises a single-stranded oligonucleotide. In embodiments, the oligonucleotide tag does not hybridize with the target nucleic acid. In embodiments, the detection probe comprises, in 5′- to 3′-order: a sequence that is complementary to a second region of the target nucleic acid that is adjacent to the first region, and a detectable label or binding partner thereof. In embodiments, the 5′ end of the targeting probe is phosphorylated and is adjacent to the 3′ hydroxyl of the detection probe when the targeting and detection probes are hybridized to the target nucleic acid, such that the ends of the targeting and detection probes are ligated by formation of a phosphodiester bond. In embodiments, the 5′ end of the detection probe is phosphorylated and is adjacent to the 3′ hydroxyl of the targeting probe when the targeting and detection probes are hybridized to the target nucleic acid, such that the ends of the targeting and detection probes are ligated by formation of a phosphodiester bond. In embodiments, the targeting probe and/or the detection probe comprises a modified nucleotide, e.g., a locked nucleic acid (LNA).

In embodiments, the targeting and detection probes are ligated using a template-dependent ligase, for example, a DNA ligase such as E. coli DNA ligase, T4 DNA ligase, T aquaticus (Taq) ligase, T Thermophilus DNA ligase, or Pyrococcus DNA ligase. In embodiments, the ligase is a thermostable ligase. In embodiments, the targeting and detection probes are ligated by chemical ligation. In embodiments, the hybridization and ligation are performed in a combined step, for example, using multiple thermocycles and a thermostable ligase.

In embodiments where the method detects a coronavirus nucleic acid (e.g., the SARS-CoV-2 N gene or the N1, N2, and/or N3 regions thereof), the targeting probe hybridizes to the target nucleic acid such that the terminal 5′ nucleotide of the targeting probe hybridizes with the first region in the target nucleic acid, and the detection probe hybridizes to the second region in the target nucleic acid that is adjacent to the first region and provides a 3′ end for the ligation of the targeting and detection probes. In embodiments, the detection probe hybridizes to the target nucleic acid such that the terminal 5′ nucleotide of the detection probe hybridizes with the first region in the target nucleic acid, and the targeting probe hybridizes to the second region in the target nucleic acid that is adjacent to the first region and provides a 3′ end for the ligation of the targeting and detection probes. In embodiments, the detection probe hybridizes to the target nucleic acid such that the terminal 3′ nucleotide of the detection probe hybridizes with the first region in the target nucleic acid, and the targeting probe hybridizes to the second region of the target nucleic acid that is adjacent to the first region and provides a 5′ end for the ligation of the targeting and detection probes.

In embodiments where the method detects an SNP, the targeting probe hybridizes to the target nucleic acid such that the terminal 5′ nucleotide of the targeting probe hybridizes with the SNP in the target nucleic acid, and the detection probe hybridizes to the target nucleic acid adjacent to the SNP and provides a 3′ end for the ligation of the targeting and detection probes. In embodiments, the detection probe hybridizes to the target nucleic acid such that the terminal 5′ nucleotide of the detection probe hybridizes with the SNP in the target nucleic acid, and the targeting probe hybridizes to the target nucleic acid adjacent to the SNP and provides a 3′ end for the ligation of the targeting and detection probes. In embodiments, the detection probe hybridizes to the target nucleic acid such that the terminal 3′ nucleotide of the detection probe hybridizes with the SNP in the target nucleic acid, and the targeting probe hybridizes to the target nucleic acid adjacent to the SNP and provides a 5′ end for the ligation of the targeting and detection probes.

In embodiments, the method further comprises providing a blocking probe during the ligating of the targeting and detection probes. In embodiments, a blocking probe reduces non-specific bridging background during the ligation reaction. In embodiments, the blocking probe comprises a single stranded oligonucleotide that is complementary to the target nucleic acid and straddles the ligation site but does not comprise an oligonucleotide tag or a detectable label or binding partner thereof. In embodiments, the blocking probe comprises a single stranded oligonucleotide that is complementary to a probe designed to hybridize to the target nucleic acid. Without being bound by theory, it is believed that the presence of a blocking probe can reduce formation of complexes in which the target nucleic acid functions as a “bridge” for probes that are annealed to the target nucleic acid, but not ligated to one another, such that the complex can generate a false signal. In embodiments, a pair of blocking probes is provided during the ligating. In embodiments, one or more blocking probes is provided during the ligating in excess over the corresponding targeting and/or detection probes. In embodiments, the blocking probe comprises a sequence as shown in Table 12 or Table 16.

In embodiments, the detection probe comprises a detectable label. In embodiments, the detection probe comprises a binding partner of a detectable label. Detectable labels are described herein. In embodiments, the detectable label is an electrochemiluminescence (ECL) label. In embodiments, the detection probe comprises biotin, and the detectable label comprises an ECL label linked to avidin or streptavidin. In embodiments, the detection probe comprises avidin or streptavidin, and the detectable label comprises an ECL label linked to biotin. Additional non-limiting examples of binding partners that can be on the detection probe and detectable label are provided herein.

In embodiments, the target nucleic acid in the sample comprises a coronavirus nucleic acid. In embodiments, the target nucleic acid in the sample comprises an RT-PCR product, e.g., cDNA generated from the coronavirus nucleic acid. In embodiments, the method further comprises amplifying the target nucleic acid prior to contacting with the oligonucleotide probes. In embodiments, the method does not comprise amplifying the target nucleic acid. In embodiments, the nucleic acid is coronavirus RNA, and the method comprises reverse transcribing the coronavirus RNA into cDNA prior to step (a). In embodiments, the targeting probe and/or detection probe hybridize to the cDNA strand comprising the SNP of interest. In embodiments where the SNP of interest is in a protein coding sequence, the targeting probe and/or detection probe hybridize to the cDNA strand comprising the protein coding sequence. In embodiments, the targeting probe and/or detection probe hybridize to the cDNA strand comprising a complement of the SNP of interest. In embodiments wherein the SNP of interest is in a protein coding sequence, the targeting probe and/or detection probe hybridize to the cDNA strand comprising the complementary strand of the protein coding sequence. In embodiments, the coronavirus is SARS-CoV-2.

In embodiments, the region between SARS-CoV-2 genome locations 28250 and 28400, or between locations 28280 and 28390, or between locations 28300 and 28980, or between locations 28303 and 29374 is reverse transcribed prior to step (a). In embodiments, the region between SARS-CoV-2 genome locations 29000 and 29300, or between locations 29100 and 29280, or between locations 29150 and 29250, or between locations 29180 and 29246 is reverse transcribed prior to step (a). In embodiments, the region between SARS-CoV-2 genome locations 28500 and 28800, or between locations 28550 and 28790, or between locations 28600 and 28780, or between locations 28697 and 28768 is reverse transcribed prior to step (a). In embodiments, the cDNA formed by the reverse transcription is amplified by PCR. Exemplary PCR primers for amplification are shown in Table 8 and described in Lu et al., Emerg Infect Dis 26(8):1654-1665 (2020).

In embodiments, the region between SARS-CoV-2 genome locations 20000 and 25000, or between locations 21000 and 24500, or between locations 21500 and 24000, or between locations 21661 and 23812, or between locations 21739 and 23707, or between locations 21000 and 23000, or between 21500 and 22500, or between locations 21706 and 22341, or between locations 22000 and 24000, or between locations 22500 and 23500, or between locations 22624 and 23321, or between locations 23000 and 25000, or between locations 23000 and 24500, or between locations 23442 and 24103, or between locations 21662-21918, or between locations 22017-22341, or between locations 22851-23128, or between locations 23542-23813, or between locations 21706-21873, or between locations 22014-22252, or between locations 22624-22871, or between locations 22879-23099, or between locations 23296-23647, or between locations 23576-23815, or between locations 21600-21871, or between locations 22016-22331, or between locations 22849-23134, or between locations 23434-23755, or between locations 22589-22860, or between locations 22641-22865, or between locations 22561-22860, or between locations 22561-22865 is reverse transcribed prior to step (a). In embodiments, the cDNA formed by the reverse transcription is amplified by PCR. Exemplary PCR primers for amplification are shown in Table 13. In embodiments, the method comprises detecting an SNP in a synthetic oligonucleotide template. Exemplary synthetic oligonucleotide template sequences are shown in Table 11.

In embodiments, the region surrounding a SARS-CoV-2 genome location described in Table 1A and/or Table 1C is reverse transcribed prior to step (a). In embodiments, the region surrounding the SARS-CoV-2 S gene, N gene, E gene, 5′ UTR, nsp3 gene, Orf1ab gene, Orf1a gene, RdRp gene, Orf3a gene, Orf8 gene, or Orf10 gene is reverse transcribed prior to step (a). In embodiments, the region surrounding a particular genomic location includes about 10 to about 1000 nucleotides in length, about 20 to about 900 nucleotides in length, about 30 to about 800 nucleotides in length, about 40 to about 700 nucleotides in length, about 50 to about 600 nucleotides in length, about 60 to about 500 nucleotides in length, about 70 to about 400 nucleotides in length, about 80 to about 300 nucleotides in length, about 90 to about 200 nucleotides in length, or about 100 to about 150 nucleotides in length.

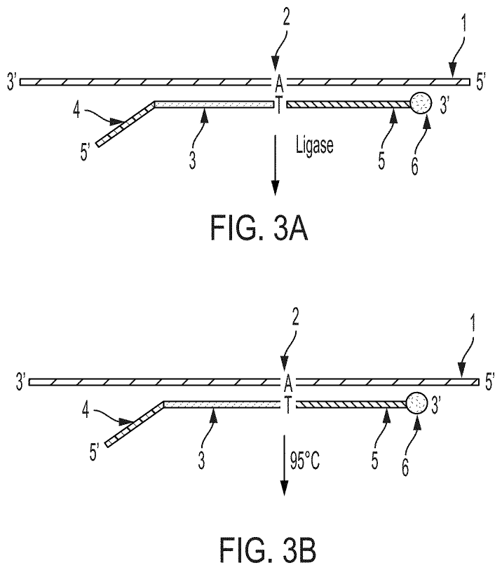

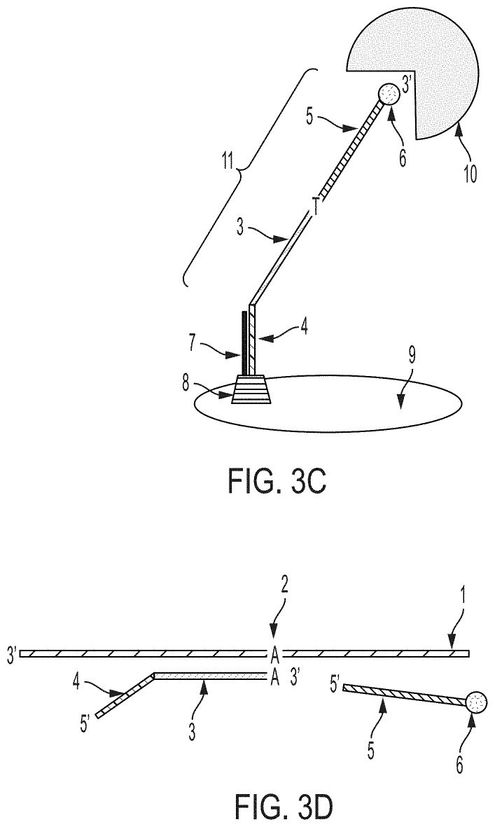

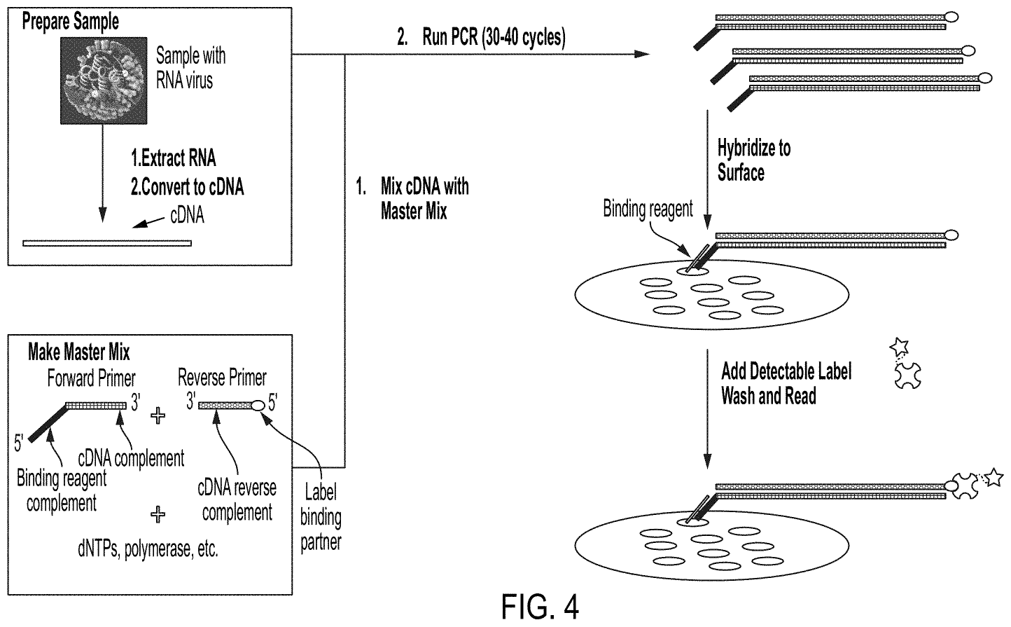

An embodiment of the OLA method for detecting an SNP described herein is represented schematically in FIG. 3. In FIGS. 3A-3C, a target nucleic acid (1) that comprises an SNP (2) is contacted with: a targeting probe (3) that comprises an oligonucleotide tag (4) and a sequence that is complementary to the SNP, and a detection probe (5) that comprises detectable label (6). The targeting and detection probes (3, 5) hybridize to the target nucleic acid, and the targeting and detection probes that hybridize with perfect complementarity at the SNP are ligated to form a ligated target complement (11) comprising the oligonucleotide tag and detectable label. The reaction mixture containing the ligated target complement is contacted with a surface comprising one or more binding reagents (7) immobilized in one or more binding domains (9). A signal (10) is detected if the ligated target complement is immobilized on the surface via hybridization of the complementary oligonucleotides in the oligonucleotide tag and the binding reagent. In FIG. 3D, the targeting probe has a mismatch with the SNP in the target nucleic acid, and thus, hybridization and ligation do not occur.

In embodiments, the method is a multiplexed OLA method. In embodiments where the method detects a coronavirus nucleic acid, the biological sample is contacted with one or more targeting probes and one or more detection probes to different regions of the coronavirus nucleic acid to form a plurality of ligated target complements. In embodiments, targeting probes for individual coronavirus nucleic acid regions comprise oligonucleotide tags corresponding to the individual coronavirus nucleic acid regions. In embodiments, the targeting probes for different coronavirus nucleic acid regions have substantially the same melting temperatures (T_{M}), e.g., within about 5° C., within about 4° C., within about 3° C., within about 2° C., or within about 1° C. In embodiments, the targeting probes for different coronavirus nucleic acid regions have substantially the same melting temperatures (T_{M}), e.g., within about 5° C., within about 4° C., within about 3° C., within about 2° C., or within about 1° C. In embodiments, the surface comprises a plurality of binding reagents capable of hybridizing to the different oligonucleotide tags. In embodiments, a plurality of binding complexes, each comprising a ligated target complement and its corresponding binding reagent, are formed on the surface, and the binding complexes are detected, thereby detecting, identifying, and/or quantifying each of the different coronavirus nucleic acid regions. In embodiments, the coronavirus is SARS-CoV-2. In embodiments, the coronavirus nucleic acid is RNA. In embodiments, the different coronavirus regions comprise the N1, N2, and N3 regions of SARS-CoV-2.

In embodiments where the method detects an SNP, the biological sample is contacted with one or more SNP-specific targeting probes and one or more detection probes to form a plurality of ligated target complements. In embodiments, the detection probes comprise identical sequences. In embodiments, each of the one or more SNP-specific targeting probes hybridizes to a different SNP at the target nucleic acid (e.g., any of the SARS-CoV-2 genome locations and SNPs in Tables 1A and 1C herein). In embodiments, the targeting probe and detection probe for detecting a SNP in Tables 1A and 1C comprises a sequence described in Table 10. In embodiments, the blocking oligonucleotide for detecting the SNPs in Tables 1A and 1C comprises a sequence described in Table 12. In embodiments, targeting probes for different SNPs comprise different oligonucleotide tags. In embodiments, the targeting probes for different SNPs have substantially the same melting temperatures (T_{M}), e.g., within about 5° C., within about 4° C., within about 3° C., within about 2° C., or within about 1° C. In embodiments, the surface comprises a plurality of binding reagents capable of hybridizing to the different oligonucleotide tags. In embodiments, a plurality of binding complexes, each comprising a ligated target complement and its corresponding binding reagent, are formed on the surface, and the binding complexes are detected, thereby detecting, identify, and/or quantifying each of the SNPs at the polymorphic site of the coronavirus nucleic acid. In embodiments, the coronavirus is SARS-CoV-2. In embodiments, the coronavirus nucleic acid is RNA.

In embodiments, the multiplexed OLA method simultaneously detects at least 2, at least 3, at least 4, at least 5, at least 6, at least 7, at least 8, at least 9, or at least 10 coronavirus nucleic acids as described herein, e.g., the SARS-CoV-2 N1, N2, and N3 regions. In embodiments, the multiplexed OLA method simultaneously detects at least 2, at least 3, at least 4, at least 5, at least 6, at least 7, at least 8, at least 9, or at least 10 SNPs, e.g., the SNPs at SARS-CoV-2 genome locations 8782, 11083, 23403, and 28144. In embodiments, the multiplexed OLA method simultaneously detects the SNPs at SARS-CoV-2 genome locations 21765-21770, 22132, 22206, 22813, 22812, 22917, 23012, 23063, 23604, and 23664, which correspond to amino acid residues 69-70, R190, D215, K417, K417, L452, E484, N501, P681, and A701, respectively, of the SARS-CoV-2 S protein. In embodiments, the multiplexed OLA method comprises contacting the biological sample with a surface comprising at least 2, at least 3, at least 4, at least 5, at least 6, at least 7, at least 8, at least 9, or at least 10 distinct binding domains, wherein each binding domain comprises a unique binding reagent, each unique binding reagent capable of recognizing a different oligonucleotide tag as described herein.

In embodiments, the method further comprises detecting a control gene. In embodiments, the control gene comprises an endogenous gene of the subject from which the biological sample was obtained. In embodiments, the control gene comprises the human RPP30 gene. In embodiments, the targeting probe for the human RPP30 gene comprises SEQ ID NO:35 or 37. In embodiments, the detection probe for the human RPP30 gene comprises SEQ ID NO:36 or 38. In embodiments, the blocking probe for the human RPP30 gene comprises any one of SEQ ID NOs:51-54.

In embodiments, the invention provides a method for detecting a coronavirus nucleic acid in a biological sample. In embodiments, the method comprises: (a) contacting the biological sample with (i) a polymerase; (ii) a forward primer, wherein the forward primer binds to a first region of a target nucleic acid (e.g., the coronavirus nucleic acid or an RT-PCR product described herein), and wherein the forward primer comprises an oligonucleotide tag; and (iii) a reverse primer, wherein the reverse primer binds to a second region of the target nucleic acid; (b) amplifying the target nucleic acid using the polymerase to form an amplified target nucleic acid comprising the oligonucleotide tag; (c) hybridizing the amplified target nucleic acid with an internal detection probe that is complementary to at least a portion of the amplified target nucleic acid, thereby forming a hybridized target; (d) contacting the hybridized target with a surface comprising a binding reagent immobilized in one or more binding domains, wherein the binding reagent comprises an oligonucleotide complementary to the oligonucleotide tag; (e) forming a binding complex on the surface between the binding reagent and the hybridized target; and (0 detecting the binding complex, thereby detecting, identifying, and/or quantifying the coronavirus nucleic acid. In embodiments, the coronavirus is SARS-CoV-2. In embodiments, the coronavirus nucleic acid is RNA. In embodiments, the target nucleic acid is the N1, N2, and/or N3 regions of SARS-CoV-2. In embodiments, the sample comprises the coronavirus nucleic acid. In embodiments, the sample comprises an RT-PCR product, e.g., cDNA that is generated from the coronavirus nucleic acid.

In embodiments, the method further comprises amplifying the target nucleic acid prior to contacting with the oligonucleotide probes. In embodiments, the nucleic acid is coronavirus RNA, and the method comprises reverse transcribing the coronavirus RNA into cDNA prior to step (a). In embodiments, the coronavirus is SARS-CoV-2. In embodiments, the method comprises, prior to step (a), reverse transcribing the region between SARS-CoV-2 genome locations 28250 and 28400, or between locations 28280 and 28390, or between locations 28300 and 28980, or between locations 28303 and 29374. In embodiments, the method comprises, prior to step (a), reverse transcribing the region between SARS-CoV-2 genome locations 29000 and 29300, or between locations 29100 and 29280, or between locations 29150 and 29250, or between locations 29180 and 29246. In embodiments, the method comprises, prior to step (a), reverse transcribing the region between SARS-CoV-2 genome locations 28500 and 28800, or between locations 28550 and 28790, or between locations 28600 and 28780, or between locations 28697 and 28768.

In embodiments, the internal detection probe comprises a detectable label. In embodiments, the internal detection probe comprises a binding partner of a detectable label. Detectable labels are described herein. In embodiments, the detectable label is an electrochemiluminescence (ECL) label. In embodiments, the internal detection probe comprises biotin, and the detectable label comprises an ECL label linked to avidin or streptavidin. In embodiments, the internal detection probe comprises avidin or streptavidin, and the detectable label comprises an ECL label linked to biotin. Additional non-limiting examples of binding partners that can be on the internal detection probe and detectable label are provided herein.

In embodiments, the method further comprises detecting a control gene. In embodiments, the control gene comprises an endogenous gene of the subject from which the biological sample was obtained. Suitable control genes are known to those of skill in the art. In embodiments, the control gene comprises the human RPP30 gene. In embodiments, the forward primer for the human RPP30 gene comprises SEQ ID NO:64. In embodiments, the reverse primer for the human RPP30 gene comprises SEQ ID NO:65. In embodiments, the internal detection probe for the human RPP30 gene comprises SEQ ID NO:66.

In embodiments, the invention provides a multiplexed OLA method for detecting SARS-CoV-2 strains, comprising detecting the SARS-CoV-2 genome location 21765-21770 (corresponding to amino acid residues 69-70 of the S protein), location 22132 (corresponding to amino acid residue 190 of the S protein), location 22206 (corresponding to amino acid residue 215 of the S protein), location 22812 (corresponding to amino acid residue 417 of the S protein), location 22813 (corresponding to amino acid residue 417 of the S protein), location 22917 (corresponding to amino acid residue 452 of the S protein), location 23012 (corresponding to amino acid residue 484 of the S protein), location 23063 (corresponding to amino acid residue 501 of the S protein), location 23403 (corresponding to amino acid residue 614 of the S protein), location 23604 (corresponding to amino acid residue 681 of the S protein), and/or location 23664 (corresponding to amino acid residue 701 of the S protein). In embodiments, the method further comprises detecting a control gene. In embodiments, the control gene comprises a gene or region that is conserved across SARS-CoV-2 strains. In embodiments, the control gene is SARS-CoV-2 N1.

In embodiments, the multiplexed OLA method detects: a deletion at SARS-CoV-2 genome location 21765-21770 (corresponding to a deletion of residues 69-70 of the S protein), a G>T SNP at SARS-CoV-2 genome location 22132 (corresponding to an R190S mutation in the S protein), an A>G SNP at SARS-CoV-2 genome location 22206 (corresponding to a D215G mutation in the S protein), an A>G SNP at SARS-CoV-2 genome location 22320 (corresponding to a D253G mutation in the S protein), an A>C SNP at SARS-CoV-2 genome location 22812 (corresponding to a K417T mutation in the S protein), a G>T SNP at SARS-CoV-2 genome location 22813 (corresponding to a K417N mutation in the S protein), a T>G SNP at SARS-CoV-2 genome location 22917 (corresponding to an L452R mutation in the S protein), a G>A SNP at SARS-CoV-2 genome location 23012 (corresponding to a E484K mutation in the S protein), an A>T SNP at SARS-CoV-2 genome location 23063 (corresponding to a N501Y mutation the S protein), an A>G SNP at SARS-CoV-2 genome location 23403 (corresponding to a D614G mutation in the S protein), a C>A SNP at SARS-CoV-2 genome location 23604 (corresponding to a P681H mutation in the S protein), and/or a C>T SNP at SARS-CoV-2 genome location 23664 (corresponding to an A701V mutation in the S protein). In embodiments, the multiplexed OLA method detects any combination of the SNPs in Table 1A.

In embodiments, the multiplexed OLA method detects multiple variants of SARS-CoV-2. In embodiments, the multiplexed OLA method detects multiple variants of SARS-CoV-2. As used herein, “variant” refers to a strain that has one or more mutations relative to the SARS-CoV-2 reference strain NC_045512. In embodiments, the multiplexed OLA method is conducted in a multi-well plate, wherein each well comprises ten binding domains (“spots”) in an arrangement as shown in FIG. 39B. In embodiments, Spot 1 comprises a binding reagent for detecting a deletion at 21765-21770 (S protein Δ69-70 deletion); Spot 2 comprises a binding reagent for detecting 22132T (S protein R190S mutation); Spot 3 comprises a binding reagent for detecting 22206G (S protein D215G mutation); Spot 4 comprises a binding reagent for detecting 22917G (S protein L452R mutation); Spot 6 comprises a binding reagent for detecting 23012A (S protein E484K mutation); Spot 7 comprises a binding reagent for detecting 23063T (S protein N501Y mutation); Spot 8 comprises a binding reagent for detecting 23403G (S protein D614G mutation); Spot 9 comprises a binding reagent for detecting 23604A (S protein P681H mutation); Spot 10 comprises a binding reagent for detecting 23664T (S protein A701V mutation); and Spot 5 comprises a binding reagent for detecting a control gene of SARS-CoV-2 that is conserved across all SARS-CoV-2 strains, e.g., the N1 gene.

In embodiments, the multiplexed OLA method simultaneously detects the reference SARS-CoV-2 strain and one or more variants, e.g., by detecting both the wild-type nucleotide and the variant SNP at a genome location. In embodiments, the multiplexed OLA method is conducted in a multi-well plate, wherein each well comprises ten binding domains (“spots”) in an arrangement as shown in FIG. 39B. In embodiments, Spot 1 comprises a binding reagent for detecting a deletion at 21765-21770 (S protein 469-70 deletion), and Spot 6 comprises a binding reagent for detecting the wild-type sequence at 21765-21770 (S protein residues 69-70); Spot 2 comprises a binding reagent for detecting 22132T (S protein R190S mutation), and Spot 7 comprises a binding reagent for detecting 22132G (S protein R190); Spot 3 comprises a binding reagent for detecting 22206G (S protein D215G mutation), and Spot 8 comprises a binding reagent for detecting 22206A (S protein D215); Spot 4 comprises a binding reagent for detecting 22917G (S protein L452R mutation), and Spot 9 comprises a binding reagent for detecting 22917T (S protein L452).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting 23012G (S protein E484), and Spot 6 comprises a binding reagent for detecting 23012A (S protein E484K mutation); Spot 2 comprises a binding reagent for detecting 23063A (S protein N501), and Spot 7 comprises a binding reagent for detecting 23063T (S protein N501Y mutation); Spot 3 comprises a binding reagent for detecting 23403A (S protein D614), and Spot 8 comprises a binding reagent for detecting 23403G (S protein D614G mutation); Spot 4 comprises a binding reagent for detecting 23604C (S protein P681), and Spot 9 comprises a binding reagent for detecting 23604A (S protein P681H mutation); Spot 5 comprises a binding reagent for detecting 23664C (S protein A701), and Spot 10 comprises a binding reagent for detecting 23664T (S protein A701V mutation).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting a deletion at 21765-21770 (S protein 469-70 deletion), and Spot 6 comprises a binding reagent for detecting the wild-type sequence at 21765-21770 (S protein residues 69-70); Spot 2 comprises a binding reagent for detecting 23063A (S protein N501), and Spot 7 comprises a binding reagent for detecting 23063T (S protein N501Y mutation); Spot 4 comprises a binding reagent for detecting 23604C (S protein P681), and Spot 9 comprises a binding reagent for detecting 23604A (S protein P681H mutation); Spot 7 comprises a binding reagent for detecting 22132G (S protein R190), and Spot 2 comprises a binding reagent for detecting 22132T (S protein R190S mutation); Spot 8 comprises a binding reagent for detecting 22206A (S protein D215), and Spot 3 comprises a binding reagent for detecting 22206G (S protein D215G mutation); Spot 9 comprises a binding reagent for detecting 22917T (S protein L452), and Spot 4 comprises a binding reagent for detecting 22917G (S protein L452R mutation). In embodiments, Spot 1 comprises a binding reagent for detecting 23012G (S protein E484), and Spot 6 comprises a binding reagent for detecting 23012A (S protein E484K mutation); Spot 5 comprises a binding reagent for detecting 23664C (S protein A701), and Spot 10 comprises a binding reagent for detecting 23664T (S protein A701V mutation), or Spot 5 comprises a binding reagent for detecting 22813G (S protein K417), and Spot 10 comprises a binding reagent for detecting 22813T (S protein K417N mutation); Spot 7 comprises a binding reagent for detecting 22812A (S protein K417), and Spot 2 comprises a binding reagent for detecting 22812C (S protein K417T mutation).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting a deletion at 21765-21770 (S protein 469-70 deletion), and Spot 6 comprises a binding reagent for detecting the wild-type sequence at 21765-21770 (S protein residues 69-70); Spot 2 comprises a binding reagent for detecting 23063A (S protein N501), and Spot 7 comprises a binding reagent for detecting 23063T (S protein N501Y mutation); Spot 3 comprises a binding reagent for detecting 23403A (S protein D614), and Spot 8 comprises a binding reagent for detecting 23403G (S protein D614G mutation); Spot 4 comprises a binding reagent for detecting 23604C (S protein P681), and Spot 9 comprises a binding reagent for detecting 23604A (S protein P681H mutation); and Spot 5 comprises a binding reagent for detecting 22813G (S protein K417), and Spot 10 comprises a binding reagent for detecting 22813T (S protein K417N mutation).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting 23012G (S protein E484), and Spot 6 comprises a binding reagent for detecting 23012A (S protein E484K mutation); Spot 7 comprises a binding reagent for detecting 22812A (S protein K417), and Spot 2 comprises a binding reagent for detecting 22812C (S protein K417T mutation); Spot 8 comprises a binding reagent for detecting 22206A (S protein D215), and Spot 3 comprises a binding reagent for detecting 22206G (S protein D215G mutation); Spot 9 comprises a binding reagent for detecting 22917T (S protein L452), and Spot 4 comprises a binding reagent for detecting 22917G (S protein L452R mutation); and Spot 10 comprises a binding reagent for detecting 22320A (S protein D253) and Spot 5 comprises a binding reagent for detecting 22320G (S protein D253G mutation).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 2 comprises a binding reagent for detecting 23012C (S protein E484Q mutation). In embodiments, Spot 3 comprises a binding reagent for detecting 21618C (S protein T19), and Spot 8 comprises a binding reagent for detecting 21618G (S protein T19R mutation). In embodiments, Spot 7 comprises a binding reagent for detecting 23604G (S protein P681R mutation). In embodiments, Spot 5 comprises a binding reagent for detecting 24775A (S protein Q1071), and Spot 10 comprises a binding reagent for detecting 24775T (S protein Q1071H mutation). In embodiments, Spot 1 comprises a binding reagent for detecting 22995C (S protein T478), and Spot 6 comprises a binding reagent for detecting 22995A (S protein T478K mutation). In embodiments, Spot 2 comprises a binding reagent for detecting 24224T (S protein F888), and Spot 7 comprises a binding reagent for detecting 24224C (S protein F888L mutation). In embodiments, Spot 3 comprises a binding reagent for detecting 25088G (S protein V1167), and Spot 8 comprises a binding reagent for detecting 25088T (S protein V1167F mutation). In embodiments, Spot 4 comprises a binding reagent for detecting 23593G (S protein Q677), and Spot 9 comprises a binding reagent for detecting 23593T or 23593C (S protein Q677H mutation). In embodiments, Spot 5 comprises a binding reagent for detecting 21991-21993 (S protein Y144), and Spot 10 comprises a binding reagent for detecting a deletion at 21991-21993 (S protein Y144 del mutation).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting a deletion at 21765-21770 (S protein 469-70 deletion), and Spot 6 comprises a binding reagent for detecting the wild-type sequence at 21765-21770 (S protein residues 69-70); Spot 2 comprises a binding reagent for detecting 23063A (S protein N501), and Spot 7 comprises a binding reagent for detecting 23063T (S protein N501Y mutation); Spot 3 comprises a binding reagent for detecting 23403A (S protein D614), and Spot 8 comprises a binding reagent for detecting 23403G (S protein D614G mutation); Spot 4 comprises a binding reagent for detecting 23593G (S protein Q677), and Spot 9 comprises a binding reagent for detecting 23593T or 23593C (S protein Q677H mutation); and Spot 5 comprises a binding reagent for detecting 22813G (S protein K417), and Spot 10 comprises a binding reagent for detecting 22813T (S protein K417N mutation).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting 22995C (S protein T478), and Spot 6 comprises a binding reagent for detecting 22995A (S protein T478K mutation); Spot 2 comprises a binding reagent for detecting 22813T (S protein K417N mutation), and Spot 7 comprises a binding reagent for detecting 22813G (S protein K417); Spot 3 comprises a binding reagent for detecting 22206G (S protein D215G mutation), and Spot 8 comprises a binding reagent for detecting 22206A (S protein D215); Spot 4 comprises a binding reagent for detecting 22917G (S protein L452R mutation), and Spot 9 comprises a binding reagent for detecting 22917T (S protein L452); and Spot 5 comprises a binding reagent for detecting 22320G (S protein D253G mutation), and Spot 10 comprises a binding reagent for detecting 22320A (S protein D253).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting 23012G (S protein E484), Spot 6 comprises a binding reagent for detecting 23012A (S protein E484K mutation), and Spot 2 comprises a binding reagent for detecting 23012C (S protein E484Q mutation); Spot 3 comprises a binding reagent for detecting 21618C (S protein T19), and Spot 8 comprises a binding reagent for detecting 21618G (S protein T19R mutation); Spot 4 comprises a binding reagent for detecting 23604C (S protein P681), Spot 7 comprises a binding reagent for detecting 23604G (S protein P681R mutation), and Spot 9 comprises a binding reagent for detecting 23604A (S protein P681H mutation); and Spot 5 comprises a binding reagent for detecting 24775A (S protein Q1071), and Spot 10 comprises a binding reagent for detecting 24775T (S protein Q1071H mutation).

In embodiments, the multiplexed OLA method for detecting one or more variants of SARS-CoV-2 is conducted in a multi-well plate, wherein each well comprises ten binding domains (“spots”) in an arrangement as shown in FIG. 39B. In embodiments, Spot 1 comprises a binding reagent for detecting a deletion at 21765-21770 (S protein 469-70 deletion); Spot 2 comprises a binding reagent for detecting 22132T (S protein R190S mutation); Spot 3 comprises a binding reagent for detecting 22206G (S protein D215G mutation); Spot 4 comprises a binding reagent for detecting 22917G (S protein L452R mutation); Spot 5 comprises a binding reagent for detecting 22320G (S protein D253G mutation); Spot 6 comprises a binding reagent for detecting 23012A (S protein E484K mutation); Spot 7 comprises a binding reagent for detecting 23063T (S protein N501Y mutation); Spot 8 comprises a binding reagent for detecting 23403G (S protein D614G mutation); Spot 9 comprises a binding reagent for detecting 23604A (S protein P681H mutation); Spot 10 comprises a binding reagent for detecting 22813T (S protein K417N mutation).

In embodiments, the multiplexed OLA method for detecting one or more variants of SARS-CoV-2 is conducted in a multi-well plate, wherein each well comprises ten binding domains (“spots”) in an arrangement as shown in FIG. 39B. In embodiments, Spot 4 comprises a binding reagent for detecting 24224T (S protein F888). In embodiments, Spot 9 comprises a binding reagent for detecting 24224C (S protein F888L). In embodiments, Spot 4 comprises a binding reagent for detecting 24138C (S protein T859). In embodiments, Spot 9 comprises a binding reagent for detecting 24138A (S protein T859N).

In embodiments, the multiplexed OLA method for detecting a reference strain and one or more variants of SARS-CoV-2 is conducted in a multi-well plate, wherein each well comprises ten binding domains (“spots”) in an arrangement as shown in FIG. 39B. In embodiments, the method is capable of detecting the SARS-CoV-2 B.1.1.529 (“Omicron”) variant, e.g., with greater than 90% accuracy, greater than 95% accuracy, greater than 96% accuracy, greater than 97% accuracy, greater than 98% accuracy, greater than 99% accuracy, or greater than 99.9% accuracy. In embodiments, Spot 1 comprises a binding reagent for detecting a deletion at 21765-21770 (S protein 469-70 deletion), and Spot 6 comprises a binding reagent for detecting the wild-type sequence at 21765-21770 (S protein residues 69-70); Spot 2 comprises a binding reagent for detecting 21846C (S protein T95), and Spot 7 comprises a binding reagent for detecting 21846T (S protein T95I mutation); Spot 3 comprises a binding reagent for detecting 22578G (S protein G339), and Spot 8 comprises a binding reagent for detecting 22578A (S protein G339D mutation); Spot 4 comprises a binding reagent for detecting 23525C (S protein H655), and Spot 9 comprises a binding reagent for detecting 23525T (S protein H655Y); and Spot 5 comprises a binding reagent for detecting 22813G (S protein K417), and Spot 10 comprises a binding reagent for detecting 22813T (S protein K417N mutation). Exemplary targeting and detection probe sequences are provided in Table 10. Exemplary synthetic oligonucleotide template sequences are provided in Table 11. Exemplary blocking oligonucleotide sequences are provided in Table 12.

### Manual and Automated Embodiments

The methods herein can be performed manually, using automated technology, or both. Automated technology may be partially automated, e.g., one or more modular instruments, or a fully integrated, automated instrument. Exemplary automated systems and apparatuses are described in WO 2018/017156, WO 2017/015636, and WO 2016/164477. In embodiments, the methods herein are performed in an automated cartridge reader as described herein. Manual and automated systems for use with the methods and kits described herein are known in the art and described, e.g., in U.S. Publication No. 2022/0003766 and U.S. Publication No. 2021/0349104.

### Antibody and Composition

In embodiments, the invention provides an antibody or antigen-binding fragment thereof that specifically binds a viral antigen described herein, e.g., a SARS-CoV-2 protein. In embodiments, the invention provides an antibody or antigen-binding fragment thereof that specifically binds a SARS-CoV-2 N protein or a SARS-CoV-2 S protein. In embodiments, the invention provides an antibody or antigen-binding fragment thereof that specifically binds SARS-CoV-2 S1, S2, S-ECD, S-NTD, or S-RBD. In embodiments, the invention provides an antibody or antigen-binding fragment thereof that specifically binds a SARS-CoV-2 S protein or subunit or fragment thereof that comprises any of the mutations in Tables 1A and 1B. In embodiments, the invention provides an antibody or antigen-binding fragment thereof that specifically binds an S protein from SARS-CoV-2, an S protein from SARS-CoV, an S protein from MERS-CoV, an S protein from HCoV-HKU1, an S protein from HCoV-OC43, an S protein from HCoV-NL63, an S protein from HCoV-229E, an N protein from SARS-CoV-2, an N protein from SARS-CoV, an N protein from MERS-CoV, an N protein from HCoV-HKU1, an N protein from HCoV-OC43, an N protein from HCoV-NL63, an N protein from HCoV-229E, an HA from influenza B, an HA from influenza A H1, an HA from influenza A H3, an HA from influenza A H7, an F protein from RSV, or a combination thereof.

In embodiments, the antibody or antigen-binding fragment thereof is a binding reagent, e.g., as disclosed herein, for an assay described herein, e.g., for detecting a viral component in a sample. In embodiments, the antibody or antigen-binding fragment thereof is capable of being immobilized onto a surface, e.g., as disclosed herein. In embodiments, the invention provides a composition comprising: (i) the antibody or antigen-binding fragment thereof; and (ii) a surface. In embodiments, the antibody or antigen-binding fragment thereof is immobilized onto the surface.

In embodiments, the antibody or antigen-binding fragment thereof is a detection reagent, e.g., as disclosed herein, for an assay described herein, e.g., for detecting a viral component in a sample. In embodiments, the antibody or antigen-binding fragment thereof comprises a detectable label. In embodiments, the antibody or antigen-binding fragment is capable of being conjugated with a detectable label. In embodiments, the invention provides a composition comprising: (i) the antibody or antigen-binding fragment thereof; (ii) a detectable label, e.g., as disclosed herein; and (iii) a reagent for conjugating the detectable label to the antibody or antigen-binding fragment thereof. In embodiments, the detectable label is an ECL label. In embodiments, the antibody or antigen-binding fragment thereof comprises a nucleic acid probe. In embodiments, the antibody or antigen-binding fragment is capable of being conjugated with a nucleic acid probe. In embodiments, the invention provides a composition comprising: (i) the antibody or antigen-binding fragment thereof (ii) a nucleic acid probe, e.g., as disclosed herein; and (iii) a reagent for conjugating the nucleic acid probe to the antibody or antigen-binding fragment thereof.

In embodiments, the antibody or antigen-binding fragment thereof is a calibration reagent, e.g., as disclosed herein, for a serology assay, e.g., a classical, bridging, or competitive serology assay, e.g., as disclosed herein. In embodiments, the antibody or antigen-binding fragment thereof is a competitor for a competitive serology assay.

In embodiments, the invention provides a therapeutic composition comprising the antibody or antigen-binding fragment thereof. In embodiments, the therapeutic composition is capable of treating or preventing infection by a virus described herein, e.g., SARS-CoV-2 and/or a variant thereof.

In embodiments, the invention provides a composition comprising (i) the antibody or antigen-binding fragment thereof and (ii) a viral antigen that specifically binds the antibody or antigen-binding fragment.

In general, an antibody (used interchangeably with the term “immunoglobulin”) comprises at least the variable domain of a heavy chain; typically, an antibody comprises the variable domains of a heavy chain and a light chain. Both the heavy and light chains are divided into regions of structural and functional homology. Generally, the variable domain of a heavy chain (V_{H}) or light chain (V_{L}) determines antigen recognition and specificity, and the constant domain of a heavy chain (C_{H1}, C_{H2}, or C_{H3}) or light chain (C_{L}) confers biological properties such as secretion, receptor binding, complement binding, and the like. Generally, the N-terminal portion of an antibody chain is a variable portion, and the C-terminal portion is a constant region; the C_{H3} and C_{L} domains typically comprise the C-terminus of the heavy chain and light chain, respectively.

In general, antibodies are encoded by immunoglobulin genes or fragments of immunoglobulin genes. The recognized immunoglobulin genes include the kappa, lambda, alpha, gamma, delta, epsilon, and mu constant region genes, as well as immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

In general, the variable region allows the antibody to selectively recognize and specifically bind epitopes on antigens. Thus, the V_{L} domain and V_{H} domain, or a subset of the complementarity determining regions (CDR) within these variable domains, of an antibody combine to form the variable region that forms an antigen binding domain. The antigen binding domain is typically defined by three CDRs on each of the V_{L} and V_{H} domains. The six “complementarity determining regions” or “CDRs” typically present in each antigen binding domain are short, non-contiguous sequences of amino acids that are specifically positioned to form the antigen binding domain. The antigen binding domain formed by the positioned CDRs defines a surface complementary to the epitope on the antigen. This complementary surface promotes the non-covalent binding of the antibody to its cognate epitope.

In embodiments, the antibody or antigen-binding fragment thereof comprises a constant region comprising an IgA, IgD, IgE, IgG, or IgM domain. In embodiments, the antibody or antigen-binding fragment thereof comprises an IgG domain. In embodiments, the antibody or antigen-binding fragment thereof is an IgG1, IgG2, IgG3, or IgG4 isotype antibody or antigen-binding fragment thereof. In embodiments, the antibody or antigen-binding fragment thereof is IgG2a, IgG2b, or IgG2c subclass antibody or antigen-binding fragment thereof.

In embodiments, the antibody or antigen-binding fragment thereof is derived from a mouse, rat, goat, rabbit, chicken, guinea pig, hamster, horse, sheep, ferret, minx, or bat. In embodiments, the antibody or antigen-binding fragment thereof is humanized. In embodiments, the antibody or antigen-binding fragment thereof is capable of being administered to a human or an animal subject described herein, e.g., mouse, rat, ferret, minx, bat, a domestic animal, or an NHP. In embodiments, the antibody or antigen-binding fragment thereof is non-immunogenic to a human or an animal subject described herein, e.g., mouse, rat, ferret, minx, bat, a domestic animal, or an NHP.

### Kits

In embodiments, the invention provides a kit comprising, in one or more vials, containers, or compartments: (a) a viral antigen that specifically binds a biomarker, e.g., an antibody biomarker; and (b) a detection reagent that specifically binds the biomarker, e.g., the antibody biomarker. In embodiments, the kit further comprises a surface. Antibody biomarkers and their binding partners, e.g., viral antigens, are described herein. In embodiments, the detection reagent is an antibody or antigen-binding fragment thereof. In embodiments, the detection reagent is a second copy of the viral antigen.

In embodiments, the viral antigen is a respiratory virus antigen. In embodiments, the respiratory virus is a coronavirus, an influenza virus, a paramyxovirus, an adenovirus, a bocavirus, a pneumovirus, an enterovirus, a rhinovirus, or a combination thereof. In embodiments, the viral antigen is a coronavirus S protein or fragment thereof. In embodiments, the coronavirus is SARS-CoV, MERS-CoV, SARS-CoV-2, HCoV-OC43, HCoV-229E, HCoV-NL63, HCoV-HKU1, or a combination thereof. In embodiments, the viral antigen is SARS-CoV-2 S protein, S1 subunit, S2 subunit, S-RBD, M protein, E protein, N protein, or a combination thereof.

In embodiments, the invention provides a kit comprising, in one or more vials, containers, or compartments: (a) a binding reagent that specifically binds a biomarker, e.g., an inflammatory or tissue damage response biomarker; and (b) a detection reagent that specifically binds the biomarker, e.g., the inflammatory or tissue damage response biomarker. In embodiments, the kit further comprises a surface. Inflammatory and tissue damage response biomarkers and binding and detection reagents therefor are described herein. In embodiments, the binding reagent is an antibody or antigen-binding fragment. In embodiments, the detection reagent is an antibody or antigen-binding fragment thereof.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 3 of FIG. 39B comprises an N protein from immobilized SARS-CoV-2, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, and Spots 2, 4, 5, 6, and 10 of FIG. 39B each comprises an immobilized BSA. In embodiments, the S protein mutations from these SARS-CoV-2 strains are described in Table 1D.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an S-D614G from SARS-CoV-2, Spot 3 of FIG. 39B comprises an N protein from immobilized SARS-CoV-2, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, Spot 10 of FIG. 39B comprises a wild-type S-RBD from SARS-CoV-2, and Spots 4, 5, and 6 of FIG. 39B each comprises an immobilized BSA. In embodiments, the S protein mutations from these SARS-CoV-2 strains are described in Table 1D.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an S-D614G from SARS-CoV-2, an N protein from SARS-CoV-2, an S protein from SARS-CoV-2 strain P.1, an S protein from SARS-CoV-2 strain B.1.1.7, and an S protein from SARS-CoV-2 strain 501Y.V2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an S-D614G from SARS-CoV-2, Spot 3 of FIG. 39B comprises an N protein from immobilized SARS-CoV-2, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, and Spots 4, 5, 6, and 10 of FIG. 39B each comprises an immobilized BSA. In embodiments, the S protein mutations from these SARS-CoV-2 strains are described in Table 1D.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an S-RBD from SARS-CoV-2 strain 501Y.V2, an N protein from SARS-CoV-2, an S-RBD from SARS-CoV-2 strain P.1, an S-RBD from SARS-CoV-2 strain B.1.1.7, an S protein from SARS-CoV-2 strain P.1, an S protein from SARS-CoV-2 strain B.1.1.7, an S protein from SARS-CoV-2 strain 501Y.V2, and a wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an S-RBD from SARS-CoV-2 strain 501Y.V2, Spot 3 of FIG. 39B comprises an N protein from immobilized SARS-CoV-2, Spot 4 of FIG. 39B comprises an S-RBD from SARS-CoV-2 strain P.1, Spot 6 of FIG. 39B comprises an S-RBD from SARS-CoV-2 strain B.1.1.7, Spot 7 of FIG. 39B comprises an S protein from SARS-CoV-2 strain P.1, Spot 8 of FIG. 39B comprises an S protein from SARS-CoV-2 strain B.1.1.7, Spot 9 of FIG. 39B comprises an S protein from SARS-CoV-2 strain 501Y.V2, Spot 10 of FIG. 39B comprises a wild-type S-RBD from SARS-CoV-2, and Spot 5 of FIG. 39B each comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an S-RBD from SARS-CoV-2 strain B.1.429, an N protein from SARS-CoV-2, an S-RBD from SARS-CoV-2 strain B.1.526/E484K, an S-RBD from SARS-CoV-2 strain B.1.526/S477N, an S protein from SARS-CoV-2 strain B.1.526/E484K, an S protein from SARS-CoV-2 strain B.1.526/S477N, an S protein from SARS-CoV-2 strain B.1.429, and a wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2, Spot 4 of FIG. 39B comprises an immobilized S-RBD from SARS-CoV-2 strain B.1.526/E484K, Spot 6 of FIG. 39B comprises an immobilized S-RBD from SARS-CoV-2 strain B.1.526/S477N, Spot 7 of FIG. 39B comprises an immobilized S protein from SARS-CoV-2 strain B.1.526/E484K, Spot 8 of FIG. 39B comprises an immobilized S protein from SARS-CoV-2 strain B.1.526/S477N, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spot 5 of FIG. 39B comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, the invention provides a kit comprising: (a) one or more binding reagents that specifically bind one or more antibody biomarkers, wherein each antibody biomarker specifically binds to: one or more of a wild-type S protein from SARS-CoV-2, an S-RBD from SARS-CoV-2 strain B.1.429, an N protein from SARS-CoV-2, an S-RBD from SARS-CoV-2 strain B.1.526, an S-RBD from SARS-CoV-2 strain B.1.526.2, an S protein from SARS-CoV-2 strain B.1.526, an S protein from SARS-CoV-2 strain B.1.429, and a wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a wild-type S protein from SARS-CoV-2, an S-RBD from SARS-CoV-2 strain B.1.429, an N protein from SARS-CoV-2, an S-RBD from SARS-CoV-2 strain B.1.526, an S-RBD from SARS-CoV-2 strain B.1.526.2, an S protein from SARS-CoV-2 strain B.1.526, an S protein from SARS-CoV-2 strain B.1.429, and a wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a wild-type S protein from SARS-CoV-2, an S-RBD from SARS-CoV-2 strain B.1.429, an N protein from SARS-CoV-2, an S-RBD from SARS-CoV-2 strain B.1.526, an S-RBD from SARS-CoV-2 strain B.1.526.2, an S protein from SARS-CoV-2 strain B.1.526, an S protein from SARS-CoV-2 strain B.1.429, and a wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized wild-type S protein from SARS-CoV-2, Spot 2 of FIG. 39B comprises an immobilized S-RBD from SARS-CoV-2 strain B.1.429, Spot 3 of FIG. 39B comprises an immobilized N protein from SARS-CoV-2, Spot 4 of FIG. 39B comprises an immobilized S-RBD from SARS-CoV-2 strain B.1.526, Spot 6 of FIG. 39B comprises an immobilized S-RBD from SARS-CoV-2 strain B.1.526.2, Spot 8 of FIG. 39B comprises an immobilized S protein from SARS-CoV-2 strain B.1.526, Spot 9 of FIG. 39B comprises an immobilized S protein from SARS-CoV-2 strain B.1.429, Spot 10 of FIG. 39B comprises an immobilized wild-type S-RBD from SARS-CoV-2, and Spots 5 and 7 of FIG. 39B each comprises an immobilized BSA. In embodiments, the S protein mutations from these SARS-CoV-2 strains are described in Table 1D. In embodiments, the S-RBD mutations from these SARS-CoV-2 strains are described in Table 1E.

In embodiments, the invention provides a kit comprising: (a) one or more binding reagents that specifically bind one or more antibody biomarkers, wherein each antibody biomarker specifically binds to: one or more of a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises an N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD, wherein all mutations are relative to wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises an N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD, wherein all mutations are relative to wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises an N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD, wherein all mutations are relative to wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S-RBD that comprises an L452R mutation, Spot 2 of FIG. 39B comprises an immobilized SARS-CoV-2 S-RBD that comprises K417N, E484K, and N501Y mutations, Spot 3 of FIG. 39B comprises an immobilized SARS-CoV-2 S-RBD that comprises an E484K mutation, Spot 4 of FIG. 39B comprises an immobilized SARS-CoV-2 S-RBD that comprises K417T, E484K, and N501Y mutations, Spot 5 of FIG. 39B comprises an immobilized SARS-CoV-2 S-RBD that comprises an S477N mutation, Spot 6 of FIG. 39B comprises an immobilized SARS-CoV-2 S-RBD that comprises an N501Y mutation, Spot 7 of FIG. 39B comprises an immobilized SARS-CoV-2 S-RBD that comprises E484K and N501Y mutations, Spot 8 of FIG. 39B comprises an immobilized SARS-CoV-2 S-RBD that comprises L452R and E484Q mutations, Spot 9 of FIG. 39B comprises an immobilized SARS-CoV-2 S-RBD that comprises Q414K and N450K mutations, and Spot 10 of FIG. 39B comprises an immobilized wild-type SARS-CoV-2 S-RBD, wherein all mutations are relative to wild-type S-RBD from SARS-CoV-2.

In embodiments, the invention provides a kit comprising: (a) one or more binding reagents that specifically bind one or more antibody biomarkers, wherein each antibody biomarker specifically binds to: one or more of a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises N501Y and A570D mutations; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD, wherein all mutations are relative to wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises N501Y and A570D mutations; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD, wherein all mutations are relative to wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises N501Y and A570D mutations; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD, wherein all mutations are relative to wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises an immobilized SARS-CoV-2 S-RBD that comprises an L452R mutation, Spot 2 of FIG. 39B comprises an immobilized SARS-CoV-2 S-RBD that comprises K417N, E484K, and N501Y mutations, Spot 3 of FIG. 39B comprises an immobilized SARS-CoV-2 S-RBD that comprises an E484K mutation, Spot 4 of FIG. 39B comprises an immobilized SARS-CoV-2 S-RBD that comprises K417T, E484K, and N501Y mutations, Spot 5 of FIG. 39B comprises an immobilized SARS-CoV-2 S-RBD that comprises an S477N mutation, Spot 6 of FIG. 39B comprises an immobilized SARS-CoV-2 S-RBD that comprises N501Y and A570D mutations, Spot 7 of FIG. 39B comprises an immobilized SARS-CoV-2 S-RBD that comprises E484K and N501Y mutations, Spot 8 of FIG. 39B comprises an immobilized SARS-CoV-2 S-RBD that comprises L452R and E484Q mutations, Spot 9 of FIG. 39B comprises an immobilized SARS-CoV-2 S-RBD that comprises Q414K and N450K mutations, and Spot 10 of FIG. 39B comprises an immobilized wild-type SARS-CoV-2 S-RBD, wherein all mutations are relative to wild-type S-RBD from SARS-CoV-2.

In embodiments, the invention provides a kit comprising: (a) one or more binding reagents that specifically bind one or more antibody biomarkers, wherein each antibody biomarker specifically binds to: one or more of a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B, respectively, comprise the following immobilized antigens: a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2.

In embodiments, the invention provides a kit comprising: (a) one or more binding reagents that specifically bind one or more antibody biomarkers, wherein each antibody biomarker specifically binds to: one or more of a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B, respectively, comprise the following immobilized antigens: a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD, wherein all mutations are relative to wild-type S-RBD from SARS-CoV-2.

In embodiments, the invention provides a kit comprising: (a) one or more binding reagents that specifically bind one or more antibody biomarkers, wherein each antibody biomarker specifically binds to: one or more of a Spike protein from the following SARS-CoV-2 strains: wild type; P.2; B.1.617.1; B.1.617.2; B.1.617.3; B.1.617; P.1; B.1.1.7; B.1.351; and B.1.526.1; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the one or more detection reagents specifically binds to the antibody biomarker. In embodiments, the one or more detection reagents specifically binds IgA, IgG, or IgM. In embodiments, the one or more detection reagents comprises a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B, respectively, comprise the following immobilized antigens: a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the Spike protein mutations from these SARS-CoV-2 strains are described in Table 1D. In embodiments, the Spike protein from SARS-CoV-2 strain B.1.617.2 comprises the mutations T19R, Δ157/158, L452R, T478K, D614G, P681R, and D950N.

In embodiments, the invention provides a kit comprising: (a) one or more binding reagents that specifically bind one or more antibody biomarkers, wherein each antibody biomarker specifically binds to: one or more of a Spike protein from the following SARS-CoV-2 strains: wild type; A.23.1; A.VOI.V2; B.1.617.2; C.37; R.1; P.3; B.1.525; B.1.1.519; and BV-1; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a Spike protein from the following SARS-CoV-2 strains: wild type; A.23.1; A.VOI.V2; B.1.617.2; C.37; R.1; P.3; B.1.525; B.1.1.519; and BV-1. In embodiments, the one or more detection reagents specifically binds to the antibody biomarker. In embodiments, the one or more detection reagents specifically binds IgA, IgG, or IgM. In embodiments, the one or more detection reagents comprises a Spike protein from the following SARS-CoV-2 strains: wild type; A.23.1; A.VOI.V2; B.1.617.2; C.37; R.1; P.3; B.1.525; B.1.1.519; and BV-1. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B, respectively, comprise the following immobilized antigens: a Spike protein from the following SARS-CoV-2 strains: wild type; A.23.1; A.VOI.V2; B.1.617.2; C.37; R.1; P.3; B.1.525; B.1.1.519; and BV-1. In embodiments, the Spike protein mutations from these SARS-CoV-2 strains are described in Table 1D. In embodiments, the Spike protein from SARS-CoV-2 strain B.1.617.2 comprises the mutations T19R, G142D, A156/157, R158G, L452R, T478K, D614G, P681R, and D950N.

In embodiments, the invention provides a kit comprising: (a) one or more binding reagents that specifically bind one or more antibody biomarkers, wherein each antibody biomarker specifically binds to: one or more of a Spike protein from the following SARS-CoV-2 strains: wild type; AY.1, AY.2, B.1.617.2 plus deletion of Y144; B.1.620; B.1.258.17; B.1.466.2; B.1.1.7 plus the E484K mutation; B.1.351.1; and B.1.618; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a Spike protein from the following SARS-CoV-2 strains: wild type; AY.1, AY.2, B.1.617.2 plus deletion of Y144; B.1.620; B.1.258.17; B.1.466.2; B.1.1.7 plus the E484K mutation; B.1.351.1; and B.1.618. In embodiments, the one or more detection reagents specifically binds to the antibody biomarker. In embodiments, the one or more detection reagents specifically binds IgA, IgG, or IgM. In embodiments, the one or more detection reagents comprises a Spike protein from the following SARS-CoV-2 strains: wild type; AY.1, AY.2, B.1.617.2 plus deletion of Y144; B.1.620; B.1.258.17; B.1.466.2; B.1.1.7 plus the E484K mutation; B.1.351.1; and B.1.618. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B, respectively, comprise the following immobilized antigens: a Spike protein from the following SARS-CoV-2 strains: wild type; AY.1, AY.2, B.1.617.2 plus deletion of Y144; B.1.620; B.1.258.17; B.1.466.2; B.1.1.7 plus the E484K mutation; B.1.351.1; and B.1.618. In embodiments, the Spike protein mutations from these SARS-CoV-2 strains are described in Table 1D. In embodiments, the Spike protein from SARS-CoV-2 strain B.1.617.2 plus deletion of Y144 comprises the mutations T19R, ΔY144, A157/158, L452R, T478K, D614G, P681R, and D950N.

In embodiments, the invention provides a kit comprising: (a) one or more binding reagents that specifically bind one or more antibody biomarkers, wherein each antibody biomarker specifically binds to: one or more of a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD, wherein all mutations are relative to wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises: a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD, wherein all mutations are relative to wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises: a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD, wherein all mutations are relative to wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-4 of FIG. 39B comprise, respectively, the following immobilized antigens: a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; Spots 7-10 of FIG. 39B comprise, respectively, the following immobilized antigens: a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD, wherein all mutations are relative to wild-type S-RBD from SARS-CoV-2; and Spots 5-6 of FIG. 39B each comprises BSA.

In embodiments, the invention provides a kit comprising: (a) one or more binding reagents that specifically bind one or more antibody biomarkers, wherein each antibody biomarker specifically binds to: one or more of a wild-type S protein from SARS-CoV-2; an S-D614G from SARS-CoV-2; an N protein from SARS-CoV-2; an S protein from SARS-CoV-2 strain B.1.617.2; an S protein from SARS-CoV-2 strain P.1; an S protein from SARS-CoV-2 strain B.1.1.7; an S protein from SARS-CoV-2 strain B.1.351; and a wild-type S-RBD from SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises: a wild-type S protein from SARS-CoV-2; an S-D614G from SARS-CoV-2; an N protein from SARS-CoV-2; an S protein from SARS-CoV-2 strain B.1.617.2; an S protein from SARS-CoV-2 strain P.1; an S protein from SARS-CoV-2 strain B.1.1.7; an S protein from SARS-CoV-2 strain B.1.351; and a wild-type S-RBD from SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises: a wild-type S protein from SARS-CoV-2; an S-D614G from SARS-CoV-2; an N protein from SARS-CoV-2; an S protein from SARS-CoV-2 strain B.1.617.2; an S protein from SARS-CoV-2 strain P.1; an S protein from SARS-CoV-2 strain B.1.1.7; an S protein from SARS-CoV-2 strain B.1.351; and a wild-type S-RBD from SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spot 1 of FIG. 39B comprises a wild-type S protein from SARS-CoV-2; Spot 2 of FIG. 39B comprises an S-D614G from SARS-CoV-2; Spot 3 of FIG. 39B comprises an N protein from SARS-CoV-2; Spot 4 of FIG. 39B comprises an S protein from SARS-CoV-2 strain B.1.617.2; Spot 7 of FIG. 39B comprises an S protein from SARS-CoV-2 strain P.1; Spot 8 of FIG. 39B comprises an S protein from SARS-CoV-2 strain B.1.1.7; Spot 9 of FIG. 39B comprises an S protein from SARS-CoV-2 strain B.1.351; Spot 10 of FIG. 39B comprises a wild-type S-RBD from SARS-CoV-2; and Spots 5 and 6 of FIG. 39B comprise BSA. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, the invention provides a kit comprising: (a) one or more binding reagents that specifically bind one or more antibody biomarkers, wherein each antibody biomarker specifically binds to: one or more of a Spike protein from the following SARS-CoV-2 strains: wild type; P.2; B.1.617.1; B.1.617.2; B.1.617.3; B.1.617; P.1; B.1.1.7; B.1.351; and B.1.526.1; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the one or more detection reagents specifically binds to the antibody biomarker. In embodiments, the one or more detection reagents specifically binds IgA, IgG, or IgM. In embodiments, the one or more detection reagents comprises a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B, respectively, comprise the following immobilized antigens: a Spike protein from the following SARS-CoV-2 strains: wild type; P.2; B.1.617.1; B.1.617.2; B.1.617.3; B.1.617; P.1; B.1.1.7; B.1.351; and B.1.526.1. In embodiments, the Spike protein mutations from these SARS-CoV-2 strains are described in Table 1D. In embodiments, the Spike protein from SARS-CoV-2 strain B.1.617.2 comprises the mutations T19R, T95I, G142D, Δ156/157, R158G, L452R, T478K, D614G, P681R, and D950N.

In embodiments, the invention provides a kit comprising: (a) one or more binding reagents that specifically bind one or more antibody biomarkers, wherein each antibody biomarker specifically binds to: one or more of a Spike protein from the following SARS-CoV-2 strains: wild type; P.2; B.1.617.1; B.1.617.3; B.1.617; P.1; and B.1.1.7; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises: a Spike protein from the following SARS-CoV-2 strains: wild type; P.2; B.1.617.1; B.1.617.3; B.1.617; P.1; and B.1.1.7. In embodiments, the one or more detection reagents specifically binds to the antibody biomarker. In embodiments, the one or more detection reagents specifically binds IgA, IgG, or IgM. In embodiments, the one or more detection reagents comprises: a Spike protein from the following SARS-CoV-2 strains: wild type; P.2; B.1.617.1; B.1.617.3; B.1.617; P.1; and B.1.1.7. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-3 of FIG. 39B comprise, respectively, an immobilized Spike protein from the following SARS-CoV-2 strains: wild type; P.2; and B.1.617.1; Spot 4 of FIG. 39B comprises BSA; and Spots 5-10 of FIG. 39B comprise, respectively, an immobilized Spike protein from the following SARS-CoV-2 strains: B.1.617.3; B.1.617; P.1; and B.1.1.7. In embodiments, the Spike protein mutations from these SARS-CoV-2 strains are described in Table 1D.

In embodiments, the invention provides a kit comprising: (a) one or more binding reagents that specifically bind one or more antibody biomarkers, wherein each antibody biomarker specifically binds to: one or more of a Spike protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14); and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises: a Spike protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14). In embodiments, the one or more detection reagents specifically binds to the antibody biomarker. In embodiments, the one or more detection reagents specifically binds IgA, IgG, or IgM. In embodiments, the one or more detection reagents comprises: a Spike protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14). In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, an immobilized Spike protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14). In embodiments, the Spike protein mutations from these SARS-CoV-2 strains are described in Table 1D. In embodiments, the Spike protein from SARS-CoV-2 strain AY.2 comprises the mutations T19R, V70F, G142D, E156G, Δ157/158, A222V, K417N, L452R, T478K, D614G, P681R, and D950N. In embodiments, the Spike protein from SARS-CoV-2 strain B.1.617.2 (AY.4) comprises the mutations T19R, T95I, G142D, Δ156/157, R158G, L452R, T478K, D614G, P681R, and D950N. In embodiments, the Spike protein from SARS-CoV-2 strain AY.1 comprises the mutations T19R, T95I, G142D, E156G, Δ157/158, W258L, K417N, L452R, T478K, D614G, P681R, and D950N. In embodiments, the Spike protein from SARS-CoV-2 strain B.1.617.2 (AY.3, AY.5, AY.6, AY.7, AY.14) comprises the mutations T19R, G142D, Δ156/157, R158G, L452R, T478K, D614G, P681R, and D950N.

In embodiments, the invention provides a kit comprising: (a) one or more binding reagents that specifically bind one or more antibody biomarkers, wherein each antibody biomarker specifically binds to: one or more of a Spike protein from the following SARS-CoV-2 strains: wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises: a Spike protein from the following SARS-CoV-2 strains: wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2. In embodiments, the one or more detection reagents specifically binds to the antibody biomarker. In embodiments, the one or more detection reagents specifically binds IgA, IgG, or IgM. In embodiments, the one or more detection reagents comprises: a Spike protein from the following SARS-CoV-2 strains: wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, an immobilized Spike protein from the following SARS-CoV-2 strains: wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2. In embodiments, the Spike protein mutations from these SARS-CoV-2 strains are described in Table 1D. In embodiments, the Spike protein from SARS-CoV-2 strain B.1.617.2 (+K417N/N439K/E484K/N501Y) comprises the mutations T19R, G142D, Δ156/157, R158G, K417N, N439K, L452R, T478K, E484K, N501Y, D614G, P681R, and D950N. In embodiments, the Spike protein from SARS-CoV-2 strain B.1.617.2 (+K417N/E484K/N501Y) comprises the mutations T19R, G142D, Δ156/157, R158G, K417N, L452R, T478K, E484K, N501Y, D614G, P681R, and D950N. In embodiments, the Spike protein from SARS-CoV-2 strain B.1.617.2 (+E484K/N501Y) comprises the mutations T19R, G142D, Δ156/157, R158G, L452R, T478K, E484K, N501Y, D614G, P681R, and D950N. In embodiments, the Spike protein from SARS-CoV-2 strain B.1.617.2 (+E484K) comprises the mutations T19R, G142D, del156/157, R158G, L452R, T478K, E484K, D614G, P681R, and D950N. In embodiments, the Spike protein from SARS-CoV-2 strain B.1.617.2 comprises the mutations T19R, G142D, Δ156/157, R158G, L452R, T478K, D614G, P681R, and D950N.

In embodiments, the invention provides a kit comprising: (a) one or more binding reagents that specifically bind one or more antibody biomarkers, wherein each antibody biomarker specifically binds to: one or more of a Spike protein from the following SARS-CoV-2 strains: wild-type; B.1.1.529; AY.4.2; AY.4; P.1; B.1.1.7; B.1.351; and B.1.617.2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises: a Spike protein from the following SARS-CoV-2 strains: wild-type; B.1.1.529; AY.4.2; AY.4; P.1; B.1.1.7; B.1.351; and B.1.617.2. In embodiments, the one or more detection reagents specifically binds to the antibody biomarker. In embodiments, the one or more detection reagents specifically binds IgA, IgG, or IgM. In embodiments, the one or more detection reagents comprises: a Spike protein from the following SARS-CoV-2 strains: wild-type; B.1.1.529; AY.4.2; AY.4; P.1; B.1.1.7; B.1.351; and B.1.617.2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-4 of FIG. 39B comprise, respectively, an immobilized Spike protein from the following SARS-CoV-2 strains: wild-type; B.1.1.529; AY.4.2; AY.4; Spots 5-6 each comprises BSA; and Spots 7-10 comprise, respectively, an immobilized Spike protein from the following SARS-CoV-2 strains: P.1; B.1.1.7; B.1.351; and B.1.617.2. In embodiments, the Spike protein mutations from these SARS-CoV-2 strains are described in Table 1D. In embodiments, the Spike protein from SARS-CoV-2 strain B.1.617.2 comprises the mutations T19R, G142D, Δ156/157, R158G, L452R, T478K, D614G, P681R, and D950N.

In embodiments, the invention provides a kit comprising: (a) one or more binding reagents that specifically bind one or more antibody biomarkers, wherein each antibody biomarker specifically binds to: one or more of an S-RBD from the following SARS-CoV-2 strains: B.1.1.529; B.1.351; P.1; B.1.1.7; B.1.617.2; and wild-type; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises: an S-RBD from the following SARS-CoV-2 strains: B.1.1.529; B.1.351; P.1; B.1.1.7; B.1.617.2; and wild-type. In embodiments, the one or more detection reagents specifically binds to the antibody biomarker. In embodiments, the one or more detection reagents specifically binds IgA, IgG, or IgM. In embodiments, the one or more detection reagents comprises an S-RBD from the following SARS-CoV-2 strains: B.1.1.529; B.1.351; P.1; B.1.1.7; B.1.617.2; and wild-type. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1 and 2 of FIG. 39B comprise, respectively, an immobilized S-RBD from the following SARS-CoV-2 strains: B.1.1.529 and B.1.351; Spot 3 comprises BSA; Spot 4 comprises an immobilized S-RBD from SARS-CoV-2 strain P.1; Spot 5 comprises BSA; Spot 6 comprises an immobilized S-RBD from SARS-CoV-2 strain B.1.1.7; Spots 7 and 8 each comprise BSA; Spot 9 comprises an immobilized S-RBD from SARS-CoV-2 strain B.1.617.2; and Spot 10 comprises an immobilized S-RBD from wild type SARS-CoV-2. In embodiments, the S-RBD mutations from these SARS-CoV-2 strains are described in Table 1E.

In embodiments, the invention provides a kit comprising: (a) one or more binding reagents that specifically bind one or more antibody biomarkers, wherein each antibody biomarker specifically binds to: an S protein from the following SARS-CoV-2 strains: wild-type, B.1.1.529, AY.4, P.1, B.1.1.7, B.1.351; an N protein from wild-type SARS-CoV-2; and an S-RBD from wild-type SARS-CoV-2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises: an S protein from the following SARS-CoV-2 strains: wild-type, B.1.1.529, AY.4, P.1, B.1.1.7, B.1.351; an N protein from wild-type SARS-CoV-2; and an S-RBD from wild-type SARS-CoV-2. In embodiments, the one or more detection reagents specifically binds to the antibody biomarker. In embodiments, the one or more detection reagents specifically binds IgA, IgG, or IgM. In embodiments, the one or more detection reagents comprises an S protein from the following SARS-CoV-2 strains: wild-type, B.1.1.529, AY.4, P.1, B.1.1.7, B.1.351; an N protein from wild-type SARS-CoV-2; and an S-RBD from wild-type SARS-CoV-2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1 and 2 of FIG. 39B comprise, respectively, an immobilized S protein from the following SARS-CoV-2 strains: wild-type and B.1.1.529; Spot 3 comprises an immobilized N protein from wild-type SARS-CoV-2; Spot 4 comprises an immobilized S protein from SARS-CoV-2 strain AY.4; Spots 5 and 6 each comprises BSA; Spots 7-9 comprise, respectively, an immobilized S protein from the following SARS-CoV-2 strains: P.1; B.1.1.7; and B.1.351; and Spot 10 comprises an immobilized S-RBD from wild type SARS-CoV-2. In embodiments, the S protein mutations from these SARS-CoV-2 strains are described in Table 1D.

In embodiments, the invention provides a kit comprising: (a) one or more binding reagents that specifically bind one or more antibody biomarkers, wherein each antibody biomarker specifically binds to: an S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises: an S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2. In embodiments, the one or more detection reagents specifically binds to the antibody biomarker. In embodiments, the one or more detection reagents specifically binds IgA, IgG, or IgM. In embodiments, the one or more detection reagents comprises an S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-10 of FIG. 39B comprise, respectively, an immobilized S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2. In embodiments, the S protein mutations from these SARS-CoV-2 strains are described in Table 1D.

In embodiments, the invention provides a kit comprising: (a) one or more binding reagents that specifically bind one or more antibody biomarkers, wherein each antibody biomarker specifically binds to: an S-RBD from the following SARS-CoV-2 strains: B.1.1.529 (BA.1); B.1.351; BA.2; P.1; B.1.1.7; BA.1.1; B.1.617.2; and wild-type; and (b) one or more detection reagents. In embodiments, the one or more antibody biomarkers comprises IgG, IgA, IgM, or a combination thereof. In embodiments, the IgG, IgA, and/or IgM is from a human, mouse, rat, ferret, minx, bat, or combination thereof. In embodiments, the one or more binding reagents comprises: an S-RBD from the following SARS-CoV-2 strains: B.1.1.529 (BA.1); B.1.351; BA.2; P.1; B.1.1.7; BA.1.1; B.1.617.2; and wild-type. In embodiments, the one or more detection reagents specifically binds to the antibody biomarker. In embodiments, the one or more detection reagents specifically binds IgA, IgG, or IgM. In embodiments, the one or more detection reagents comprises an S-RBD from the following SARS-CoV-2 strains: B.1.1.529 (BA.1); B.1.351; BA.2; P.1; B.1.1.7; BA.1.1; B.1.617.2; and wild-type. In embodiments, the one or more detection reagents comprises ACE2. In embodiments, the kit further comprises a surface. In embodiments, the surface comprises a single assay plate. In embodiments, the surface comprises a multi-well assay plate, wherein each well comprises ten distinct binding domains. In embodiments, the assay plate is a 96-well assay plate. An embodiment of a well in a 96-well assay plate, comprising ten binding domains (“spots”), is shown in FIG. 39B. In embodiments, Spots 1-4 of FIG. 39B comprise, respectively, an immobilized S-RBD from the following SARS-CoV-2 strains: B.1.1.529 (BA.1); B.1.351; BA.2; and P.1; Spot 6 comprises an immobilized S-RBD from SARS-CoV-2 strain B.1.1.7; Spots 8-10 comprise, respectively, an immobilized S-RBD from the following SARS-CoV-2 strains: BA.1.1; B.1.617.2; and wild-type; and Spots 5 and 7 each comprises an immobilized BSA. In embodiments, the S-RBD mutations from these SARS-CoV-2 strains are described in Table 1E.

In embodiments, the surface of the kits described herein comprises a multi-well assay plate. In embodiments, the surface comprises avidin or streptavidin. In embodiments, each binding reagent comprises biotin. In embodiments, the surface comprises a targeting agent. In embodiments, the kit further comprises a linking agent connected to a targeting agent complement. In embodiments, each binding reagent comprises a supplemental linking agent. In embodiments, the targeting agent and targeting agent complement comprise complementary oligonucleotides. In embodiments, the linking agent comprises avidin or streptavidin, and the supplemental linking agent comprises biotin. Targeting agents, targeting agent complements, linking agents, and supplemental linking agents are further described herein.

In embodiments, the invention provides a kit comprising: (a) one or more binding reagents, each binding reagent binding specifically to: (1) a viral component; (2) a host antibody biomarker; or (3) a host inflammatory and/or tissue damage response biomarker; and (b) one or more detection reagents, each detection reagent binding specifically to the viral component, host antibody biomarker, or the host inflammatory and/or tissue damage response biomarker. In embodiments, the detection reagent that binds to the host antibody biomarker binds IgA, IgG, or IgM. In embodiments, the detection reagent is ACE2. In embodiments, the viral component is a viral protein. In embodiments, the viral component is a viral nucleic acid. In embodiments, the virus is a coronavirus. In embodiments, the virus is SARS-CoV-2. In embodiments, the kit further comprises a surface.

In embodiments, the invention provides a combination of any of the kits described herein. In embodiments, the combination of kits is provided as a single kit, comprising the components of each of the individual kits.

In embodiments, the binding reagent is an antibody or antigen-binding fragment thereof. In embodiments, the detection reagent is an antibody or antigen-binding fragment thereof. In embodiments, any of the detection reagents described herein comprises a detectable label as described herein. In embodiments, the detection reagent comprises a nucleic acid probe as described herein. In embodiments, the kit comprises first and second detection reagents, and the first and second detection reagents respectively comprise first and second nucleic acid probes as described herein. In embodiments, the kit further comprises a reagent for conjugating the detection reagent to a detectable label or a nucleic acid probe.

In embodiments, the detection reagent is lyophilized. In embodiments, the detection reagent is provided in solution. In embodiments, the binding reagent is immobilized on the binding domain. In embodiments, the binding reagent is provided in solution. In embodiments, the reagents and other components of the kit are provided separately. In embodiments, they are provided separately according to their optimal shipping or storage temperatures.

Reagents and methods for immobilizing binding reagents to surfaces, e.g., via targeting agents/targeting agent complements, linking agents/supplemental linking agents, and bridging agents are described herein. In embodiments, the surface is a plate. In embodiments, the surface is a multi-well plate. Non-limiting examples of plates include the MSD® SECTOR™ and MSD QUICKPLEX® assay plates, e.g., MSD® GOLD™ 96-well Small Spot Streptavidin plate. In embodiments, the surface is a particle. In some embodiments, the particle comprises a microsphere. In embodiments, the particle comprises a paramagnetic bead. In embodiments, the surface is a cartridge. In embodiments, the surface comprises an electrode. In embodiments, the electrode is a carbon ink electrode.

In embodiments, the kit further comprises a calibration reagent. In embodiments, the calibration reagent comprises a known quantity of the virus, viral component, or biomarker as described herein. In embodiments, multiple calibration reagents comprise a range of concentrations of the virus, viral component, or biomarker. In embodiments, the multiple calibration reagents comprise concentrations of the virus, viral component, or biomarker near the upper and lower limits of quantitation for the immunoassay. In embodiments, the multiple concentrations of the calibration reagent span the entire dynamic range of the immunoassay. In embodiments, the calibration reagent comprises an antibody biomarker. In embodiments, the antibody biomarker is a neutralizing antibody as described herein. In embodiments, the neutralizing antibody is a monoclonal antibody. In embodiments, the calibration reagent comprises a neutralizing antibody that specifically binds the SARS-CoV S protein, the SARS-CoV-2 S protein, or both. In embodiments, the calibration reagent is derived from human serum known to contain one or more antibodies that specifically bind to one or more viral antigens described herein. In embodiments, the one or more antibodies is human IgG, human IgM, or a combination thereof. In embodiments, the calibration reagent comprises an antibody that specifically binds the SARS-CoV S protein, an antibody that specifically binds SARS-CoV-2 S-NTD, an antibody that specifically binds SARS-CoV-2 S protein, an antibody that specifically binds SARS-CoV-2 S-RBD, an antibody that specifically binds SARS-CoV-2 N protein, an antibody that specifically binds HCoV-OC43 S protein, an antibody that specifically binds HCoV-HKU1 S protein, an antibody that specifically binds MERS-CoV S protein, an antibody that specifically binds HCoV-NL63 S protein, an antibody that specifically binds HCoV-229E S protein, an antibody that specifically binds influenza A/Hong Kong H3 HA protein, an antibody that specifically binds influenza B/Brisbane HA protein, an antibody that specifically binds influenza A/Shanghai H7 HA protein, an antibody that specifically binds influenza A/Michigan H1 HA protein, an antibody that specifically binds influenza B/Phuket HA protein, and an antibody that specifically binds RSV pre-fusion F protein. In embodiments, the calibration reagent comprises an IgG that specifically binds to SARS-CoV-2 S protein, an IgG that specifically binds to SARS-CoV-2 N protein, an IgG that specifically binds to SARS-CoV-2 S-RBD, an IgM that specifically binds to SARS-CoV-2 S protein, an IgM that specifically binds to SARS-CoV-2 N protein, an IgM that specifically binds to SARS-CoV-2 S-RBD, an IgA that specifically binds to SARS-CoV-2 S protein, an IgA that specifically binds to SARS-CoV-2 N protein, and an IgA that specifically binds to SARS-CoV-2 S-RBD.

In embodiments, the calibration reagents are provided in the kit at the following concentrations: about 1 to about 10 BAU/mL of an IgG that specifically binds to SARS-CoV-2 S protein, about 0.1 to about 5 BAU/mL of an IgG that specifically binds to SARS-CoV-2 N protein, about 5 to about 20 BAU/mL of an IgG that specifically binds to SARS-CoV-2 S-RBD, about 0.1 to about 2 BAU/mL of an IgM that specifically binds to SARS-CoV-2 S protein, about 1 to about 5 BAU/mL of an IgM that specifically binds to SARS-CoV-2 N protein, about 0.1 to about 2 BAU/mL of an IgM that specifically binds to SARS-CoV-2 S-RBD, about 1 to about 5 BAU/mL of an IgA that specifically binds to SARS-CoV-2 S protein, about 1 to about 10 BAU/mL of an IgA that specifically binds to SARS-CoV-2 N protein, and about 0.1 to about 5 BAU/mL of an IgA that specifically binds to SARS-CoV-2 S-RBD. Concentrations of the calibration reagents provided herein are defined according to the “First WHO International Standard for anti-SARS-CoV-2 immunoglobulin” (NIBSC code: 20/136).

In embodiments, the calibration reagents are provided in the kit at the following concentrations: about 6.31 BAU/mL of an IgG that specifically binds to SARS-CoV-2 S protein, about 1.89 BAU/mL of an IgG that specifically binds to SARS-CoV-2 N protein, about 8.16 BAU/mL of an IgG that specifically binds to SARS-CoV-2 S-RBD, about 0.867 BAU/mL of an IgM that specifically binds to SARS-CoV-2 S protein, about 2.64 BAU/mL of an IgM that specifically binds to SARS-CoV-2 N protein, about 0.466 BAU/mL of an IgM that specifically binds to SARS-CoV-2 S-RBD, about 3.09 BAU/mL of an IgA that specifically binds to SARS-CoV-2 S protein, about 5.57 BAU/mL of an IgA that specifically binds to SARS-CoV-2 N protein, and about 1.56 BAU/mL of an IgA that specifically binds to SARS-CoV-2 S-RBD.

In embodiments, the calibration reagent is a positive control reagent. In embodiments, the calibration reagent is a negative control reagent. In embodiments, the positive or negative control reagent is used to provide a basis of comparison for the biological sample to be tested with the methods of the present invention. In embodiments, the positive control reagent comprises multiple concentrations of the virus, viral component, or biomarker. In embodiments, the positive control reagent comprises an antibody. In embodiments, the positive control reagent comprises human IgG, IgM, IgA, or a combination thereof. In embodiments, the positive control reagent comprises an antibody that specifically binds the SARS-CoV-2 S protein, SARS-CoV-2 N protein, SARS-CoV-2 S-RBD, or a combination thereof. In embodiments, the positive control reagent comprises an IgG that specifically binds to SARS-CoV-2 S protein, an IgG that specifically binds to SARS-CoV-2 N protein, an IgG that specifically binds to SARS-CoV-2 S-RBD, an IgM that specifically binds to SARS-CoV-2 S protein, an IgM that specifically binds to SARS-CoV-2 N protein, an IgM that specifically binds to SARS-CoV-2 S-RBD, an IgA that specifically binds to SARS-CoV-2 S protein, an IgA that specifically binds to SARS-CoV-2 N protein, and an IgA that specifically binds to SARS-CoV-2 S-RBD.

In embodiments, the positive control reagent is provided in the kit at the following concentrations: about 0.005 to about 1 BAU/mL of an IgG that specifically binds to SARS-CoV-2 S protein; about 0.001 to about 0.1 BAU/mL of an IgG that specifically binds to SARS-CoV-2 N protein; about 0.005 to about 1 BAU/mL of an IgG that specifically binds to SARS-CoV-2 S-RBD; about 0.001 to about 0.1 BAU/mL of an IgM that specifically binds to SARS-CoV-2 S protein; about 0.01 to about 0.1 BAU/mL of an IgM that specifically binds to SARS-CoV-2 N protein; about 0.001 to about 0.1 BAU/mL of an IgM that specifically binds to SARS-CoV-2 S-RBD; about 0.005 to about 0.5 BAU/mL of an IgA that specifically binds to SARS-CoV-2 S protein; about 0.005 to about 0.5 BAU/mL of an IgA that specifically binds to SARS-CoV-2 N protein; and about 0.001 to about 0.1 BAU/mL of an IgA that specifically binds to SARS-CoV-2 S-RBD. In embodiments, the positive control reagents are provided in the kit at the following concentrations: about 0.1504, about 0.0372, and about 0.0133 BAU/mL of an IgG that specifically binds to SARS-CoV-2 S protein; about 0.0457, about 0.0078, and about 0.0025 BAU/mL of an IgG that specifically binds to SARS-CoV-2 N protein; about 0.1952, about 0.0576, and about 0.0148 BAU/mL of an IgG that specifically binds to SARS-CoV-2 S-RBD; about 0.0187, about 0.0054, and about 0.0077 BAU/mL of an IgM that specifically binds to SARS-CoV-2 S protein; about 0.061, about 0.030, and about 0.0285 BAU/mL of an IgM that specifically binds to SARS-CoV-2 N protein; about 0.011, about 0.0047, and about 0.0068 BAU/mL of an IgM that specifically binds to SARS-CoV-2 S-RBD; about 0.0768, about 0.023, and about 0.0103 BAU/mL of an IgA that specifically binds to SARS-CoV-2 S protein; about 0.1414, about 0.0237, and about 0.0379 BAU/mL of an IgA that specifically binds to SARS-CoV-2 N protein; and about 0.0394, about 0.0131, and about 0.0085 BAU/mL of an IgA that specifically binds to SARS-CoV-2 S-RBD.

In embodiments, the calibration reagent is lyophilized. In embodiments, the calibration reagent is provided in solution. In embodiments, the calibration reagent is provided as a stock concentration that is 5×, 10×, 20×, 30×, 40×, 50×, 60×, 70×, 80×, 90×, 100×, 125×, 150× or higher fold concentrations of the highest working concentration of the calibration reagent. In embodiments, the kit further comprises a diluent for preparing multiple concentrations of the calibration reagent. In embodiments, the calibration reagent provided in the kit is diluted 1:10, 1:20, 1:30, 1:40, 1:50, 1:60, 1:70, 1:80, 1:90, 1:100, 1:140, 1:160, 1:200, 1:300, 1:400, 1:500, 1:600, 1:700, 1:800, 1:900, 1:1000, 1:1500, 1:2000, 1:2500, 1:3000, 1:3500, 1:4000, 1:4500, 1:5000, 1:5500, 1:6000, 1:6500, 1:7000, 1:7500, 1:8000, 1:8500, 1:9000, 1:9500, 1:10000, 1:20000, 1:30000, 1:40000, or 1:50000 to provide multiple concentrations of the calibration reagent. In embodiments, the kit comprises multiple calibration reagents at multiple concentrations, e.g., two or more, three or more, four or more, or five or more concentrations. In embodiments, the multiple concentrations of calibration reagents are used to calculate a standard curve. In embodiments, the multiple concentrations of calibration reagents provide thresholds indicating low, medium, or high levels of the virus, viral component, or biomarker being measured.

In embodiments, the kit further comprises a sample collection device. In embodiments, the sample collection device is an applicator stick. In embodiments, the sample collection device is a swab. In embodiments, the sample collection device is a tissue scraper. In embodiments, the sample collection device is a vial or container for collecting a liquid sample.

In embodiments, the kit further comprises one or more of a buffer, e.g., assay buffer, reconstitution buffer, storage buffer, read buffer, wash buffer and the like; a diluent; a blocking solution; an assay consumable, e.g., assay modules, vials, tubes, liquid handling and transfer devices such as pipette tips, covers and seals, racks, labels, and the like; an assay instrument; and/or instructions for carrying out the assay.

In embodiments, the kit comprises lyophilized reagents, e.g., detection reagent and/or calibration reagent. In embodiments, the kit comprises one or more solutions to reconstitute the lyophilized reagents.

In embodiments, a kit comprising the components above include stock concentrations of the components that are 5×, 10×, 20×, 30×, 40×, 50×, 60×, 70×, 80×, 90×, 100×, 125×, 150× or higher fold concentrations of the working concentrations of the immunoassays herein.

In embodiments, the invention provides a kit for collecting a biological sample. In embodiments, the kit for collecting a biological sample can be provided to a subject for collecting the subject's own sample, e.g., saliva sample. The collected sample can then be provided by the subject, e.g., delivered in person or via postal service, to a laboratory for analysis. In embodiments, the kit further comprises an assay instrument, e.g., an assay cartridge and/or a cartridge reader, for the subject to analyze the collected sample. In embodiments, the kit comprises a sample collection device, e.g., an applicator stick, a swab, a tissue scraper, or a vial or container for collecting a liquid sample. In embodiments, the sample collection device comprises a straw for collecting a saliva sample. In embodiments, the sample collection device comprises a storage solution that stabilizes the sample. In embodiments, the sample collection device comprises a unique sample identifier, e.g., a barcode. In embodiments, the kit further comprises instructions for collecting the sample and/or for analyzing the sample in an assay instrument. In embodiments, the kit further comprises an absorbent material, e.g., a tissue. In embodiments, the kit further comprises a secondary container (e.g., a bag) to secure the sample collection device. In embodiments, the kit further comprises a pre-paid postage label or a pre-paid envelope or box for mailing the collected sample.

All references cited herein, including patents, patent applications, papers, textbooks and the like, and the references cited therein, to the extent that they are not already, are hereby incorporated herein by reference in their entirety.

### Exemplary Embodiments

The following Group A, Group B, and Group C are exemplary embodiments of the inventions disclosed herein.

Group A exemplary embodiments:

A method for determining a SARS-CoV-2 strain in a sample, comprising:

detecting at least a first antibody biomarker in the sample that binds to an antigen from a first SARS-CoV-2 strain and at least a second antibody biomarker in the sample that binds to an antigen from a second SARS-CoV-2 strain, wherein the detecting comprises contacting the sample with a surface comprising at least two binding domains, wherein the antigen from the first SARS-CoV-2 strain is immobilized on a first binding domain, and the antigen from the second SARS-CoV-2 strain is immobilized on a second binding domain; and determining a ratio of the first antibody biomarker to the second antibody biomarker, thereby determining the SARS-CoV-2 strain.

The method according to Group A, wherein the method detects 1 to 10 distinct antibody biomarkers in the sample, wherein each antibody biomarker binds to an antigen from a unique SARS-CoV-2 strain, and wherein the antigen from each unique SARS-CoV-2 strain is immobilized on a distinct binding domain on the surface.

The method according to Group A, wherein the antigen comprises an S protein, an N protein, an S-RBD, or a combination thereof.

The method according to Group A, wherein each antigen is immobilized on a distinct binding domain on the surface, and wherein the antigens comprise:

an S protein, an S-RBD, and/or an N protein from a SARS-CoV-2 strain selected from: wild-type; P.1; P.2; P.3; B.1.1.519; B.1.1.529; B.1.1.529 (+R346K); B.1.1.529 (+L452R); BA.1; BA.1.1; BA.2; BA.3; B.1.1.7; B.1.1.7 (+E484K); B.1.258.17; B.1.351; B.1.351.1; B.1.429; B.1.466.2; B.1.525; B.1.526/E484K; B.1.526/S477N; B.1.526.1; B.1.617; B.1.617.1; B.1.617.2; B.1.617.2 (+ΔY144); B.1.617.2 (+E484K); B.1.617.2 (+E484K/N501Y); B.1.617.2 (+K417N/N439K/E484K/N501Y); B.1.617.2 (+K417N/E484K/N501Y); AY.1; AY.2; AY.3, AY.4; AY.5, AY.6, AY.7, AY.4.2; AY.12; AY.14; B.1.617.3; B.1.618; B.1.620; B.1.621; B.1.640.2; BV-1; A.23.1; A.VOI.V2; C.37; and R.1; and/or an S protein and/or an S-RBD from SARS-CoV-2 comprising one or more mutations selected from: R346K, V367F; Q414K, K417N, K417T, N439K, N450K, L452R, L452Q, S477N, T478K, T478R, E484K, E484Q, F490S, Q493R, N501Y.

The method according to Group D, wherein the antigen comprises an S-RBD comprising: a V367F mutation; an N439K mutation; an L452R mutation; an S477N mutation; a T478K mutation, an E484K mutation; an N501Y mutation; L452R and E484Q mutations; L452R and T478K mutations; L452Q and F490S mutations; S477N and E484K mutations; E484K and N501Y mutations; Q414K and N450K mutations; Q493R and N501Y mutations; R346K, T478R, and E484K mutations; K417N, E484K, and N501Y mutations; K417N, L452R, and T478K mutations; or K417T, E484K, and N501Y mutations.

The method according to Group D, wherein the detecting comprises:

(a) forming a binding complex in each binding domain that comprises the antigen and an antibody biomarker that binds to the antigen;

(b) contacting the binding complex in each binding domain with a detection reagent; and

(c) detecting the binding complexes on the surface.

The method according to Group A, wherein the detection reagent comprises a detection antibody, a detection antigen, or an ACE detection reagent.

The method according to Group A, wherein the detection reagent comprises an electrochemiluminescent (ECL) label.

The method according to Group A, wherein the sample is a saliva sample.

The method according to Group A, wherein the sample is from one or more individuals, wherein the one or more individuals are currently infected or previously infected with SARS-CoV-2.

The method according to Group A, wherein the sample comprises a pooled sample from at least two individuals.

The method according to Group A, wherein the method further comprises comparing the SARS-CoV-2 strain from one or more samples from one or more individuals located in one or more geographical regions, thereby tracking spread of the SARS-CoV-2 strain in the one or more geographical regions.

The method according to Group A, wherein the method further comprises comparing the SARS-CoV-2 strain from one or more samples from one or more individuals obtained at different time points, thereby tracking spread of the SARS-CoV-2 strain over time.

The method according to Group A, wherein the SARS-CoV-2 strain is determined by inputting the ratio of the first antibody biomarker to the second antibody biomarker into a classification algorithm.

The method according to Group A, further comprising training the classification algorithm, wherein the training comprises:

measuring the amount of antibody biomarkers in a sample from a subject infected with a known SARS-CoV-2 strain that bind to an antigen from one or more SARS-CoV-2 strains, wherein the one or more SARS-CoV-2 strains comprise the known SARS-CoV-2 strain;

normalizing the amount of measured antibody biomarker that bind to an antigen from the known SARS-CoV-2 strain against the amount of measured antibody biomarker that bind to an antigen from a further SARS-CoV-2 strain; and providing the normalized antibody biomarker amount to the classification algorithm.

A method for determining a SARS-CoV-2 strain in a sample, comprising:

(a) detecting at least a first antibody biomarker in the sample that binds to an antigen from a first SARS-CoV-2 strain and at least a second antibody biomarker in the sample that binds to an antigen from a second SARS-CoV-2 strain, wherein the detecting comprises contacting the sample with a surface comprising at least two binding domains, wherein the antigen from the first SARS-CoV-2 strain is immobilized on a first binding domain, and the antigen from the second SARS-CoV-2 strain is immobilized on a second binding domain;

wherein each antigen is immobilized on a distinct binding domain on the surface, and wherein the antigens comprise:

an S protein, an S-RBD, and/or an N protein from a SARS-CoV-2 strains selected from: wild-type; P.1; P.2; P.3; B.1.1.519; B.1.1.529; B.1.1.529 (+R346K); B.1.1.529 (+L452R); BA.1; BA.1.1; BA.2; BA.3; B.1.1.7; B.1.1.7 (+E484K); B.1.258.17; B.1.351; B.1.351.1; B.1.429; B.1.466.2; B.1.525; B.1.526/E484K; B.1.526/S477N; B.1.526.1; B.1.617; B.1.617.1; B.1.617.2; B.1.617.2 (+ΔY144); B.1.617.2 (+E484K); B.1.617.2 (+E484K/N501Y); B.1.617.2 (+K417N/N439K/E484K/N501Y); B.1.617.2 (+K417N/E484K/N501Y); AY.1; AY.2; AY.3, AY.4; AY.5, AY.6, AY.7, AY.4.2; AY.12; AY.14; B.1.617.3; B.1.618; B.1.620; B.1.621; B.1.640.2; BV-1; A.23.1; A.VOI.V2; C.37; and R.1; and/or an S protein and/or an S-RBD from SARS-CoV-2 comprising one or more mutations selected from: R346K, V367F; Q414K, K417N, K417T, N439K, N450K, L452R, L452Q, S477N, T478K, T478R, E484K, E484Q, F490S, Q493R, N501Y, wherein the sample is from one or more individuals, wherein the one or more individuals are currently infected or previously infected with SARS-CoV-2, and optionally wherein the one or more individuals are located in one or more geographical regions and/or the samples are obtained at different times;

(b) determining a ratio of the first antibody biomarker to the second antibody biomarker;

(c) inputting the ratio of the first antibody biomarker to the second antibody biomarker into a classification algorithm, wherein the classification algorithm is trained by a training method comprising:
measuring the amount of antibody biomarkers in a sample from a subject infected with a known SARS-CoV-2 strain that bind to an antigen from at least two SARS-CoV-2 strains, wherein the at least two SARS-CoV-2 strains comprise the known SARS-CoV-2 strain and a further SARS-CoV-2 strain;
normalizing the amount of measured antibody biomarker that bind to an antigen from the known SARS-CoV-2 strain against the amount of measured antibody biomarker that bind to an antigen from the further SARS-CoV-2 strain; and
providing the normalized antibody biomarker amount to the classification algorithm;

(d) determining the SARS-CoV-2 strain based on the classification algorithm; and

(e) optionally, tracking spread of the SARS-CoV-2 strain in the one or more geographical region, tracking spread of the SARS-CoV-2 strain over time, or a combination thereof.

Group B exemplary embodiments:

A method for detecting a single nucleotide polymorphism (SNP) in a target nucleic acid, wherein the target nucleic acid is a SARS-CoV-2 nucleic acid, comprising:

(a) contacting a sample comprising the target nucleic acid with (i) a targeting probe, wherein the targeting probe comprises a first region complementary to a polymorphic site of the target nucleic acid that comprises the SNP, and wherein the targeting probe comprises an oligonucleotide tag; and (ii) a detection probe, wherein the detection probe comprises a second region complementary to an adjacent region of the target nucleic acid comprising the polymorphic site, and wherein the detection probe comprises a detectable label, wherein the targeting probe and the detection probe each independently comprises a sequence as shown in Table 10 or Table 14;

(b) hybridizing the targeting and detection probes to the target nucleic acid;

(c) ligating the targeting and detection probes that hybridize with perfect complementarity at the polymorphic site to form a ligated target complement comprising the oligonucleotide tag and the detectable label;

(d) contacting the product of (c) with a surface comprising an immobilized binding reagent, wherein the binding reagent comprises an oligonucleotide complementary to the oligonucleotide tag;

(e) forming a binding complex on the surface, wherein the binding complex comprises the binding reagent and the ligated target complement; and

(f) detecting the binding complex, thereby detecting the SNP at the polymorphic site.

The method according to Group B, wherein the targeting probe hybridizes to the target nucleic acid such that a terminal 5′ nucleotide of the targeting probe hybridizes with the SNP, and the detection probe hybridizes to the target nucleic acid adjacent to the SNP and provides a 3′ end for ligating the targeting and the detection probes.

The method according to Group B, wherein the detection probe hybridizes to the target nucleic acid such that a terminal 5′ nucleotide of the detection probe hybridizes with the SNP, and the targeting probe hybridizes to the target nucleic acid adjacent to the SNP and provides a 3′ end for ligating the targeting and the detection probes.

The method according to Group B, wherein the detection probe hybridizes to the target nucleic acid such that a terminal 3′ nucleotide of the detection probe hybridizes with the SNP, and the targeting probe hybridizes to the target nucleic acid adjacent to the SNP and provides a 5′ end for ligating the targeting and the detection probes.

The method according to Group B, further comprising providing a blocking probe during the ligating, wherein the blocking probe comprises a sequence as shown in Table 12 or Table 16.

The method according to Group B, wherein the detectable label comprises an ECL label.

Group C exemplary embodiments:

A kit for detecting one or more antibody biomarkers of interest in a sample, the kit comprising, in one or more vials, containers, or compartments:

(a) a surface comprising one or more binding domains, wherein each binding domain comprises an antigen immobilized thereon, and wherein the antigens comprise:

(i) a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises an N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD;

(ii) a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD;

(iii) a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD;

(iv) an S protein from the following SARS-CoV-2 strains: wild type; P.2; B.1.617.1; B.1.617.2; B.1.617.3; B.1.617; P.1; B.1.1.7; B.1.351; and B.1.526.1;

(v) an S protein from the following SARS-CoV-2 strains: wild type; A.23.1; A.VOI.V2; B.1.617.2; C.37; R.1; P.3; B.1.525; B.1.1.519; and BV-1;

(vi) an S protein from the following SARS-CoV-2 strains: wild type; AY.1, AY.2, B.1.617.2 (+ΔY144); B.1.620; B.1.258.17; B.1.466.2; B.1.1.7 (+E484K(; B.1.351.1; and B.1.618;

(vii) a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD;

(viii) a wild-type S protein from SARS-CoV-2; an S-D614G from SARS-CoV-2; an N protein from SARS-CoV-2; an S protein from SARS-CoV-2 strain B.1.617.2; an S protein from SARS-CoV-2 strain P.1; an S protein from SARS-CoV-2 strain B.1.1.7; an S protein from SARS-CoV-2 strain B.1.351; and a wild-type S-RBD from SARS-CoV-2;

(ix) an S protein from the following SARS-CoV-2 strains: wild type; P.2; B.1.617.1; B.1.617.3; B.1.617; P.1; and B.1.1.7;

(x) an S protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14);

(xi) an S protein from the following SARS-CoV-2 strains: wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2;

(xii) an S protein from the following SARS-CoV-2 strains: wild-type; B.1.1.529; AY.4.2; AY.4; P.1; B.1.1.7; B.1.351; and B.1.617.2;

(xii) an S-RBD from the following SARS-CoV-2 strains: B.1.1.529; B.1.351; P.1; B.1.1.7; B.1.617.2; and wild-type;

(xiv) an S protein from the following SARS-CoV-2 strains: wild-type, B.1.1.529, AY.4, P.1, B.1.1.7, B.1.351; an N protein from wild-type SARS-CoV-2; and an S-RBD from wild-type SARS-CoV-2;

(xv) an S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2;

or

(xvi) an S-RBD from the following SARS-CoV-2 strains: B.1.1.529 (BA.1); B.1.351; BA.2; P.1; B.1.1.7; BA.1.1; B.1.617.2; and wild-type; and

(b) one or more detection reagents, wherein each detection reagent comprises a detection antibody, a detection antigen, or an ACE detection reagent.

The kit according to Group C, wherein the detection reagent comprises an electrochemiluminescent (ECL) label.

The kit according to Group C, wherein the surface comprises an electrode.

The kit according to Group C, wherein the surface comprises a well of a multi-well plate, and wherein each well comprises 1 to 10 binding domains.

A method of detecting one or more antibody biomarkers of interest in a sample, comprising:

(a) contacting the sample with a surface comprising one or more binding domains, wherein each binding domain comprises an antigen immobilized thereon, and wherein the antigens comprise:

(i) a SARS-CoV-2 S-RBD that comprises an L452R mutation; a SARS-CoV-2 S-RBD that comprises K417N, E484K, and N501Y mutations; a SARS-CoV-2 S-RBD that comprises an E484K mutation; a SARS-CoV-2 S-RBD that comprises K417T, E484K, and N501Y mutations; a SARS-CoV-2 S-RBD that comprises an S477N mutation; a SARS-CoV-2 S-RBD that comprises an N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises Q414K and N450K mutations; and a wild-type SARS-CoV-2 S-RBD;

(ii) a SARS-CoV-2 S-RBD that comprises a L452R mutation; a SARS-CoV-2 S-RBD that comprises K417N, E484K, and N501Y mutations; a SARS-CoV-2 S-RBD that comprises a E484K mutation; a SARS-CoV-2 S-RBD that comprises K417T, E484K, and N501Y mutations; a SARS-CoV-2 S-RBD that comprises a S477N mutation; a SARS-CoV-2 S-RBD that comprises a N501Y mutation; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD;

(iii) a SARS-CoV-2 S-RBD that comprises a V367F mutation; a SARS-CoV-2 S-RBD that comprises L452Q and F490S mutations; a SARS-CoV-2 S-RBD that comprises a E484K mutation; a SARS-CoV-2 S-RBD that comprises Q493R and N501Y mutations; a SARS-CoV-2 S-RBD that comprises a T478K mutation; a SARS-CoV-2 S-RBD that comprises R346K, T478R, and E484K mutations; a SARS-CoV-2 S-RBD that comprises E484K and N501Y mutations; a SARS-CoV-2 S-RBD that comprises L452R and E484Q mutations; a SARS-CoV-2 S-RBD that comprises L452R and T478K mutations; and a wild type SARS-CoV-2 S-RBD;

(iv) an S protein from the following SARS-CoV-2 strains: wild type; P.2; B.1.617.1; B.1.617.2; B.1.617.3; B.1.617; P.1; B.1.1.7; B.1.351; and B.1.526.1;

(v) an S protein from the following SARS-CoV-2 strains: wild type; A.23.1; A.VOI.V2; B.1.617.2; C.37; R.1; P.3; B.1.525; B.1.1.519; and BV-1;

(vi) an S protein from the following SARS-CoV-2 strains: wild type; AY.1, AY.2, B.1.617.2 (+ΔY144); B.1.620; B.1.258.17; B.1.466.2; B.1.1.7 (+E484K(; B.1.351.1; and B.1.618;

(vii) a SARS-CoV-2 S-RBD that comprises K417N, L452R, and T478K mutations; a SARS-CoV-2 S-RBD that comprises K417N, E484K, and N501Y mutations; a SARS-CoV-2 S-RBD that comprises a E484K mutation; a SARS-CoV-2 S-RBD that comprises S477N and E484K mutations; a SARS-CoV-2 S-RBD that comprises E484K and N501Y mutations; a SARS-CoV-2 S-RBD that comprises a N439K mutation; a SARS-CoV-2 S-RBD that comprises L452R and T478K mutations; and a wild type SARS-CoV-2 S-RBD;

(viii) a wild-type S protein from SARS-CoV-2; an S-D614G from SARS-CoV-2; an N protein from SARS-CoV-2; an S protein from SARS-CoV-2 strain B.1.617.2; an S protein from SARS-CoV-2 strain P.1; an S protein from SARS-CoV-2 strain B.1.1.7; an S protein from SARS-CoV-2 strain B.1.351; and a wild-type S-RBD from SARS-CoV-2;

(ix) an S protein from the following SARS-CoV-2 strains: wild type; P.2; B.1.617.1; B.1.617.3; B.1.617; P.1; and B.1.1.7;

(x) an S protein from the following SARS-CoV-2 strains: wild-type; B.1.621; AY.2; B.1.617.2 (AY.4); C.37; AY.12; P.1; AY.1; B.1.351; and B.1.617.2 (AY.3, AY.5, AY.6, AY.7, AY.14);

(xi) an S protein from the following SARS-CoV-2 strains: wild-type; B.1.617.2 (+K417N/N439K/E484K/N501Y); B.1.617.2 (+K417N/E484K/N501Y); AY.4; B.1.617.2 (+E484K/N501Y); B.1.617.2 (+E484K); P.1; B.1.1.7; B.1.351; and B.1.617.2;

(xii) an S protein from the following SARS-CoV-2 strains: wild-type; B.1.1.529; AY.4.2; AY.4; P.1; B.1.1.7; B.1.351; and B.1.617.2;

(xii) an S-RBD from the following SARS-CoV-2 strains: B.1.1.529; B.1.351; P.1; B.1.1.7; B.1.617.2; and wild-type;

(xiv) an S protein from the following SARS-CoV-2 strains: wild-type, B.1.1.529, AY.4, P.1, B.1.1.7, B.1.351; an N protein from wild-type SARS-CoV-2; and an S-RBD from wild-type SARS-CoV-2;

(xv) an S protein from the following SARS-CoV-2 strains: wild-type; B.1.1.529; BA.2; AY.4; BA.3; B.1.1.529 (+R346K); B.1.1.529 (+L452R); B.1.1.7; B.1.351; and B.1.640.2;

or

(xvi) an S-RBD from the following SARS-CoV-2 strains: B.1.1.529 (BA.1); B.1.351; BA.2; P.1; B.1.1.7; BA.1.1; B.1.617.2; and wild-type;

(b) forming a binding complex in each binding domain, wherein the binding complex comprises the antigen and an antibody biomarker that binds to the antigen;
(c) contacting the binding complex in each binding domain with a detection reagent; and
(d) detecting the binding complexes on the surface, thereby detecting the one or more antibody biomarkers in the sample.

The method according to Group C, wherein the detection reagent comprises a detection antibody, a detection antigen, or an ACE detection reagent.

The method according to Group C, wherein the detection reagent comprises an ECL label.

The method according to Group C, wherein the surface comprises an electrode.

The method according to Group C, wherein the surface comprises a well of a multi-well plate, and wherein each well comprises 1 to 10 binding domains.

The method according to Group C, wherein the detection reagent comprises an ECL label, the surface comprises an electrode, and the detecting comprises applying a voltage to the surface and measuring an ECL signal generated from the ECL label on the detection reagent.

### EXAMPLES

### Example 1. Bridging Serology Assay for SARS-CoV-2 on Patient Samples

A bridging serology assay to detect SARS-CoV-2 antibodies was performed using the SARS-CoV-2 S-RBD antigen. The bridging serology assay used the simultaneous binding of antibodies to immobilized viral antigen and detection tag-labeled viral antigen, leading to a highly specific isotype-independent measurement of immune reactivity. The tested samples were from the same COVID-19 positive and normal patients as described in Example 4, except the samples were diluted 10-fold or 100-fold.

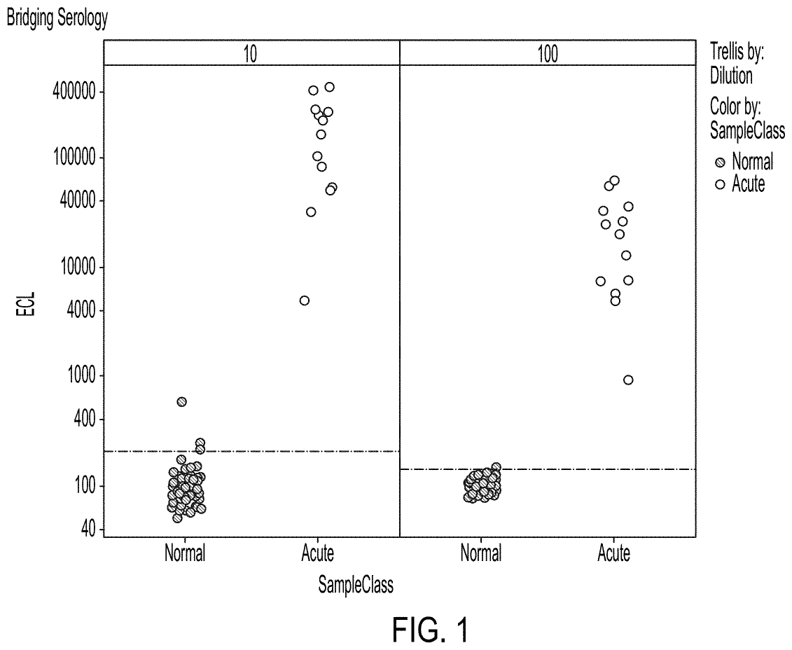

Results are shown in FIG. 1. For both 10-fold and 100-fold sample dilutions, clear separation of signal was observed between the COVID-19 patient and normal samples. High signals detected in the COVID-19 sera indicates the presence of anti-S-RBD antibodies.

### Example 2. Neutralization Serology Assay for SARS-CoV-2 on Patient Samples

As described herein, neutralization serology assays (also termed “competitive serology assay”) can allow assessment of the potential protective serological response present in the patient. A neutralization serology assay was performed to test SARS-CoV-2 antibody binding to immobilized SARS-CoV-2 S protein in the presence of the host cell protein receptor, ACE2, as competitor for the antigen. The ACE2 is labeled with a detection label. The tested samples were from the same COVID-19 positive and normal patients as described in Example 4, except the samples were diluted 10-fold or 100-fold.

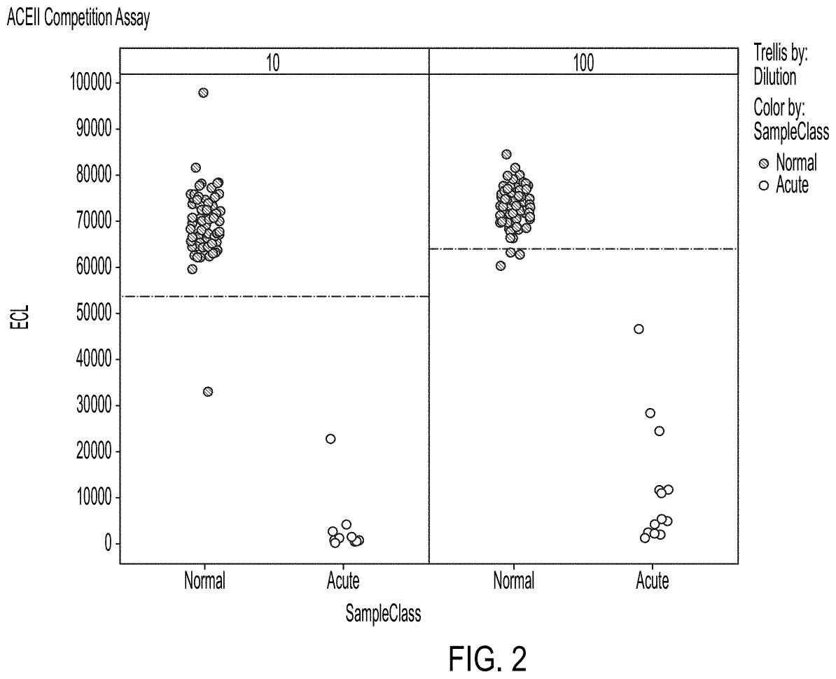

Results are shown in FIG. 2. For both the 10-fold and 100-fold sample dilutions, clear separation of signal was observed between the COVID-19 patients and normal samples. Lower signal detected in the COVID-19 sera indicates inhibition of the interaction between the SARS-CoV-2 S protein and its cognate receptor, ACE2, in COVID-19 patient samples.

### Example 3. One-Step RT-PCR for Amplifying Viral Nucleic Acid

An exemplary protocol for reverse transcribing and amplifying a viral RNA (e.g., SARS-CoV-2 RNA) is described in this Example.

1) Extract RNA from a sample containing the virus of interest, e.g., SARS-CoV-2;

2) Prepare a Master Mix containing a single primer pair or multiplexed primer pairs and a one-step RT-PCR mix (e.g., from a commercially available source);

3) Add the sample RNA to the Master Mix;

4) Program a thermocycler to run the following steps (Table 3):

TABLE 3
Thermocycler Conditions

| Step | Cycles | Temp | | Time | |
|---|---|---|---|---|---|
| UNG incubation | 1 | 25° | C. | 2 | min |
| RT incubation | 1 | 50° | C. | 15 | min |
| Polymerase activation | 1 | 95° | C. | 2 | min |
| Amplification | 45 | 95° | C. | 3 | sec |
| | | 55° | C. | 30 | min |

| | | | | |
|---|---|---|---|---|
| Hold | 1 | 4° | C. | Hold |

The number of amplification cycles and annealing temperature can be adjusted based on the experiment (e.g., length of nucleic acid to be amplified).

The PCR product is ready to be tested with the nucleic acid detection methods described herein.

### Example 4. Correlation of Different Serology Assay Formats

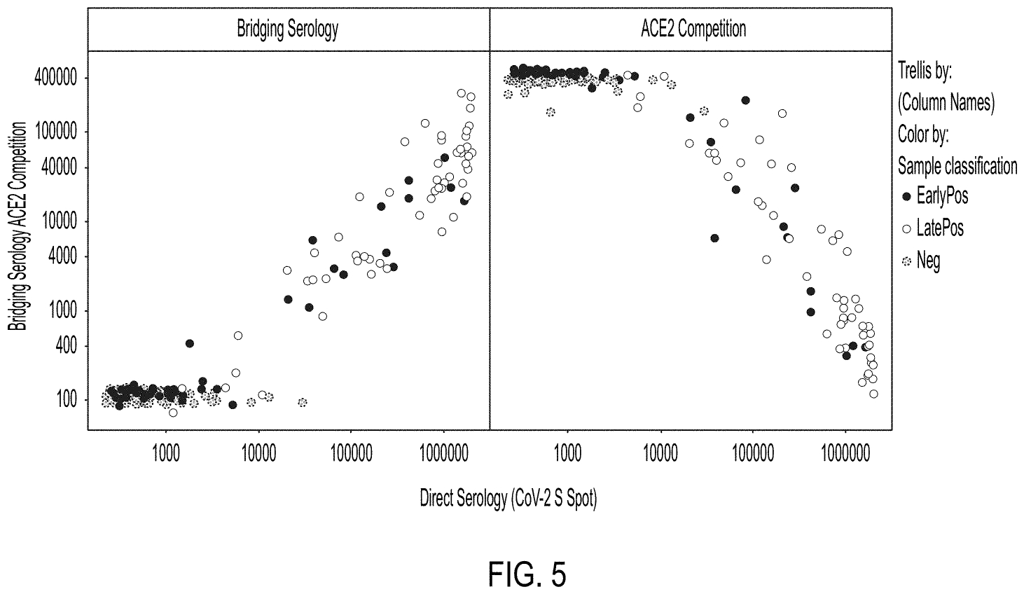

Results of a bridging serology assay and a neutralization serology assay were plotted against results of a classical serology assay to determine the correlation between the different serology assay formats. The plot is shown in FIG. 5, indicating the results between different assay formats are well-correlated.

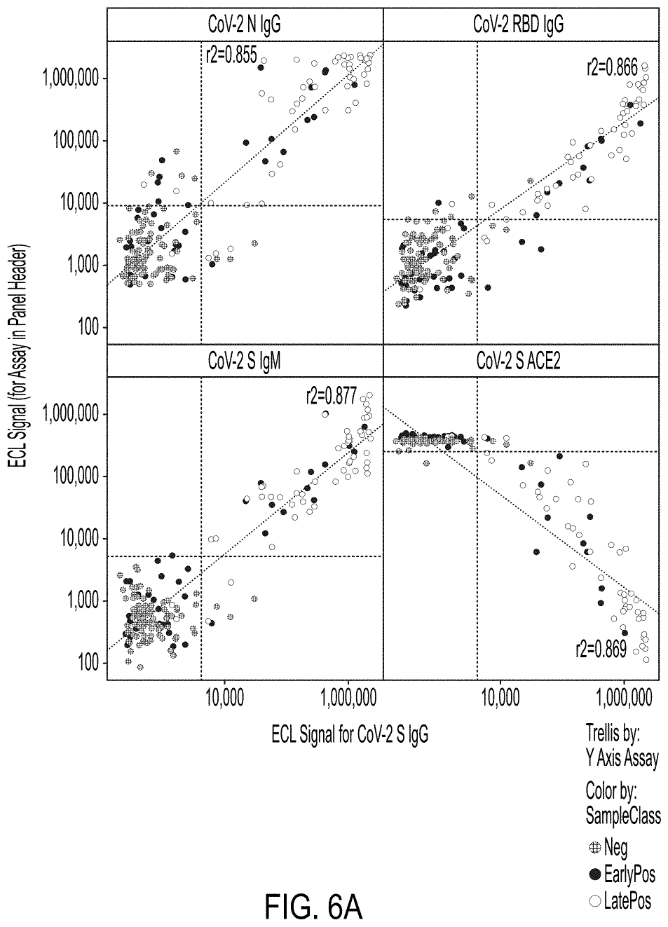

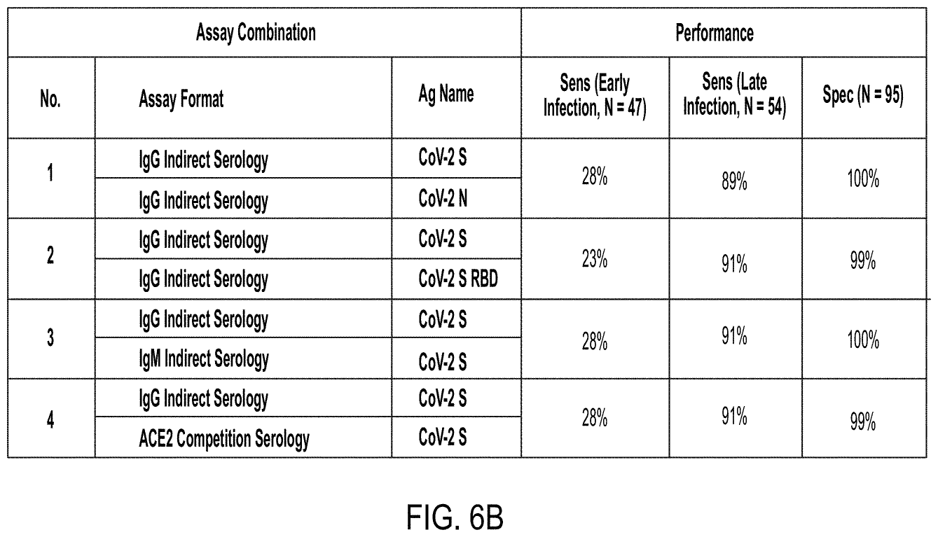

FIG. 6A shows the correlation of the indirect serology assays for IgG against SARS-CoV-2 S with four other serology assays (IgG against SARS-CoV-2 N, IgG against SARS-CoV-2 S-RBD, IgM against SARS-CoV-2 S, and ACE2 competitor assay). Elevated levels of IgG against SARS-CoV-2 S was associated with elevated levels of IgG against SARS-CoV-2 N (r²=0.86) and RBD (r²=0.87). Correlation was only observed if samples from infected patients were included in the analysis. Association between the assay signals was weak if the analysis was limited to just the naïve samples, in which case the r² values dropped to 0.07 and 0.17, respectively. A similar result was obtained by comparing the measured IgG and IgM response to SARS-CoV-2 S, which provided an r² value of 0.88 for the full sample set and 0.00 when limited to the naïve samples. Finally, generation of IgG antibodies that could bind SARS-CoV-2 S as measured by the indirect format was generally associated with the production of antibodies targeting the ACE2 binding site as measured by the ACE2 competition format (r²=0.87).

The weak correlation of signals for naïve samples provides a potential approach to improving assay performance by combining the results from multiple assays. For example, in the correlation plot for IgG against N vs. IgG against S (top left panel of FIG. 6A), there were some naïve samples that provided signals above the selected threshold for N but not S (top left area of panel) or S but not N (bottom right are of panel). The classification accuracy was further evaluated by requiring two assays to call a sample positive, as a method for reducing the impact of false positive signals for individual assays. FIG. 6B shows the classification performance for the possible pairings of the assays shown in FIG. 6A. In all four cases shown in the table in FIG. 6B, the combined assay result improved the specificity for classification of the naïve samples to 99% or 100% (0 or 1 false positive out of 95 samples). The improvement in specificity was accompanied by a small decrease in sensitivity: the sensitivities for classifying the late infection samples ranged from 89% to 91% for the two-assay combination compared to a range of 91% to 94% for the individual assays. In particular, combining the measurements of the IgG response to SARS-CoV-2 N and S proteins provided sensitivities of 28% and 89% for classifying the early and late infection samples, respectively, and a specificity of 100% for classifying the naïve samples.

### Example 5. Evaluation of Assay Performance

The sensitivity and specificity of serology assays were determined for IgG and IgM using SARS-CoV-2 S, S-RBD, and N antigens, and ACE2 competitor (neutralization) serology assays using SARS-CoV-2 S and S-RBD antigens. ROC analysis was used to identify the threshold for each combination of format and antigen that maximized the sum of the specificity and sensitivity for separating the late infections from the normal controls. This threshold was then applied to both the early infection and late infection data sets.

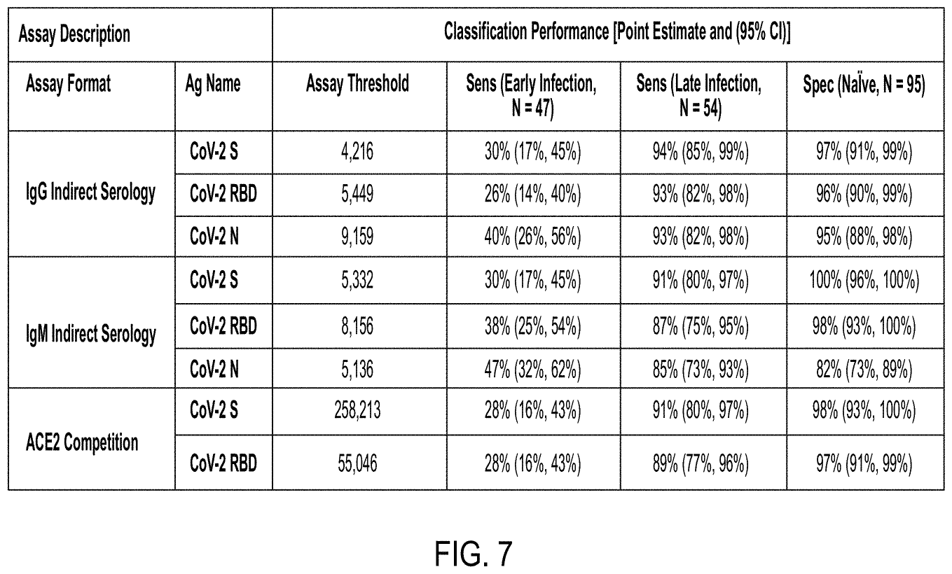

Using the optimal thresholds, the different assays shown in FIG. 7 provided similar sensitivity and specificity values, with largely overlapping 95% confidence limits. All the assays provided point estimates for specificity that were greater than or equal to 95%, except for the measurement of IgM against SARS-CoV-2 S protein. Measurements of IgG using the indirect serology format provided the highest point estimates for sensitivity in classifying late infections (point estimates of sensitivity for S, RBD and N ranged from 93% to 94%). Point estimates for measurement of IgM using the indirect serology format or inhibitory antibodies using the ACE2 competition format were slightly lower ranging from 85% to 91%, with the best sensitivity in both formats provided by the SARS-CoV-2 S antigen (91% sensitivity in both the IgM and ACE2 formats). All the assays provided lower sensitivity for measuring early infection with point estimates for sensitivity ranging from 26% to 47%. There was no evidence that measurement of IgM provided statistically higher sensitivity for classification of the early infection samples, relative to measurement of IgG.

### Example 6. SARS-CoV-2 Single Nucleotide Polymorphism (SNP) Assay

Singleplex and multiplex oligonucleotide ligation assays (OLA) were performed to detect single nucleotide polymorphisms (SNPs) at SARS-CoV-2 genome locations 8782 (C>T mutation), 11083 (G>T mutation), 23403 (A>G mutation), and 28144 (T>C mutation). A pair of targeting probes and detection probes for each polymorphic site were designed to allow for single-base discrimination at the SNP site, as described in embodiments herein. The targeting probes have unique 5′ oligonucleotide tag sequences that are complementary to binding reagents on specific binding domains on a multi-well plate. The detection probes have a 5′ phosphate group for ligation and a 3′ biotin. Taq DNA ligase was used to join the targeting probe and detection probe that aligned correctly on the sample. Fragments of unmodified template complements were added to prevent bridging of unligated probes. The OLA cycling conditions were: 2 minutes at 95° C., then 30 cycles of 30 seconds at 95° C. and 2 minutes at 65° C. The plates were blocked with a blocking solution for 30 minutes at 37° C. during the OLA cycling.

The plate was washed, and the ligated probes were hybridized to a binding reagent on a plate. In the singleplex format, each well only contained the binding reagents specific for one SNP. In the multiplex format, each well of contained ten binding domains (“spots”), wherein a unique binding reagents was immobilized in each spot, allowing for detection of up to five SNPs per well. The hybridization was performed in hybridization buffer (50 μL per well), with a one-hour incubation at 37° C.

The hybridized ligated probes were then detected by streptavidin-SULFO-TAG™. Briefly, following the hybridization, the plate was washed, and a detection solution was added (50 μL per well) and incubated for 30 minutes at 37° C. The plate was washed, and read buffer was added (150 μL), and the plate was then read using a plate reader.

Ten (10) nasal swabs from COVID-19-positive patients obtained from BOCA Biolistics (Deerfield Beach, Fla.) were tested using both the singleplex and multiplex OLA assay formats. RNA was extracted using the MAGMAX™ Viral/Pathogen Ultra Nucleic Acid Isolation Kit (Applied Biosystems). RT-PCR was conducted using site-specific primers using TAQPATH™ 1-step RT-qPCR Master Mix (Applied Biosystems). Reference strain RNAs were obtained from BEI Resources, NIAID, NIH: Genomic RNA from SARS-Related Coronavirus 2, Isolate USA-WA1/2020, NR-52285, GenBankMN985325, deposited by the Centers for Disease Control and Prevention; Genomic RNA from SARS-Related Coronavirus 2, Isolate Hong Kong/VM20001061/2020, NR-52388, GenBankMT547814, deposited by the University of Hong Kong.

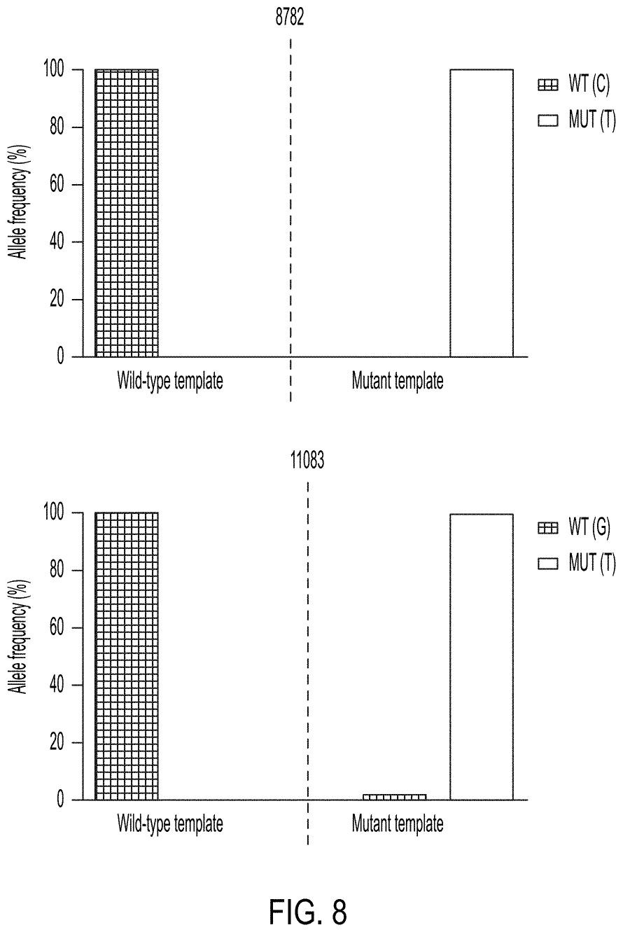

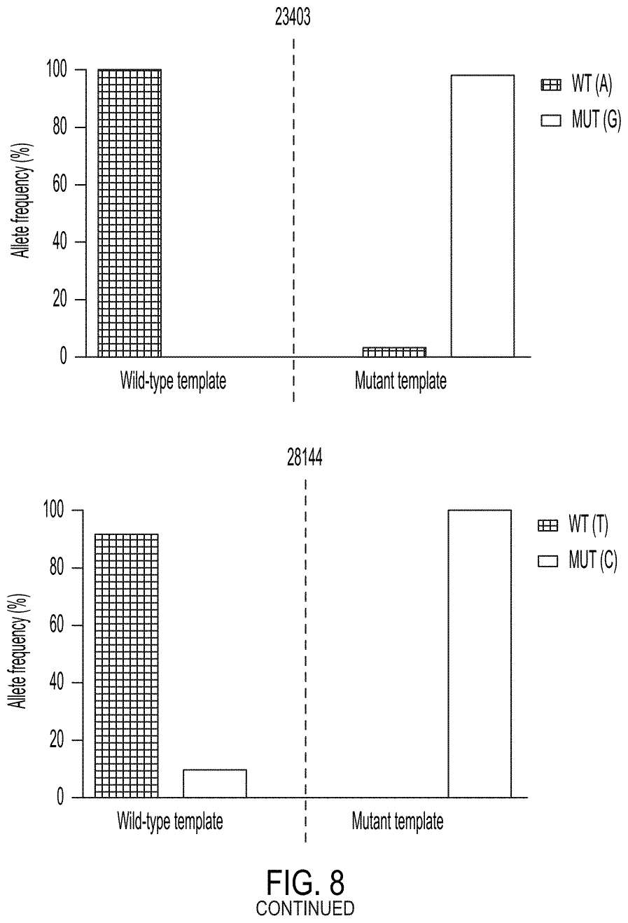

Results from a test assay using synthetic oligonucleotide template containing the SNPs of interest are shown FIG. 8. The synthetic oligonucleotide templates showed high specificity for the appropriate allele using ligation temperature of 65° C.

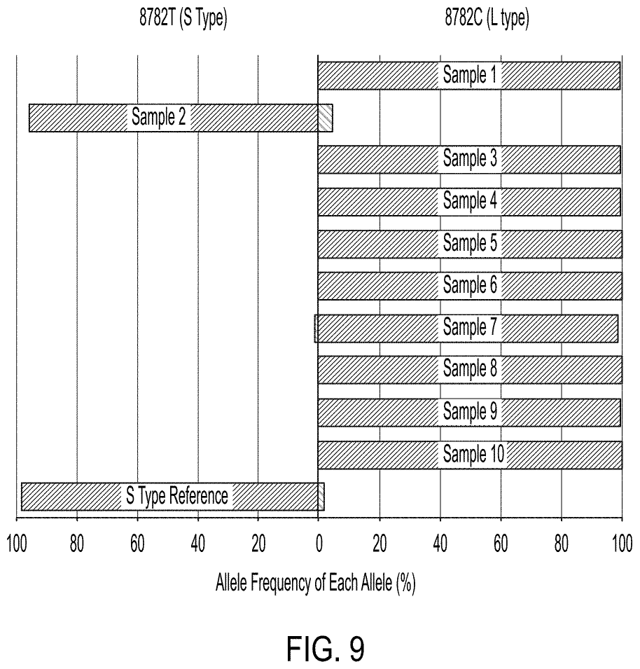

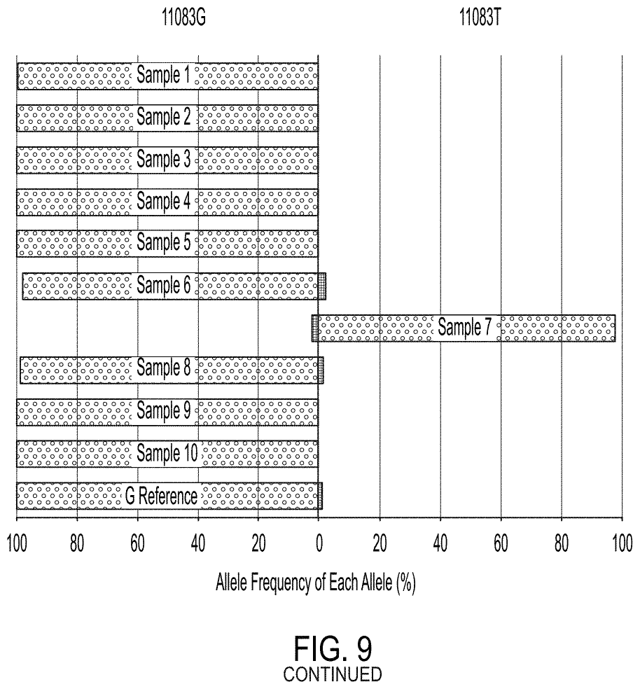

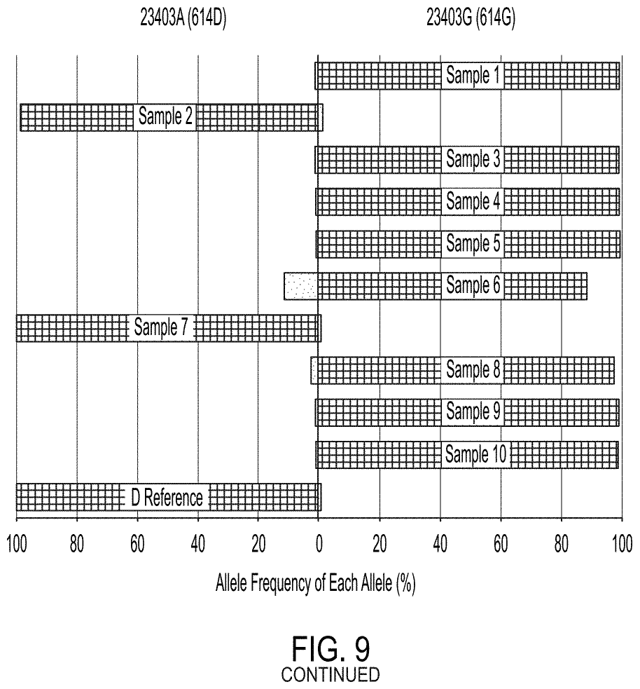

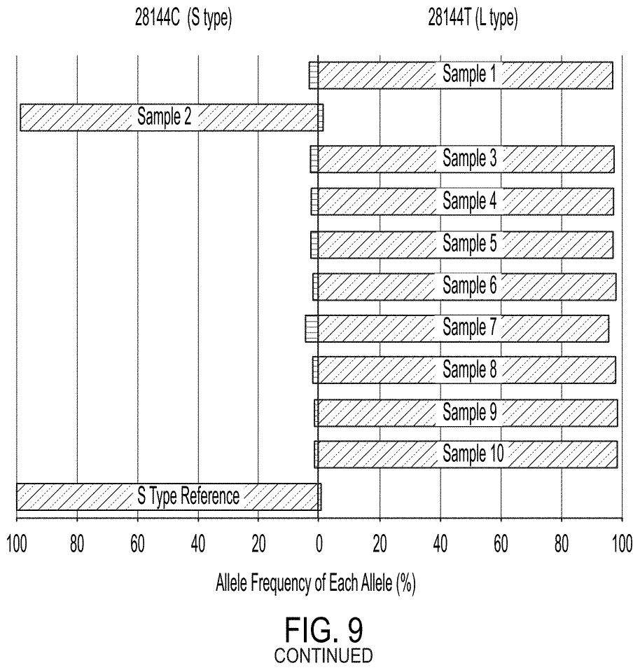

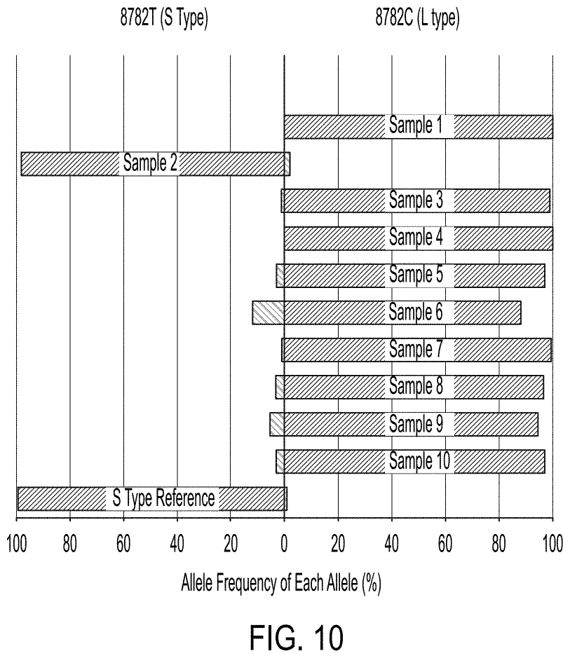

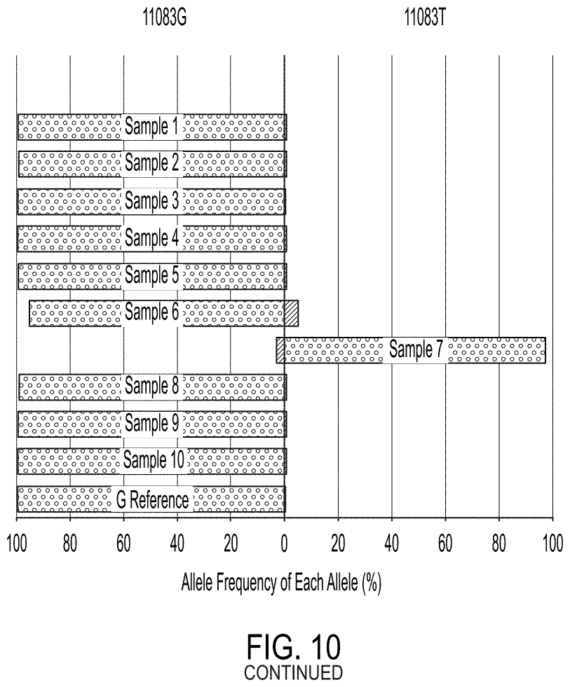

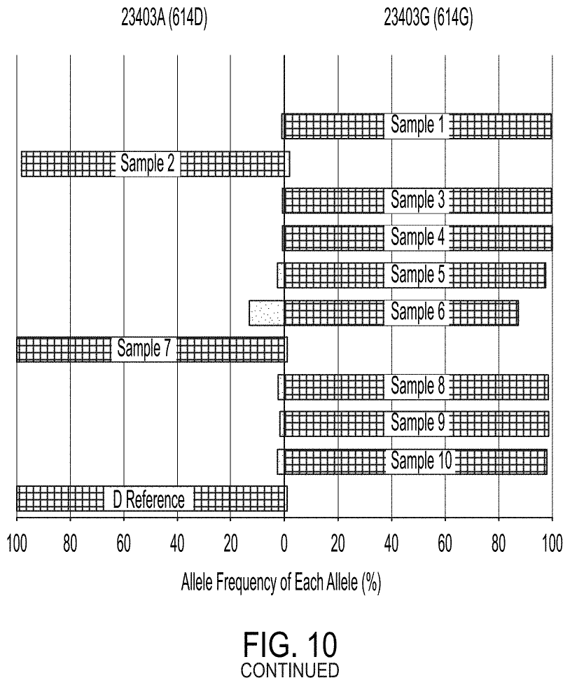

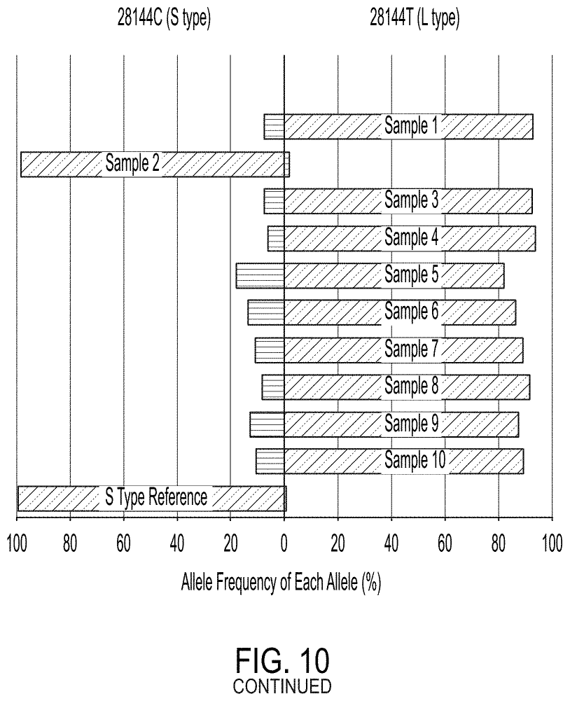

Results from the singleplex assay format with patient samples are shown in FIG. 9. There was a high prevalence of the L strain type (8782C and 28144T) that also have the D614G mutation. Only one strain had the “asymptomatic” allele (11083T). Results were also compared to a fully sequenced reference strain, with all results matching the published sequences for these strains. Results from the multiplex assay format with patient samples are shown in FIG. 10 and were consistent with the results from the singleplex assays shown in FIG. 9.

Reproducibility of the multiplex assay was also assessed. The multiplex RT-PCR products from the samples shown in FIG. 10 were subjected to three separate multiplexed OLA reactions on three different days to determine the allele frequency reproducibility from the SNP assays. The mean, standard deviation (STD), coefficient of variation (CV), and total number (N) of readings are shown in Table 4 for the samples that either had the WT or mutant (MUT) allele for the given SNP. As shown in Table 4, all assays had good allele frequency reproducibility, and the SNP determinations for all ten samples and the reference strain were consistent between the runs for all three experiments.

TABLE 4
OLA Reproducibility

| | WT samples (WT allele) | | | | MUT samples (MUT allele) | | | |
|---|---|---|---|---|---|---|---|---|
| | | | | Total | | | | Total |
| Site | Mean | STD | CV | N | Mean | STD | CV | N |
| 8782 | 97.8 | 3.1 | 0.03 | 54 | 98.6 | 0.3 | 0.003 | 6 |
| 11083 | 99.3 | 2.8 | 0.03 | 54 | 98.1 | 1.0 | 0.01 | 6 |
| 23403 | 99.2 | 0.4 | 0.004 | 12 | 100.3 | 5.4 | 0.05 | 48 |
| 28144 | 96.3 | 10.0 | 0.1 | 54 | 98.7 | 0.8 | 0.01 | 6 |

### Example 7. Detection of SARS-CoV-2 Nucleocapsid Protein in Human Samples

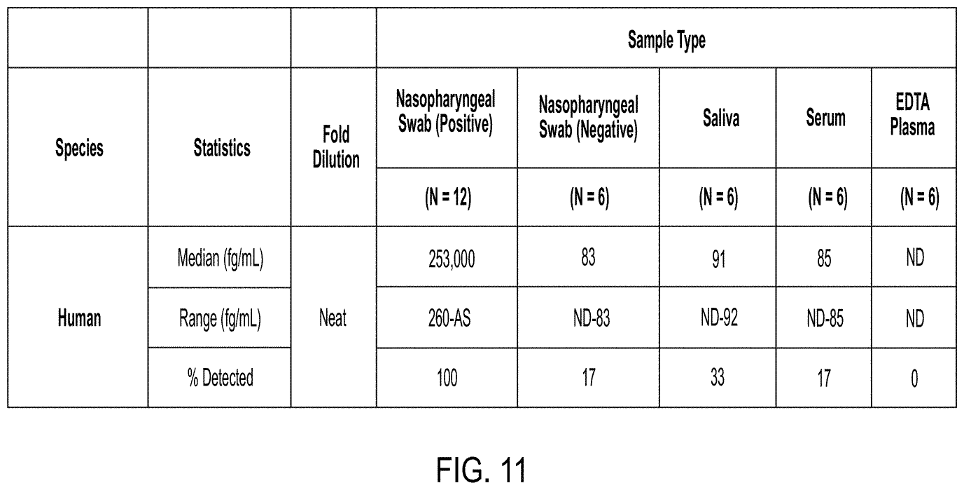

A detection assay was used to test for SARS-CoV-2 N protein in the following samples: nasopharyngeal swabs from 12 patients who tested positive for COVID-19, nasopharyngeal swabs from 6 patients who tested negative for COVID-19, and normal (COVID-19 negative) human saliva, serum, and EDTA plasma. The detection assay was performed as follows: To each well of a 96-well plate containing immobilized anti-Nucleocapsid capture antibody, add 25 μL labeled anti-Nucleocapsid detection antibody labeled with nucleic acid probe, and 25 μL sample. Incubate for 1 hour at room temperature with shaking. Wash plate and add extension solution. Incubate for 15 minutes at room temperature with shaking. Wash plate and add detection solution. Incubate for 45 minutes at room temperature with shaking. Wash plate, add 150 μL ECL Read Buffer, and read plate. Total protocol time is approximately 2 hours. The samples were all tested without dilution. Assay results of the SARS-CoV-2 N protein concentration are shown in FIG. 11. The results for the negative nasopharyngeal swab and normal saliva, serum, and EDTA plasma were comparable, while the positive nasopharyngeal swab samples had significantly higher concentration of SARS-CoV-2 N protein.

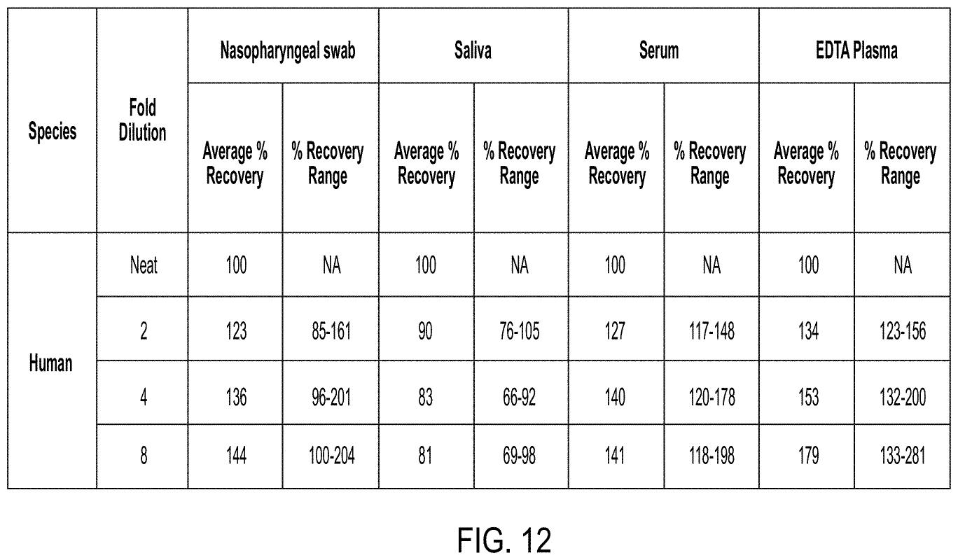

The dilution linearity and spike recovery of the assay were also tested to determine whether the assay is affected by component(s) that may be present in the biological sample matrix (also known as the “sample matrix effect”). For dilution linearity, the normal human serum, EDTA plasma, saliva, and COVID-19 negative human nasopharyngeal swab samples were spiked with calibrator and tested at different dilutions. Percent recovery at each dilution level was normalized to the dilution-adjusted, neat concentration, and shown in FIG. 12.

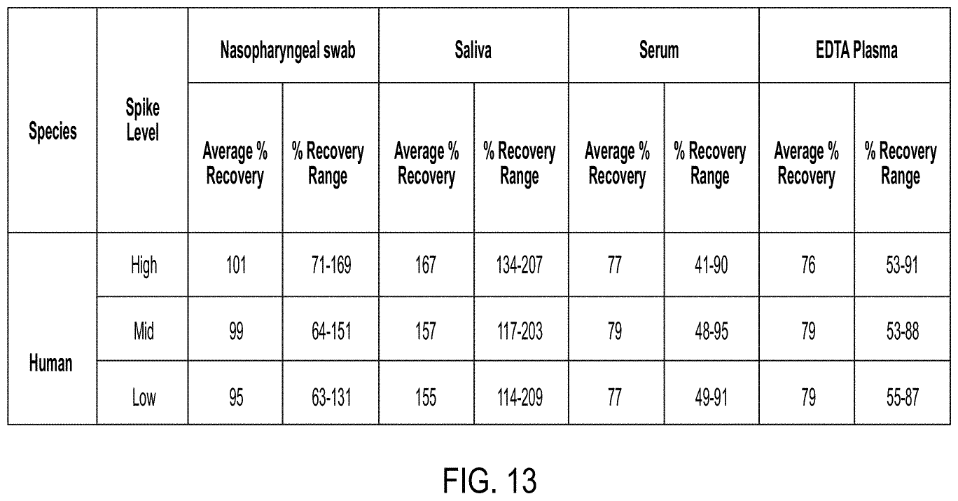

For spike recovery, normal human serum, EDTA plasma, saliva, and COVID-19 negative human nasopharyngeal swab samples were spiked with calibrator at three levels. Spiked samples were tested neat. Percent recovery is shown in FIG. 13. Based on the results in FIGS. 12 and 13, samples may be diluted to reduce sample matrix effects.

### Example 8. SARS-CoV-2 Nucleic Acid Detection Assay-OLA

The oligonucleotide ligation assay (OLA) described in Example 6 was also used to detect SARS-CoV-2 nucleic acid. Targeting and detection probes were designed for the SARS-CoV-2 N1, N2, and N3 regions (described in Lu et al., Emerg Infect Dis 26(8):1654-1665 (2020)) and the human RPP30 gene as control. The targeting probes have unique 5′ oligonucleotide tag sequences that are complementary to binding reagents on specific binding domains on a multi-well plate. The detection probes have a 5′ phosphate group for ligation and a 3′ biotin. OLA was used to ligate targeting and detection probes that aligned perfectly on the SARS-CoV-2 target nucleic acid. The ligated probes were then hybridized to the multi-well plate and detected by adding streptavidin-labeled SULFO-TAG™ and reading the signal with a plate reader. The plates were 10-spot, 96-well plates with the spot layout as shown in FIG. 39B. The binding reagents were immobilized in the spots as follows: Spot 4: binding reagent for N1; Spot 5: binding reagent for N2, Spot 9: binding reagent for N3; Spot 10: binding reagent for RPP30. The remaining spots were blank.

Probes were designed against both the viral RNA strand and the cDNA strand that is synthesized upon reverse transcription. The assay was first performed with a synthetic DNA template that included the target region (SARS-CoV-2 N1, N2, N3, and human RPP30). For viral RNA, one-step RT-PCR was performed to reverse transcribe and amplify the region surrounding each target. The OLA conditions are provided in Table 5.

TABLE 5
OLA Cycling Conditions

| | | | | |
|---|---|---|---|---|
| 95° | C. | 2 | min | |
| 95° | C. | 30 | sec | ×30 cycles |
| 65° | C. | 2 | min | |

| | | |
|---|---|---|
| 4° | C. | Hold |

Two sets of targeting and detection probes for each site were tested, designated as “OLA1” or “OLA2.” The probes sequences are shown in Tables 6 and 7.

TABLE 6
Probes

| Probe Name | Probe sequence |
|---|---|
| N1 Targeting probe OLA1 | GTGGCAACAGAAATCAGGTGGTGA cgaaatgcac cccgcattac |
| | (SEQ ID NO: 22) |
| N1 Detection probe OLA1 | /5phos/gtttggtgga ccctcagatt c/3bio/ |
| | (SEQ ID NO: 23) |
| N1 Targeting probe OLA2 | GTGGCAACAGAAATCAGGTGGTGA gaatctgagggtccaccaaac |
| | (SEQ ID NO: 24) |
| N1 Targeting probe OLA2 | TAGATGCCGCTGCAGGTATGGAAA gaatctgagggtccaccaaac |
| | (SEQ ID NO: 25) |
| N1 Detection probe OLA2 | /5phos/gtaatgcggggtgcatttcg/3bio/ |
| | (SEQ ID NO: 26) |
| N2 Targeting probe OLA1 | TAGATGCCGCTGCAGGTATGGAAA gc aaattgcaca atttgcccc |
| | (SEQ ID NO: 27) |
| N2 Detection probe OLA1 | /5Phos/c agcgcttcag cgttctt/3bio/ |
| | (SEQ ID NO: 28) |
| N2 Targeting probe OLA2 | TAGATGCCGCTGCAGGTATGGAAA gaacgctgaagcgctgg |
| | (SEQ ID NO: 29) |
| N2 Detection probe OLA2 | /5phos/gggcaaattgtgcaatttgcg/3bio/ |
| | (SEQ ID NO: 30) |
| N3 Targeting probe OLA1 | AGAGGACTGCTAAAGGTTTGTAGG ccaa aagaycacat tggcacc |
| | (SEQ ID NO: 31) |
| N3 Detection probe OLA1 | /5phos/cgc aatcctgcta acaatgctg/3bio/ |
| | (SEQ ID NO: 32) |
| N3 Targeting probe OLA2 | AGAGGACT GCT AAAGGTTT GT AGG gcattgttagcaggattgcgg |
| | (SEQ ID NO: 33) |
| N3 Detection probe OLA2 | /5phos/gtgccaatgtgrtcttttggtg/3bio/ |
| | (SEQ ID NO: 34) |
| RPP30 Targeting probe OLA1 | CGTACCATTGAATCTGGAGACCTT gagcgggtt ctgacctga |
| | (SEQ ID NO: 35) |
| RPP30 Detection probe OLA1 | /5phos/aggctctgcgc ggact/3bio/ |
| | (SEQ ID NO: 36) |
| RPP30 Targeting probe OLA2 | CGTACCATTGAATCTGGAGACCTT caagtccgcgcagagcc |
| | (SEQ ID NO: 37) |
| RPP30 Detection probe OLA2 | /5phos/ttcaggtcagaacccgct/3bio/ |
| | (SEQ ID NO: 38) |

TABLE 7
Blocking Oligonucleotides

| Blocking | Blocking |
|---|---|
| Oligonucleotide Name | Oligonucleotide Sequence |
| N1 Targeting OLA1 BO | cgaaatgcac cccgcattac (SEQ ID NO: 39) |
| N1 Detection OLA1 BO | gtttggtgga ccctcagatt c (SEQ ID NO: 40) |
| N1 Targeting OLA2 BO | gaatctgagggtccaccaaac (SEQ ID NO: 41) |
| N1 Detection OLA2 BO | gtaatgcggggtgcatttcg (SEQ ID NO: 42) |
| N2 Targeting OLA1 BO | gc aaattgcaca atttgcccc (SEQ ID NO: 43) |
| N2 Detection OLA1 BO | c agcgcttcag cgttctt (SEQ ID NO: 44) |
| N2 Targeting OLA2 BO | gaacgctgaagcgctgg (SEQ ID NO: 45) |
| N2 Detection OLA2 BO | gggcaaattgtgcaatttgcg (SEQ ID NO: 46) |
| N3 Targeting OLA1 BO | ccaa aagaycacat tggcacc (SEQ ID NO: 47) |
| N3 Detection OLA1 BO | cgc aatcctgcta acaatgctg (SEQ ID NO: 48) |
| N3 Targeting OLA2 BO | gcattgttagcaggattgcgg (SEQ ID NO: 49) |
| N3 Detection OLA2 BO | gtgccaatgtgrtcttttggtg (SEQ ID NO: 50) |
| RPP30 Targeting OLA1 BO | gagcgggtt ctgacctga (SEQ ID NO: 51) |
| RPP30 Detection OLA1 BO | aggctctgcgc ggact (SEQ ID NO: 52) |
| RPP30 Targeting OLA2 BO | caagtccgcgcagagcc (SEQ ID NO: 53) |
| RPP30 Detection OLA2 BO | ttcaggtcagaacccgct (SEQ ID NO: 54) |

Primers for amplification of the SARS-CoV-2 N1, N2, N3 regions and human RPP30 are described in Lu et al., Emerg Infect Dis 26(8):1654-1665 (2020) and reproduced in Table 8.

TABLE 8
Amplification Primers

| Primer Name | Primer Sequence |
|---|---|
| N1 Forward | GACCCCAAAATCAGCGAAAT |
| | (SEQ ID NO: 67) |
| N1 Reverse | TCTGGTTACTGCCAGTTGAATCTG |
| | (SEQ ID NO: 68) |
| N2 Forward | TTACAAACATTGGCCGCAAA |
| | (SEQ ID NO: 69) |
| N2 Reverse | GCGCGACATTCCGAAGAA |
| | (SEQ ID NO: 70) |
| N3 Forward | GGGAGCCTTGAATACACCAAAA |
| | (SEQ ID NO: 71) |
| N3 Reverse | TGTAGCACGATTGCAGCATTG |
| | (SEQ ID NO: 72) |
| RPP30 Forward | AGATTTGGACCTGCGAGCG |
| | (SEQ ID NO: 73) |
| RPP30 Reverse | GAGCGGCTGTCTCCACAAGT |
| | (SEQ ID NO: 74) |

The OLA assays were performed on RNA extracted from swab samples of SARS-CoV-2 positive (n=15) or negative (n=6) patients. Detectable signals for all of the same targets were observed as from the results of a qRT-PCR assay. The negative control samples did not have appreciable signal, indicating good specificity to SARS-CoV-2. N1 and N3 showed the best results. RPP30 signals were high in all samples (SARS-CoV-2 positive and negative), which was expected and confirmed consistency in RNA extraction.

### Example 9. SARS-CoV-2 Nucleic Acid Detection Assay-Internal Detection Probe

A SARS-CoV-2 nucleic acid detection assay that amplifies regions of interest (N1, N2, and N3) and contacts the amplified regions with a single internal detection probe was developed. The forward primer was tagged with a 5′ oligonucleotide tag. The internal detection probes have a 3′ biotin. The SARS-CoV-2 N1, N2, and N3 regions and the human RPP30 gene (control) were amplified using multiplexed PCR, followed by hybridization of the PCR products with the internal detection probes to form hybridized. The hybridized products were then immobilized to the multi-well plate and detected by adding streptavidin-labeled SULFO-TAG™ and reading the signal with a plate reader. The plates were 10-spot, 96-well plates with the spot layout as shown in FIG. 39B. The binding reagents were immobilized in the spots as follows: Spot 8: binding reagent for N1; Spot 9: binding reagent for N2, Spot 10: binding reagent for N3; Spot 1: binding reagent for RPP30. The remaining spots were blank.

Sequences of the forward primers, reverse primers, and the internal detection probes are shown in Table 9.

TABLE 9
Primers and Internal Detection
Probe Sequences

| | Forward | Reverse | Internal |
|---|---|---|---|
| Assay | primer | Primer | Probe |
| N1 | AATCCGT | TCTGGTT | ACC CCG CAT TAC GTT |
| | CGACTAG | ACTGC | TGG TGG ACC/3Bio/ |
| | CCTGAGA | CAGTTGA | (SEQ ID NO: 57) |
| | AT | ATCTG | |
| | TGACCCC | (SEQ ID | |
| | AAAATCA | NO: 56) | |
| | GCGAAAT | | |
| | (SEQ ID | | |
| | NO: 55) | | |
| N2 | AGAGGAC | GCGCGAC | ACA ATT TGC CCC CAG |
| | TGCTAAA | ATTCC | CGC TTC AG/3Bio/ |
| | GGTTTGT | GAAGAA | (SEQ ID NO: 60) |
| | AGG | (SEQ ID | |
| | TTACAAA | NO: 59) | |
| | CATTGGC | | |
| | CGCAAA | | |
| | (SEQ ID | | |
| | NO: 58) | | |
| N3 | CGTACCA | TGTAGCA | AYC ACA TTG GCA CCC |
| | TTGAATC | CGATT | GCA ATC CTG/3Bio/ |
| | TGGAGAC | GCAGCAT | (SEQ ID NO: 63) |
| | CTT | TG | |
| | GGGAGCC | (SEQ ID | |
| | TTGAATA | NO: 62) | |
| | CACCAAA | | |
| | A | | |
| | (SEQ ID | | |
| | NO: 61) | | |
| RPP30 | ACTGGTA | GAGCGGC | TTC TGA CCT GAA GGC |
| | ACCCAGA | TGTCT | TCT GCG CG/3Bio/ |
| | CATGATC | CCACAAG | (SEQ ID NO: 66) |
| | G | T | |
| | GTAGATT | (SEQ ID | |
| | TGGACCT | NO: 65) | |
| | GCGAGCG | | |
| | (SEQ ID | | |
| | NO: 64) | | |

### Example 10. SARS-CoV-2 Strain Typing and SNP Detection

The oligonucleotide ligation assay (OLA) as described in Examples 9, 13, and 23 is performed to detect mutations of the SARS-CoV-2 S protein. SARS-CoV-2 strains and the associated SNPs are shown in Table 1A and include genome locations 21765-21770, 23063, 23604, 22132, 22206, 22917, 23012, 23664, 22813, 22812, 22227, 28932, 29645, 1059, 25563, 21991-21993, 23271, 23709, 24506, 24914, 241, 3037, 14408, 26144, 29095, 22865, 22320, 21618, 23604, 24775, 22995, 24224, 25088, 23593, 24138, 21846, 22578, and 23525. Two sets of targeting and detection probes (“OLA1” or “OLA2”) for each genome location were designed. Further, two sets of targeting (“US”) probes for each genome location, one for the reference strain (“WT”) and one for the variant, were designed. The same detection (“DS”) probe for each genome location was designed for both the reference strain and the variant. The sequences for the targeting and detection probes are shown in Table 10. The sequences of synthetic templates containing the SARS-CoV-2 genome regions of interest are shown in Table 11. The sequences for blocking oligonucleotides are shown in Table 12. The primers for amplifying the target regions are shown in Table 13.

The OLA as described above is also performed to detect single polynucleotide polymorphisms (SNPs) at SARS-CoV-2 genome locations 8782, 28144, 23403, and 11083. As described herein, SNPs at genome locations 8782 and 28144 differentiate the L and S strains of the SARS-CoV-2. The A>G SNP at genome location 23403 encodes the D614G mutation in the SARS-CoV-2 S protein. The G>T SNP at genome location 11083 is associated with asymptomatic infection by SARS-CoV-2. The sequences for the targeting and detection probes are shown in Table 14. The sequences of synthetic templates containing the SARS-CoV-2 genome regions of interest are shown in Table 15. The sequences for blocking oligonucleotides are shown in Table 16.

TABLE 10
Targeting and Detection Probes-SARS-CoV-2 Protein Mutations

| | | Protein / |
|---|---|---|
| Probe Name | Probe sequence | Amino Acid |
| 21765-21770 WT US | ACTCCCTGTGGGTGAGCTTAATGG gttac ttggttccat gctatacatg | S protein |
| Probe OLA1 | (SEQ ID NO: 75) | Δ69-70 |
| 21765-21770 Del US | ACTGGTAACCCAGACATGATCGGT ccaatgttac ttggttccat gcta | |
| Probe OLA1 | (SEQ ID NO: 76) | |
| 21765-21770 DS | /5Phos/tctctgggac caatggtact aag/3bio/ | |
| Probe OLA1 | (SEQ ID NO: 77) | |
| 21765-21770 WT US | ACTCCCTGTGGGTGAGCTTAATGG ccattg gtcccagagacatgta | |
| Probe OLA2 | (SEQ ID NO: 78) | |
| 21765-21770 Del US | ACTGGTAACCCAGACATGATCGGT cttagtaccattg gtcccagaga | |
| Probe OLA2 | (SEQ ID NO: 79) | |
| 21765-21770 DS | /5Phos/tagcatggaaccaagtaacattgg/3Bio/ | |
| Probe OLA2 (ver1) | (SEQ ID NO: 80) | |
| 21765-21770 DS | /5Phos/taRcatggaaccaagtaacattgg/3Bio/ | |
| Probe OLA2 (ver2) | (SEQ ID NO: 655) | |
| 23063A US | CATTTGGTCATTGGTTCAAGACGA a atcatatggt ttccaaccca cta | S protein |
| Probe OLA1 | (SEQ ID NO: 81) | N501Y |
| 23063T US | CTGGTCCGTTGTGGTCCTTCTAAC a atcatatggt ttccaaccca ctt | |
| Probe OLA1 | (SEQ ID NO: 82) | |
| 23063 DS | /5Phos/atggtgt tggttaccaa ccatac/3Bio/ | |
| Probe OLA1 | (SEQ ID NO: 83) | |
| 23063A US | CATTTGGTCATTGGTTCAAGACGA tatggttggtaaccaacaccatt | |
| Probe OLA2 | (SEQ ID NO: 84) | |
| 23063T US | CTGGTCCGTTGTGGTCCTTCTAAC tatggttggtaaccaacaccata | |
| Probe OLA2 | (SEQ ID NO: 85) | |
| 23063 DS | /5Phos/agtgggttggaaaccatatgat tg/3Bio/ | |
| Probe OLA2 | (SEQ ID NO: 86) | |
| 23604C US | GTGGCAACAGAAATCAGGTGGTGA ctag ttatcagact cagactaatt ctcc | S protein |
| Probe OLA1 | (SEQ ID NO: 87) | P681H |
| 23604A US | AGAGGACTGCTAAAGGTTTGTAGG ctag ttatcagact cagactaatt ctca | |
| Probe OLA1 | (SEQ ID NO: 88) | |
| 23604 DS | /5Phos/tcggcg ggcacgtag/3Bio/ | |
| Probe OLA1 | (SEQ ID NO: 89) | |
| 23604C US | GTGGCAACAGAAATCAGGTGGTGA ctacgtgcccgccgag | |
| Probe OLA2 | (SEQ ID NO: 90) | |
| 23604A US | AGAGGACTGCTAAAGGTTTGTAGG ctacgtgcccgccgat | |
| Probe OLA2 | (SEQ ID NO: 91) | |
| 23604 DS | /5Phos/gagaattagtctgagtctgataa ctagc/3Bio/ | |
| Probe OLA2 (ver1) | (SEQ ID NO: 92) | |
| 23604 DS | /5Phos/gagaattagtVKgagtctgataa ctagc/3Bio/ | |
| Probe OLA2 (ver2) | (SEQ ID NO: 656) | |
| 22132G US | CTGGTCCGTTGTGGTCCTTCTAAC ggaaaaca gggtaatttc aaaaatctta gg | S protein |
| Probe OLA1 | (SEQ ID NO: 93) | R190S |
| 22132T US | CATTTGGTCATTGGTTCAAGACGA ggaaaaca gggtaatttc aaaaatctta gt | |
| Probe OLA1 | (SEQ ID NO: 94) | |
| 22132 DS | /5Phos/gaatttgt gtttaagaat attgatggtt attttaaaat at/3Bio/ | |
| Probe OLA1 | (SEQ ID NO: 95) | |
| 22132G US | CTGGTCCGTTGTGGTCCTTCTAAC taaaataaccatcaatattcttaaacacaaattcc | |
| Probe OLA2 | (SEQ ID NO: 96) | |
| 22132T US | CATTTGGTCATTGGTTCAAGACGA taaaataaccatcaatattcttaaacacaaattca | |
| Probe OLA2 | (SEQ ID NO: 97) | |
| 22132 DS | /5Phos/ctaagatttttgaaattaccctgttttcct/3Bio/ | |
| Probe OLA2 | (SEQ ID NO: 98) | |
| 22206A US | AATCCGTCGACTAGCCTGAGAATT acgccta ttaatttagt gcgtga | S protein |
| Probe OLA1 | (SEQ ID NO: 99) | D215G |
| 22206G US | CTAATAGCTCCTGTGCCCTCGTAT acgccta ttaatttagt gcgtgg | |
| Probe OLA1 | (SEQ ID NO: 100) | |
| 22206 DS | /5Phos/tctc cctcagggtt tttcgg/3Bio/ | |
| Probe OLA1 | (SEQ ID NO: 101) | |
| 22206A US | AATCCGTCGACTAGCCTGAGAATT ccgaaaaaccctgagggagat | |
| Probe OLA2 | (SEQ ID NO: 102) | |
| 22206G US | CTAATAGCTCCTGTGCCCTCGTAT ccgaaaaaccctgagggagac | |
| Probe OLA2 | (SEQ ID NO: 103) | |
| 22206 DS | /5Phos/cacgc actaaattaataggcgtg/3Bio/ | |
| Probe OLA2 | (SEQ ID NO: 104) | |
| 22917T US | AGAGGACTGCTAAAGGTTTGTAGG attc taaggttggt ggtaattata attacct | S protein |
| Probe OLA1 | (SEQ ID NO: 105) | L452R |
| 22917GUS | GTGGCAACAGAAATCAGGTGGTGA attc taaggttggt ggtaattata attaccg | |
| Probe OLA1 | (SEQ ID NO: 106) | |
| 22917 DS | /5Phos/gta tagattgttt aggaagtcta atctcaaac/3Bio/ | |
| Probe OLA1 | (SEQ ID NOT07) | |
| 22917T US | AGAGGACTGCTAAAGGTTTGTAGG gtttgagattagacttcctaaacaatcta taca | |
| Probe OLA2 (ver1) | (SEQ ID NO: 108) | |
| 22917T US | AGAGGACTGCTAAAGGTTTGTAGG gtttgagattagacttcctaaacaatcta tac+a | |
| Probe OLA2 (ver2) | (SEQ ID NO: 657) | |
| 22917GUS | GTGGCAACAGAAATCAGGTGGTGA gtttgagattagacttcctaaacaatcta tacc | |
| Probe OLA2 (ver1) | (SEQ ID NO: 109) | |
| 22917G US | GTGGCAACAGAAATCAGGTGGTGA gtttgagattagacttcctaaacaatcta tac+c | |
| Probe OLA2 (ver2) | (SEQ ID NO: 658) | |
| 22917 DS | /5Phos/ggtaattataattaccaccaaccttagaat/3Bio/ | |
| Probe OLA2 | (SEQ ID NO: 110) | |
| 23012G US | ACTGGTAACCCAGACATGATCGGT agcacacctt gtaatggtgt tg | S protein |
| Probe OLA1 | (SEQ ID NO: 111) | E484K |
| 23012A US | ACTCCCTGTGGGTGAGCTTAATGG agcacacctt gtaatggtgt ta | |
| Probe OLA1 | (SEQ ID NO: 112) | |
| 23012 DS | /5Phos/aaggtttt aattgttact ttcctttaca atc/3Bio/ | |
| Probe OLA1 | (SEQ ID NO: 113) | |
| 23012G US | ACTGGTAACCCAGACATGATCGGT tgat tgtaaaggaaagtaacaattaaaacctto | |
| Probe OLA2 | (SEQ ID NO: 114) | |
| 23012A US | ACTCCCTGTGGGTGAGCTTAATGG tgat tgtaaaggaaagtaacaattaaaaccttt | |
| Probe OLA2 | (SEQ ID NO: 115) | |
| 23012 DS | /5Phos/aacaccattacaaggtgtgcta/3Bio/ | |
| Probe OLA2 (ver1) | (SEQ ID NO: 116) | |
| 23012 DS | /5Phos/aacaccattacaaggtKtgYta/3Bio/ | |
| Probe OLA2 (ver2) | (SEQ ID NO: 659) | |
| 23664C US | TAGATGCCGCTGCAGGTATGGAAA gcctacact atgtcacttg gtgc | S protein |
| Probe OLA1 | (SEQ ID NO: 117) | A701V |
| 23664T US | CGTACCATTGAATCTGGAGACCTT gcctacact atgtcacttg gtgt | |
| Probe OLA1 | (SEQ ID NO: 118) | |
| 23664 DS | /5Phos/agaaaa ttcagttgct tactctaata actct/3Bio/ | |
| Probe OLA1 | (SEQ ID NO: 119) | |
| 23664C US | TAGATGCCGCTGCAGGTATGGAAA agagttattagagtaagcaactgaattttctg | |
| Probe OLA2 | (SEQ ID NO: 120) | |
| 23664T US | CGTACCATTGAATCTGGAGACCTT agagttattagagtaagcaactgaattttcta | |
| Probe OLA2 | (SEQ ID NO: 121) | |
| 23664 DS | /5Phos/caccaagtgacatagtgtaggc/3Bio/ | |
| Probe OLA2 | (SEQ ID NO: 122) | |
| 22813G US | TAGATGCCGCTGCAGGTATGGAAA gctccagg gcaaactgga aMg | S protein |
| Probe OLA1 | (SEQ ID NO: 123) | K417N |
| 22813T US | CGTACCATTGAATCTGGAGACCTT gctccagg gcaaactgga aMt | |
| Probe OLA1 | (SEQ ID NO: 124) | |
| 22813 DS | /5Phos/attgctg attataatta taaattacca gatgatttta c/3Bio/ | |
| Probe OLA1 | (SEQ ID NO: 125) | |
| 22813G US | TAGATGCCGCTGCAGGTATGGAAA gtaaaatcatctggtaatttataa | |
| Probe OLA2 | ttataatcagcaatc (SEQ ID NO: 126) | |
| 22813T US | CGTACCATTGAATCTGGAGACCTT gtaaaatcatctggtaatttataa | |
| Probe OLA2 | ttataatcagcaata (SEQ ID NO: 127) | |
| 22813 DS | /5Phos/Kttccagtttgc cctggagc/3Bio/ | |
| Probe OLA2 | (SEQ ID NO: 128) | |
| 22812A US | CTGGTCCGTTGTGGTCCTTCTAAC gctccagg gcaaactgga aa | S protein |
| Probe OLA1 | (SEQ ID NO: 129) | K417T |
| 22812C US | CATTTGGTCATTGGTTCAAGACGA gctccagg gcaaactgga ac | |
| Probe OLA1 | (SEQ ID NO: 130) | |
| 22812 DS | /5Phos/Kattgctg attataatta taaattacca gatgatttta c/3Bio/ | |
| Probe OLA1 | (SEQ ID NO: 131) | |
| 22812A US | CTGGTCCGTTGTGGTCCTTCTAAC atcatctggtaatttataattataatcagcaatMt | |
| Probe OLA2 | (SEQ ID NO: 132) | |
| 22812C US | CATTTGGTCATTGGTTCAAGACGA atcatctggtaatttataattataatcagcaatMg | |
| Probe OLA2 | (SEQ ID NO: 133) | |
| 22812 DS | /5Phos/ttccagtttgc cctggagc/3Bio/ | |
| Probe OLA2 | (SEQ ID NO: 134) | |
| 22227C US | tc cctcagggtt tttcggc | S protein |
| Probe OLA1 | (SEQ ID NO: 227) | A222V |
| 22227T US | tc cctcagggtt tttcggt | |
| Probe OLA1 | (SEQ ID NO: 228) | |
| 22227 DS | /5Phos/ttt agaaccattg gtagatttgc ca/3Bio/ | |
| Probe OLA1 | (SEQ ID NO: 229) | |
| 22227C US | ggcaaatctaccaatggttctaaag | |
| Probe OLA2 | (SEQ ID NO: 230) | |
| 22227T US | ggcaaatctaccaatggttctaaaa | |
| Probe OLA2 | (SEQ ID NO: 231) | |
| 22227 DS | /5Phos/ccgaaaaaccctgagggaga/3Bio/ | |
| Probe OLA2 | (SEQ ID NO: 232) | |
| 28932C US | cggtga tgctgctctt gc | N protein |
| Probe OLA1 | (SEQ ID NO: 233) | A220V |
| 28932T US | cggtga tgctgctctt gt | |
| Probe OLA1 | (SEQ ID NO: 234) | |
| 28932 DS | /5Phos/tttgctgc tgcttgacag attg/3Bio/ | |
| Probe OLA1 | (SEQ ID NO: 235) | |
| 28932C US | caatctgtcaagcagcagcaaag | |
| Probe OLA2 | (SEQ ID NO: 236) | |
| 28932T US | caatctgtcaagcagcagcaaaa | |
| Probe OLA2 | (SEQ ID NO: 237) | |
| 28932 DS | /5Phos/caagagcagcatcaccgc/3Bio/ | |
| Probe OLA2 | (SEQ ID NO: 238) | |
| 29645G US | tg aattctcgta actacatagc acaag | Orf10 |
| Probe OLA1 | (SEQ ID NO: 239) | V30L |
| 29645T US | tg aattctcgta actacatagc acaat | |
| Probe OLA1 | (SEQ ID NO: 240) | |
| 29645 DS | /5Phos/tagat gtagttaact ttaatctcac atagc/3Bio/ | |
| Probe OLA1 | (SEQ ID NO: 241) | |
| 29645G US | gctatgtgagattaaagttaactacatctac | |
| Probe OLA2 | (SEQ ID NO: 242) | |
| 29645T US | gctatgtgagattaaagttaactacatctaa | |
| Probe OLA2 | (SEQ ID NO: 243) | |
| 29645 DS | /5Phos/ttgtgctatgtagttacgagaattca/3Bio | |
| Probe OLA2 | (SEQ ID NO: 244) | |
| 1059C US | tt gaaattaaat tggcaaagaa atttgacac | Orf1a |
| Probe OLA1 | (SEQ ID NO: 245) | T265I |
| 1059T US | tt gaaattaaat tggcaaagaa atttgacat | |
| Probe OLA1 | (SEQ ID NO: 246) | |
| 1059 DS | /5Phos/c ttcaatgggg aatgtccaaa ttttg/3Bio/ | |
| Probe OLA1 | (SEQ ID NO: 247) | |
| 1059C US | aaaatttggacattccccattgaagg | |
| Probe OLA2 | (SEQ ID NO: 248) | |
| 1059T US | aaaatttggacattccccattgaaga | |
| Probe OLA2 | (SEQ ID NO: 249) | |
| 1059 DS | /5Phos/tgtcaaatttctttgccaatttaatttcaaa/3Bio/ | |
| Probe OLA2 | (SEQ ID NO: 250) | |
| 25563G US | g ttgcacttct tgctgttttt cag | Orf3a |
| Probe OLA1 | (SEQ ID NO: 251) | Q57H |
| 25563T US | g ttgcacttct tgctgttttt cat | |
| Probe OLA1 | (SEQ ID NO: 252) | |
| 25563 DS | /5Phos/agcgctt ccaaaatcat aaccct/3Bio/ | |
| Probe OLA1 | (SEQ ID NO: 253) | |
| 25563G US | gggttatgattttggaagcgctc | |
| Probe OLA2 | (SEQ ID NO: 254) | |
| 25563T US | gggttatgattttggaagcgcta | |
| Probe OLA2 | (SEQ ID NO: 255) | |
| 25563 DS | /5Phos/tgaaaaacagcaagaagtgcaac/3Bio/ | |
| Probe OLA2 | (SEQ ID NO: 256) | |
| 21991-21993 WT US | ttttgt aatgatccat ttttgggtgt tta | S protein |
| Probe OLA1 | (SEQ ID NO: 257) | Y144 del |
| 21991-21993 del US | aattttgt aatgatccat ttttgggtgt | |
| Probe OLA1 | (SEQ ID NO: 258) | |
| 21991-21993 DS | /5Phos/ttaccac aaaaacaaca aaagttggat/3Bio/ | |
| Probe OLA1 | (SEQ ID NO: 259) | |
| 21991-21993 WT US | ccaacttttgttgtttttgtggtaataa | |
| Probe OLA2 | (SEQ ID NO: 260) | |
| 21991-21993 del US | atccaacttttgttgtttttgtggtaa | |
| Probe OLA2 | (SEQ ID NO: 261) | |
| 21991-21993 DS | /5Phos/acacccaaaaatggatcattacaaaatt/3Bio/ | |
| Probe OLA2 | (SEQ ID NO: 262) | |
| 23271C US | cca acaatttggc agagacattg c | S protein |
| Probe OLA1 | (SEQ ID NO: 263) | A570D |
| 23271A US | cca acaatttggc agagacattg a | |
| Probe OLA1 | (SEQ ID NO: 264) | |
| 23271 DS | /5Phos/tgacactac tgatgctgtc cg/3Bio/ | |
| Probe OLA1 | (SEQ ID NO: 265) | |
| 23271C US | cggacagcatca gtagtgtcag | |
| Probe OLA2 | (SEQ ID NO: 266) | |
| 23271A US | cggacagcatca gtagtgtcat | |
| Probe OLA2 | (SEQ ID NO: 267) | |
| 23271 DS | /5Phos/caatgtctctgccaaattgttgga/3Bio/ | |
| Probe OLA2 | (SEQ ID NO: 268) | |
| 23709C US | tctaata actctattgc catacccac | S protein |
| Probe OLA1 | (SEQ ID NO: 269) | T716I |
| 23709T US | tctaata actctattgc catacccat | |
| Probe OLA1 | (SEQ ID NO: 270) | |
| 23709 DS | /5Phos/a aattttacta ttagtgttac cacagaaatt c/3Bio/ | |
| Probe OLA1 | (SEQ ID NO: 271) | |
| 23709C US | gaatttctgtggtaacactaatagtaaaatttg | |
| Probe OLA2 | (SEQ ID NO: 272) | |
| 23709T US | gaatttctgtggtaacactaatagtaaaattta | |
| Probe OLA2 | (SEQ ID NO: 273) | |
| 23709 DS | /5Phos/tgggtatg gcaatagagttattagagtaa/3Bio/ | |
| Probe OLA2 | (SEQ ID NO: 274) | |
| 24506T US | gcaat ttcaagtgtt ttaaatgata tccttt | S protein |
| Probe OLA1 | (SEQ ID NO: 275) | S982A |
| 24506G US | gcaat ttcaagtgtt ttaaatgata tccttg | |
| Probe OLA1 | (SEQ ID NO: 276) | |
| 24506 DS | /5Phos/cacg tcttgacaaa gttgaggc/3Bio/ | |
| Probe OLA1 | (SEQ ID NO: 277) | |
| 24506T US | gcctcaactttgtcaagacgtga | |
| Probe OLA2 | (SEQ ID NO: 278) | |
| 24506G US | gcctcaactttgtcaagacgtgc | |
| Probe OLA2 | (SEQ ID NO: 279) | |
| 24506 DS | /5Phos/aaggatatcatttaaaacacttgaa attgca/3Bio/ | |
| Probe OLA2 | (SEQ ID NO: 280) | |
| 24914G US | gaattttt atgaaccaca aatcattact acag | S protein |
| Probe OLA1 | (SEQ ID NO: 281) | D1118H |
| 24914C US | gaattttt atgaaccaca aatcattact acac | |
| Probe OLA1 | (SEQ ID NO: 282) | |
| 24914 DS | /5Phos/acaaca catttgtgtc tggtaact/3Bio/ | |
| Probe OLA1 | (SEQ ID NO: 283) | |
| 24914G US | gttaccagacacaaatgtgttgtc | |
| Probe OLA2 | (SEQ ID NO: 284) | |
| 24914C US | gttaccagacacaaatgtgttgtg | |
| Probe OLA2 | (SEQ ID NO: 285) | |
| 24914 DS | /5Phos/tgtagtaatgatt tgtggttcataaaaattcc/3Bio/ | |
| Probe OLA2 | (SEQ ID NO: 286) | |
| 241C US | cga tcatcagcac atctaggtttc | 5′UTR |
| Probe OLA1 | (SEQ ID NO: 287) | |
| 241T US | cga tcatcagcac atctaggtttt | |
| Probe OLA1 | (SEQ ID NO: 288) | |
| 241 DS | /5Phos/gtccgggtg tgaccgaaag/3Bio/ | |
| Probe OLA1 | (SEQ ID NO: 289) | |
| 241C US | cggtcacacccggacg | |
| Probe OLA2 | (SEQ ID NO: 290) | |
| 241T US | cggtcacacccggaca | |
| Probe OLA2 | (SEQ ID NO: 291) | |
| 241 DS | /5Phos/aaacctagatgtgctgatgatcg/3Bio/ | |
| Probe OLA2 | (SEQ ID NO: 292) | |
| 3037C US | a ttggcttcac atatgtattg ttctttc | nsp3 |
| Probe OLA1 | (SEQ ID NO: 293) | F924F |
| 3037T US | a ttggcttcac atatgtattg ttctttt | |
| Probe OLA1 | (SEQ ID NO: 294) | |
| 3037 DS | /5Phos/tac cctccagatg aggatgaaga/3Bio/ | |
| Probe OLA1 | (SEQ ID NO: 295) | |
| 3037C US | cttcatcctcatctggagggtag | |
| Probe OLA2 | (SEQ ID NO: 296) | |
| 3037T US | cttcatcctcatctggagggtaa | |
| Probe OLA2 | (SEQ ID NO: 297) | |
| 3037 DS | /5Phos/aaagaacaatacatatgtgaagccaatt/3Bio/ | |
| Probe OLA2 | (SEQ ID NO: 298) | |
| 14408C US | ttttatt ctctacagtg ttcccacc | RdRp |
| Probe OLA1 | (SEQ ID NO: 299) | P323L |
| 14408T US | ttttatt ctctacagtg ttcccact | |
| Probe OLA1 | (SEQ ID NO: 300) | |
| 14408 DS | /5Phos/ta caagttttgg accactagtg aga/3Bio/ | |
| Probe OLA1 | (SEQ ID NO: 301) | |
| 14408C US | ctcactagtggtccaaaacttgtag | |
| Probe OLA2 | (SEQ ID NO: 302) | |
| 14408T US | ctcactagtggtccaaaacttgtaa | |
| Probe OLA2 | (SEQ ID NO: 303) | |
| 14408 DS | /5Phos/gtgggaacactgtagagaataaaact/3Bio/ | |
| Probe OLA2 | (SEQ ID NO: 304) | |
| 26144G US | gtccaaatt cacacaatcg acgg | Orf3a |
| Probe OLA1 | (SEQ ID NO: 305) | G251V |
| 26144T US | gtccaaatt cacacaatcg acgt | |
| Probe OLA1 | (SEQ ID NO: 306) | |
| 26144 DS | /5Phos/ttcatc cggagttgtt aatccag/3Bio/ | |
| Probe OLA1 | (SEQ ID NO: 307) | |
| 26144G US | ctggattaacaactccggatgaac | |
| Probe OLA2 | (SEQ ID NO: 308) | |
| 26144T US | ctggattaacaactccggatgaaa | |
| Probe OLA2 | (SEQ ID NO: 309) | |
| 26144 DS | /5Phos/cgtcgattgtgtgaatttggaca/3Bio/ | |
| Probe OLA2 | (SEQ ID NO: 310) | |
| 29095C US | a agcatacaat gtaacacaag ctttc | N protein |
| Probe OLA1 | (SEQ ID NO: 311) | F274F |
| 29095T US | a agcatacaat gtaacacaag ctttt | |
| Probe OLA1 | (SEQ ID NO: 312) | |
| 29095 DS | /5Phos/ggcag acgtggtcca gaa/3Bio/ | |
| Probe OLA1 | (SEQ ID NO: 313) | |
| 29095C US | ctggaccacgtctgccg | |
| Probe OLA2 | (SEQ ID NO: 314) | |
| 29095T US | ctggaccacgtctgcca | |
| Probe OLA2 | (SEQ ID NO: 315) | |
| 29095 DS | /5Phos/aaagcttgtgttacattgtatgcttta/3Bio/ | |
| Probe OLA2 | (SEQ ID NO: 316) | |
| 22865G US | ca gatgatttta caggctgcgt tatag | S protein |
| Probe OLA1 | (SEQ ID NO: 317) | A435S |
| 22865T US | ca gatgatttta caggctgcgt tatat | |
| Probe OLA1 | (SEQ ID NO: 318) | |
| 22865 DS | /5Phos/cttgg aattctaaca atcttgattc taagg/3Bio/ | |
| Probe OLA1 | (SEQ ID NO: 319) | |
| 22865G US | cttagaatcaagattgttagaattccaagc | |
| Probe OLA2 | (SEQ ID NO: 320) | |
| 22865T US | cttagaatcaagattgttagaattccaaga | |
| Probe OLA2 | (SEQ ID NO: 321) | |
| 22865 DS | /5Phos/tataacgcagcctgtaaaatcatct/3Bio/ | |
| Probe OLA2 | (SEQ ID NO: 322) | |
| 22320A US | CGTACCATTGAATCTGGAGACCTT acataga agttatttga ctcctggtg a | S protein |
| Probe OLA1 | (SEQ ID NO: 533) | D253G |
| 22320G US | TAGATGCCGCTGCAGGTATGGAAA acataga agttatttga ctcctggtg g | |
| Probe OLA1 | (SEQ ID NO: 534) | |
| 22320 DS | /5Phos/ttcttcttca ggttggacag ctg/3Bio/ | |
| Probe OLA1 | (SEQ ID NO: 535) | |
| 22320A US | CGTACCATTGAATCTGGAGACCTT ag ctgtccaacc tgaagaagaa t | |
| Probe OLA2 | (SEQ ID NO: 536) | |
| 22320G US | TAGATGCCGCTGCAGGTATGGAAA ag ctgtccaacc tgaagaagaa c | |
| Probe OLA2 | (SEQ ID NO: 537) | |
| 22320 DS | /5Phos/caccaggag tcaaataact tctatgtaaa/3Bio/ | |
| Probe OLA2 | (SEQ ID NO: 538) | |
| 23012C US | CATTTGGTCATTGGTTCAAGACGA agcacacctt gtaatggtgt to | S protein |
| Probe OLA1 | (SEQ ID NO: 549) | E484Q |
| 23012C US | CATTTGGTCATTGGTTCAAGACGA tgat tgtaaaggaaagtaacaattaaaaccttg | |
| Probe OLA2 | (SEQ ID NO: 550) | |
| 23012 DS | /5Phos/aacaccattacaaggtgtgcta/3Bio/ | |
| Probe OLA2 (ver1) | (SEQ ID NO: 116) | |
| 23012 DS | /5Phos/aacaccattacaaggtKtgYta/3Bio/ | |
| Probe OLA2 (ver2) | (SEQ ID NO: 659) | |
| 21618C US | CTAATAGCTCCTGTGCCCTCGTAT actagtctctagtcagtgtgttaatctta c | S protein |
| Probe OLA1 | (SEQ ID NO: 551) | T19R |
| 21618G US | AATCCGTCGACTAGCCTGAGAATT actagtctctagtcagtgtgttaatctta g | |
| Probe OLA1 | (SEQ ID NO: 552) | |
| 21618 DS | /5Phos/aa ccagaactca attaccccct/3Bio/ | |
| Probe OLA1 | (SEQ ID NO: 553) | |
| 21618C US | CTAATAGCTCCTGTGCCCTCGTAT agggggtaattgagttctggtt g | |
| Probe OLA2 (ver1) | (SEQ ID NO: 554) | |
| 21618C US | CTAATAGCTCCTGTGCCCTCGTAT agRgggtaat tgagttctgK tt g | |
| Probe OLA2 (ver2) | (SEQ ID NO: 660) | |
| 21618GUS | AATCCGTCGACTAGCCTGAGAATT agggggtaattgagttctggtt c | |
| Probe OLA2 (ver1) | (SEQ ID NO: 555) | |
| 21618GUS | AATCCGTCGACTAGCCTGAGAATT agRgggtaat tgagttctgK tt c | |
| Probe OLA2 (ver2) | (SEQ ID NO: 661) | |
| 21618 DS | /5Phos/taagatt aacacactga ctagagacta gt/3Bio/ | |
| Probe OLA2 (ver1) | (SEQ ID NO: 556) | |
| 21618 DS | /5Phos/taaRatt aacacactga Mtagagacta gt/3Bio/ | |
| Probe OLA2 (ver2) | (SEQ ID NO: 662) | |
| 23604GUS | CTGGTCCGTTGTGGTCCTTCTAAC ctag ttatcagact cagactaatt ctcg | S protein |
| Probe OLA1 | (SEQ ID NO: 557) | P681R |
| 23604GUS | CTGGTCCGTTGTGGTCCTTCTAAC ctacgtgcccgccgac | |
| Probe OLA2 | (SEQ ID NO: 558) | |
| 23604 DS | /5Phos/gagaattagtctgagtctgataa ctagc/3Bio/ | |
| Probe OLA2 (ver1) | (SEQ ID NO: 92) | |
| 23604 DS | /5Phos/gagaattagtVKgagtctgataa ctagc/3Bio/ | |
| Probe OLA2 (ver2) | (SEQ ID NO: 656) | |
| 24775A US | TAGATGCCGCTGCAGGTATGGAAA catgtgact tatgtccctg caca a | S protein |
| Probe OLA1 | (SEQ ID NO: 559) | Q1071H |
| 24775T US | CGTACCATTGAATCTGGAGACCTT catgtgact tatgtccctg caca t | |
| Probe OLA1 | (SEQ ID NO: 560) | |
| 24775 DS | /5Phos/gaaaa gaacttcaca actgctcctg /3Bio/ | |
| Probe OLA1 | (SEQ ID NO: 561) | |
| 24775A US | TAGATGCCGCTGCAGGTATGGAAA caggagcagt tgtgaagttc ttttc t | |
| Probe OLA2 | (SEQ ID NO: 562) | |
| 24775T US | CGTACCATTGAATCTGGAGACCTT caggagcagt tgtgaagttc ttttc a | |
| Probe OLA2 | (SEQ ID NO: 563) | |
| 24775 DS | /5Phos/tgtg cagggacata agtcacat/3Bio/ | |
| Probe OLA2 | (SEQ ID NO: 564) | |
| 22995C US | ACTGGTAACCCAGACATGATCGG gaaatcta tcaggccggt aRca c | S protein |
| Probe OLA1 | (SEQ ID NO: 565) | T478K |
| 22995A US | ACTCCCTGTGGGTGAGCTTAATGG gaaatcta tcaggccggt aRca a | |
| Probe OLA1 | (SEQ ID NO: 566) | |
| 22995 DS | /5Phos/acctt gtaatggtgt tVaaggtttt aa/3Bio/ | |
| Probe OLA1 | (SEQ ID NO: 567) | |
| 22995C US | ACTGGTAACCCAGACATGATCGG tt aaaaccttBa acaccattac aaggt g | |
| Probe OLA2 | (SEQ ID NO: 568) | |
| 22995A US | ACTCCCTGTGGGTGAGCTTAATGG tt aaaaccttBa acaccattac aaggt t | |
| Probe OLA2 | (SEQ ID NO: 569) | |
| 22995 DS | /5Phos/tgYt accggcctga tagattt/3Bio/ | |
| Probe OLA2 | (SEQ ID NO: 570) | |
| 24224T US | CATTTGGTCATTGGTTCAAGACGA acaatcac ttctggttgg acct | S protein |
| Probe OLA1 | (SEQ ID NO: 571) | F888L |
| 24224C US | CTGGTCCGTTGTGGTCCTTCTAAC acaatcac ttctggttgg accc | |
| Probe OLA1 | (SEQ ID NO: 572) | |
| 24224 DS | /5Phos/ttggtg caggtgctgc a/3Bio/ | |
| Probe OLA1 | (SEQ ID NO: 573) | |
| 24224T US | CATTTGGTCATTGGTTCAAGACGA gcagcacctgcaccaaa | |
| Probe OLA2 | (SEQ ID NO: 574) | |
| 24224C US | CTGGTCCGTTGTGGTCCTTCTAAC gcagcacctgcaccaag | |
| Probe OLA2 | (SEQ ID NO: 575) | |
| 24224 DS | /5Phos/ggtccaaccagaagtgattgtac/3Bio/ | |
| Probe OLA2 | (SEQ ID NO: 576) | |
| 24224T US | GTGGCAACAGAAATCAGGTGGTGA acaatcac ttctggttgg acct | |
| Probe OLA1.1 | (SEQ ID NO: 666) | |
| 24224C US | AGAGGACTGCTAAAGGTTTGTAGG acaatcac ttctggttgg accc | |
| Probe OLA1.1 | (SEQ ID NO: 667) | |
| 24224T US | GTGGCAACAGAAATCAGGTGGTGA gcagcacctgcaccaaa | |
| Probe OLA2.1 | (SEQ ID NO: 668) | |
| 24224C US | AGAGGACTGCTAAAGGTTTGTAGG gcagcacctgcaccaag | |
| Probe OLA2.1 | (SEQ ID NO: 669) | |
| 25088G US | CTAATAGCTCCTGTGCCCTCGTAT acatct ctggcattaa tgcttcag | S protein |
| Probe OLA1 | (SEQ ID NO: 577) | VH67F |
| 25088T US | AATCCGTCGACTAGCCTGAGAATT acatct ctggcattaa tgcttcat | |
| Probe OLA1 | (SEQ ID NO: 578) | |
| 25088 DS | /5Phos/tt gtaaacattc aaaaagaaat tgaccgc/3Bio/ | |
| Probe OLA1 | (SEQ ID NO: 579) | |
| 25088G US | CTAATAGCTCCTGTGCCCTCGTAT gcggtcaatttctttttgaatgtttacaac | |
| Probe OLA2 | (SEQ ID NO: 580) | |
| 25088T US | AATCCGTCGACTAGCCTGAGAATT gcggtcaatttctttttgaatgtttacaaa | |
| Probe OLA2 | (SEQ ID NO: 581) | |
| 25088 DS | /5Phos/tgaagca ttaatgccagagatgtc/3Bio/ | |
| Probe OLA2 | (SEQ ID NO: 582) | |
| 23593G US | GTGGCAACAGAAATCAGGTGGTGA ta tatgcgctag ttatcagact cMg | S protein |
| Probe OLA1 | (SEQ ID NO: 583) | Q677H |
| 23593T US | AGAGGACTGCTAAAGGTTTGTAGG ta tatgcgctag ttatcagact cMt | |
| Probe OLA1 | (SEQ ID NO: 584) | |
| 23593C US | AGAGGACTGCTAAAGGTTTGTAGG ta tatgcgctag ttatcagact cMc | |
| Probe OLA1 | (SEQ ID NO: 585) | |
| 23593 DS | /5Phos/actaatt ctcVtcggcg gg/3Bio/ | |
| Probe OLA1 | (SEQ ID NO: 586) | |
| 23593G US | GTGGCAACAGAAATCAGGTGGTGA cccgccgaBgagaattagtc | |
| Probe OLA2 | (SEQ ID NO: 587) | |
| 23593T US | AGAGGACTGCTAAAGGTTTGTAGG cccgccgaBgagaattagta | |
| Probe OLA2 | (SEQ ID NO: 588) | |
| 23593C US | AGAGGACTGCTAAAGGTTTGTAGG cccgccgaBgagaattagtg | |
| Probe OLA2 | (SEQ ID NO: 589) | |
| 23593 DS | /5Phos/Kgagtctgataa ctagcgcatata/3Bio/ | |
| Probe OLA2 | (SEQ ID NO: 590) | |
| 21991-21993 WT US | TAGATGCCGCTGCAGGTATGGAAA ttttgt aatgatccat ttttgggtgt tta | S protein |
| Probe OLA1 | (SEQ ID NO: 591) | Y144 del |
| 21991-21993 del US | CGTACCATTGAATCTGGAGACCTT aattttgt aatgatccat ttttgggtgt | |
| Probe OLA1 | (SEQ ID NO: 592) | |
| 21991-21993 DS | /5Phos/ttaccac aaaaacaaca aaagttggat/3Bio/ | |
| Probe OLA1 | (SEQ ID NO: 593) | |
| 21991-21993 WT US | TAGATGCCGCTGCAGGTATGGAAA ccaacttttgttgtttttgtggtaataa | |
| Probe OLA1 | (SEQ ID NO: 594) | |
| 21991-21993 del US | CGTACCATTGAATCTGGAGACCTT atccaacttttgttgtttttgtggtaa | |
| Probe OLA1 | (SEQ ID NO: 595) | |
| 21991-21993 DS | /5Phos/acacccaaaaatggatcattacaaaatt/3Bio/ | |
| Probe OLA1 | (SEQ ID NO: 596) | |
| 24138C US | GTGGCAACAGAAATCAGGTGGTGA tgcaca aaagtttaaY ggccttac | S protein |
| Probe OLA1 | (SEQ ID NO: 670) | T859N |
| 24138A US | AGAGGACTGCTAAAGGTTTGTAGG tgcaca aaagtttaaY ggccttaa | |
| Probe OLA1 | (SEQ ID NO: 671) | |
| 24138 DS | /5Phos/tg ttttgccacc tttgctcac/3Bio/ | |
| Probe OLA1 | (SEQ ID NO: 672) | |
| 24138C US | GTGGCAACAGAAATCAGGTGGTGA GTGAGCAAAGGTGGCAAAACAG | |
| Probe OLA2 | (SEQ ID NO: 673) | |
| 24138A US | AGAGGACTGCTAAAGGTTTGTAGG GTGAGCAAAGGTGGCAAAACAT | |
| Probe OLA2 | (SEQ ID NO: 674) | |
| 24138 DS | /5Phos/TAAGGCC RTTAAACTTT TGTGCA/3Bio/ | |
| Probe OLA2 | (SEQ ID NO: 675) | |
| 21846C US | CATTTGGTCATTGGTTCAAGACGA atttaatgatggtgtttattttgcttcca c | S protein |
| Probe OLA1 | (SEQ ID NO: 714) | T95I |
| 21846T US | CTGGTCCGTTGTGGTCCTTCTAAC atttaatgatggtgtttattttgcttcca t | |
| Probe OLA1 | (SEQ ID NO: 715) | |
| 21846 DS | /5Phos/tgagaagtYtaacataataagaggctgg/3Bio/ | |
| Probe OLA1 | (SEQ ID NO: 716) | |
| 21846C US | CATTTGGTCATTGGTTCAAGACGA cagcctcttattatgttaRacttctca g | |
| Probe OLA2 | (SEQ ID NO: 717) | |
| 21846T US | CTGGTCCGTTGTGGTCCTTCTAAC cagcctcttattatgttaRacttctca a | |
| Probe OLA2 | (SEQ ID NO: 718) | |
| 21846 DS | /5Phos/tggaagcaaaataaacaccatcattaaatg/3Bio/ | |
| Probe OLA2 | (SEQ ID NO: 719) | |
| 22578G US | CTAATAGCTCCTGTGCCCTCGTAT ctaatattacaaacttgtgcccttttg g | S protein |
| Probe OLA1 | (SEQ ID NO: 720) | G339D |
| 22578A US | AATCCGTCGACTAGCCTGAGAATT ctaatattacaaacttgtgcccttttg a | |
| Probe OLA1 | (SEQ ID NO: 721) | |
| 22578 DS | /5Phos/tgaagtttttaacgccaccaRatttg/3Bio/ | |
| Probe OLA1 | (SEQ ID NO: 722) | |
| 22578G US | CTAATAGCTCCTGTGCCCTCGTAT aaatYtggtggcgttaaaaacttca c | |
| Probe OLA2 | (SEQ ID NO: 723) | |
| 22578A US | AATCCGTCGACTAGCCTGAGAATT aaatYtggtggcgttaaaaacttca t | |
| Probe OLA2 | (SEQ ID NO: 724) | |
| 22578 DS | /5Phos/caaaagggcacaagtttgtaatattagg/3Bio/ | |
| Probe OLA2 | (SEQ ID NO: 725) | |
| 23525C US | GTGGCAACAGAAATCAGGTGGTGA ggctgtt taataggggY tgaa c | S protein |
| Probe OLA1 | (SEQ ID NO: 726) | H655Y |
| 23525T US | AGAGGACTGCTAAAGGTTTGTAGG ggctgtt taataggggY tgaa t | |
| Probe OLA1 | (SEQ ID NO: 727) | |
| 23525 DS | /5Phos/atgtY aaYaactcat atgagtgtga ca/3Bio/ | |
| Probe OLA1 | (SEQ ID NO: 728) | |
| 23525C US | GTGGCAACAGAAATCAGGTGGTGA g tcacactcat atgagttRtt Racat g | |
| Probe OLA2 | (SEQ ID NO: 729) | |
| 23525T US | AGAGGACTGCTAAAGGTTTGTAGG g tcacactcat atgagttRtt Racat a | |
| Probe OLA2 | (SEQ ID NO: 730) | |
| 23535 DS | /5Phos/ttca Rcccctatta aacagcct/3Bio/ | |
| Probe OLA2 | (SEQ ID NO: 731) | |

+Denotes a locked nucleic acid (LNA) base

TABLE 11
Synthetic Templates-SARS-CoV-2 Protein Mutations

| | | Protein/ |
|---|---|---|
| Oligo Name | Sequence | Amino Acid |
| 21765-21770 WT | cctttctttt ccaatgttac ttggttccat gcta tacatg tctctgggac | S protein |
| | caatggtact aagaggtttg ataac (SEQ ID NO: 135) | |
| 21765-21770del | a cctttctttt ccaatgttac ttggttccat gcta tctctgggac | A69-70 |
| template strand | caatggtact aagaggtttg ataaccctgt (SEQ ID NO: 136) | |
| 21765-21770 WT cDNA | ttat caaacctctt agtaccattg gtcccagaga catgta tagcatggaa | |
| strand | ccaagtaaca ttggaaaaga aaggt (SEQ ID NO: 137) | |
| 21765-21770del cDNA | agggttat caaacctctt agtaccattg gtcccagaga tagcatggaa | |
| strand | ccaagtaaca ttggaaaaga aaggtaa (SEQ ID NO: 138) | strand |
| 23063A template strand | ttact ttcctttaca atcatatggt ttccaaccca ct a atggtgt | S protein |
| | tggttaccaa ccatacagag tagtagtact (SEQ ID NO: 139) | |
| 23063T template strand | ttact ttcctttaca atcatatggt ttccaaccca ct t atggtgt | N501Y |
| | tggttaccaa ccatacagag tagtagtact (SEQ ID NO: 140) | |
| 23063A cDNA strand | agtacta ctactctgta tggttggtaa ccaacaccat t agtgggttgg | |
| | aaaccatatg attgtaaagg aaagtaa (SEQ ID NO: 141) | |
| 23063T cDNA strand | agtacta ctactctgta tggttggtaa ccaacaccat a agtgggttgg | |
| | aaaccatatg attgtaaagg aaagtaa (SEQ ID NO: 142) | |
| 23604C template strand | aggta tatgcgctag ttatcagact cagactaatt ctc c tcggcg | S protein |
| | ggcacgtagt gtagctagtc aatccatcat (SEQ ID NO: 143) | P681H |
| 23604A template strand | aggta tatgcgctag ttatcagact cagactaatt ctc a tcggcg | |
| | ggcacgtagt gtagctagtc aatccatcat (SEQ ID NO: 144) | |
| 23604C cDNA strand | atgatggatt gactagctac actacgtgcc cgccga g gagaattagt | |
| | ctgagtctga taactagcgc atatacct (SEQ ID NO: 145) | |
| 23604A cDNA strand | atgatggatt gactagctac actacgtgcc cgccga t gagaattagt | |
| | ctgagtctga taactagcgc atatacct (SEQ ID NO: 146) | |
| 22132G template strand | accttg aaggaaaaca gggtaatttc aaaaatctta g g gaatttgt | S protein |
| | gtttaagaat attgatggtt attttaaaatat (SEQ ID NO: 147) | R190S |
| 22132T template strand | accttg aaggaaaaca gggtaatttc aaaaatctta g t gaatttgt | |
| | gtttaagaat attgatggtt attttaaaatat (SEQ ID NO: 148) | |
| 22132G cDNA strand | ttttaaaat aaccatcaat attcttaaac acaaattc c ctaagatttt | |
| | tgaaattacc ctgttttcct tcaaggt (SEQ ID NO: 149) | |
| 22132T cDNA strand | ttttaaaat aaccatcaat attcttaaac acaaattc a ctaagatttt | |
| | tgaaattacc ctgttttcct tcaaggt (SEQ ID NO: 150) | |
| 22206A template strand | at atattctaag cacacgccta ttaatttagt gcgtg a tctc | S protein |
| | cctcagggtt tttcggcttt agaaccattg gta (SEQ ID NO: 151) | D215G |
| 22206G template strand | at atattctaag cacacgccta ttaatttagt gcgtg g tctc | |
| | cctcagggtt tttcggcttt agaaccattg gta (SEQ ID NO: 152) | |
| 22206A cDNA strand | taccaat ggttctaaag ccgaaaaacc ctgagggaga t cacgc | |
| | actaaattaa taggcgtgtg cttagaatat at (SEQ ID NO: 153) | |
| 22206G cDNA strand | taccaat ggttctaaag ccgaaaaacc ctgagggaga c cacgc | |
| | actaaattaa taggcgtgtg cttagaatat at (SEQ ID NO: 154) | |
| 22917T template strand | a atcttgattc taaggttggt ggtaattata attacc t gta | S protein |
| | tagattgttt aggaagtcta atctcaaacc tttt (SEQ ID NO: 155) | L452R |
| 22917G template strand | a atcttgattc taaggttggt ggtaattata attacc g gta | |
| | tagattgttt aggaagtcta atctcaaacc tttt (SEQ ID NO: 156) | |
| 22917T cDNA strand | aaaa ggtttgagat tagacttcct aaacaatcta tac a | |
| | ggtaattata attaccacca accttagaat caagatt (SEQ ID NO: 157) | |
| 22917G cDNA strand | aaaa ggtttgagat tagacttcct aaacaatcta tac c | |
| | ggtaattata attaccacca accttagaat caagatt (SEQ ID NO: 158) | |
| 23012G template strand | aatcta tcaggccggt agcacacctt gtaatggtgt t g aaggtttt | S protein |
| | aattgttact ttcctttaca atcatatggt (SEQ ID NO: 159) | E484K |
| 23012A template strand | aatcta tcaggccggt agcacacctt gtaatggtgt t a aaggtttt | |
| | aattgttact ttcctttaca atcatatggt (SEQ ID NO: 160) | |
| 23012G cDNA strand | ccatatg attgtaaagg aaagtaacaa ttaaaacctt c aacaccatta | |
| | caaggtgtgc taccggcctg atagatt (SEQ ID NO: 161) | |
| 23012A cDNA strand | ccatatg attgtaaagg aaagtaacaa ttaaaacctt t aacaccatta | |
| | caaggtgtgc taccggcctg atagatt (SEQ ID NO: 162) | |
| 23664C template strand | agtc aatccatcat tgcctacact atgtcacttg gtg c agaaaa | S protein |
| | ttcagttgct tactctaata actctattgc c (SEQ ID NO: 163) | A701V |
| 23664T template strand | agtc aatccatcat tgcctacact atgtcacttg gtg t agaaaa | |
| | ttcagttgct tactctaata actctattgc c (SEQ ID NO: 164) | |
| 23664C cDNA strand | ggcaata gagttattag agtaagcaac tgaattttct g caccaagtga) | |
| | catagtgtag gcaatgatgg attgact (SEQ ID NO: 165 | |
| 23664T cDNA strand | ggcaata gagttattag agtaagcaac tgaattttct a caccaagtga | |
| | catagtgtag gcaatgatgg attgact (SEQ ID NO: 166) | |
| 22813G template strand | atgaa gtcagacaaa tcgctccagg gcaaactgga aM g attgctg | S protein |
| | attataatta taaattacca gatgatttta (SEQ ID NO: 167) | K417N |
| 22813T template strand | atgaa gtcagacaaa tcgctccagg gcaaactgga aM t attgctg | |
| | attataatta taaattacca gatgatttta (SEQ ID NO: 168) | |
| 22813C cDNA strand | taaaatc atctggtaat ttataattat aatcagcaat c Kttccagttt | |
| | gccctggagc gatttgtctg acttcat (SEQ ID NO: 169) | |
| 22813T cDNA strand | taaaatc atctggtaat ttataattat aatcagcaat a Kttccagttt | |
| | gccctggagc gatttgtctg acttcat (SEQ ID NO: 170) | |
| 22812A template strand | gatgaa gtcagacaaa tcgctccagg gcaaactgga a a Kattgctg | S protein |
| | attataatta taaattacca gatgatttt (SEQ ID NO: 171) | K417T |
| 22812C template strand | gatgaa gtcagacaaa tcgctccagg gcaaactgga a c Kattgctg | |
| | attataatta taaattacca gatgatttt (SEQ ID NO: 172) | |
| 22812A cDNA strand | aaaatca tctggtaatt tataattata atcagcaatM t ttccagtttg | |
| | ccctggagcg atttgtctga cttcatc (SEQ ID NO: 173) | |
| 22812C cDNA strand | aaaatca tctggtaatt tataattata atcagcaatM g ttccagtttg | |
| | ccctggagcg atttgtctga cttcatc (SEQ ID NO: 174) | |
| 22227C template strand | a ttaatttagt gcgtgatctc cctcagggtt tttcgg c ttt | S protein |
| | agaaccattg gtagatttgc caataggtat taac (SEQ ID NO: 323) | A222V |
| 22227T template strand | a ttaatttagt gcgtgatctc cctcagggtt tttcgg t ttt | |
| | agaaccattg gtagatttgc caataggtat taac (SEQ ID NO: 324) | |
| 22227C cDNA strand | gttaata cctattggca aatctaccaa tggttctaaa g ccgaaaaacc | |
| | ctgagggaga tcacgcacta aattaat (SEQ ID NO: 325) | |
| 22227T cDNA strand | gttaata cctattggca aatctaccaa tggttctaaa a ccgaaaaacc | |
| | ctgagggaga tcacgcacta aattaat (SEQ ID NO: 326) | |
| 28932C template strand | gctaga atggctggca atggcggtga tgctgctctt g c tttgctgc | N protein |
| | tgcttgacag attgaaccag cttgagagc (SEQ ID NO: 327) | A220V |
| 28932T template strand | gctaga atggctggca atggcggtga tgctgctctt g t tttgctgc | |
| | tgcttgacag attgaaccag cttgagagc (SEQ ID NO: 328) | |
| 28932C cDNA strand | gctctca agctggttca atctgtcaag cagcagcaaa g caagagcagc | |
| | atcaccgcca ttgccagcca ttctagc (SEQ ID NO: 329) | |
| 28932T cDNA strand | gctctca agctggttca atctgtcaag cagcagcaaa a caagagcagc | |
| | atcaccgcca ttgccagcca ttctagc (SEQ ID NO: 330) | |
| 29645G template strand | ctt gtgcagaatg aattctcgta actacatagc acaa g tagat | Orf 10 |
| | gtagttaact ttaatctcac atagcaatct tt (SEQ ID NO: 331) | V30L |
| 29645T template strand | ctt gtgcagaatg aattctcgta actacatagc acaa t tagat | |
| | gtagttaact ttaatctcac atagcaatct tt (SEQ ID NO: 332) | |
| 29645G cDNA strand | aaagatt gctatgtgag attaaagtta actacatcta c ttgtgctatg | |
| | tagttacgag aattcattct gcacaag (SEQ ID NO: 333) | |
| 29645T cDNA strand | aaagatt gctatgtgag attaaagtta actacatcta a ttgtgctatg | |
| | tagttacgag aattcattct gcacaag (SEQ ID NO: 334) | |
| 1059C template strand | acacctttt gaaattaaat tggcaaagaa atttgaca c c ttcaatgggg | Orf 1a |
| | aatgtccaaa ttttgtattt ccctta (SEQ ID NO: 335) | T265I |
| 1059T template strand | acacctttt gaaattaaat tggcaaagaa atttgaca t c ttcaatgggg | |
| | aatgtccaaa ttttgtattt ccctta (SEQ ID NO: 336) | |
| 1059C cDNA strand | taaggga aatacaaaat ttggacattc cccattgaag g tgtcaaattt | |
| | ctttgccaat ttaatttcaa aaggtgt (SEQ ID NO: 337) | |
| 1059T cDNA strand | taaggga aatacaaaat ttggacattc cccattgaag a tgtcaaattt | |
| | ctttgccaat ttaatttcaa aaggtgt (SEQ ID NO: 338) | |
| 25563G template strand | ggctt attgttggcg ttgcacttct tgctgttttt ca g agcgctt | Orf3aQ57H |
| | ccaaaatcat aaccctcaaa aagagatggc (SEQ ID NO: 339) | |
| 25563T template strand | ggctt attgttggcg ttgcacttct tgctgttttt ca t agcgctt | |
| | ccaaaatcat aaccctcaaa aagagatggc (SEQ ID NO: 340) | |
| 25563G cDNA strand | gccatct ctttttgagg gttatgattt tggaagcgct c tgaaaaacag | |
| | caagaagtgc aacgccaaca ataagcc (SEQ ID NO: 341) | |
| 25563T cDNA strand | gccatct ctttttgagg gttatgattt tggaagcgct a tgaaaaacag | |
| | caagaagtgc aacgccaaca ataagcc (SEQ ID NO: 342) | |
| 21991-21993 WT template | tgaatt tcaattttgt aatgatccat ttttgggtgt tta ttaccac | S protein |
| strand | aaaaacaaca aaagttggat ggaaagtga (SEQ ID NO: 343) | Y144 del |
| 21991-21993 deltemplate | gtgaatt tcaattttgt aatgatccat ttttgggtgt ttaccac | |
| strand | aaaaacaaca aaagttggat ggaaagtgag t (SEQ ID NO: 344) | |
| 21991-21993 WT cDNA | tcactt tccatccaac ttttgttgtt tttgtggtaa taa acacccaaaa | |
| strand | atggatcatt acaaaattga aattca (SEQ ID NO: 345) | |
| 21991-21993 del cDNA | actcactt tccatccaac ttttgttgtt tttgtggtaa acacccaaaa | |
| strand | atggatcatt acaaaattga aattcac (SEQ ID NO: 346) | |
| 23271C template strand | aagtttc tgcctttcca acaatttggc agagacattg c tgacactac | S protein |
| | tgatgctgtc cgtgatccac agacactt (SEQ ID NO: 347) | A570D |
| 23271A template strand | aagtttc tgcctttcca acaatttggc agagacattg a tgacactac | |
| | tgatgctgtc cgtgatccac agacactt (SEQ ID NO: 348) | |
| 23271C cDNA strand | aagtgtc tgtggatcac ggacagcatc agtagtgtca g caatgtctct | |
| | gccaaattgt tggaaaggca gaaactt (SEQ ID NO: 349) | |
| 23271A cDNA strand | aagtgtc tgtggatcac ggacagcatc agtagtgtca t caatgtctct | |
| | gccaaattgt tggaaaggca gaaactt (SEQ ID NO: 350) | |
| 23709C template strand | tcagttgct tactctaata actctattgc cataccca c a aattttacta | S protein |
| | ttagtgttac cacagaaatt ctacca (SEQ ID NO: 351) | T716I |
| 23709T template strand | tcagttgct tactctaata actctattgc cataccca t a aattttacta | |
| | ttagtgttac cacagaaatt ctacca (SEQ ID NO: 352) | |
| 23709C cDNA strand | tggtaga atttctgtgg taacactaat agtaaaattt g tgggtatggc | |
| | aatagagtta ttagagtaag caactga (SEQ ID NO: 353) | |
| 23709T cDNA strand | tggtaga atttctgtgg taacactaat agtaaaattt a tgggtatggc | |
| | aatagagtta ttagagtaag caactga (SEQ ID NO: 354) | |
| 24506T template strand | tt ttggtgcaat ttcaagtgtt ttaaatgata tcctt t cacg | S protein |
| | tcttgacaaa gttgaggctg aagtgcaaat att (SEQ ID NO: 355) | S982A |
| 24506G template strand | tt ttggtgcaat ttcaagtgtt ttaaatgata tcctt g cacg | |
| | tcttgacaaa gttgaggctg aagtgcaaat att (SEQ ID NO: 356) | |
| 24506T cDNA strand | aatattt gcacttcagc ctcaactttg tcaagacgtg a aaggatatca | |
| | tttaaaacac ttgaaattgc accaaaa (SEQ ID NO: 357) | |
| 24506G cDNA strand | aatattt gcacttcagc ctcaactttg tcaagacgtg c aaggatatca | |
| | tttaaaacac ttgaaattgc accaaaa (SEQ ID NO: 358) | |
| 24914G template strand | acaa aggaattttt atgaaccaca aatcattact aca g acaaca | S protein |
| | catttgtgtc tggtaactgt gatgttgtaa t (SEQ ID NO: 359) | D1118H |
| 24914C template strand | acaa aggaattttt atgaaccaca aatcattact aca c acaaca | |
| | catttgtgtc tggtaactgt gatgttgtaa t (SEQ ID NO: 360) | |
| 24914G cDNA strand | attacaa catcacagtt accagacaca aatgtgttgt c tgtagtaatg | |
| | atttgtggtt cataaaaatt cctttgt (SEQ ID NO: 361) | |
| 24914C cDNA strand | attacaa catcacagtt accagacaca aatgtgttgt g tgtagtaatg | |
| | atttgtggtt cataaaaatt cctttgt (SEQ ID NO: 362) | |
| 241C template strand | gtccgtg ttgcagccga tcatcagcac atctaggttt c gtccgggtg | 5′UTR |
| | tgaccgaaag gtaagatgga gagccttg (SEQ ID NO: 363) | |
| 24IT template strand | gtccgtg ttgcagccga tcatcagcac atctaggttt t gtccgggtg | |
| | tgaccgaaag gtaagatgga gagccttg (SEQ ID NO: 364) | |
| 241C cDNA strand | caaggct ctccatctta cctttcggtc acacccggac g aaacctagat | |
| | gtgctgatga tcggctgcaa cacggac (SEQ ID NO: 365) | |
| 24IT cDNA strand | caaggct ctccatctta cctttcggtc acacccggac a aaacctagat | |
| | gtgctgatga tcggctgcaa cacggac (SEQ ID NO: 366) | |
| 3037C template strand | g tgagtttaaa ttggcttcac atatgtattg ttcttt c tac | nsp3 |
| | cctccagatg aggatgaaga agaaggtgat tgtg (SEQ ID NO: 367) | F924F |
| 3037T template strand | g tgagtttaaa ttggcttcac atatgtattg ttcttt t tac | |
| | cctccagatg aggatgaaga agaaggtgat tgtg (SEQ ID NO: 368) | |
| 3037C cDNA strand | cacaatc accttcttct tcatcctcat ctggagggta g aaagaacaat | |
| | acatatgtga agccaattta aactcac (SEQ ID NO: 369) | |
| 3037T cDNA strand | cacaatc accttcttct tcatcctcat ctggagggta a aaagaacaat | |
| | acatatgtga agccaattta aactcac (SEQ ID NO: 370) | |
| 14408C template strand | gcaaacttta atgttttatt ctctacagtg ttcccac c ta | RdRp |
| | caagttttgg accactagtg agaaaaatat ttgtt (SEQ ID NO: 371) | |
| 14408T template strand | gcaaacttta atgttttatt ctctacagtg ttcccac t ta | P323L |
| | caagttttgg accactagtg agaaaaatat ttgtt (SEQ ID NO: 372) | |
| 14408C cDNA strand | aacaaat atttttctca ctagtggtcc aaaacttgta g gtgggaacac | |
| | tgtagagaat aaaacattaa agtttgc (SEQ ID NO: 373) | |
| 14408T cDNA strand | aacaaat atttttctca ctagtggtcc aaaacttgta a gtgggaacac | |
| | tgtagagaat aaaacattaa agtttgc (SEQ ID NO: 374) | |
| 26144G template strand | gagc ctgaagaaca tgtccaaatt cacacaatcg acg g ttcatc | Orf3a |
| | cggagttgtt aatccagtaa tggaaccaat t (SEQ ID NO: 375) | G251V |
| 26144T template strand | gagc ctgaagaaca tgtccaaatt cacacaatcg acg t ttcatc | |
| | cggagttgtt aatccagtaa tggaaccaat t (SEQ ID NO: 376) | |
| 26144G cDNA strand | aattggt tccattactg gattaacaac tccggatgaa c cgtcgattgt | |
| | gtgaatttgg acatgttctt caggctc (SEQ ID NO: 377) | |
| 26144T cDNA strand | aattggt tccattactg gattaacaac tccggatgaa a cgtcgattgt | |
| | gtgaatttgg acatgttctt caggctc (SEQ ID NO: 378) | |
| 29095C template strand | gta ctgccactaa agcatacaat gtaacacaag cttt c ggcag | N protein |
| | acgtggtcca gaacaaaccc aaggaaattt tg (SEQ ID NO: 379) | F274F |
| 29095T template strand | gta ctgccactaa agcatacaat gtaacacaag cttt t ggcag | |
| | acgtggtcca gaacaaaccc aaggaaattt tg (SEQ ID NO: 380) | |
| 29095C cDNA strand | caaaatt tccttgggtt tgttctggac cacgtctgcc g aaagcttgtg | |
| | ttacattgta tgctttagtg gcagtac (SEQ ID NO: 381) | |
| 29095T cDNA strand | caaaatt tccttgggtt tgttctggac cacgtctgcc a aaagcttgtg | |
| | ttacattgta tgctttagtg gcagtac (SEQ ID NO: 382) | |
| 22865G template strand | tta taaattacca gatgatttta caggctgcgt tata g cttgg) | S protein |
| | aattctaaca atcttgattc taaggttggt gg (SEQ ID NO: 383 | A435S |
| 22865T template strand | tta taaattacca gatgatttta caggctgcgt tata t cttgg | |
| | aattctaaca atcttgattc taaggttggt gg (SEQ ID NO: 384) | |
| 22865G cDNA strand | ccaccaa ccttagaatc aagattgtta gaattccaag c tataacgcag | |
| | cctgtaaaat catctggtaa tttataa (SEQ ID NO: 385) | |
| 22865T cDNA strand | ccaccaa ccttagaatc aagattgtta gaattccaag a tataacgcag | |
| | cctgtaaaat catctggtaa tttataa (SEQ ID NO: 386) | |
| 22320A template strand | ttacttgc tttacataga agttatttga ctcctggtg a ttcttcttca | S protein |
| | ggttggacag ctggtgctgc agcttat (SEQ ID NO: 539) | D253G |
| 22320G template strand | ttacttgc tttacataga agttatttga ctcctggtg g ttcttcttca | |
| | ggttggacag ctggtgctgc agcttat (SEQ ID NO: 540) | |
| 22320A cDNA strand | ataagct gcagcaccag ctgtccaacc tgaagaagaa t caccaggag | |
| | tcaaataact tctatgtaaa gcaagtaa (SEQ ID NO: 541) | |
| 22320G cDNA strand | ataagct gcagcaccag ctgtccaacc tgaagaagaa c caccaggag | |
| | tcaaataact tctatgtaaa gcaagtaa (SEQ ID NO: 542) | |
| 23012C template strand | aatcta tcaggccggt agcacacctt gtaatggtgt t c aaggtttt | S protein |
| | aattgttact ttcctttaca atcatatggt (SEQ ID NO: 597) | E484Q |
| 23012C cDNA strand | ccatatg attgtaaagg aaagtaacaa ttaaaacctt g aacaccatta | |
| | caaggtgtgc taccggcctg atagatt (SEQ ID NO: 598) | |
| 21618C template strand | ttattgccac tagtctctag tcagtgtgtt aatctta c aa ccagaactca | S protein |
| | attaccccct gcatacacta attct (SEQ ID NO: 599) | T19R |
| 21618G template strand | ttattgccac tagtctctag tcagtgtgtt aatctta g aa ccagaactca | |
| | attaccccct gcatacacta attct (SEQ ID NO: 600) | |
| 21618C cDNA strand | agaat tagtgtatgc agggggtaat tgagttctgg tt g taagatt | |
| | aacacactga ctagagacta gtggcaataa (SEQ ID NO: 601) | |
| 21618G cDNA strand | agaat tagtgtatgc agggggtaat tgagttctgg tt c taagatt | |
| | aacacactga ctagagacta gtggcaataa (SEQ ID NO: 602) | |
| 23604G template strand | aggta tatgcgctag ttatcagact cagactaatt ctc g tcggcg | S protein |
| | ggcacgtagt gtagctagte aatccatcat (SEQ ID NO: 603) | P681R |
| 23604G cDNA strand | atgatggatt gactagctac actacgtgcc cgccga c gagaattagt | |
| | ctgagtctga taactagcgc atatacct (SEQ ID NO: 604) | |
| 24775A template strand | gtg tagtcttctt gcatgtgact tatgtccctg caca a gaaaa | S protein |
| | gaacttcaca actgctcctg ccatttgtca tg (SEQ ID NO: 605) | Q1071H |
| 24775T template strand | gtg tagtcttctt gcatgtgact tatgtccctg caca t gaaaa | |
| | gaacttcaca actgctcctg ccatttgtca tg (SEQ ID NO: 606) | |
| 24775A cDNA strand | ca tgacaaatgg caggagcagt tgtgaagttc ttttc t tgtg | |
| | cagggacata agtcacatgc aagaagacta cac (SEQ ID NO: 607) | |
| 24775T cDNA strand | ca tgacaaatgg caggagcagt tgtgaagttc ttttc a tgtg | |
| | cagggacata agtcacatgc aagaagacta cac (SEQ ID NO: 608) | |
| 22995C template strand | aga gatatttcaa ctgaaatcta tcaggccggt aRca c acctt | S protein |
| | gtaatggtgt tVaaggtttt aattgttact tt (SEQ ID NO: 609) | T478K |
| 22995A template strand | aga gatatttcaa ctgaaatcta tcaggccggt aRca a acctt | |
| | gtaatggtgt tVaaggtttt aattgttact tt (SEQ ID NO: 610) | |
| 22995C cDNA strand | aa agtaacaatt aaaaccttBa acaccattac aaggt g tgYt | |
| | accggcctga tagatttcag ttgaaatatc tct (SEQ ID NO: 611) | |
| 22995A cDNA strand | aa agtaacaatt aaaaccttBa acaccattac aaggt t tgYt | |
| | accggcctga tagatttcag ttgaaatatc tct (SEQ ID NO: 612) | |
| 24224T template strand | tgca ctgttagcgg gtacaatcac ttctggttgg acc t ttggtg | S protein |
| | caggtgctgc attacaaata ccatttgcta t (SEQ ID NO: 613) | F888L |
| 24224C template strand | tgca ctgttagcgg gtacaatcac ttctggttgg acc c ttggtg | |
| | caggtgctgc attacaaata ccatttgcta t (SEQ ID NO: 614) | |
| 24224T cDNA strand | a tagcaaatgg tatttgtaat gcagcacctg caccaa a ggt | |
| | ccaaccagaa gtgattgtac ccgctaacag tgca (SEQ ID NO: 615) | |
| 24224C cDNA strand | a tagcaaatgg tatttgtaat gcagcacctg caccaa g ggt | |
| | ccaaccagaa gtgattgtac ccgctaacag tgca (SEQ ID NO: 616) | |
| 25088G template strand | tgttgattta ggtgacatct ctggcattaa tgcttca g tt | S protein |
| | gtaaacattc aaaaagaaat tgaccgcctc aatga (SEQ ID NO: 617) | VH67F |
| 25088T template strand | tgttgattta ggtgacatct ctggcattaa tgcttca t tt | |
| | gtaaacattc aaaaagaaat tgaccgcctc aatga (SEQ ID NO: 618) | |
| 25088G cDNA strand | tcatt gaggcggtca atttcttttt gaatgtttac aa c | |
| | tgaagca ttaatgccag agatgtcacc taaatcaaca (SEQ ID NO: 619) | |
| 25088T cDNA strand | tcatt gaggcggtca atttcttttt gaatgtttac aa a | |
| | tgaagca ttaatgccag agatgtcacc taaatcaaca (SEQ ID NO: 620) | |
| 23593G template strand | ccatt ggtgcaggta tatgcgctag ttatcagact cM g actaatt | S protein |
| | ctcVtcggcg ggcacgtagt gtagctagtc (SEQ ID NO: 621) | Q677H |
| 23593T template strand | ccatt ggtgcaggta tatgcgctag ttatcagact cM t actaatt | |
| | ctcVtcggcg ggcacgtagt gtagctagtc (SEQ ID NO: 622) | |
| 23593C template strand | ccatt ggtgcaggta tatgcgctag ttatcagact cM c actaatt | |
| | ctcVtcggcg ggcacgtagt gtagctagtc (SEQ ID NO: 623) | |
| 23593GcDNA strand | gactagctac actacgtgcc cgccgaBgag aattagt c Kg agtctgataa | |
| | ctagcgcata tacctgcacc aatgg (SEQ ID NO: 624) | |
| 23593T cDNA strand | gactagctac actacgtgcc cgccgaBgag aattagt a Kg agtctgataa | |
| | ctagcgcata tacctgcacc aatgg (SEQ ID NO: 625) | |
| 23593C cDNA strand | gactagctac actacgtgcc cgccgaBgag aattagt g Kg agtctgataa | |
| | ctagcgcata tacctgcacc aatgg (SEQ ID NO: 626) | |
| 24138C template strand | agagacctca tttgtgcaca aaagtttaaY ggcctta c tg ttttgccacc | S protein |
| | tttgctcaca gatgaaatga ttgct (SEQ ID NO: 676) | T859N |
| 24138A template strand | agagacctca tttgtgcaca aaagtttaaY ggcctta a tg ttttgccacc | |
| | tttgctcaca gatgaaatga ttgct (SEQ ID NO: 677) | |
| 24138C cDNA strand | AGCAA TCATTTCATC TGTGAGCAAA GGTGGCAAAA CA G TAAGGCC | |
| | RTTAAACTTT TGTGCACAAA TGAGGTCTCT (SEQ ID NO: 678) | |
| 24138A cDNA strand | AGCAA TCATTTCATC TGTGAGCAAA GGTGGCAAAA CA T TAAGGCC | |
| | RTTAAACTTT TGTGCACAAA TGAGGTCTCT (SEQ ID NO: 679) | |
| 21846C template strand | gt cctaccattt aatgatggtg tttattttgc ttcca c tgag aagtctaaca | S protein |
| | taataagagg ctggattttt ggt (SEQ ID NO: 732) | T95I |
| 21846T template strand | gt cctaccattt aatgatggtg tttattttgc ttcca t tgag aagtctaaca | |
| | taataagagg ctggattttt ggt (SEQ ID NO: 733) | |
| 21846C cDNA strand | acc aaaaatccag cctcttatta tgttagactt ctca g tggaa gcaaaataaa | |
| | caccatcatt aaatggtagg ac (SEQ ID NO: 734) | |
| 21846T cDNA strand | acc aaaaatccag cctcttatta tgttagactt ctca a tggaa gcaaaataaa | |
| | caccatcatt aaatggtagg ac (SEQ ID NO: 735) | |
| 22578G template strand | ttagatttc ctaatattac aaacttgtgc ccttttg g tg aagtttttaa | S protein |
| | cgccaccaga tttgcatctg tttatg (SEQ ID NO: 736) | G339D |
| 22578A template strand | ttagatttc ctaatattac aaacttgtgc ccttttg a tg aagtttttaa | |
| | cgccaccaga tttgcatctg tttatg (SEQ ID NO: 737) | |
| 22578G cDNA strand | ataaa cagatgcaaa tctggtggcg ttaaaaactt ca c caaaagg | |
| | gcacaagttt gtaatattag gaaatctaac (SEQ ID NO: 738) | |
| 22578A cDNA strand | ataaa cagatgcaaa tctggtggcg ttaaaaactt ca t caaaagg | |
| | gcacaagttt gtaatattag gaaatctaac (SEQ ID NO: 739) | |
| 23525C template strand | ttt tcaaacacgt gcaggctgtt taataggggc tgaa c atgtc aacaactcat | S protein |
| | atgagtgtga catacccatt gg (SEQ ID NO: 740) | H655Y |
| 23525T template strand | ttt tcaaacacgt gcaggctgtt taataggggc tgaa t atgtc aacaactcat | |
| | atgagtgtga catacccatt gg (SEQ ID NO: 741) | |
| 23525C cDNA strand | cc aatgggtatg tcacactcat atgagttgtt gacat g ttca gcccctatta | |
| | aacagcctgc acgtgtttga aaa (SEQ ID NO: 742) | |
| 23525T cDNA strand | cc aatgggtatg tcacactcat atgagttgtt gacat a ttca gcccctatta | |
| | aacagcctgc acgtgtttga aaa (SEQ ID NO: 743) | |

TABLE 12
Blocking Oligonucleotides-SARS-CoV-2 Protein Mutations

| Blocking | | Protein/ |
|---|---|---|
| Oligo Name | Sequence | Amino Acid |
| 21765-21770 | gttac ttggttccat gctatacatg (SEQ ID NO: 175) | S protein |
| BO1-1 OLA1 | | 69-70 |
| 21765-21770 | ccaatgttac ttggttccat gcta (SEQ ID NO: 176) | |
| BO1-2 OLA1 | | |
| 21765-21770 | tctctgggac caatggtact aag (SEQ ID NO: 177) | |
| BO2 OLA1 | | |
| 21765-21770 | ccattg gtcccagagacatgta (SEQ ID NO: 178) | |
| BO1-1 OLA2 | | |
| 21765-21770 | cttagtaccattg gtcccagaga (SEQ ID NO: 179) | |
| BO1-2 OLA2 | | |
| 21765-21770 | tagcatggaaccaagtaacattgg (SEQ ID NO: 180) | |
| BO2 OLA2 | | |
| 23063 BO1 OLA1 | a atcatatggt ttccaaccca ct Y (SEQ ID NO: 181) | S protein |
| 23063 BO2 OLA1 | atggtgt tggttaccaa ccatac (SEQ ID NO: 182) | N501Y |
| 23063 BO1 OLA2 | tatggttggtaaccaacaccat Y (SEQ ID NO: 183) | |
| 23063 BO2 OLA2 | agtgggttggaaaccatatgat tg (SEQ ID NO: 184) | |
| 23604 BO1 OLA1 | ctag ttatcagact cagactaatt ctc M (SEQ ID NO: 185) | S protein |
| 23604 BO2 OLA1 | tcggcg ggcacgtag (SEQ ID NO: 186) | P681H |
| 23604 BO1 OLA2 | ctacgtgcccgccga B (SEQ ID NO: 187) | |
| 23604 BO2 OLA2 | gagaattagtctgagtctgataa ctagc (SEQ ID NO: 188) | |
| 22132 BO1 OLA1 | ggaaaaca gggtaatttc aaaaatctta g K (SEQ ID NO: 189) | S protein |
| 22132 B02 OLA1 | gaatttgt gtttaagaat attgatggtt attttaaaat at (SEQ ID NO: 190) | R190S |
| 22132 BO1 OLA2 | taaaataaccatcaatattcttaaac acaaattc M (SEQ ID NO: 191) | |
| 22132 BO2 OLA2 | ctaagatttttgaaattaccctgttttcct (SEQ ID NO: 192) | |
| 22206 BO1 OLA1 | acgccta ttaatttagt gcgtg R (SEQ ID NO: 193) | S protein |
| 22206 BO2 OLA1 | tctc cctcagggtt tttcgg (SEQ ID NO: 194) | D215G |
| 22206 BO1 OLA2 | ccgaaaaaccctgagggaga Y (SEQ ID NO: 195) | |
| 22206 BO2 OLA2 | cacgc actaaattaataggcgtg (SEQ ID NO: 196) | |
| 22917 BO1 OLA1 | attc taaggttggt ggtaattata attacc K (SEQ ID NO: 197) | S protein |
| 22917 BO2 OLA1 | gta tagattgttt aggaagtcta atctcaaac (SEQ ID NO: 198) | L452R |
| 22917 BO1 OLA2 | gtttgagattagacttcctaaacaatcta tac M (SEQ ID NO: 199) | |
| 22917 BO2 OLA2 | ggtaattataattaccaccaaccttagaat (SEQ ID NO: 200) | |
| 23012 BO1 OLA1 | agcacacctt gtaatggtgt t R (SEQ ID NO: 201) | S protein |
| 23012 BO2 OLA1 | aaggtttt aattgttact ttcctttaca ate (SEQ ID NO: 202) | E484K |
| 23012 BO1 OLA2 | tgat tgtaaaggaaagtaacaattaaaacctt Y (SEQ ID NO: 203) | |
| 23012 BO2 OLA2 | aacaccattacaaggtgtgcta (SEQ ID NO: 204) | |
| 23664 BO1 OLA1 | gcctacact atgtcacttg gtg Y (SEQ ID NO: 205) | S protein |
| 23664 BO2 OLA1 | agaaaa ttcagttgct tactctaata actct (SEQ ID NO: 206) | A701V |
| 23664 BO1 OLA2 | agagttattagagtaagcaactgaattttct R (SEQ ID NO: 207) | |
| 23664 BO2 OLA2 | caccaagtgacatagtgtaggc (SEQ ID NO: 208) | |
| 22813 BO1 OLA1 | gctccagg gcaaactgga aM K (SEQ ID NO: 209) | S protein |
| 22813 BO2 OLA1 | attgctg attataatta taaattacca gatgatttta c (SEQ ID NO: 210) | K417N |
| 22813 BO1 OLA2 | gtaaaatcatctggtaatttataattataatcagcaat M (SEQ ID NO: 211) | |
| 22813 BO2 OLA2 | Kttccagtttgc cctggagc (SEQ ID NO: 212) | |
| 22812 BO1 OLA1 | gctccagg gcaaactgga a M (SEQ ID NO: 213) | S protein |
| 22812 BO2 OLA1 | Kattgctg attataatta taaattacca gatgatttta c (SEQ ID NO: 214) | K417T |
| 22812 BO1 OLA2 | atcatctggtaatttataattataatcagcaatMK (SEQ ID NO: 215) | |
| 22812 BO2 OLA2 | ttccagtttgc cctggagc (SEQ ID NO: 216) | |
| 22227 BO1 OLA1 | tc cctcagggtt tttcgg Y (SEQ ID NO: 387) | S protein |
| 22227 BO2 OLA1 | ttt agaaccattg gtagatttgc ca (SEQ ID NO: 388) | A222V |
| 22227 BO1 OLA2 | ggcaaatctaccaatggttctaaa R (SEQ ID NO: 389) | |
| 22227 BO2 OLA2 | ccgaaaaaccctgagggaga (SEQ ID NO: 390) | |
| 28932 BO1 OLA1 | cggtga tgctgctctt g Y (SEQ ID NO: 391) | N protein |
| 28932 BO2 OLA1 | tttgctgc tgcttgacag attg (SEQ ID NO: 392) | A220V |
| 28932 BO1 OLA2 | caatctgtcaagcagcagcaaaR (SEQ ID NO: 393) | |
| 28932 BO2 OLA2 | caagagcagcatcaccgc (SEQ ID NO: 394) | |
| 29645 BO1 OLA1 | tg aattctcgta actacatagc acaa K (SEQ ID NO: 395) | Orf 10 |
| 29645 BO2 OLA1 | tagat gtagttaact ttaatctcac atagc (SEQ ID NO: 396) | V30L |
| 29645 BO1 OLA2 | gctatgtgagattaaagttaactacatcta M (SEQ ID NO: 397) | |
| 29645 BO2 OLA2 | ttgtgctatgtagttacgagaattca (SEQ ID NO: 398) | |
| 1059 BO1 OLA1 | tt gaaattaaat tggcaaagaa atttgaca Y (SEQ ID NO: 399) | Orf1a |
| 1059 BO2 OLA1 | c ttcaatgggg aatgtccaaa ttttg (SEQ ID NO: 400) | T265I |
| 1059 BO1 OLA2 | aaaatttggacattccccattgaag R (SEQ ID NO: 401) | |
| 1059 BO2 OLA2 | tgtcaaatttctttgccaatttaatttcaaa (SEQ ID NO: 402) | |
| 25563 BO1 OLA1 | g ttgcacttct tgctgttttt ca K (SEQ ID NO: 403) | Orf3a |
| 25563 BO2 OLA1 | agcgctt ccaaaatcat aaccct (SEQ ID NO: 404) | Q57H |
| 25563 BO1 OLA2 | gggttatgattttggaagcgct M (SEQ ID NO: 405) | |
| 25563 BO2 OLA2 | tgaaaaacagcaagaagtgcaac (SEQ ID NO: 406) | |
| 21991-21993 | ttttgt aatgatccat ttttgggtgt tta (SEQ ID NO: 407) | S protein |
| BO1-1 OLA1 | | Y144 del |
| 21991-21993 | aattttgt aatgatccat ttttgggtgt (SEQ ID NO: 408) | |
| BO1-2 OLA1 | | |
| 21991-2199 3BO1 | ttaccac aaaaacaaca aaagttggat (SEQ ID NO: 409) | |
| OLA1 | | |
| 21991-21993 | ccaacttttgttgtttttgtggtaataa (SEQ ID NO: 410) | |
| BO1-1 OLA2 | | |
| 21991-21993 | atccaacttttgttgtttttgtggtaa (SEQ ID NO: 411) | |
| BO1-2 OLA2 | | |
| 21991-21993 | /5Phos/acacccaaaaatggatcattacaaaatt/3Bio/ (SEQ ID NO: 412) | |
| BO2 OLA2 | | |
| 23271 BO1 OLA1 | cca acaatttggc agagacattg M (SEQ ID NO: 413) | S protein |
| 23271 B02 OLA1 | tgacactac tgatgctgtc cg (SEQ ID NO: 414) | A570D |
| 23271 BO1 OLA2 | cggacagcatca gtagtgtca K (SEQ ID NO: 415) | |
| 23271 BO2 OLA2 | caatgtctctgccaaattgttgga (SEQ ID NO: 416) | |
| 23709 BO1 OLA1 | tctaata actctattgc cataccca Y (SEQ ID NO: 417) | S protein |
| 23709 BO2 OLA1 | a aattttacta ttagtgttac cacagaaatt c (SEQ ID NO: 418) | T716I |
| 23709 BO1 OLA2 | gaatttctgtggtaacactaatagtaaaattt R (SEQ ID NO: 419) | |
| 23709 BO2 OLA2 | tgggtatg gcaatagagttattagagtaa (SEQ ID NO: 420) | |
| 24506 BO1 OLA1 | gcaat ttcaagtgtt ttaaatgata tcctt K (SEQ ID NO: 421) | S protein |
| 24506 BO2 OLA1 | cacg tcttgacaaa gttgaggc (SEQ ID NO: 422) | S982A |
| 24506 BO1 OLA2 | gcctcaactttgtcaagacgtg M (SEQ ID NO: 423) | |
| 24506 BO2 OLA2 | aaggatatcatttaaaacacttgaa attgca (SEQ ID NO: 424) | |
| 24914 BO1 OLA1 | gaattttt atgaaccaca aatcattact aca S (SEQ ID NO: 425) | S protein |
| 24914 BO2 OLA1 | acaaca catttgtgtc tggtaact (SEQ ID NO: 426) | D1118H |
| 24914 BO1 OLA2 | gttaccagacacaaatgtgttgt S (SEQ ID NO: 427) | |
| 24914 BO2 OLA2 | tgtagtaatgatt tgtggttcataaaaattcc (SEQ ID NO: 428) | |
| 241 BO1 OLA1 | cga tcatcagcac atctaggttt Y (SEQ ID NO: 429) | 5′UTR |
| 241 BO2 OLA1 | gtccgggtg tgaccgaaag (SEQ ID NO: 430) | |
| 241 BO1 OLA2 | cggtcacacccggac R (SEQ ID NO: 431) | |
| 241 BO2 OLA2 | aaacctagatgtgctgatgatcg (SEQ ID NO: 432) | |
| 3037 BO1 OLA1 | a ttggcttcac atatgtattg ttcttt Y (SEQ ID NO: 433) | nsp3 |
| 3037 BO2 OLA1 | tac cctccagatg aggatgaaga (SEQ ID NO: 434) | F924F |
| 3037 BO1 OLA2 | cttcatcctcatctggagggta R (SEQ ID NO: 435) | |
| 3037 BO2 OLA2 | aaagaacaatacatatgtgaagccaatt (SEQ ID NO: 436) | |
| 14408 BO1 OLA1 | ttttatt ctctacagtg ttcccac Y (SEQ ID NO: 437) | RdRp |
| 14408 BO2 OLA1 | ta caagttttgg accactagtg aga (SEQ ID NO: 438) | P323L |
| 14408 BO1 OLA2 | ctcactagtggtccaaaacttgta R (SEQ ID NO: 439) | |
| 14408 BO2 OLA2 | gtgggaacactgtagagaataaaact (SEQ ID NO: 440) | |
| 26144 BO1 OLA1 | gtccaaatt cacacaatcg acg K (SEQ ID NO: 441) | Orf3a |
| 26144 BO2 OLA1 | ttcatc cggagttgtt aatccag (SEQ ID NO: 442) | G251V |
| 26144 BO1 OLA2 | ctggattaacaactccggatgaa M (SEQ ID NO: 443) | |
| 26144 BO2 OLA2 | cgtcgattgtgtgaatttggaca (SEQ ID NO: 444) | |
| 29095 BO1 OLA1 | a agcatacaat gtaacacaag cttt Y (SEQ ID NO: 445) | N protein |
| 29095 BO2 OLA1 | ggcag acgtggtcca gaa (SEQ ID NO: 446) | F274F |
| 29095 BO1 OLA2 | ctggaccacgtctgcc R (SEQ ID NO: 447) | |
| 29095 BO2 OLA2 | aaagcttgtgttacattgtatgcttta (SEQ ID NO: 448) | |
| 22865 BO1 OLA1 | ca gatgatttta caggctgcgt tata K (SEQ ID NO: 449) | S protein |
| 22865 BO2 OLA1 | cttgg aattctaaca atcttgattc taagg (SEQ ID NO: 450) | A435S |
| 22865 BO1 OLA2 | cttagaatcaagattgttagaattccaag M (SEQ ID NO: 451) | |
| 22865 BO2 OLA2 | tataacgcagcctgtaaaatcatct (SEQ ID NO: 452) | |
| 22320 BO1 OLA1 | acataga agttatttga ctcctggtg R (SEQ ID NO: 543) | S protein |
| 22320 BO2 OLA1 | ttcttcttca ggttggacag ctg (SEQ ID NO: 544) | D253G |
| 22320 BO1 OLA2 | ag ctgtccaacc tgaagaagaa Y (SEQ ID NO: 545) | |
| 22320 BO2 OLA2 | caccaggag tcaaataact tctatgtaaa (SEQ ID NO: 546) | |
| 23012 BO1 OLA1 | agcacacctt gtaatggtgt t S (SEQ ID NO: 627) | S protein |
| (for E484Q) | | E484Q |
| 23012 BO1 OLA2 | tgat tgtaaaggaaagtaacaattaaaacctt S (SEQ ID NO: 628) | |
| (for E484Q) | | |
| 21618 BO1 OLA1 | actagtctctagtcagtgtgttaatctta S (SEQ ID NO: 629) | S protein |
| 21618 BO2 OLA1 | aa ccagaactca attaccccct (SEQ ID NO: 630) | T19R |
| 21618 BO1 OLA2 | agggggtaattgagttctggtt S (SEQ ID NO: 631) | |
| 21618 BO2 OLA2 | taagatt aacacactga ctagagacta gt (SEQ ID NO: 632) | |
| 23012 BO1 OLA1 | ctag ttatcagact cagactaatt ctc S (SEQ ID NO: 633) | S protein |
| (for P681R) | | P681R |
| 23012 BO1 OLA2 | ctacgtgcccgccga S (SEQ ID NO: 634) | |
| (for P681R) | | |
| 24775 BO1 OLA1 | catgtgact tatgtccctg caca W (SEQ ID NO: 635) | S protein |
| 24775 BO2 OLA1 | gaaaa gaacttcaca actgctcctg (SEQ ID NO: 636) | |
| 24775 BO1 OLA2 | caggagcagt tgtgaagttc ttttc W (SEQ ID NO: 637) | Q1071H |
| 24775 BO2 OLA2 | tgtg cagggacata agtcacat (SEQ ID NO: 638) | |
| 22995 BO1 OLA1 | gaaatcta tcaggccggt aRca M (SEQ ID NO: 639) | S protein |
| 22995 BO2 OLA1 | acctt gtaatggtgt tVaaggtttt aa (SEQ ID NO: 640) | T478K |
| 22995 BO1 OLA2 | tt aaaaccttBa acaccattac aaggt K (SEQ ID NO: 641) | |
| 22995 BO2 OLA2 | tgYt accggcctga tagattt (SEQ ID NO: 642) | |
| 24224 BO1 OLA1 | acaatcac ttctggttgg acc Y (SEQ ID NO: 643) | S protein |
| 24224 BO2 OLA1 | ttggtg caggtgctgc a (SEQ ID NO: 644) | F888L |
| 24224 BO1 OLA2 | gcagcacctgcaccaa R (SEQ ID NO: 645) | |
| 24224 BO2 OLA2 | ggtccaaccagaagtgattgtac (SEQ ID NO: 646) | |
| 25088 BO1 OLA1 | acatct ctggcattaa tgcttca K (SEQ ID NO: 647) | S protein |
| 25088 BO2 OLA1 | tt gtaaacattc aaaaagaaat tgaccgc (SEQ ID NO: 648) | V1167F |
| 25088 BO1 OLA2 | gcggtcaatttctttttgaatgtttacaa M (SEQ ID NO: 649) | |
| 25088 BO2 OLA2 | tgaagca ttaatgccagagatgtc (SEQ ID NO: 650) | |
| 23593 BO1 OLA1 | ta tatgcgctag ttatcagact cM B (SEQ ID NO: 651) | S protein |
| 23593 BO2 OLA1 | actaatt ctcVtcggcg gg (SEQ ID NO: 652) | Q677H |
| 23593 BO1 OLA2 | cccgccgaBgagaattagt V (SEQ ID NO: 653) | |
| 23593 BO2 OLA2 | Kgagtctgataa ctagcgcatata (SEQ ID NO: 654) | |
| 24138 BO1 OLA1 | tgcaca aaagtttaaY ggcctta M (SEQ ID NO: 680) | S protein |
| 24138 BO2 OLA1 | tg ttttgccacc tttgctcac (SEQ ID NO: 681) | T859N |
| 24138 BO1 OLA2 | GTGAGCAAAGGTGGCAAAACAK (SEQ ID NO: 682) | |
| 24138 BO2 OLA2 | TAAGGCC RTTAAACTTT TGTGCA (SEQ ID NO: 683) | |
| 21846 BO1 OLA1 | atttaatgatggtgtttattttgcttcca Y (SEQ ID NO: 744) | T95I |
| 21846 BO2 OLA1 | tgagaagtYtaacataataagaggctgg (SEQ ID NO: 745) | |
| 21846 BO1 OLA2 | cagcctcttattatgttaRacttctca R (SEQ ID NO: 746) | |
| 21846 BO2 OLA2 | tggaagcaaaataaacaccatcattaaatg (SEQ IDNO: 747) | |
| 22578 BO1 OLA1 | ctaatattacaaacttgtgcccttttg R (SEQ ID NO: 748) | G339D |
| 22578 B02 OLA1 | tgaagtttttaacgccaccaRatttg (SEQ IDNO: 749) | |
| 22578 BO1 OLA2 | aaatYtggtggcgttaaaaacttca Y (SEQ ID NO: 750) | |
| 22578 BO2 OLA2 | caaaagggcacaagtttgtaatattagg (SEQ ID NO: 751) | |
| 23525 BO1 OLA1 | ggctgtt taataggggY tgaa Y (SEQ ID NO: 752) | H655Y |
| 23525 BO2 OLA1 | atgtY aaYaactcat atgagtgtga ca (SEQ ID NO: 753) | |
| 23525 BO1 OLA2 | g tcacactcat atgagttRtt Racat R (SEQ ID NO: 754) | |
| 23525 BO2 OLA2 | ttca Rcccctatta aacagcct (SEQ ID NO: 755) | |

TABLE 13
Primers-S Protein with Potential Mutations

| | | Protein/Amplified |
|---|---|---|
| Oligo Name | Sequence | for AA change |
| S panel long range F primer 1 | acgtggtgtttattaccctgac (SEQ ID NO: 217) | S protein |
| (21661-23812) | | 69-70del-A701V |
| S panel long range R primer 1 | gctgcattcagttgaatcacca (SEQ ID NO: 218) | |
| (21661-23812) | | |
| S panel long range F primer 2 | TTCCAATGTTACTTGGTTCCATGCT (SEQ ID NO: 219) | S protein |
| (21739-23707) | | 69-70del-A701V |
| S panel long range R primer 2 | GGGTATGGCAATAGAGTTATTAGAGTAAGC (SEQ ID NO: 220) | |
| S panel medium F primer 1 | ACATTCAACTCAGGACTTGTTCTTACC (SEQ ID NO: 221) | S protein |
| (21706-22341) | | 69-70del, |
| S panel medium R primer 1 | GCTGTCCAACCTGAAGAAGAATCAC (SEQ ID NO: 222) | R190S, D215G |
| (21706-22341) | | |
| S panel medium F primer 2 | CAGGAAGAGAATCAGCAACTGTGTT (SEQ ID NO: 223) | S protein |
| (22624-23321) | | K417N, K417T, |
| S panel medium R primer 2 | TGTCAAGAATCTCAAGTGTCTGTGGAT (SEQ ID NO: 224) | L452R, E484K, |
| (22624-23321) | | N501Y |
| S panel medium F primer 3 | ATCAACTTACTCCTACTTGGCGTGTT (SEQ ID NO: 225) | S protein |
| (23442-24103) | | D614G, P681H, |
| S panel medium R primer 3 | TCTAGCAGCAATATCACCAAGGCAAT (SEQ ID NO: 226) | A701V |
| (23442-24103) | | |
| S panel short F primer 1 | CGTGGTGTTTATTACCCTGACA (SEQ ID NO: 453) | S protein |
| (21662-21918) | | 69-70 del |
| S panel short R primer 1 | ATAAGTAGGGACTGGGTCTTCG (SEQ ID NO: 454) | |
| (21662-21918) | | |
| S panel short F primer 2 | GGATGGAAAGTGAGTTCAGAGT (SEQ ID NO: 455) | S protein |
| (22017-22341) | | R190S, D215G |
| S panel short R primer 2 | GCTGTCCAACCTGAAGAAGAAT (SEQ ID NO: 456) | |
| S panel short F primer 3 | CAGGCTGCGTTATAGCTTGGAA (SEQ ID NO: 457) | S protein |
| (22851-23128) | | L452R, E484K, |
| S panel short R primer 3 | TGCTGGTGCATGTAGAAGTTCA (SEQ ID NO: 458) | N501Y |
| S panel short F primer 4 | TGAGTGTGACATACCCATTGGT (SEQ ID NO: 459) | S protein |
| | | P681H, A701V |
| S panel short R primer 4 | TGCTGCATTCAGTTGAATCACC (SEQ ID NO: 460) | |
| (23542-23813) | | |
| S panel short F primer 5 | ACATTCAACTCAGGACTTGTTCTTACC (SEQ IDNO: 461) | S protein |
| | | 69-70 del |
| S panel short R primer 5 | CAGCCTCTTATTATGTTAGACTTCTCAGTG (SEQ ID NO: 462) | |
| (21706-21873) | | |
| S panel short F primer 6 | GTTGGATGGAAAGTGAGTTCAGAGTTT (SEQ ID NO: 463) | S protein |
| S panel short R primer 6 | TGGCAAATCTACCAATGGTTCTAAAGC (SEQ ID NO: 464) | R190S, D215G |
| (22014-22252) | | |
| S panel short F primer 7 | CAGGAAGAGAATCAGCAACTGTGTT (SEQ ID NO: 465) | S protein |
| | | K417N, K417T |
| S panel short R primer 7 | TCCAAGCTATAACGCAGCCTGTAA (SEQ ID NO: 466) | |
| (22624-22871) | | |
| S panel short F primer 8 | CAATCTTGATTCTAAGGTTGGTGGT (SEQ ID NO: 467) | S protein |
| | | L452R, E484K, |
| S panel short R primer 8 | GTACTACTACTCTGTATGGTTGGTAAC (SEQ ID NO: 468) | N501Y |
| (22879-23099) | | |
| S panel short F primer 9 | TCCACAGACACTTGAGATTCTTGACAT (SEQ ID NO: 469) | S protein |
| | | D614G |
| S panel short R primer 9 | AACACGCCAAGTAGGAGTAAGTTGAT (SEQ ID NO: 470) | |
| (23296-23647) | | |
| S panel short F primer 10 | GCTAGTTATCAGACTCAGACTAATTCTCCTC (SEQ ID NO: 471) | S protein |
| | | P681H, A701V |
| S panel short R primer 10 | ATTGCTGCATTCAGTTGAATCACCAC (SEQ ID NO: 472) | |
| (23576-23815) | | |
| S panel short F primer 11 | GTCAGTGTGTTAATCTTACAACCAGAACTC (SEQ ID NO: 473) | S protein |
| | | 69-70 del |
| S panel short R primer 11 | GCCTCTTATTATGTTAGACTTCTCAGTGGA (SEQ ID NO: 474) | |
| (21600-21871) | | |
| S panel short F primer 12 | TGGATGGAAAGTGAGTTCAGAGTTT (SEQ ID NO: 475) | S protein |
| (22016-22331) | | R190S, D215G |
| S panel short R primer 12 | CTGAAGAAGAATCACCAGGAGTCAA (SEQ ID NO: 476) | |
| (22016-22331) | | |
| S panel short F primer 13 | TACAGGCTGCGTTATAGCTTGGAA (SEQ ID NO: 477) | S protein |
| (22849-23134) | | L452R, E484K, |
| S panel short R primer 13 | AACAGTTGCTGGTGCATGTAGAAG (SEQ ID NO: 478) | N501Y |
| (22849-23134) | | |
| S panel short F primer 14 | TCATGCAGATCAACTTACTCCTACTTGG (SEQ ID NO: 479) | S protein |
| (23434-23755) | | P681H, A701V |
| S panel short R primer 14 | CATAGACACTGGTAGAATTTCTGTGGTAAC (SEQ ID NO: 480) | |
| (23434-23755) | | |
| S panel short F primer 15 | aacgccaccagatttgcatc (SEQ ID NO: 481) | S protein |
| (22589-22860) | | K417N, K417T |
| S panel short R primer 15 | acgcagcctgtaaaatcatct (SEQ ID NO: 482) | |
| (22589-22860) | | |
| S panel short F primer 16 | actgtgttgctgattattctgtc (SEQ ID NO: 483) | S protein |
| (22641-22865) | | K417N, K417T |
| S panel short R primer 16 | ctataacgcagcctgtaaaatca (SEQ ID NO: 484) | |
| (22641-22865) | | |
| S panel short F primer 17 | aaacttgtgcccttttggtg (SEQ ID NO: 485) | S protein |
| (22561 alt start) | | K417N, K417T |
| Full S gene F primer 1 | aggggtactgctgttatgtct (SEQ ID NO: 486) | S protein |
| Full S gene F primer l v2 | a + gg + ggta + ctgctgttatgtct | |
| | (SEQ ID NO: 756) | |
| Full S gene F primer | aggggtactgctgttatgtc N (SEQ ID NO: 663) | |
| 1_wobble_4 base | | |
| Full S gene R primer 1 | gcagtagcgcgaacaaaatc (SEQ ID NO: 487) | |
| Full S gene R primer | gcatccttgatttcaccttg N (SEQ ID NO: 664) | |
| 2_wobble_4 base | | |
| Full S gene R primer | gcatccttgatttcaccttg M (SEQ ID NO: 665) | |
| 2_wobble_2 base | | |
| Full S gene F primer 2 | atttgacatgagtaaatttcccctt (SEQ ID NO: 488) | |
| Full S gene R primer 2 | gcatccttgatttcaccttgc (SEQ ID NO: 489) | |
| Full S gene R primer 2 v2 | agtagcatccttgatttcaccttg (SEQ ID NO: 684) | |
| Full S gene R primer 2 v3 | + gcat + c + cttgatttcaccttgc | |
| | (SEQ ID NO: 757) | |
| Full S gene R primer 2 v4 | + gcat + c + ctt + gatttcaccttgc | |
| | (SEQ ID NO: 758) | |
| Full S gene F primer 3 | ttaaattaaggggtactgctgttatg (SEQ ID NO: 490) | |
| Full S gene R primer 3 | cgcgaacaaaatctgaaggagt (SEQ ID NO: 491) | |
| Region 1 Forward Primer #1 | AGGGGTACTGCTGTTATGTCT (SEQ ID NO: 685) | S protein |
| Region 1 Reverse Primer #1 | GATGCAAATCTGGTGGCGTT (SEQ ID NO: 686) | Region 1 |
| Region 1 Forward Primer #2 | GGGGTACTGCTGTTATGTCTTTA (SEQ ID NO: 687) | |
| Region 1 Reverse Primer #2 | TGCAAATCTGGTGGCGTTAAA (SEQ ID NO: 688) | |
| Region 1 Reverse Primer #3 | CTGAGAGAGGGTCAAGTGCA (SEQ ID NO: 689) | |
| Region 2 Forward Primer #1 | ACTTTAGAGTCCAACCAACAGAA (SEQ ID NO: 690) | S protein |
| Region 2 Reverse Primer #1 | GCCCCTATTAAACAGCCTGC (SEQ ID NO: 691) | Region 2 |
| Region 2 Forward Primer #2 | TTTAACGCCACCAGATTTGC (SEQ ID NO: 692) | |
| Region 2 Reverse Primer #2 | AGCCTGCACGTGTTTGAAAA (SEQ ID NO: 693) | |
| Region 2 Forward Primer #3 | ACTTTAGAGTCCAACCAACAGAA (SEQ ID NO: 694) | |
| Region 2 Reverse Primer #3 | ACCTGTAGAATAAACACGCCA (SEQ ID NO: 695) | |
| Region 2 Forward Primer #4 | AACGCCACCAGATTTGCATC (SEQ ID NO: 696) | |
| Region 3 Forward Primer #1 | GCAGGCTGTTTAATAGGGGC (SEQ ID NO: 697) | S protein |
| Region 3 Reverse Primer #1 | AGTCTGCCTGTGATCAACCT (SEQ ID NO: 698) | Region 3 |
| Region 3 Forward Primer #2 | TTTTCAAACACGTGCAGGCT (SEQ ID NO: 699) | |
| Region 3 Reverse Primer #2 | TGCACTTCAGCCTCAACTTT (SEQ ID NO: 700) | |
| Region 3 Forward Primer #3 | AACACGTGCAGGCTGTTTAA (SEQ ID NO: 701) | |
| Region 3 Reverse Primer #3 | TCAATTTGCACTTCAGCCTCA (SEQ ID NO: 702) | |
| Region 3 Forward Primer #4 | ACGTGCAGGCTGTTTAATAGG (SEQ ID NO: 703) | |
| Region 3 Reverse Primer #4 | CTTTGAAGTCTGCCTGTGATCA (SEQ ID NO: 704) | |
| Region 3 Reverse Primer #5 | CACTTCAGCCTCAACTTTGTCA (SEQ IDNO: 705) | |
| Region 4 Forward Primer #1 | AGGTTGATCACAGGCAGACT (SEQ ID NO: 706) | S protein |
| Region 4 Reverse Primer #1 | GTGCAACGCCAACAATAAGC (SEQ ID NO: 707) | Region 4 |
| Region 4 Forward Primer #2 | ACAAAGTTGAGGCTGAAGTGC (SEQ ID NO: 708) | |
| Region 4 Reverse Primer #2 | ACGCCAACAATAAGCCATCC (SEQ ID NO: 709) | |
| Region 4 Forward Primer #3 | CTTGACAAAGTTGAGGCTGAAG (SEQ ID NO: 710) | |
| Region 4 Reverse Primer #3 | AGGGAGTGAGGCTTGTATCG (SEQ ID NO: 711) | |
| Region 4 Forward Primer #4 | GCTGAAGTGCAAATTGATAGGT (SEQ ID NO: 712) | |
| Region 4 Reverse Primer #4 | GCCAACAATAAGCCATCCGAA (SEQ ID NO: 713) | |

+: Denotes a locked nucleic acid (LNA) base

TABLE 14
Targeting and Detection Probes-Locations 8782,28144, 23403, and 11083

| Probe Name | Probe sequence |
|---|---|
| S 8782 US Probe OLA1 | ACTGGTAACCCAGACATGATCGGT gctgattttgacacatggtttagt (SEQ ID NO: 492) |
| L 8782 US Probe OLA1 | ACTCCCTGTGGGTGAGCTTAATGG gctgattttgacacatggtttagc (SEQ ID NO: 493) |
| 8782 DS Probe OLA1 | /5phos/cagcgtggtggtagttatact/3bio/ (SEQ ID NO: 494) |
| S 28144 US Probe OLA1 | CATTTGGTCATTGGTTCAAGACGA gatatcggtaattatacagtttcctgttc (SEQ ID NO: 495) |
| L 28144 US Probe OLA1 | CTGGTCCGTTGTGGTCCTTCTAAC gatatcggtaattatacagtttcctgttt (SEQ ID NO: 496) |
| 28144 DS Probe OLA1 | /5Phos/accttttacaattaattgccaggaac/3bio/ (SEQ ID NO: 497) |
| 23403A US Probe OLA1 | CTAATAGCTCCTGTGCCCTCGTAT CAGGTTGCTGTTCTTTATCAGG A (SEQ ID NO: 498) |
| 23403G US Probe 0LA1 | AATCCGTCGACTAGCCTGAGAATT CAGGTTGCTGTTCTTTATCAGG G (SEQ ID NO: 499) |
| 23403 DS Probe OLA1 | /5Phos/TGTTAACTGCACAGAAGTCCCT/3Bio/ (SEQ ID NO: 500) |
| 11083G US Probe OLA1 | GTGGCAACAGAAATCAGGTGGTGA agtactc aatggtcttt gttctttttt ttg (SEQ ID NO: 501) |
| 11083T US Probe OLA1 | AGAGGACTGCTAAAGGTTTGTAGG agtactc aatggtcttt gttctttttt ttt (SEQ ID NO: 502) |
| 11083 DS Probe OLA1 | /5Phos/tatgaaa atgccttttt accttttgct a/3Bio/ (SEQ ID NO: 503) |
| 11083G US Probe OLA2 | GTGGCAACAGAAATCAGGTGGTGA gcaaaaggtaaaaaggcattttcatac (SEQ ID NO: 504) |
| 11083T US Probe OLA2 | AGAGGACTGCTAAAGGTTTGTAGG gcaaaaggtaaaaaggcattttcataa (SEQ ID NO: 505) |
| 11083 DS Probe OLA2 | /5Phos/aaaaaaaagaacaaagaccattgagtactc/3Bio/ (SEQ ID NO: 506) |

TABLE 15
Synthetic Templates-Locations 8782, 28144, 23403, and 11083

| Oligo Name | Sequence |
|---|---|
| S 8782 Template | ttgcta acaaacatgc tgattttgac acatggttta gtcagcgtgg tggtagttat actaatgaca |
| Strand | aagcttgcc (SEQ ID NO: 507) |
| L 8782 Template | ttgcta acaaacatgc tgattttgac acatggttta gccagcgtgg tggtagttat actaatgaca |
| Strand | aagcttgcc (SEQ ID NO: 508) |
| S 8782 cDNA Strand | GGCAAGCTT TGTCATTAGT ATAACTACCA CCACGCTGAC TAAACCATGT GTCAAAATCA |
| | GCATGTTTGT TAGCAA (SEQ ID NO: 509) |
| L 8782 cDNA Strand | GGCAAGCTT TGTCATTAGT ATAACTACCA CCACGCTGGC TAAACCATGT GTCAAAATCA |
| | GCATGTTTGT TAGCAA (SEQ ID NO: 510) |
| S 28144 Template | cagt acatcgatat cggtaattat acagtttcct gttcaccttt tacaattaat tgccaggaac |
| Strand | ctaaattggg t (SEQ ID NO: 511) |
| L 28144 Template | cagt acatcgatat cggtaattat acagtttcct gtttaccttt tacaattaat tgccaggaac |
| Strand | ctaaattggg t (SEQ ID NO: 512) |
| S 28144 cDNA Strand | A CCCAATTTAG GTTCCTGGCA ATTAATTGTA AAAGGTGAAC AGGAAACTGT ATAATTACCG |
| | ATATCGATGT ACTG (SEQ ID NO: 513) |
| L 28144 cDNA Strand | A CCCAATTTAG GTTCCTGGCA ATTAATTGTA AAAGGTAAAC AGGAAACTGT ATAATTACCG |
| | ATATCGATGT ACTG (SEQ ID NO: 514) |
| 23403A Template | acaaa tacttctaac caggttgctg ttctttatca gg a tgttaac tgcacagaag tccctgttgc |
| Strand | tattcatgca (SEQ ID NO: 515) |
| 23403G Template | acaaa tacttctaac caggttgctg ttctttatca gg g tgttaac tgcacagaag tccctgttgc |
| Strand | tattcatgca (SEQ ID NO: 516) |
| 23403A cDNA Strand | TGCATGAATA GCAACAGGGA CTTCTGTGCA GTTAACA T CC TGATAAAGAA CAGCAACCTG |
| | GTTAGAAGTA TTTGT (SEQ ID NO: 517) |
| 23403G cDNA Strand | TGCATGAATA GCAACAGGGA CTTCTGTGCA GTTAACA C CC TGATAAAGAACAGCAACCTG |
| | GTTAGAAGTA TTTGT (SEQ ID NO: 5 18) |
| 11083G Template | tactc cagagtactc aatggtcttt gttctttttt tt g tatgaaa atgccttttt accttttgct |
| Strand | atgggtatta (SEQ ID NO: 519) |
| 11083T Template | tactc cagagtactc aatggtcttt gttctttttt tt t tatgaaa atgccttttt accttttgct |
| Strand | atgggtatta (SEQ ID NO: 520) |
| 11083G cDNA Strand | taatacccatagcaaaaggtaaaaaggcattttcata c aaaaaaaagaacaaagaccattgagtactctgg |
| | acta (SEQ ID NO: 521) |
| 11083T cDNA Strand | taatacccatagcaaaaggtaaaaaggcattttcata a aaaaaaaagaacaaagaccattgagtactctgg |
| | acta (SEQ ID NO: 522) |

TABLE 16
Blocking Oligonucleotides-Locations 8782,28144, 23403, and 11083

| Blocking Oligo Name | Sequence |
|---|---|
| 8782 US OLA1 BO | gctgattttgacacatggtttagY (SEQ ID NO: 523) |
| 8782 DS OLA1 BO | cagcgtggtggtagttatact (SEQ ID NO: 524) |
| 28144 US OLA1 BO | gatatcggtaattatacagtttcctgttY (SEQ ID NO: 525) |
| 28144 DS OLA1 BO | accttttacaattaattgccaggaac (SEQ ID NO: 526) |
| 23403 US OLA1 BO | CAGGTTGCTGTTCTTTATCAGG R (SEQ ID NO: 527) |
| 23403 DS OLA1 BO | TGTTAACTGCACAGAAGTCCCT (SEQ ID NO: 528) |
| 11083 US OLA1 BO | agtactc aatggtcttt gttctttttt tt K (SEQ ID NO: 529) |
| 11083 DS OLA1 BO | tatgaaa atgccttttt accttttgct a (SEQ ID NO: 530) |
| 11083 US OLA2 BO | gcaaaaggtaaaaaggcattttcata M (SEQ ID NO: 531) |
| 11083 DS OLA2 BO | aaaaaaaagaacaaagaccattgagtactc (SEQ ID NO: 532) |

### Example 11. Clinical Characterization of Serology Assay Panels and Formats

Clinical characterization of assay panels and formats as shown below was carried out using 200 pre-2019 COVID-19-negative serum samples and 200 PCR-confirmed COVID-19-positive serum samples. The samples were grouped from time of serum collection relative to positive PCR test (0 to 14, 15 to 28, 29 to 56, or 57+ days post-diagnosis (Dx).

Assay panels and formats:

(1) Indirect (Classical) IgG Serology and ACE2 Competition:

Antigens in Panel A: SARS-CoV-2 N protein, S protein, and S-RBD;

Antigens in Panel B: Wild type (WT) SARS-CoV-2 N protein; S protein and S-RBD from WT SARS-CoV-2 and SARS-CoV-2 variants B.1.1.7, B.1.351, and P.1;

(2) Bridging Serology:

Antigens in Panel C: SARS-CoV-2 N protein and S-RBD.

Summary of results for WT antigens:

The WT antigens provided excellent clinical performance, even for early positive samples. The measured specificities for the different antigens/formats ranged from 98.5% to 100%, measured sensitivities for late positives (15+ days from Dx) ranged from 93.8% to 98.3%, measured sensitivities for early positives(≤14 days from Dx) ranged from 65.8% to 92.1%. Assay signals for the Bridging Serology assay format increased with time across the time groups, indicating the Bridging Serology assay format may measure affinity maturation of antibody responses over time.

All antigens and assay formats provided excellent separation of the negative and positive samples. While sensitivity for detecting early (≤14 day) infection was good, for most assay panels and formats there was a significant increase in concentration and sensitivity for samples collected >14 days after diagnosis. Interestingly, while antibody concentrations measured by the Indirect Serology and ACE2 Competition assay formats generally plateaued for time points longer than 14 days, concentrations measured by the Bridging Serology assay format showed significant increases with time across the measured time range. Since the Bridging Serology assay format requires two antibody-antigen binding interactions, it is generally biased towards measuring only high affinity antibodies and may be more sensitive in measuring changes in average antibody affinities as antibody responses mature over time.

The ROC curves showed near ideal separation of negative and late positive samples, as demonstrated by AUC values ranging from 0.983 to 0.992 (an AUC of 1 indicates perfect separation).

All assay formats and antigens showed excellent clinical performance. Point estimates for sensitivity for convalescent samples (15+ days post diagnosis) ranged from 93.8% to 98.3%, with the highest sensitivities obtained by the Indirect IgG Serology and ACE2 Competition assay formats using SARS-CoV-2 S protein as the antigen. Sensitivity during acute infection (0 to 14 days post diagnosis) was lower, which was expected since the antibody response is still developing during this period, but still quite good with point estimates ranging from 65.8% to 92.1%. The Indirect IgG Serology and ACE2 Competition assay formats with SARS-CoV-2 S protein as the antigen were also the most sensitive for detecting acute infection. The assays were very specific with point estimates for specificity ranging from 98.5% to 100%. The performance of all the assays was well within the requirements stated in the United States Food and Drug Administration (FDA) guidance or use of COVID-19 serology tests under an Emergency Use Authorization (EUA).

Summary of results for SARS-CoV-2 variants/mutant antigens:

Panels that include antigens from SARS-CoV-2 variant lineages can provide important tools for assessing the immunity of individuals to the different variants. Assays with the S-RBD antigen from the B.1.351 and P.1 variants, which contain the K417N/T or E484K mutations were performed, and the antibody response from the COVID-19-positive samples against the mutant S-RBD were lower as compared to the antibody response against S-RBD without the K417N/T or E484K mutations (i.e., wild type or B.1.1.7 variant). The difference in antibody responses to the wild type vs. mutant S-RBD was much larger when measured by the ACE2 Competition assay relative to the Indirect IgG Serology assay format. These results suggest that individuals infected with a wild type or wild type-like variant may have reduced ability to avoid infection with the B.1.351 or P.1 variants.

Antibody binding to antigens from the four variants was elevated in COVID-positive vs. COVID-negative subjects, although levels were generally lower for the variant antigens, in particular the S-RBD from the B.1.351 and P.1 variants. The difference may stem from the fact that the COVID-19 positive samples were collected in late 2020 and early 2021 in the US when the wild-type lineage was predominant and there was little evidence in the US of the mutations found in the variants. While the number of mutations in the variant antigens is small, at least some of the mutations are likely present in epitopes that are sufficiently immune-dominant to account for a measurable proportion of the antibody response.

### Example 12. SARS-CoV-2 Serology Assays with Variant Panels

Samples from SARS-CoV-2-infected individuals in the United States in early 2020 (known to be infected with wild-type SARS-CoV-2 (“Wuhan”)); SARS-CoV-2-infected individuals in the United Kingdom (dominating strain: SARS-CoV-2 strain B.1.1.7); or SARS-CoV-2-infected individuals in South Africa (dominating strain: SARS-CoV-2 strain 501Y.V2, also known as B.1.351) were tested using a viral antigen panel that included the wild-type S protein from SARS-CoV-2, S-RBD from SARS-CoV-2 strain 501Y.V2, N protein from SARS-CoV-2, S-RBD from SARS-CoV-2 strain P.1, S-RBD from SARS-CoV-2 strain B.1.1.7, S protein from SARS-CoV-2 strain P.1, S protein from SARS-CoV-2 strain B.1.1.7, S protein from SARS-CoV-2 strain 501Y.V2, and wild-type S-RBD from SARS-CoV-2. The viral antigens were immobilized in a 96-well plate or within an assay cartridge. Detection was performed using an anti-IgG antibody labeled with an electrochemiluminescence (ECL) label.

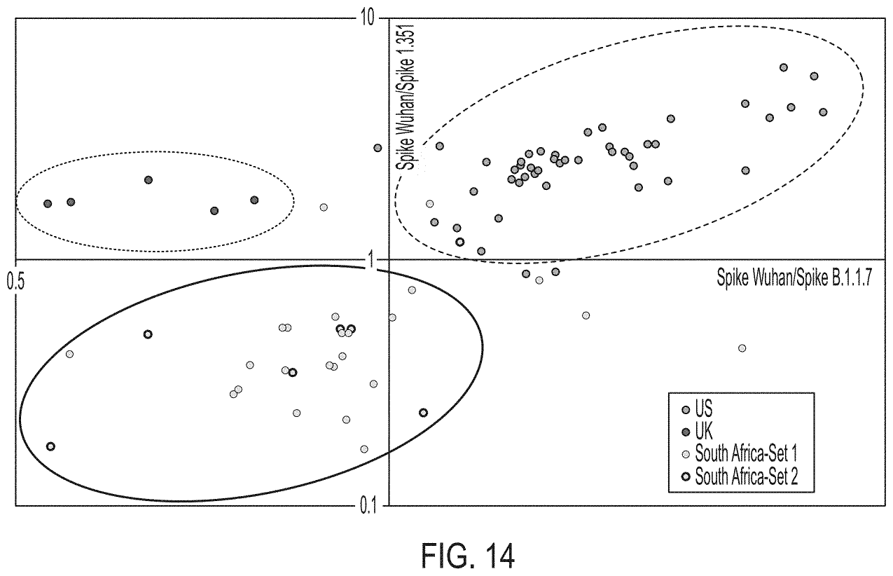

The results are shown in FIG. 14. The measured ratios of antibodies against wild-type SARS-CoV-2 versus SARS-CoV-2 strain B.1.1.7 were plotted on the x-axis, and the measured ratios of antibodies against wild-type SARS-CoV-2 versus SARS-CoV-2 strain 501Y.V2 were plotted on the y-axis. As shown in FIG. 14, samples from the wild-type SARS-CoV-2-infected patients clustered in the top right quadrant; samples from the UK clustered in the top quadrant; and samples from South Africa clustered in the lower left quadrant. Thus, the samples can be differentiated by geographical region based on binding to the S protein or S-RBD in the serology panel. This approach can be used in epidemiology studies to determine the circulating strain in a population or geographical region.

### Example 13. Matched Fingerstick Blood and Saliva Sample Testing

Specimen from 132 individuals who self-collected saliva and/or finger-stick samples were obtained. Matched saliva and finger-stick blood was provided by 125 of these donors. Six donors only provided saliva samples. The saliva sample from one donor with a PCR confirmed diagnosis of COVID-19 did not have sufficient quantity for analysis and was therefore not included. The individuals also completed a survey on COVID-19 diagnosis, exposure, and symptoms, with results summarized in Table 17.

TABLE 17
COVID-19 Survey Responses

| SARS-CoV-2 | Positive | | | |
|---|---|---|---|---|
| Exposure | PCR | Household | COVID-19 | Number of |
| Category | Test | Exposure | Symptoms | Donors |
| Presumed Naïve (PN) | No | No | No | 107 |
| Positive COVID-19 | Yes | Yes | Yes | 6 |
| PCR (PCR+) | | or No | or No | |
| Possibly Non-Naïve | | | | |
| (PNN) | | | | |
| Household exposure | No | Yes | Yes | 3 |
| with symptoms | | | | |
| (HEWS) | | | | |
| Household exposure | No | Yes | No | 4 |
| with no symptoms | | | | |
| (HENS) | | | | |
| COVID-19 | No | No | Yes | 7 |
| Symptomatic (CS) | | | | |
| No Survey (NS) | No | No | No | 5 |
| | response | response | response | |

Methods

The saliva samples were self-collected by donors in a 2 mL tube and frozen at ≤−70° C. without additional processing. The finger-stick blood samples were self-collected by donors using a Mitra collection kit, which contained a swab on which the blood dried shortly after collection. For reconstitution of the dried blood, swabs were placed into 2 mL microcentrifuge tubes containing 200 μL of diluent and extracted for 1 hour at room temperature with gentle shaking at 700 RPM. After 1 hour, the swab was removed and discarded. The microcentrifuge tube containing extracted whole blood was capped and frozen at ≤−70° C.

The samples were subjected to the multiplexed indirect serology panel shown as Coronavirus Panel 2 in Example 3 to measure IgG, IgM, and IgA antibody responses. On the day of testing, saliva and extracted finger-stick blood were thawed at room temperature. Saliva was centrifuged briefly to pull down any food particles or mucus. Prior to analysis, saliva samples were diluted five-fold by combining 20 μL of sample with 80 μL of a sample diluent. Extracted finger-stick blood was diluted 100-fold by combining 10 μL of sample with 990 μL of a different diluent.

Total levels of IgG, IgM, and IgA immunoglobulin were measured using the Isotyping Panel 1 Human/NHP Kit (Meso Scale Diagnostics, Rockville, Md.). Extracted finger-stick blood was run at a dilution of 5,000-fold. Saliva was run at a dilution of 1,000-fold. Calibration and quantitation were carried out as described above for the indirect serology measurements.

### Sample Verification

The samples were tested for quality, e.g., whether there was deterioration of antibodies and/or high levels of food particles or phlegm. Quality of saliva samples was assessed by visual inspection and by measuring salivary antibody content. Saliva samples differed widely in appearance and volume.

The samples were verified to contain expected levels of immunoglobulins as a basic indicator of sample integrity. Median concentrations of total salivary immunoglobulin were 1.5 μg/mL, 2.9 μg/mL, and 83 μg/mL for IgG, IgM, and IgA, respectively. Concentrations of total salivary immunoglobulins were similar to published ranges measured using different assays and collection methods (IgG range=0.4-93 μg/mL; IgM=0.5-13.0 μg/mL; IgA=50.2±19.1 μg/mL). Median concentration of salivary IgG was 100-fold lower than measured in our diluted finger-stick blood samples and 7,300-fold lower than reported for undiluted serum. The variation in total immunoglobulin concentrations across donors was higher in saliva than in finger-stick blood. The ratio of the 75^{th} percentile to the 25^{th} percentile for IgG levels was 4.7 for saliva compared to a ratio of 1.6 for finger-stick blood.

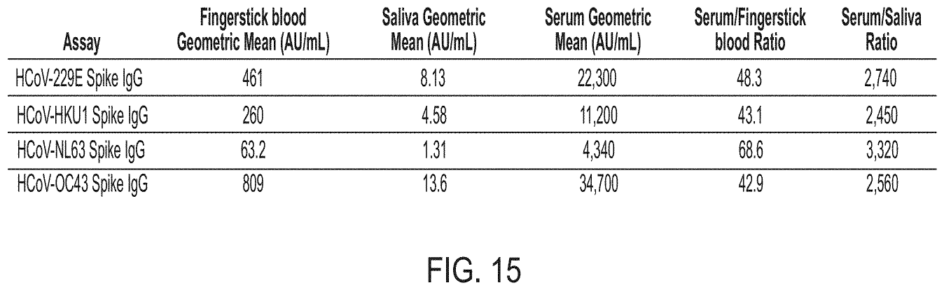

As an additional assessment of sample quality, the levels of antibodies to spike proteins for circulating coronaviruses were measured. Prior infection with these endemic viruses is common, and thus all donors were expected to have high levels of antibodies to at least one of the four circulating coronaviruses on the panel. See, e.g., Gaunt et al., J Clin Microbiol 48:2940-2947 (2010); Killerby et al., J Clin Virol 101:52-56 (2018); and Westerhuis et al., medRxiv 2020.08.21.20177857 (2020). Consistent with the levels of total immunoglobulin in finger-stick blood and saliva relative to serum discussed above, serum levels of antibodies to circulating coronaviruses were on average 51-fold and 2,800-fold higher than in finger-stick blood and saliva, respectively, as shown in FIG. 15.

Of the 125 donors providing matched saliva and finger-stick blood samples, two PN donors had normal levels of total immunoglobulin, and antibodies against the circulating coronaviruses in their blood sample, but not in saliva. One of these donors showed strong IgG reactivity to 229E Spike in finger-stick blood (850 AU/mL; above the 75^{th} percentile), but showed background IgG reactivity to 229E Spike in saliva. The other donor showed strong IgG reactivity to OC43 Spike in finger-stick blood (1,500 AU/mL; above the 75^{th} percentile), but showed background IgG reactivity to OC43 Spike in saliva. This result indicates a likely issue in the collection and/or handling of these samples, but also suggests that measurements of total immunoglobulin levels, or measurements of antibodies against high prevalence endemic viruses such as the circulating coronaviruses, could be used to identify problematic samples. Saliva from these two donors was excluded from analysis of SARS-CoV-2 antibody responses.

### Establishment of Normal Ranges in Non-Infected Individuals

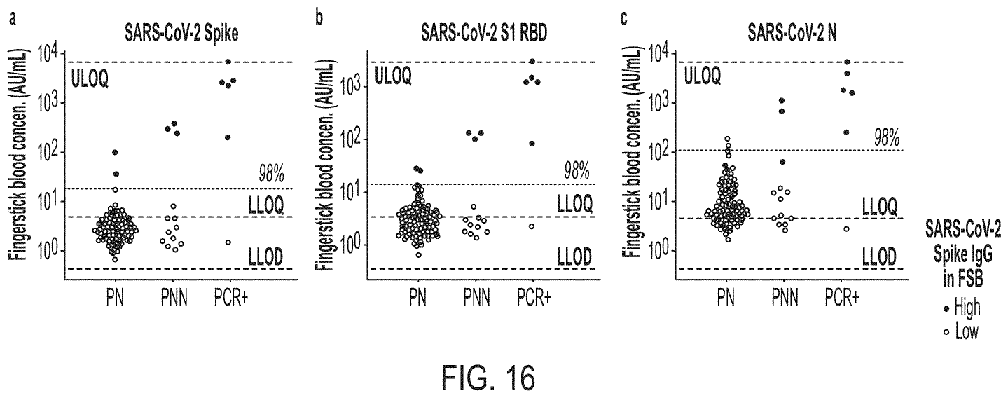

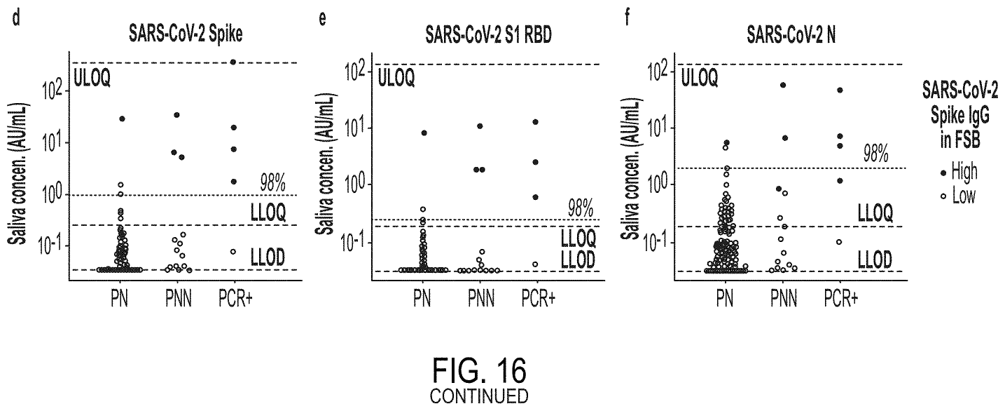

FIG. 16 shows the measured concentrations of antibodies to the SARS-CoV-2 antigens in finger-stick blood and saliva from all donors. The normal ranges for the SARS-CoV-2 serology assays were established using the samples from the 107 study donors who were unlikely to have had prior infection with COVID-19 (PN group). Preliminary threshold values for classifying individuals with prior SARS-CoV-2 infections were determined based on the 98^{th} percentile for the normal range (see Table 18). This approach provides a tolerance for a 1% to 2% rate of undetected asymptomatic infection in this PN group. Overall seropositivity at the time of this study is estimated at 4.4%, based on a study of health care personnel without patient contact within the same metropolitan area performed at approximately the same time. Since half of SARS-CoV-2 infections are thought to be asymptomatic, the expected prevalence of seropositivity resulting from asymptomatic infection is approximately 2%.

TABLE 18
Thresholds for Reactivity to SARS-CoV-2 Antigens

| | FSB Threshold | Saliva Threshold |
|---|---|---|
| Assay | (AU/mL) | (AU/mL) |
| SARS-CoV-2 N IgG | 119 | 3.18 |
| SARS-CoV-2 S1 RBD IgG | 14.3 | 0.244 |
| SARS-CoV-2 Spike IgG | 17.9 | 0.963 |

At the selected dilution, most of the saliva samples from the PN group were below the LOD for reactivity to the SARS-CoV-2 spike and RBD antigens. A higher percentage of these saliva samples had detectable reactivity against SARS-CoV-2 N antigen, which may result from the presence of cross-reactive antibodies originally induced by other coronaviruses. The selected classification thresholds for extracted finger-stick blood were 119 AU/mL, 14 AU/mL, and 18 AU/mL for IgG against SARS-CoV-2 N, RBD, and Spike, respectively. The selected classification thresholds for saliva were 3.2 AU/mL, 0.24 AU/mL, and 0.96 AU/mL for IgG against SARS-CoV-2 N, RBD, and spike, respectively.

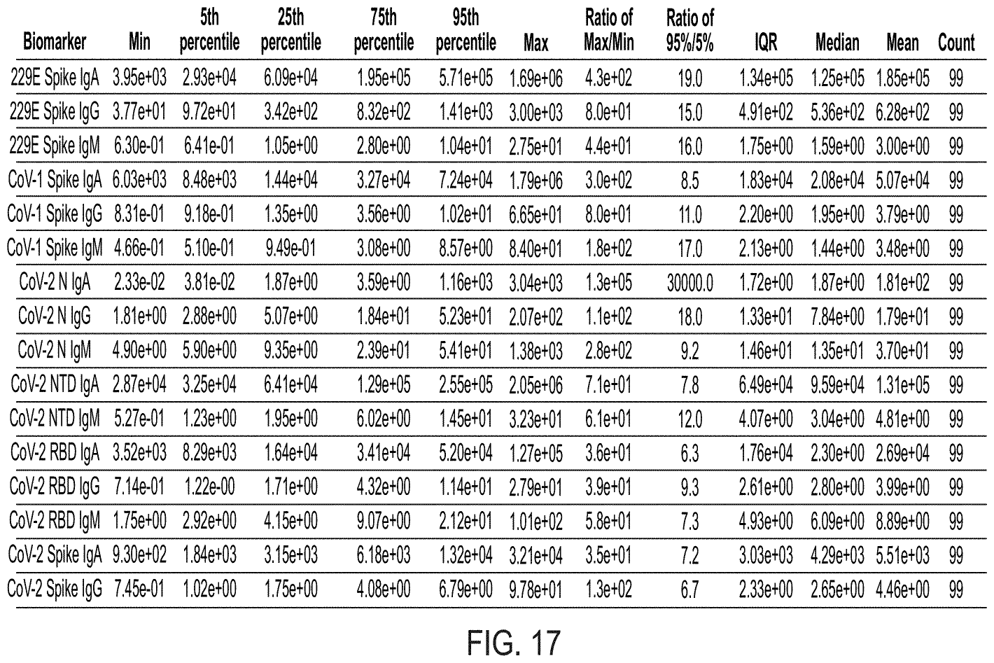

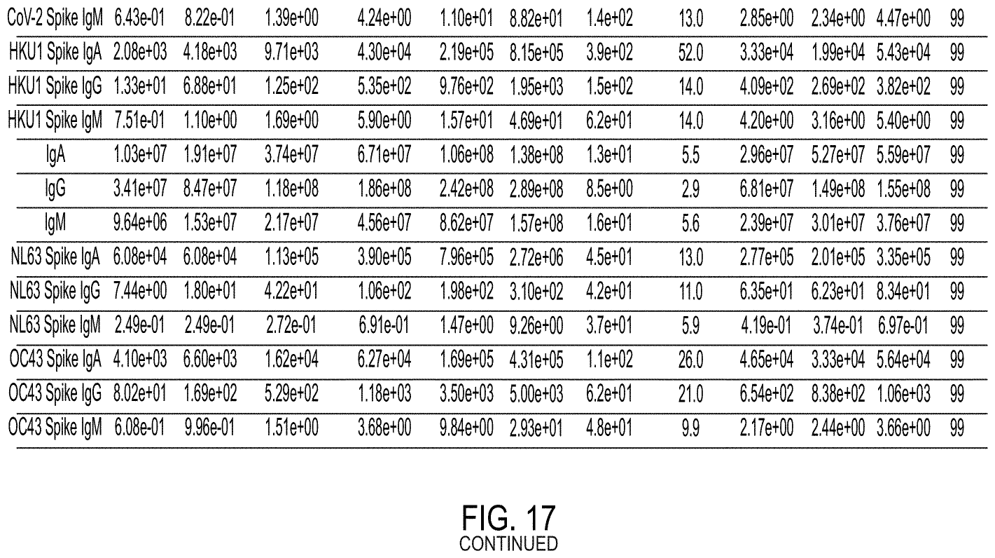

FIG. 17 shows the immunoglobulin concentrations in finger-stick blood self-collected by donors without confirmed COVID-19 diagnosis, household exposure, or recent symptoms, which were used to establish the upper limit of non-reactivity. FIG. 18 shows the immunoglobulin concentrations in saliva self-collected by donors without confirmed COVID-19 diagnosis, household exposure, or recent symptoms.

### Reactivity to SARS-CoV-2 Antigens in Finger-stick Blood Samples

FIG. 16 shows the measured levels of IgG antibodies against the three SARS-CoV-2 antigens (spike, RBD and N) in finger-stick samples, relative to the selected thresholds. By definition, as the thresholds were defined as the 98^{th} percentiles for the PN group, 2% (2 of 107) of the PN samples were classified as positive by each assay. Each of the three assays identified 5 of 6 of the PCR+(confirmed positive) donors. The PCR+ donor that was classified as negative reported an asymptomatic COVID-19 diagnosis more than 30 days previously, but had no significant reactivity to any SARS-CoV-2 antigen for any isotype. This individual had total immunoglobulin levels within the normal range as well as normal reactivity to circulating coronaviruses. A humoral response in this individual may have waned or not developed. For the PNN participants that were considered potentially non-naïve to SARS-CoV-2 based on symptoms and/or household exposure (the CS, HEWS and HENS groups), the spike and RBD assays classified 3 of 14 as positive (2 symptomatic and 1 asymptomatic donor with household contacts). The N assay also classified 2 of these 3 as positive, the third falling just under the threshold. Interestingly, none of the donors who reported possible COVID-19 symptoms, but no confirmed diagnosis or household exposure, had elevated antibody levels to SARS-CoV-2 antigens. This suggests that non-specific symptoms may be unreliable indicators of past infection. Alternatively, antibody levels may have waned faster in mild cases for which donors did not seek testing.

In FIG. 16, closed circles indicate samples provided by donors whose IgG levels in finger-stick blood exceeded the threshold for spike protein. Among the confirmed or possibly infected individuals (PCR+ and PNN groups), the same 8 finger-stick samples showed elevated reactivity to all three finger-stick SARS-CoV-2 antigens, although one of the samples was just under the threshold for the N assay.

Reactivity to SARS-CoV-2 Antigens in Saliva

FIG. 16 also shows the measured levels of IgG antibodies against the three SARS-CoV-2 antigens (spike, RBD and N) in saliva samples. For samples from donors that were confirmed or possibly infected (PCR+ and PNN groups), measurements of IgG against spike and RBD proteins in finger-stick blood and saliva samples provided complete agreement in classification. Measurement of IgG against the N protein performed similarly except for one PCR+ donor who obtained a positive result for N in blood but not saliva. The two PN samples that were classified as positive varied for the different assays and sample types, although there was one PN donor that was classified as positive based on IgG against spike and RBD in blood, and spike, RBD and N in saliva, suggesting this individual may have had an asymptomatic infection.

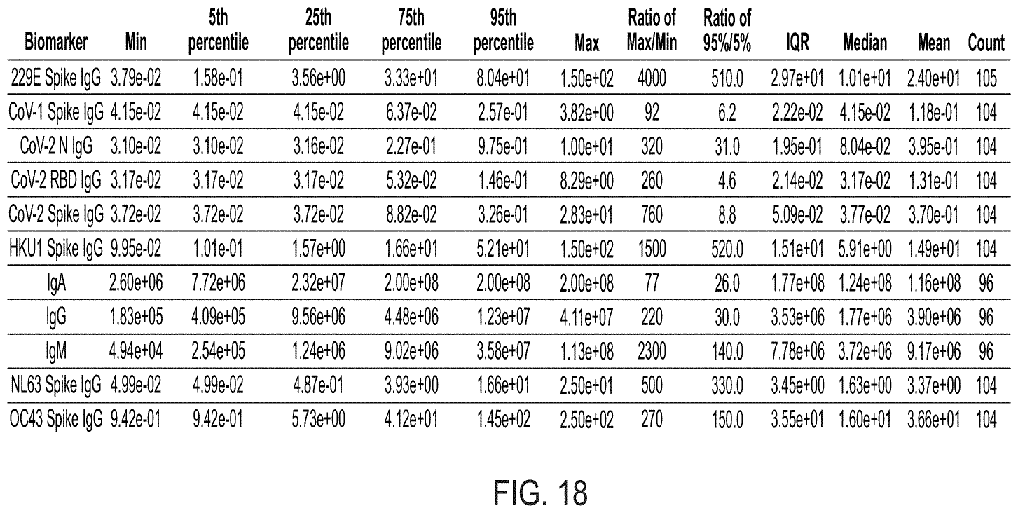

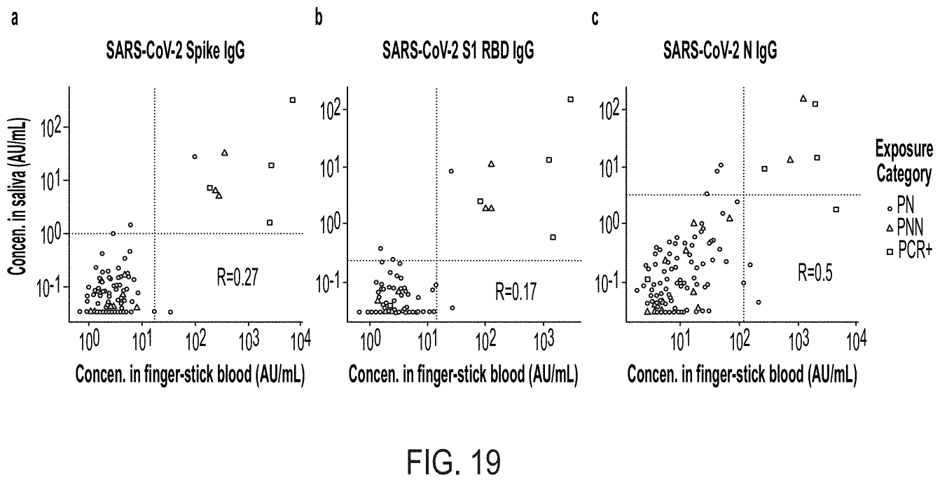

Correlations between the levels of antigen-specific IgG in saliva and blood samples are shown in FIG. 19. While the agreement between the two matrices for classification was strong, their correlation in levels was only moderate (R=0.25; p=0.005), which was consistent with previous studies (see, e.g., MacMullan et al., Scientific Reports 10:20808 (2020)). In 97.5% of donors with matched saliva and finger-stick blood, saliva and finger-stick blood measurements were concordant for classification of SARS-CoV-2 spike IgG and SARS-CoV-2 S1 RBD IgG levels as high or low relative to their matrix-specific thresholds (Cohen's κ=0.83; p=8.4e-18). 112 donors had low levels of SARS-CoV-2 Spike IgG for both saliva and finger-stick blood, and 8 donors had high levels of SARS-CoV-2 Spike IgG in both saliva and finger-stick blood. For the three discordant cases, two donors were slightly above the saliva threshold, and one donor was slightly above the finger-stick blood threshold. The concordance for the SARS-CoV-2 N IgG assays was 95.1% (Cohen's κ=0.64; p=2.5e-6).

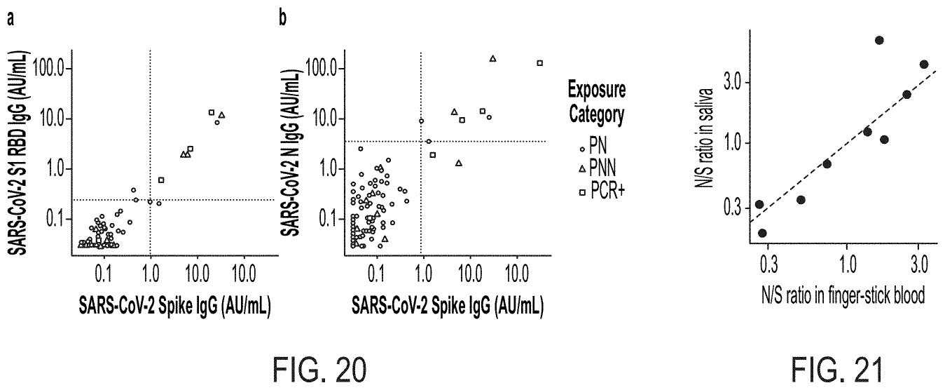

The salivary levels of antibodies to the full-length spike and RBD antigens were highly correlated (FIG. 20). Absolute signals for the full-length spike were higher than for the RBD antigen, which is expected since antibodies to the RBD are a subset of those binding to the full-length spike. The salivary levels of antibodies reactive with the N antigen were moderately correlated with antibodies for spike and RBD.

Because the relative immune responses to N versus S may be a clinically significant indicator of immune response, correlated the ratio of anti-N to anti-S levels measured in finger-stick blood versus saliva were correlated, as shown in FIG. 21. A strong correlation was found in the N to S ratio (R=0.95; p=0.001), which indicates that quantitative salivary measurements can be used to compute this ratio equivalently to finger-stick measurements.

### Transport of Samples by Mail

Due to the high stability of salivary antibodies at room temperature and without preservatives, a pilot study was performed in which donors mailed saliva specimens from Oklahoma to Maryland. Specimens (n=19) showed expected levels of antibodies to circulating coronaviruses. Although mailed-in samples spent up to two weeks in transit, the range of antibody concentrations for the circulating coronaviruses overlapped with that of locally collected samples. Anti-SARS-CoV-2 antibodies were detected only in the two samples from individuals who responded that they had previously been infected by SARS-CoV-2. Overall, this pilot study demonstrates the feasibility of a mailed test for salivary antibodies.

### DISCUSSION

This study demonstrated that self-collected saliva provides information similar to finger-stick blood even after the saliva is at ambient temperature for hours to days without preservatives. Consistent with prior studies that report a correlation between antibody levels in serum and plasma, very high concordance was observed between self-collected saliva and finger-stick blood in the identification of individuals with IgG reactivity to the RBD and full-length forms of CoV-2 spike protein.

The degree of correlation between finger-stick blood and saliva measurements (FIG. 19) depends on variations among individuals in the rate of antibody transit into the mouth, the rate of saliva flow diluting the antibody, and possibly other factors associated with the degree of compliance with instructions for sample collection such as delaying collection after eating or drinking. The almost perfect agreement we observed in the classification of serostatus using saliva and finger-stick blood suggests that the difference in the observed antibody activity in positive subjects vs. negative controls is large enough to compensate for the increased variability in saliva samples. The ratio of anti-N antibodies to anti-S antibodies in saliva and finger-stick blood (FIG. 21) correlates more strongly than the ratio of the absolute concentrations (FIG. 19), showing that the effect of variations among donors in salivary flow rates and rates of antibody transit can be reduced through normalization approaches.

### Example 14. SARS-CoV-2 Strain Identification

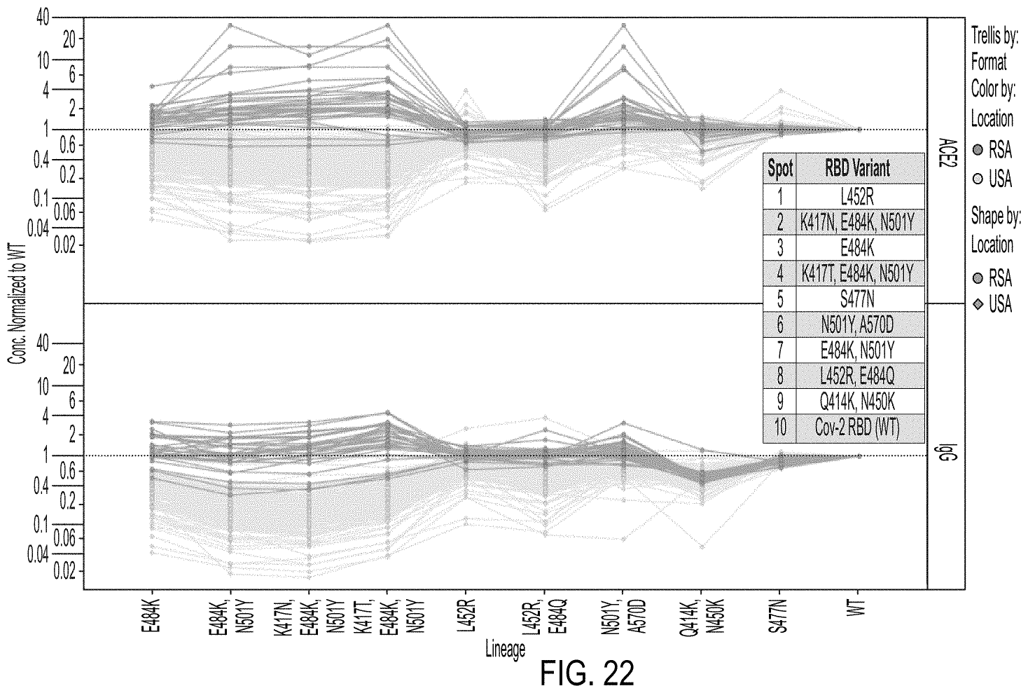

Samples from ˜200 individuals in the United States infected with wild-type SARS-CoV-2 (“Wuhan”) and 32 individuals in South Africa infected with SARS-CoV-2 strain 501Y.V2, also known as B.1.351, were tested using a 10-spot variant SARS-CoV-2 S-RBD panel as shown in FIG. 22, including the following mutations: (1) L452R; (2) K417N, E484K, and N501Y; (3) E484K; (4) K417T, E484K, and N501Y; (5) S477N; (6) N501Y and A570D; (7) E484K and N501Y; (8) L452R and E484Q; (9) Q414K and N450K mutations; and (10) wild-type SARS-CoV-2 S-RBD, wherein all mutations are relative to wild-type S-RBD from SARS-CoV-2. The panels were used in indirect IgG Serology and ACE2 competition assay formats as described in the previous Examples.

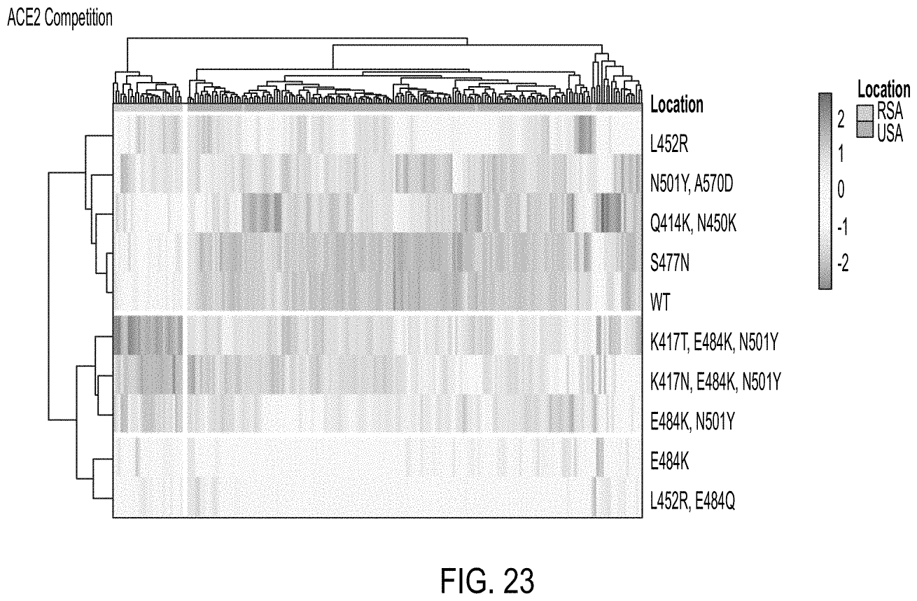

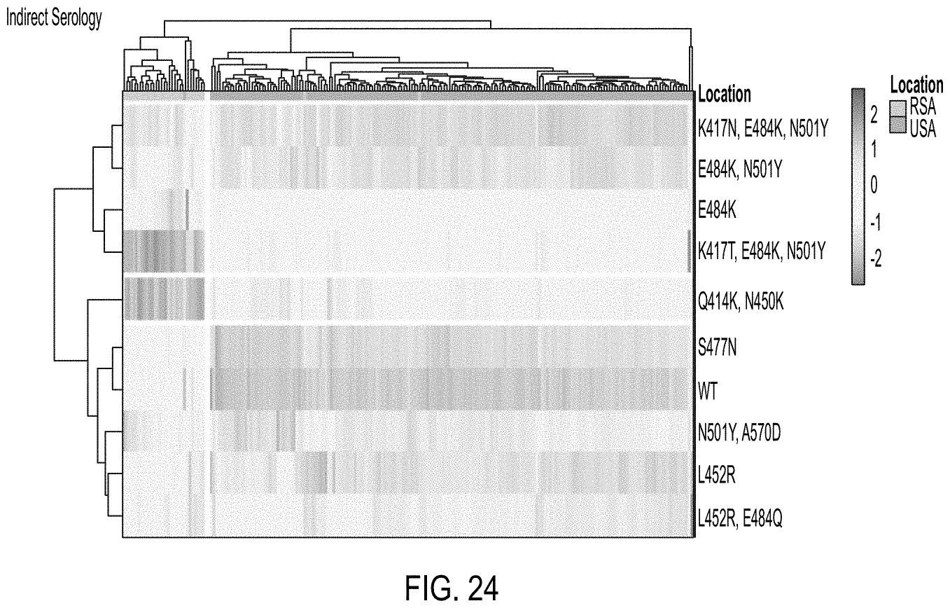

The results in FIG. 22 show the signals for each of the antigens after normalization to the signal from the wild-type SARS-CoV-2 S-RBD. Each set of connected dots shows the normalized signals from each antigen for one study subject. The upper figure shows results of the ACE-2 competition assay; the lower figure shows the serology results. The results show that the sample reactivity towards the wild-type Wuhan strain circulating in the United States at the time of sample collection can be clearly separated from the circulating B.1.351 strain in South Africa. This was further demonstrated by the heat maps from the same data in FIG. 23 (ACE2 competition assay format) and FIG. 24 (indirect IgG serology assay format), which shows a clear separation between the wild-type and B.1.351 strains.

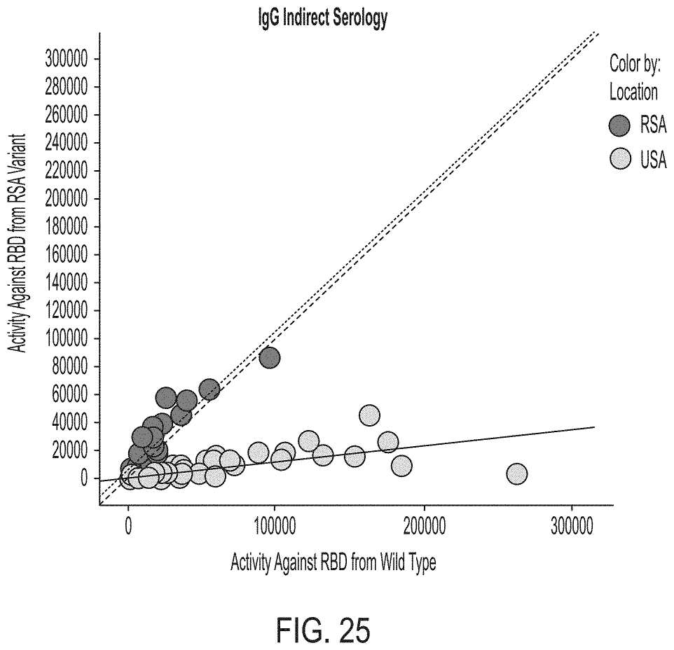

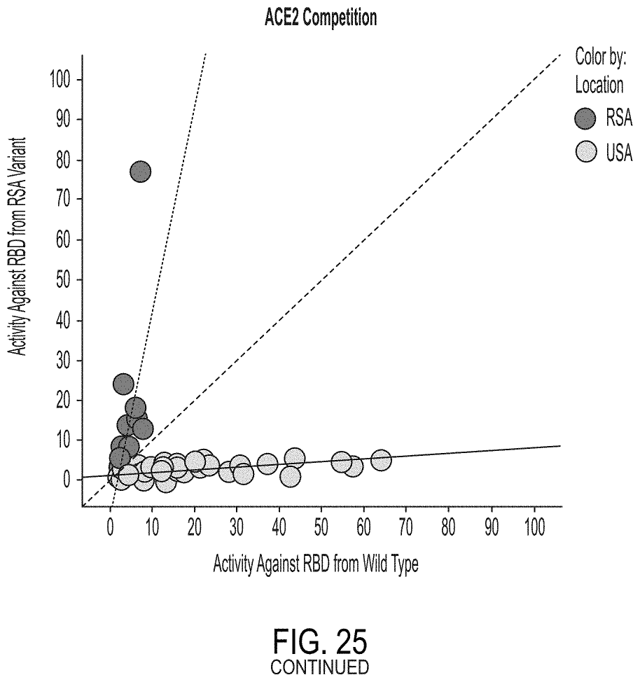

FIG. 25 shows a subset of the data in a different format: for each sample, assay signals for the wild-type SARS-CoV-2 S-RBD (x-axis) were plotted against the signals for the SARS-CoV-2 S-RBD from the B.1.351 strain. While absolute serology signals may vary from subject to subject, the ratio of the signals on the two spots remained remarkably consistent between subjects in one region (and presumably exposed to the same strain), as shown by the data points from each region falling on two different lines with two different slopes. The results in FIG. 25 show that while the signal from one spot may not be sufficient to distinguish between the wild type and B.1.351 SARS-CoV-2 infections, combining the results from two spots provided almost complete separation.

### Example 15. SARS-CoV-2 Serology Assay

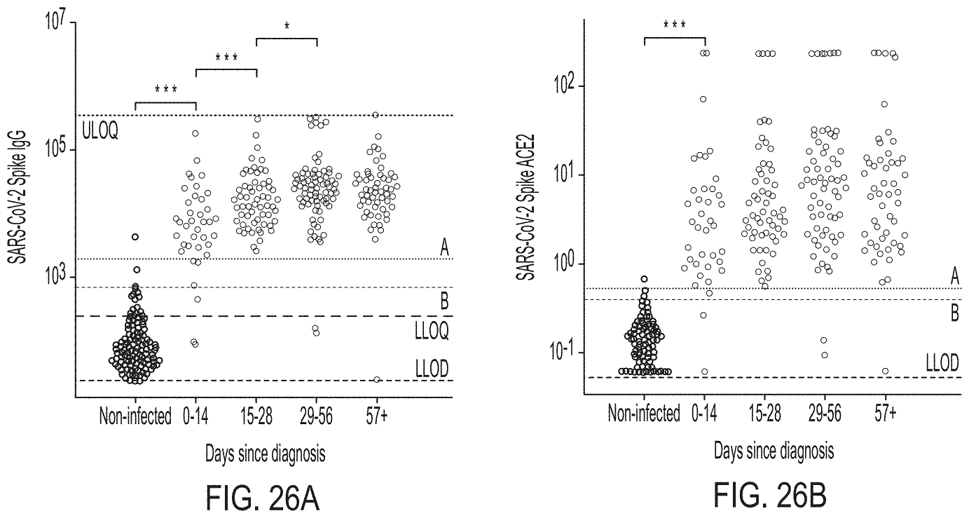

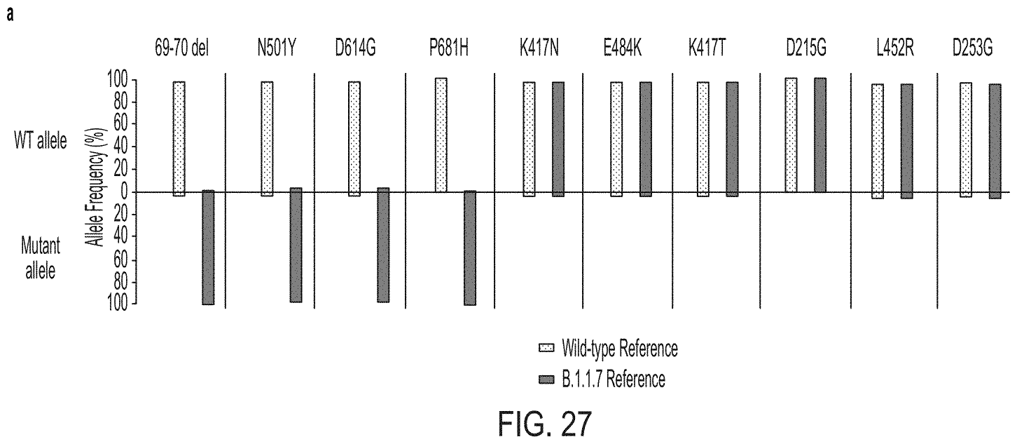

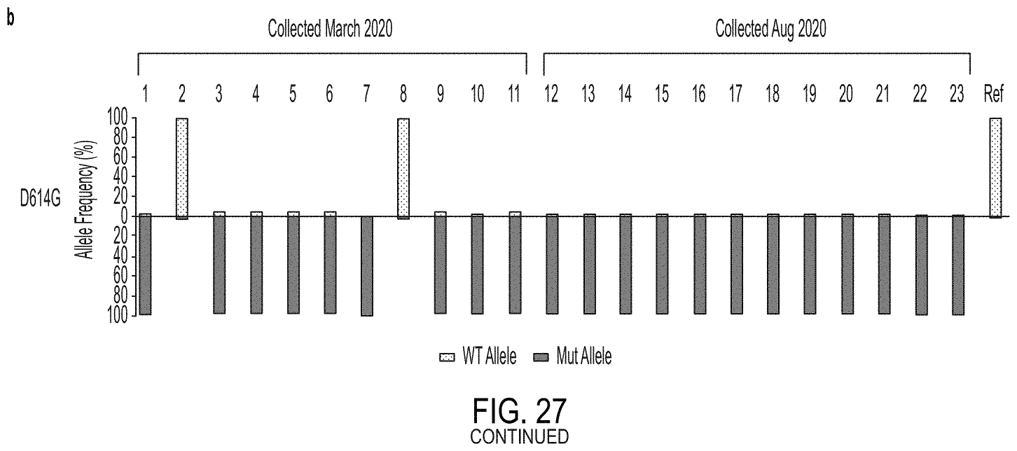

Indirect IgG serology and ACE2 competition assays were performed to measure antibodies against SARS-CoV-2 S protein in 214 serum samples collected from individuals at different time points after confirmed SARS-CoV-2 infection (diagnosis by PCR; 0-14 days, 15-28 days, 29-56 days, and 57+ days) and 200 control samples collected prior to the emergence of SARS-CoV-2 in 2020. The indirect IgG serology assay results are shown in FIG. 26A. The ACE2 competition assay results are shown in FIG. 26B. Both assays had point estimates for sensitivity of 98.3% for detecting infection 15+ days after onset, and point estimates for specificity of 99.5%. The assays also demonstrated good sensitivity for samples collected within the first two weeks after onset (84.2% for indirect serology and 92.1% for ACE2 competition).

### Example 16. SARS-CoV-2 SNP Detection

A multiplexed oligonucleotide ligation assay (OLA) was used to detect SNPs in 23 nasal swab samples from subjects who had previously tested positive for SARS-CoV-2. The mutations in the assay panels included the following: 69-70del, D215G, D253G, K417N, K417T, L452R, E484K, N501Y, D614G, and P681H. A wild-type reference SARS-CoV-2 genomic RNA sample was used for the lineage A control. A known lineage B.1.1.7 reference was also tested, which matched the known mutations as shown in FIG. 37, top panel.

The assay panels were used to assess a set of 23 samples that were collected in March or August of 2020. These samples were found not to contain the mutations in the panel, except for D614G, which was consistent with the fact that the mutations being assessed (other than D614G) were not commonly in circulation at those times. The one exception was the D614G mutation, which became prevalent very early on in the COVID-19 pandemic and is present in almost all samples. For D614G, 21/23 samples contained the D614G mutation, while the wild-type reference did not (FIG. 37, bottom panel).

### Example 17. Host Biomarker Detection in Serum and Cerebrospinal Fluid of COVID-19 Patients

Cerebrospinal fluid (CSF) and serum from acute COVID-19 patients were obtained and measured for the following panel of cytokines: IL-6, IL-10, IL-12p70, IL-4, TNF-α, IL-2, IL-1β, IFN-γ, and IL-17A. The cytokines were measured using the Format 2 immunoassay described in Example 7, i.e., each cytokine was detected using a detection reagent linked to a nucleic acid probe, wherein upon binding of the detection reagent to the cytokine, the nucleic acid probe is extended to form an extended sequence, and the extended sequence is contacted with a probe comprising a detectable label for detection.

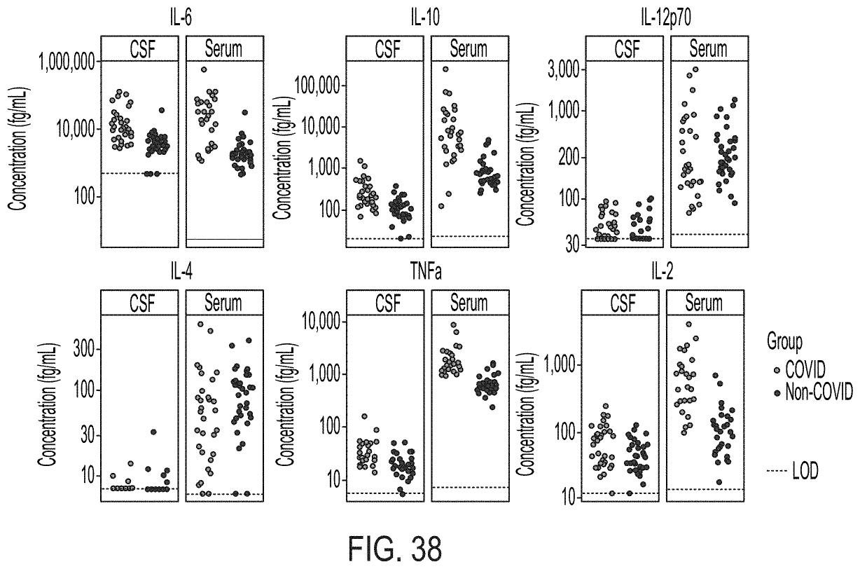

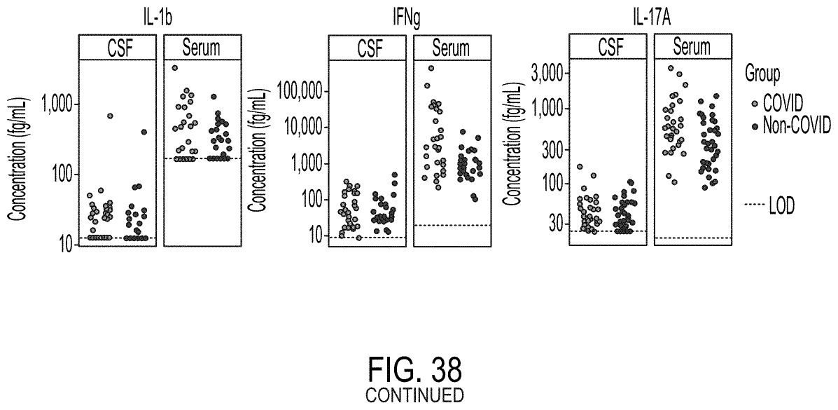

The measured cytokine levels from COVID-19 patients were compared against cytokine levels from non-COVID-19 control subjects. Results are shown in FIG. 38, with COVID-19 patients in light grey on the left side of each CSF or serum result panel, and non-COVID-19 control subjects in dark grey on the right side of each CSF or serum result panel. The results showed that IL-2, IL-6, IL-10, IFN-γ, and TNF-α levels in serum and CSF were generally higher in the acute COVID-19 patient group than the control group. In general, the cytokine concentrations in CSF were approximately an order of magnitude lower than in serum, except for IL-6, for which the concentrations in CSF and serum were comparable.

1. A method for determining a SARS-CoV-2 strain in a sample, comprising:
detecting at least a first antibody biomarker in the sample that binds to an antigen from a first SARS-CoV-2 strain and at least a second antibody biomarker in the sample that binds to an antigen from a second SARS-CoV-2 strain, wherein the detecting comprises contacting the sample with a surface comprising at least two binding domains, wherein the antigen from the first SARS-CoV-2 strain is immobilized on a first binding domain, and the antigen from the second SARS-CoV-2 strain is immobilized on a second binding domain; and
determining a ratio of the first antibody biomarker to the second antibody biomarker, thereby determining the SARS-CoV-2 strain.

2. The method of claim 1, wherein the method detects 1 to 10 distinct antibody biomarkers in the sample, wherein each antibody biomarker binds to an antigen from a unique SARS-CoV-2 strain, and wherein the antigen from each unique SARS-CoV-2 strain is immobilized on a distinct binding domain on the surface.

3. The method of claim 1, wherein the antigen comprises an S protein, an N protein, an S-RBD, or a combination thereof.

4. The method of claim 1, wherein each antigen is immobilized on a distinct binding domain on the surface, and wherein the antigens comprise:
an S protein, an S-RBD, and/or an N protein from a SARS-CoV-2 strain selected from:
wild-type; P.1; P.2; P.3; B.1.1.519; B.1.1.529; B.1.1.529 (+R346K); B.1.1.529 (+L452R); BA.1; BA.1.1; BA.2; BA.3; B.1.1.7; B.1.1.7 (+E484K); B.1.258.17; B.1.351; B.1.351.1; B.1.429; B.1.466.2; B.1.525; B.1.526/E484K; B.1.526/S477N; B.1.526.1; B.1.617; B.1.617.1; B.1.617.2; B.1.617.2 (+ΔY144); B.1.617.2 (+E484K); B.1.617.2 (+E484K/N501Y); B.1.617.2 (+K417N/N439K/E484K/N501Y); B.1.617.2 (+K417N/E484K/N501Y); AY.1; AY.2; AY.3, AY.4; AY.5, AY.6, AY.7, AY.4.2; AY.12; AY.14; B.1.617.3; B.1.618; B.1.620; B.1.621; B.1.640.2; BV-1; A.23.1; A.VOI.V2; C.37; and R.1; and/or
an S protein and/or an S-RBD from SARS-CoV-2 comprising one or more mutations selected from: R346K, V367F; Q414K, K417N, K417T, N439K, N450K, L452R, L452Q, S477N, T478K, T478R, E484K, E484Q, F490S, Q493R, N501Y.

5. The method of claim 4, wherein the antigen comprises an S-RBD comprising:
a V367F mutation; an N439K mutation; an L452R mutation; an S477N mutation; a T478K mutation, an E484K mutation; an N501Y mutation; L452R and E484Q mutations; L452R and T478K mutations; L452Q and F490S mutations; S477N and E484K mutations; E484K and N501Y mutations; Q414K and N450K mutations; Q493R and N501Y mutations; R346K, T478R, and E484K mutations; K417N, E484K, and N501Y mutations; K417N, L452R, and T478K mutations; or K417T, E484K, and N501Y mutations.

6. The method of claim 1, wherein the detecting comprises:
(a) forming a binding complex in each binding domain that comprises the antigen and an antibody biomarker that binds to the antigen;
(b) contacting the binding complex in each binding domain with a detection reagent; and
(c) detecting the binding complexes on the surface.

7. The method of claim 6, wherein the detection reagent comprises a detection antibody, a detection antigen, or an ACE detection reagent.

8. The method of claim 7, wherein the detection reagent comprises an electrochemiluminescent (ECL) label.

9. The method of claim 1, wherein the sample is a saliva sample.

10. The method of claim 1, wherein the sample is from one or more individuals, wherein the one or more individuals are currently infected or previously infected with SARS-CoV-2.

11. The method of claim 10, wherein the sample comprises a pooled sample from at least two individuals.

12. The method of claim 1, wherein the method further comprises comparing the SARS-CoV-2 strain from one or more samples from one or more individuals located in one or more geographical regions, thereby tracking spread of the SARS-CoV-2 strain in the one or more geographical regions.

13. The method of claim 1, wherein the method further comprises comparing the SARS-CoV-2 strain from one or more samples from one or more individuals obtained at different time points, thereby tracking spread of the SARS-CoV-2 strain over time.

14. The method of claim 1, wherein the SARS-CoV-2 strain is determined by inputting the ratio of the first antibody biomarker to the second antibody biomarker into a classification algorithm.

15. The method of claim 14, further comprising training the classification algorithm, wherein the training comprises:
measuring the amount of antibody biomarkers in a sample from a subject infected with a known SARS-CoV-2 strain that bind to an antigen from one or more SARS-CoV-2 strains, wherein the one or more SARS-CoV-2 strains comprise the known SARS-CoV-2 strain;
normalizing the amount of measured antibody biomarker that bind to an antigen from the known SARS-CoV-2 strain against the amount of measured antibody biomarker that bind to an antigen from a further SARS-CoV-2 strain; and
providing the normalized antibody biomarker amount to the classification algorithm.

16. A method for determining a SARS-CoV-2 strain in a sample, comprising:
(a) detecting at least a first antibody biomarker in the sample that binds to an antigen from a first SARS-CoV-2 strain and at least a second antibody biomarker in the sample that binds to an antigen from a second SARS-CoV-2 strain, wherein the detecting comprises contacting the sample with a surface comprising at least two binding domains, wherein the antigen from the first SARS-CoV-2 strain is immobilized on a first binding domain, and the antigen from the second SARS-CoV-2 strain is immobilized on a second binding domain;
wherein each antigen is immobilized on a distinct binding domain on the surface, and wherein the antigens comprise:
an S protein, an S-RBD, and/or an N protein from a SARS-CoV-2 strains selected from: wild-type; P.1; P.2; P.3; B.1.1.519; B.1.1.529; B.1.1.529 (+R346K); B.1.1.529 (+L452R); BA.1; BA.1.1; BA.2; BA.3; B.1.1.7; B.1.1.7 (+E484K); B.1.258.17; B.1.351; B.1.351.1; B.1.429; B.1.466.2; B.1.525; B.1.526/E484K; B.1.526/S477N; B.1.526.1; B.1.617; B.1.617.1; B.1.617.2; B.1.617.2 (+AY144); B.1.617.2 (+E484K); B.1.617.2 (+E484K/N501Y); B.1.617.2 (+K417N/N439K/E484K/N501Y); B.1.617.2 (+K417N/E484K/N501Y); AY.1; AY.2; AY.3, AY.4; AY.5, AY.6, AY.7, AY.4.2; AY.12; AY.14; B.1.617.3; B.1.618; B.1.620; B.1.621; B.1.640.2; BV-1; A.23.1; A.VOI.V2; C.37; and R.1; and/or
an S protein and/or an S-RBD from SARS-CoV-2 comprising one or more mutations selected from: R346K, V367F; Q414K, K417N, K417T, N439K, N450K, L452R, L452Q, S477N, T478K, T478R, E484K, E484Q, F490S, Q493R, N501Y, wherein the sample is from one or more individuals, wherein the one or more individuals are currently infected or previously infected with SARS-CoV-2, and optionally wherein the one or more individuals are located in one or more geographical regions and/or the samples are obtained at different times;
(b) determining a ratio of the first antibody biomarker to the second antibody biomarker;
(c) inputting the ratio of the first antibody biomarker to the second antibody biomarker into a classification algorithm, wherein the classification algorithm is trained by a training method comprising:
measuring the amount of antibody biomarkers in a sample from a subject infected with a known SARS-CoV-2 strain that bind to an antigen from at least two SARS-CoV-2 strains, wherein the at least two SARS-CoV-2 strains comprise the known SARS-CoV-2 strain and a further SARS-CoV-2 strain;
normalizing the amount of measured antibody biomarker that bind to an antigen from the known SARS-CoV-2 strain against the amount of measured antibody biomarker that bind to an antigen from the further SARS-CoV-2 strain; and
providing the normalized antibody biomarker amount to the classification algorithm;
(d) determining the SARS-CoV-2 strain based on the classification algorithm; and
(e) optionally, tracking spread of the SARS-CoV-2 strain in the one or more geographical region, tracking spread of the SARS-CoV-2 strain over time, or a combination thereof.

17. A method for detecting a single nucleotide polymorphism (SNP) in a target nucleic acid, wherein the target nucleic acid is a SARS-CoV-2 nucleic acid, comprising:
(a) contacting a sample comprising the target nucleic acid with (i) a targeting probe, wherein the targeting probe comprises a first region complementary to a polymorphic site of the target nucleic acid that comprises the SNP, and wherein the targeting probe comprises an oligonucleotide tag; and (ii) a detection probe, wherein the detection probe comprises a second region complementary to an adjacent region of the target nucleic acid comprising the polymorphic site, and wherein the detection probe comprises a detectable label, wherein the targeting probe and the detection probe each independently comprises a sequence as shown in Table 10 or Table 14;
(b) hybridizing the targeting and detection probes to the target nucleic acid;
(c) ligating the targeting and detection probes that hybridize with perfect complementarity at the polymorphic site to form a ligated target complement comprising the oligonucleotide tag and the detectable label;
(d) contacting the product of (c) with a surface comprising an immobilized binding reagent, wherein the binding reagent comprises an oligonucleotide complementary to the oligonucleotide tag;
(e) forming a binding complex on the surface, wherein the binding complex comprises the binding reagent and the ligated target complement; and
(f) detecting the binding complex, thereby detecting the SNP at the polymorphic site.

18. The method of claim 17, wherein the targeting probe hybridizes to the target nucleic acid such that a terminal 5′ nucleotide of the targeting probe hybridizes with the SNP, and the detection probe hybridizes to the target nucleic acid adjacent to the SNP and provides a 3′ end for ligating the targeting and the detection probes; or
wherein the detection probe hybridizes to the target nucleic acid such that a terminal 5′ nucleotide of the detection probe hybridizes with the SNP, and the targeting probe hybridizes to the target nucleic acid adjacent to the SNP and provides a 3′ end for ligating the targeting and the detection probes; or
wherein the detection probe hybridizes to the target nucleic acid such that a terminal 3′ nucleotide of the detection probe hybridizes with the SNP, and the targeting probe hybridizes to the target nucleic acid adjacent to the SNP and provides a 5′ end for ligating the targeting and the detection probes.

19. (canceled)

20. (canceled)

21. The method of claim 1, further comprising providing a blocking probe during the ligating, wherein the blocking probe comprises a sequence as shown in Table 12 or Table 16.

22. The method of claim 1, wherein the detectable label comprises an ECL label.


SEQUENCE LISTING (758 sequences)

SEQ ID NO: 1 - DNA, 44 bp, Artificial Sequence
  Synthetic probe
actggtaacccagacatgatcggttataactaccaccacgctga

SEQ ID NO: 2 - DNA, 44 bp, Artificial Sequence
  Synthetic probe
actccctgtgggtgagcttaatggtataactaccaccacgctgg

SEQ ID NO: 3 - DNA, 51 bp, Artificial Sequence
  Synthetic probe
catttggtcattggttcaagacgagttcctggcaattaattgtaaaaggtg

SEQ ID NO: 4 - DNA, 51 bp, Artificial Sequence
  Synthetic probe
ctggtccgttgtggtccttctaacgttcctggcaattaattgtaaaaggta

SEQ ID NO: 5 - DNA, 26 bp, Artificial Sequence
  Synthetic probe
ctaaaccatgtgtcaaaatcagcatg

SEQ ID NO: 6 - DNA, 28 bp, Artificial Sequence
  Synthetic probe
aacaggaaactgtataattaccgatatc

SEQ ID NO: 7 - DNA, 20 bp, Artificial Sequence
  Synthetic oligonucleotide
tataactaccaccacgctgr

SEQ ID NO: 8 - DNA, 26 bp, Artificial Sequence
  Synthetic oligonucleotide
ctaaaccatgtgtcaaaatcagcatg

SEQ ID NO: 9 - DNA, 27 bp, Artificial Sequence
  Synthetic oligonucleotide
gttcctggcaattaattgtaaaaggtr

SEQ ID NO: 10 - DNA, 28 bp, Artificial Sequence
  Synthetic oligonucleotide
aacaggaaactgtataattaccgatatc

SEQ ID NO: 11 - DNA, 21 bp, Artificial Sequence
  Synthetic primer
aggctattgatggtggtgtca

SEQ ID NO: 12 - DNA, 20 bp, Artificial Sequence
  Synthetic primer
gcacgacaaaacccacttct

SEQ ID NO: 13 - DNA, 20 bp, Artificial Sequence
  Synthetic primer
cgtggatgaggctggttcta

SEQ ID NO: 14 - DNA, 20 bp, Artificial Sequence
  Synthetic primer
tttcgctgattttggggtcc

SEQ ID NO: 15 - DNA, 46 bp, Artificial Sequence
  Synthetic probe
ctaatagctcctgtgccctcgtatgggacttctgtgcagttaacat

SEQ ID NO: 16 - DNA, 46 bp, Artificial Sequence
  Synthetic probe
aatccgtcgactagcctgagaattgggacttctgtgcagttaacac

SEQ ID NO: 17 - DNA, 23 bp, Artificial Sequence
  Synthetic probe
cctgataaagaacagcaacctgg

SEQ ID NO: 18 - DNA, 22 bp, Artificial Sequence
  Synthetic oligonucleotide
gggacttctgtgcagttaacay

SEQ ID NO: 19 - DNA, 23 bp, Artificial Sequence
  Synthetic oligonucleotide
cctgataaagaacagcaacctgg

SEQ ID NO: 20 - DNA, 20 bp, Artificial Sequence
  Synthetic primer
acactactgatgctgtccgt

SEQ ID NO: 21 - DNA, 20 bp, Artificial Sequence
  Synthetic primer
gcccctattaaacagcctgc

SEQ ID NO: 22 - DNA, 44 bp, Artificial Sequence
  Synthetic probe
gtggcaacagaaatcaggtggtgacgaaatgcaccccgcattac

SEQ ID NO: 23 - DNA, 21 bp, Artificial Sequence
  Synthetic probe
gtttggtggaccctcagattc

SEQ ID NO: 24 - DNA, 45 bp, Artificial Sequence
  Synthetic probe
gtggcaacagaaatcaggtggtgagaatctgagggtccaccaaac

SEQ ID NO: 25 - DNA, 45 bp, Artificial Sequence
  Synthetic probe
tagatgccgctgcaggtatggaaagaatctgagggtccaccaaac

SEQ ID NO: 26 - DNA, 20 bp, Artificial Sequence
  Synthetic probe
gtaatgcggggtgcatttcg

SEQ ID NO: 27 - DNA, 45 bp, Artificial Sequence
  Synthetic probe
tagatgccgctgcaggtatggaaagcaaattgcacaatttgcccc

SEQ ID NO: 28 - DNA, 18 bp, Artificial Sequence
  Synthetic probe
cagcgcttcagcgttctt

SEQ ID NO: 29 - DNA, 41 bp, Artificial Sequence
  Synthetic probe
tagatgccgctgcaggtatggaaagaacgctgaagcgctgg

SEQ ID NO: 30 - DNA, 21 bp, Artificial Sequence
  Synthetic probe
gggcaaattgtgcaatttgcg

SEQ ID NO: 31 - DNA, 45 bp, Artificial Sequence
  Synthetic probe
agaggactgctaaaggtttgtaggccaaaagaycacattggcacc

SEQ ID NO: 32 - DNA, 22 bp, Artificial Sequence
  Synthetic probe
cgcaatcctgctaacaatgctg

SEQ ID NO: 33 - DNA, 45 bp, Artificial Sequence
  Synthetic probe
agaggactgctaaaggtttgtagggcattgttagcaggattgcgg

SEQ ID NO: 34 - DNA, 22 bp, Artificial Sequence
  Synthetic probe
gtgccaatgtgrtcttttggtg

SEQ ID NO: 35 - DNA, 42 bp, Artificial Sequence
  Synthetic probe
cgtaccattgaatctggagaccttgagcgggttctgacctga

SEQ ID NO: 36 - DNA, 16 bp, Artificial Sequence
  Synthetic probe
aggctctgcgcggact

SEQ ID NO: 37 - DNA, 41 bp, Artificial Sequence
  Synthetic probe
cgtaccattgaatctggagaccttcaagtccgcgcagagcc

SEQ ID NO: 38 - DNA, 18 bp, Artificial Sequence
  Synthetic probe
ttcaggtcagaacccgct

SEQ ID NO: 39 - DNA, 20 bp, Artificial Sequence
  Synthetic oligonucleotide
cgaaatgcaccccgcattac

SEQ ID NO: 40 - DNA, 21 bp, Artificial Sequence
  Synthetic oligonucleotide
gtttggtggaccctcagattc

SEQ ID NO: 41 - DNA, 21 bp, Artificial Sequence
  Synthetic oligonucleotide
gaatctgagggtccaccaaac

SEQ ID NO: 42 - DNA, 20 bp, Artificial Sequence
  Synthetic oligonucleotide
gtaatgcggggtgcatttcg

SEQ ID NO: 43 - DNA, 21 bp, Artificial Sequence
  Synthetic oligonucleotide
gcaaattgcacaatttgcccc

SEQ ID NO: 44 - DNA, 18 bp, Artificial Sequence
  Synthetic oligonucleotide
cagcgcttcagcgttctt

SEQ ID NO: 45 - DNA, 17 bp, Artificial Sequence
  Synthetic oligonucleotide
gaacgctgaagcgctgg

SEQ ID NO: 46 - DNA, 21 bp, Artificial Sequence
  Synthetic oligonucleotide
gggcaaattgtgcaatttgcg

SEQ ID NO: 47 - DNA, 21 bp, Artificial Sequence
  Synthetic oligonucleotide
ccaaaagaycacattggcacc

SEQ ID NO: 48 - DNA, 22 bp, Artificial Sequence
  Synthetic oligonucleotide
cgcaatcctgctaacaatgctg

SEQ ID NO: 49 - DNA, 21 bp, Artificial Sequence
  Synthetic oligonucleotide
gcattgttagcaggattgcgg

SEQ ID NO: 50 - DNA, 22 bp, Artificial Sequence
  Synthetic oligonucleotide
gtgccaatgtgrtcttttggtg

SEQ ID NO: 51 - DNA, 18 bp, Artificial Sequence
  Synthetic oligonucleotide
gagcgggttctgacctga

SEQ ID NO: 52 - DNA, 16 bp, Artificial Sequence
  Synthetic oligonucleotide
aggctctgcgcggact

SEQ ID NO: 53 - DNA, 17 bp, Artificial Sequence
  Synthetic oligonucleotide
caagtccgcgcagagcc

SEQ ID NO: 54 - DNA, 18 bp, Artificial Sequence
  Synthetic oligonucleotide
ttcaggtcagaacccgct

SEQ ID NO: 55 - DNA, 44 bp, Artificial Sequence
  Synthetic primer
aatccgtcgactagcctgagaattgaccccaaaatcagcgaaat

SEQ ID NO: 56 - DNA, 24 bp, Artificial Sequence
  Synthetic primer
tctggttactgccagttgaatctg

SEQ ID NO: 57 - DNA, 24 bp, Artificial Sequence
  Synthetic probe
accccgcattacgtttggtggacc

SEQ ID NO: 58 - DNA, 44 bp, Artificial Sequence
  Synthetic primer
agaggactgctaaaggtttgtaggttacaaacattggccgcaaa

SEQ ID NO: 59 - DNA, 18 bp, Artificial Sequence
  Synthetic primer
gcgcgacattccgaagaa

SEQ ID NO: 60 - DNA, 23 bp, Artificial Sequence
  Synthetic probe
acaatttgcccccagcgcttcag

SEQ ID NO: 61 - DNA, 46 bp, Artificial Sequence
  Synthetic primer
cgtaccattgaatctggagaccttgggagccttgaatacaccaaaa

SEQ ID NO: 62 - DNA, 21 bp, Artificial Sequence
  Synthetic primer
tgtagcacgattgcagcattg

SEQ ID NO: 63 - DNA, 24 bp, Artificial Sequence
  Synthetic probe
aycacattggcacccgcaatcctg

SEQ ID NO: 64 - DNA, 43 bp, Artificial Sequence
  Synthetic primer
actggtaacccagacatgatcggtagatttggacctgcgagcg

SEQ ID NO: 65 - DNA, 20 bp, Artificial Sequence
  Synthetic primer
gagcggctgtctccacaagt

SEQ ID NO: 66 - DNA, 23 bp, Artificial Sequence
  Synthetic probe
ttctgacctgaaggctctgcgcg

SEQ ID NO: 67 - DNA, 20 bp, Artificial Sequence
  Synthetic primer
gaccccaaaatcagcgaaat

SEQ ID NO: 68 - DNA, 24 bp, Artificial Sequence
  Synthetic primer
tctggttactgccagttgaatctg

SEQ ID NO: 69 - DNA, 20 bp, Artificial Sequence
  Synthetic primer
ttacaaacattggccgcaaa

SEQ ID NO: 70 - DNA, 18 bp, Artificial Sequence
  Synthetic primer
gcgcgacattccgaagaa

SEQ ID NO: 71 - DNA, 22 bp, Artificial Sequence
  Synthetic primer
gggagccttgaatacaccaaaa

SEQ ID NO: 72 - DNA, 21 bp, Artificial Sequence
  Synthetic primer
tgtagcacgattgcagcattg

SEQ ID NO: 73 - DNA, 19 bp, Artificial Sequence
  Synthetic primer
agatttggacctgcgagcg

SEQ ID NO: 74 - DNA, 20 bp, Artificial Sequence
  Synthetic primer
gagcggctgtctccacaagt

SEQ ID NO: 75 - DNA, 49 bp, Artificial Sequence
  Synthetic probe
actccctgtgggtgagcttaatgggttacttggttccatgctatacatg

SEQ ID NO: 76 - DNA, 48 bp, Artificial Sequence
  Synthetic probe
actggtaacccagacatgatcggtccaatgttacttggttccatgcta

SEQ ID NO: 77 - DNA, 23 bp, Artificial Sequence
  Synthetic probe
tctctgggaccaatggtactaag

SEQ ID NO: 78 - DNA, 46 bp, Artificial Sequence
  Synthetic probe
actccctgtgggtgagcttaatggccattggtcccagagacatgta

SEQ ID NO: 79 - DNA, 47 bp, Artificial Sequence
  Synthetic probe
actggtaacccagacatgatcggtcttagtaccattggtcccagaga

SEQ ID NO: 80 - DNA, 24 bp, Artificial Sequence
  Synthetic probe
tagcatggaaccaagtaacattgg

SEQ ID NO: 81 - DNA, 48 bp, Artificial Sequence
  Synthetic probe
catttggtcattggttcaagacgaaatcatatggtttccaacccacta

SEQ ID NO: 82 - DNA, 48 bp, Artificial Sequence
  Synthetic probe
ctggtccgttgtggtccttctaacaatcatatggtttccaacccactt

SEQ ID NO: 83 - DNA, 23 bp, Artificial Sequence
  Synthetic probe
atggtgttggttaccaaccatac

SEQ ID NO: 84 - DNA, 47 bp, Artificial Sequence
  Synthetic probe
catttggtcattggttcaagacgatatggttggtaaccaacaccatt

SEQ ID NO: 85 - DNA, 47 bp, Artificial Sequence
  Synthetic probe
ctggtccgttgtggtccttctaactatggttggtaaccaacaccata

SEQ ID NO: 86 - DNA, 24 bp, Artificial Sequence
  Synthetic probe
agtgggttggaaaccatatgattg

SEQ ID NO: 87 - DNA, 52 bp, Artificial Sequence
  Synthetic probe
gtggcaacagaaatcaggtggtgactagttatcagactcagactaattctcc

SEQ ID NO: 88 - DNA, 52 bp, Artificial Sequence
  Synthetic probe
agaggactgctaaaggtttgtaggctagttatcagactcagactaattctca

SEQ ID NO: 89 - DNA, 15 bp, Artificial Sequence
  Synthetic probe
tcggcgggcacgtag

SEQ ID NO: 90 - DNA, 40 bp, Artificial Sequence
  Synthetic probe
gtggcaacagaaatcaggtggtgactacgtgcccgccgag

SEQ ID NO: 91 - DNA, 40 bp, Artificial Sequence
  Synthetic probe
agaggactgctaaaggtttgtaggctacgtgcccgccgat

SEQ ID NO: 92 - DNA, 28 bp, Artificial Sequence
  Synthetic probe
gagaattagtctgagtctgataactagc

SEQ ID NO: 93 - DNA, 54 bp, Artificial Sequence
  Synthetic probe
ctggtccgttgtggtccttctaacggaaaacagggtaatttcaaaaatcttagg

SEQ ID NO: 94 - DNA, 54 bp, Artificial Sequence
  Synthetic probe
catttggtcattggttcaagacgaggaaaacagggtaatttcaaaaatcttagt

SEQ ID NO: 95 - DNA, 40 bp, Artificial Sequence
  Synthetic probe
gaatttgtgtttaagaatattgatggttattttaaaatat

SEQ ID NO: 96 - DNA, 59 bp, Artificial Sequence
  Synthetic probe
ctggtccgttgtggtccttctaactaaaataaccatcaatattcttaaacacaaattcc

SEQ ID NO: 97 - DNA, 59 bp, Artificial Sequence
  Synthetic probe
catttggtcattggttcaagacgataaaataaccatcaatattcttaaacacaaattca

SEQ ID NO: 98 - DNA, 30 bp, Artificial Sequence
  Synthetic probe
ctaagatttttgaaattaccctgttttcct

SEQ ID NO: 99 - DNA, 47 bp, Artificial Sequence
  Synthetic probe
aatccgtcgactagcctgagaattacgcctattaatttagtgcgtga

SEQ ID NO: 100 - DNA, 47 bp, Artificial Sequence
  Synthetic probe
ctaatagctcctgtgccctcgtatacgcctattaatttagtgcgtgg

SEQ ID NO: 101 - DNA, 20 bp, Artificial Sequence
  Synthetic probe
tctccctcagggtttttcgg

SEQ ID NO: 102 - DNA, 45 bp, Artificial Sequence
  Synthetic probe
aatccgtcgactagcctgagaattccgaaaaaccctgagggagat

SEQ ID NO: 103 - DNA, 45 bp, Artificial Sequence
  Synthetic probe
ctaatagctcctgtgccctcgtatccgaaaaaccctgagggagac

SEQ ID NO: 104 - DNA, 23 bp, Artificial Sequence
  Synthetic probe
cacgcactaaattaataggcgtg

SEQ ID NO: 105 - DNA, 55 bp, Artificial Sequence
  Synthetic probe
agaggactgctaaaggtttgtaggattctaaggttggtggtaattataattacct

SEQ ID NO: 106 - DNA, 55 bp, Artificial Sequence
  Synthetic probe
gtggcaacagaaatcaggtggtgaattctaaggttggtggtaattataattaccg

SEQ ID NO: 107 - DNA, 32 bp, Artificial Sequence
  Synthetic probe
gtatagattgtttaggaagtctaatctcaaac

SEQ ID NO: 108 - DNA, 57 bp, Artificial Sequence
  Synthetic probe
agaggactgctaaaggtttgtagggtttgagattagacttcctaaacaatctataca

SEQ ID NO: 109 - DNA, 57 bp, Artificial Sequence
  Synthetic probe
gtggcaacagaaatcaggtggtgagtttgagattagacttcctaaacaatctatacc

SEQ ID NO: 110 - DNA, 30 bp, Artificial Sequence
  Synthetic probe
ggtaattataattaccaccaaccttagaat

SEQ ID NO: 111 - DNA, 46 bp, Artificial Sequence
  Synthetic probe
actggtaacccagacatgatcggtagcacaccttgtaatggtgttg

SEQ ID NO: 112 - DNA, 46 bp, Artificial Sequence
  Synthetic probe
actccctgtgggtgagcttaatggagcacaccttgtaatggtgtta

SEQ ID NO: 113 - DNA, 31 bp, Artificial Sequence
  Synthetic probe
aaggttttaattgttactttcctttacaatc

SEQ ID NO: 114 - DNA, 57 bp, Artificial Sequence
  Synthetic probe
actggtaacccagacatgatcggttgattgtaaaggaaagtaacaattaaaaccttc

SEQ ID NO: 115 - DNA, 57 bp, Artificial Sequence
  Synthetic probe
actccctgtgggtgagcttaatggtgattgtaaaggaaagtaacaattaaaaccttt

SEQ ID NO: 116 - DNA, 22 bp, Artificial Sequence
  Synthetic probe
aacaccattacaaggtgtgcta

SEQ ID NO: 117 - DNA, 47 bp, Artificial Sequence
  Synthetic probe
tagatgccgctgcaggtatggaaagcctacactatgtcacttggtgc

SEQ ID NO: 118 - DNA, 47 bp, Artificial Sequence
  Synthetic probe
cgtaccattgaatctggagaccttgcctacactatgtcacttggtgt

SEQ ID NO: 119 - DNA, 31 bp, Artificial Sequence
  Synthetic probe
agaaaattcagttgcttactctaataactct

SEQ ID NO: 120 - DNA, 56 bp, Artificial Sequence
  Synthetic probe
tagatgccgctgcaggtatggaaaagagttattagagtaagcaactgaattttctg

SEQ ID NO: 121 - DNA, 56 bp, Artificial Sequence
  Synthetic probe
cgtaccattgaatctggagaccttagagttattagagtaagcaactgaattttcta

SEQ ID NO: 122 - DNA, 22 bp, Artificial Sequence
  Synthetic probe
caccaagtgacatagtgtaggc

SEQ ID NO: 123 - DNA, 45 bp, Artificial Sequence
  Synthetic probe
tagatgccgctgcaggtatggaaagctccagggcaaactggaamg

SEQ ID NO: 124 - DNA, 45 bp, Artificial Sequence
  Synthetic probe
cgtaccattgaatctggagaccttgctccagggcaaactggaamt

SEQ ID NO: 125 - DNA, 38 bp, Artificial Sequence
  Synthetic probe
attgctgattataattataaattaccagatgattttac

SEQ ID NO: 126 - DNA, 63 bp, Artificial Sequence
  Synthetic probe
tagatgccgctgcaggtatggaaagtaaaatcatctggtaatttataattataatcagcaatc

SEQ ID NO: 127 - DNA, 63 bp, Artificial Sequence
  Synthetic probe
cgtaccattgaatctggagaccttgtaaaatcatctggtaatttataattataatcagcaata

SEQ ID NO: 128 - DNA, 20 bp, Artificial Sequence
  Synthetic probe
kttccagtttgccctggagc

SEQ ID NO: 129 - DNA, 44 bp, Artificial Sequence
  Synthetic probe
ctggtccgttgtggtccttctaacgctccagggcaaactggaaa

SEQ ID NO: 130 - DNA, 44 bp, Artificial Sequence
  Synthetic probe
catttggtcattggttcaagacgagctccagggcaaactggaac

SEQ ID NO: 131 - DNA, 39 bp, Artificial Sequence
  Synthetic probe
kattgctgattataattataaattaccagatgattttac

SEQ ID NO: 132 - DNA, 59 bp, Artificial Sequence
  Synthetic probe
ctggtccgttgtggtccttctaacatcatctggtaatttataattataatcagcaatmt

SEQ ID NO: 133 - DNA, 59 bp, Artificial Sequence
  Synthetic probe
catttggtcattggttcaagacgaatcatctggtaatttataattataatcagcaatmg

SEQ ID NO: 134 - DNA, 19 bp, Artificial Sequence
  Synthetic probe
ttccagtttgccctggagc

SEQ ID NO: 135 - DNA, 75 bp, Unknown
  WT template strand
cctttcttttccaatgttacttggttccatgctatacatgtctctgggaccaatggtactaagaggtttgataac

SEQ ID NO: 136 - DNA, 75 bp, Artificial Sequence
  Synthetic oligonucleotide
acctttcttttccaatgttacttggttccatgctatctctgggaccaatggtactaagaggtttgataaccctgt

SEQ ID NO: 137 - DNA, 75 bp, Unknown
  WT cDNA strand
ttatcaaacctcttagtaccattggtcccagagacatgtatagcatggaaccaagtaacattggaaaagaaaggt

SEQ ID NO: 138 - DNA, 75 bp, Artificial Sequence
  Synthetic oligonucleotide
agggttatcaaacctcttagtaccattggtcccagagatagcatggaaccaagtaacattggaaaagaaaggtaa

SEQ ID NO: 139 - DNA, 75 bp, Artificial Sequence
  Synthetic oligonucleotide
ttactttcctttacaatcatatggtttccaacccactaatggtgttggttaccaaccatacagagtagtagtact

SEQ ID NO: 140 - DNA, 75 bp, Artificial Sequence
  Synthetic oligonucleotide
ttactttcctttacaatcatatggtttccaacccacttatggtgttggttaccaaccatacagagtagtagtact

SEQ ID NO: 141 - DNA, 75 bp, Artificial Sequence
  Synthetic oligonucleotide
agtactactactctgtatggttggtaaccaacaccattagtgggttggaaaccatatgattgtaaaggaaagtaa

SEQ ID NO: 142 - DNA, 75 bp, Artificial Sequence
  Synthetic oligonucleotide
agtactactactctgtatggttggtaaccaacaccataagtgggttggaaaccatatgattgtaaaggaaagtaa

SEQ ID NO: 143 - DNA, 75 bp, Artificial Sequence
  Synthetic oligonucleotide
aggtatatgcgctagttatcagactcagactaattctcctcggcgggcacgtagtgtagctagtcaatccatcat

SEQ ID NO: 144 - DNA, 75 bp, Artificial Sequence
  Synthetic oligonucleotide
aggtatatgcgctagttatcagactcagactaattctcatcggcgggcacgtagtgtagctagtcaatccatcat

SEQ ID NO: 145 - DNA, 75 bp, Artificial Sequence
  Synthetic oligonucleotide
atgatggattgactagctacactacgtgcccgccgaggagaattagtctgagtctgataactagcgcatatacct

SEQ ID NO: 146 - DNA, 75 bp, Artificial Sequence
  Synthetic oligonucleotide
atgatggattgactagctacactacgtgcccgccgatgagaattagtctgagtctgataactagcgcatatacct

SEQ ID NO: 147 - DNA, 78 bp, Artificial Sequence
  Synthetic oligonucleotide
accttgaaggaaaacagggtaatttcaaaaatcttagggaatttgtgtttaagaatattgatggttattttaaaatat

SEQ ID NO: 148 - DNA, 78 bp, Artificial Sequence
  Synthetic oligonucleotide
accttgaaggaaaacagggtaatttcaaaaatcttagtgaatttgtgtttaagaatattgatggttattttaaaatat

SEQ ID NO: 149 - DNA, 75 bp, Artificial Sequence
  Synthetic oligonucleotide
ttttaaaataaccatcaatattcttaaacacaaattccctaagatttttgaaattaccctgttttccttcaaggt

SEQ ID NO: 150 - DNA, 75 bp, Artificial Sequence
  Synthetic oligonucleotide
ttttaaaataaccatcaatattcttaaacacaaattcactaagatttttgaaattaccctgttttccttcaaggt

SEQ ID NO: 151 - DNA, 75 bp, Artificial Sequence
  Synthetic oligonucleotide
atatattctaagcacacgcctattaatttagtgcgtgatctccctcagggtttttcggctttagaaccattggta

SEQ ID NO: 152 - DNA, 75 bp, Artificial Sequence
  Synthetic oligonucleotide
atatattctaagcacacgcctattaatttagtgcgtggtctccctcagggtttttcggctttagaaccattggta

SEQ ID NO: 153 - DNA, 75 bp, Artificial Sequence
  Synthetic oligonucleotide
taccaatggttctaaagccgaaaaaccctgagggagatcacgcactaaattaataggcgtgtgcttagaatatat

SEQ ID NO: 154 - DNA, 75 bp, Artificial Sequence
  Synthetic oligonucleotide
taccaatggttctaaagccgaaaaaccctgagggagaccacgcactaaattaataggcgtgtgcttagaatatat

SEQ ID NO: 155 - DNA, 75 bp, Artificial Sequence
  Synthetic oligonucleotide
aatcttgattctaaggttggtggtaattataattacctgtatagattgtttaggaagtctaatctcaaacctttt

SEQ ID NO: 156 - DNA, 75 bp, Artificial Sequence
  Synthetic oligonucleotide
aatcttgattctaaggttggtggtaattataattaccggtatagattgtttaggaagtctaatctcaaacctttt

SEQ ID NO: 157 - DNA, 75 bp, Artificial Sequence
  Synthetic oligonucleotide
aaaaggtttgagattagacttcctaaacaatctatacaggtaattataattaccaccaaccttagaatcaagatt

SEQ ID NO: 158 - DNA, 75 bp, Artificial Sequence
  Synthetic oligonucleotide
aaaaggtttgagattagacttcctaaacaatctataccggtaattataattaccaccaaccttagaatcaagatt

SEQ ID NO: 159 - DNA, 76 bp, Artificial Sequence
  Synthetic oligonucleotide
aatctatcaggccggtagcacaccttgtaatggtgttgaaggttttaattgttactttcctttacaatcatatggt

SEQ ID NO: 160 - DNA, 76 bp, Artificial Sequence
  Synthetic oligonucleotide
aatctatcaggccggtagcacaccttgtaatggtgttaaaggttttaattgttactttcctttacaatcatatggt

SEQ ID NO: 161 - DNA, 75 bp, Artificial Sequence
  Synthetic oligonucleotide
ccatatgattgtaaaggaaagtaacaattaaaaccttcaacaccattacaaggtgtgctaccggcctgatagatt

SEQ ID NO: 162 - DNA, 75 bp, Artificial Sequence
  Synthetic oligonucleotide
ccatatgattgtaaaggaaagtaacaattaaaacctttaacaccattacaaggtgtgctaccggcctgatagatt

SEQ ID NO: 163 - DNA, 75 bp, Artificial Sequence
  Synthetic oligonucleotide
agtcaatccatcattgcctacactatgtcacttggtgcagaaaattcagttgcttactctaataactctattgcc

SEQ ID NO: 164 - DNA, 75 bp, Artificial Sequence
  Synthetic oligonucleotide
agtcaatccatcattgcctacactatgtcacttggtgtagaaaattcagttgcttactctaataactctattgcc

SEQ ID NO: 165 - DNA, 75 bp, Artificial Sequence
  Synthetic oligonucleotide
ggcaatagagttattagagtaagcaactgaattttctgcaccaagtgacatagtgtaggcaatgatggattgact

SEQ ID NO: 166 - DNA, 75 bp, Artificial Sequence
  Synthetic oligonucleotide
ggcaatagagttattagagtaagcaactgaattttctacaccaagtgacatagtgtaggcaatgatggattgact

SEQ ID NO: 167 - DNA, 75 bp, Artificial Sequence
  Synthetic oligonucleotide
atgaagtcagacaaatcgctccagggcaaactggaamgattgctgattataattataaattaccagatgatttta

SEQ ID NO: 168 - DNA, 75 bp, Artificial Sequence
  Synthetic oligonucleotide
atgaagtcagacaaatcgctccagggcaaactggaamtattgctgattataattataaattaccagatgatttta

SEQ ID NO: 169 - DNA, 75 bp, Artificial Sequence
  Synthetic oligonucleotide
taaaatcatctggtaatttataattataatcagcaatckttccagtttgccctggagcgatttgtctgacttcat

SEQ ID NO: 170 - DNA, 75 bp, Artificial Sequence
  Synthetic oligonucleotide
taaaatcatctggtaatttataattataatcagcaatakttccagtttgccctggagcgatttgtctgacttcat

SEQ ID NO: 171 - DNA, 75 bp, Artificial Sequence
  Synthetic oligonucleotide
gatgaagtcagacaaatcgctccagggcaaactggaaakattgctgattataattataaattaccagatgatttt

SEQ ID NO: 172 - DNA, 75 bp, Artificial Sequence
  Synthetic oligonucleotide
gatgaagtcagacaaatcgctccagggcaaactggaackattgctgattataattataaattaccagatgatttt

SEQ ID NO: 173 - DNA, 75 bp, Artificial Sequence
  Synthetic oligonucleotide
aaaatcatctggtaatttataattataatcagcaatmtttccagtttgccctggagcgatttgtctgacttcatc

SEQ ID NO: 174 - DNA, 75 bp, Artificial Sequence
  Synthetic oligonucleotide
aaaatcatctggtaatttataattataatcagcaatmgttccagtttgccctggagcgatttgtctgacttcatc

SEQ ID NO: 175 - DNA, 25 bp, Artificial Sequence
  Synthetic oligonucleotide
gttacttggttccatgctatacatg

SEQ ID NO: 176 - DNA, 24 bp, Artificial Sequence
  Synthetic oligonucleotide
ccaatgttacttggttccatgcta

SEQ ID NO: 177 - DNA, 23 bp, Artificial Sequence
  Synthetic oligonucleotide
tctctgggaccaatggtactaag

SEQ ID NO: 178 - DNA, 22 bp, Artificial Sequence
  Synthetic oligonucleotide
ccattggtcccagagacatgta

SEQ ID NO: 179 - DNA, 23 bp, Artificial Sequence
  Synthetic oligonucleotide
cttagtaccattggtcccagaga

SEQ ID NO: 180 - DNA, 24 bp, Artificial Sequence
  Synthetic oligonucleotide
tagcatggaaccaagtaacattgg

SEQ ID NO: 181 - DNA, 24 bp, Artificial Sequence
  Synthetic oligonucleotide
aatcatatggtttccaacccacty

SEQ ID NO: 182 - DNA, 23 bp, Artificial Sequence
  Synthetic oligonucleotide
atggtgttggttaccaaccatac

SEQ ID NO: 183 - DNA, 23 bp, Artificial Sequence
  Synthetic oligonucleotide
tatggttggtaaccaacaccaty

SEQ ID NO: 184 - DNA, 24 bp, Artificial Sequence
  Synthetic oligonucleotide
agtgggttggaaaccatatgattg

SEQ ID NO: 185 - DNA, 28 bp, Artificial Sequence
  Synthetic oligonucleotide
ctagttatcagactcagactaattctcm

SEQ ID NO: 186 - DNA, 15 bp, Artificial Sequence
  Synthetic oligonucleotide
tcggcgggcacgtag

SEQ ID NO: 187 - DNA, 16 bp, Artificial Sequence
  Synthetic oligonucleotide
ctacgtgcccgccgab

SEQ ID NO: 188 - DNA, 28 bp, Artificial Sequence
  Synthetic oligonucleotide
gagaattagtctgagtctgataactagc

SEQ ID NO: 189 - DNA, 30 bp, Artificial Sequence
  Synthetic oligonucleotide
ggaaaacagggtaatttcaaaaatcttagk

SEQ ID NO: 190 - DNA, 40 bp, Artificial Sequence; identical to SEQ ID NO: 95
  Synthetic oligonucleotide

SEQ ID NO: 191 - DNA, 35 bp, Artificial Sequence
  Synthetic oligonucleotide
taaaataaccatcaatattcttaaacacaaattcm

SEQ ID NO: 192 - DNA, 30 bp, Artificial Sequence; identical to SEQ ID NO: 98
  Synthetic oligonucleotide

SEQ ID NO: 193 - DNA, 23 bp, Artificial Sequence
  Synthetic oligonucleotide
acgcctattaatttagtgcgtgr

SEQ ID NO: 194 - DNA, 20 bp, Artificial Sequence
  Synthetic oligonucleotide
tctccctcagggtttttcgg

SEQ ID NO: 195 - DNA, 21 bp, Artificial Sequence
  Synthetic oligonucleotide
ccgaaaaaccctgagggagay

SEQ ID NO: 196 - DNA, 23 bp, Artificial Sequence
  Synthetic oligonucleotide
cacgcactaaattaataggcgtg

SEQ ID NO: 197 - DNA, 31 bp, Artificial Sequence
  Synthetic oligonucleotide
attctaaggttggtggtaattataattacck

SEQ ID NO: 198 - DNA, 32 bp, Artificial Sequence; identical to SEQ ID NO: 107
  Synthetic oligonucleotide

SEQ ID NO: 199 - DNA, 33 bp, Artificial Sequence
  Synthetic oligonucleotide
gtttgagattagacttcctaaacaatctatacm

SEQ ID NO: 200 - DNA, 30 bp, Artificial Sequence; identical to SEQ ID NO: 110
  Synthetic oligonucleotide

SEQ ID NO: 201 - DNA, 22 bp, Artificial Sequence
  Synthetic oligonucleotide
agcacaccttgtaatggtgttr

SEQ ID NO: 202 - DNA, 31 bp, Artificial Sequence; identical to SEQ ID NO: 113
  Synthetic oligonucleotide

SEQ ID NO: 203 - DNA, 33 bp, Artificial Sequence
  Synthetic oligonucleotide
tgattgtaaaggaaagtaacaattaaaacctty

SEQ ID NO: 204 - DNA, 22 bp, Artificial Sequence
  Synthetic oligonucleotide
aacaccattacaaggtgtgcta

SEQ ID NO: 205 - DNA, 23 bp, Artificial Sequence
  Synthetic oligonucleotide
gcctacactatgtcacttggtgy

SEQ ID NO: 206 - DNA, 31 bp, Artificial Sequence; identical to SEQ ID NO: 119
  Synthetic oligonucleotide

SEQ ID NO: 207 - DNA, 32 bp, Artificial Sequence
  Synthetic oligonucleotide
agagttattagagtaagcaactgaattttctr

SEQ ID NO: 208 - DNA, 22 bp, Artificial Sequence
  Synthetic oligonucleotide
caccaagtgacatagtgtaggc

SEQ ID NO: 209 - DNA, 21 bp, Artificial Sequence
  Synthetic oligonucleotide
gctccagggcaaactggaamk

SEQ ID NO: 210 - DNA, 38 bp, Artificial Sequence; identical to SEQ ID NO: 125
  Synthetic oligonucleotide

SEQ ID NO: 211 - DNA, 39 bp, Artificial Sequence
  Synthetic oligonucleotide
gtaaaatcatctggtaatttataattataatcagcaatm

SEQ ID NO: 212 - DNA, 20 bp, Artificial Sequence
  Synthetic oligonucleotide
kttccagtttgccctggagc

SEQ ID NO: 213 - DNA, 20 bp, Artificial Sequence
  Synthetic oligonucleotide
gctccagggcaaactggaam

SEQ ID NO: 214 - DNA, 39 bp, Artificial Sequence; identical to SEQ ID NO: 131
  Synthetic oligonucleotide

SEQ ID NO: 215 - DNA, 35 bp, Artificial Sequence
  Synthetic oligonucleotide
atcatctggtaatttataattataatcagcaatmk

SEQ ID NO: 216 - DNA, 19 bp, Artificial Sequence
  Synthetic oligonucleotide
ttccagtttgccctggagc

SEQ ID NO: 217 - DNA, 22 bp, Artificial Sequence
  Synthetic primer
acgtggtgtttattaccctgac

SEQ ID NO: 218 - DNA, 22 bp, Artificial Sequence
  Synthetic primer
gctgcattcagttgaatcacca

SEQ ID NO: 219 - DNA, 25 bp, Artificial Sequence
  Synthetic primer
ttccaatgttacttggttccatgct

SEQ ID NO: 220 - DNA, 30 bp, Artificial Sequence
  Synthetic primer
gggtatggcaatagagttattagagtaagc

SEQ ID NO: 221 - DNA, 27 bp, Artificial Sequence
  Synthetic primer
acattcaactcaggacttgttcttacc

SEQ ID NO: 222 - DNA, 25 bp, Artificial Sequence
  Synthetic primer
gctgtccaacctgaagaagaatcac

SEQ ID NO: 223 - DNA, 25 bp, Artificial Sequence
  Synthetic primer
caggaagagaatcagcaactgtgtt

SEQ ID NO: 224 - DNA, 27 bp, Artificial Sequence
  Synthetic primer
tgtcaagaatctcaagtgtctgtggat

SEQ ID NO: 225 - DNA, 26 bp, Artificial Sequence
  Synthetic primer
atcaacttactcctacttggcgtgtt

SEQ ID NO: 226 - DNA, 26 bp, Artificial Sequence
  Synthetic primer
tctagcagcaatatcaccaaggcaat

SEQ ID NO: 227 - DNA, 19 bp, Artificial Sequence
  Synthetic probe
tccctcagggtttttcggc

SEQ ID NO: 228 - DNA, 19 bp, Artificial Sequence
  Synthetic probe
tccctcagggtttttcggt

SEQ ID NO: 229 - DNA, 25 bp, Artificial Sequence
  Synthetic probe
tttagaaccattggtagatttgcca

SEQ ID NO: 230 - DNA, 25 bp, Artificial Sequence
  Synthetic probe
ggcaaatctaccaatggttctaaag

SEQ ID NO: 231 - DNA, 25 bp, Artificial Sequence
  Synthetic probe
ggcaaatctaccaatggttctaaaa

SEQ ID NO: 232 - DNA, 20 bp, Artificial Sequence
  Synthetic probe
ccgaaaaaccctgagggaga

SEQ ID NO: 233 - DNA, 18 bp, Artificial Sequence
  Synthetic probe
cggtgatgctgctcttgc

SEQ ID NO: 234 - DNA, 18 bp, Artificial Sequence
  Synthetic probe
cggtgatgctgctcttgt

SEQ ID NO: 235 - DNA, 22 bp, Artificial Sequence
  Synthetic probe
tttgctgctgcttgacagattg

SEQ ID NO: 236 - DNA, 23 bp, Artificial Sequence
  Synthetic probe
caatctgtcaagcagcagcaaag

SEQ ID NO: 237 - DNA, 23 bp, Artificial Sequence
  Synthetic probe
caatctgtcaagcagcagcaaaa

SEQ ID NO: 238 - DNA, 18 bp, Artificial Sequence
  Synthetic probe
caagagcagcatcaccgc

SEQ ID NO: 239 - DNA, 27 bp, Artificial Sequence
  Synthetic probe
tgaattctcgtaactacatagcacaag

SEQ ID NO: 240 - DNA, 27 bp, Artificial Sequence
  Synthetic probe
tgaattctcgtaactacatagcacaat

SEQ ID NO: 241 - DNA, 30 bp, Artificial Sequence
  Synthetic probe
tagatgtagttaactttaatctcacatagc

SEQ ID NO: 242 - DNA, 31 bp, Artificial Sequence
  Synthetic probe
gctatgtgagattaaagttaactacatctac

SEQ ID NO: 243 - DNA, 31 bp, Artificial Sequence
  Synthetic probe
gctatgtgagattaaagttaactacatctaa

SEQ ID NO: 244 - DNA, 26 bp, Artificial Sequence
  Synthetic probe
ttgtgctatgtagttacgagaattca

SEQ ID NO: 245 - DNA, 31 bp, Artificial Sequence
  Synthetic probe
ttgaaattaaattggcaaagaaatttgacac

SEQ ID NO: 246 - DNA, 31 bp, Artificial Sequence
  Synthetic probe
ttgaaattaaattggcaaagaaatttgacat

SEQ ID NO: 247 - DNA, 26 bp, Artificial Sequence
  Synthetic probe
cttcaatggggaatgtccaaattttg

SEQ ID NO: 248 - DNA, 26 bp, Artificial Sequence
  Synthetic probe
aaaatttggacattccccattgaagg

SEQ ID NO: 249 - DNA, 26 bp, Artificial Sequence
  Synthetic probe
aaaatttggacattccccattgaaga

SEQ ID NO: 250 - DNA, 31 bp, Artificial Sequence
  Synthetic probe
tgtcaaatttctttgccaatttaatttcaaa

SEQ ID NO: 251 - DNA, 24 bp, Artificial Sequence
  Synthetic probe
gttgcacttcttgctgtttttcag

SEQ ID NO: 252 - DNA, 24 bp, Artificial Sequence
  Synthetic probe
gttgcacttcttgctgtttttcat

SEQ ID NO: 253 - DNA, 23 bp, Artificial Sequence
  Synthetic probe
agcgcttccaaaatcataaccct

SEQ ID NO: 254 - DNA, 23 bp, Artificial Sequence
  Synthetic probe
gggttatgattttggaagcgctc

SEQ ID NO: 255 - DNA, 23 bp, Artificial Sequence
  Synthetic probe
gggttatgattttggaagcgcta

SEQ ID NO: 256 - DNA, 23 bp, Artificial Sequence
  Synthetic probe
tgaaaaacagcaagaagtgcaac

SEQ ID NO: 257 - DNA, 29 bp, Artificial Sequence
  Synthetic probe
ttttgtaatgatccatttttgggtgttta

SEQ ID NO: 258 - DNA, 28 bp, Artificial Sequence
  Synthetic probe
aattttgtaatgatccatttttgggtgt

SEQ ID NO: 259 - DNA, 27 bp, Artificial Sequence
  Synthetic probe
ttaccacaaaaacaacaaaagttggat

SEQ ID NO: 260 - DNA, 28 bp, Artificial Sequence
  Synthetic probe
ccaacttttgttgtttttgtggtaataa

SEQ ID NO: 261 - DNA, 27 bp, Artificial Sequence
  Synthetic probe
atccaacttttgttgtttttgtggtaa

SEQ ID NO: 262 - DNA, 28 bp, Artificial Sequence
  Synthetic probe
acacccaaaaatggatcattacaaaatt

SEQ ID NO: 263 - DNA, 24 bp, Artificial Sequence
  Synthetic probe
ccaacaatttggcagagacattgc

SEQ ID NO: 264 - DNA, 24 bp, Artificial Sequence
  Synthetic probe
ccaacaatttggcagagacattga

SEQ ID NO: 265 - DNA, 21 bp, Artificial Sequence
  Synthetic probe
tgacactactgatgctgtccg

SEQ ID NO: 266 - DNA, 22 bp, Artificial Sequence
  Synthetic probe
cggacagcatcagtagtgtcag

SEQ ID NO: 267 - DNA, 22 bp, Artificial Sequence
  Synthetic probe
cggacagcatcagtagtgtcat

SEQ ID NO: 268 - DNA, 24 bp, Artificial Sequence
  Synthetic probe
caatgtctctgccaaattgttgga

SEQ ID NO: 269 - DNA, 26 bp, Artificial Sequence
  Synthetic probe
tctaataactctattgccatacccac

SEQ ID NO: 270 - DNA, 26 bp, Artificial Sequence
  Synthetic probe
tctaataactctattgccatacccat

SEQ ID NO: 271 - DNA, 32 bp, Artificial Sequence
  Synthetic probe
aaattttactattagtgttaccacagaaattc

SEQ ID NO: 272 - DNA, 33 bp, Artificial Sequence
  Synthetic probe
gaatttctgtggtaacactaatagtaaaatttg

SEQ ID NO: 273 - DNA, 33 bp, Artificial Sequence
  Synthetic probe
gaatttctgtggtaacactaatagtaaaattta

SEQ ID NO: 274 - DNA, 29 bp, Artificial Sequence
  Synthetic probe
tgggtatggcaatagagttattagagtaa

SEQ ID NO: 275 - DNA, 31 bp, Artificial Sequence
  Synthetic probe
gcaatttcaagtgttttaaatgatatccttt

SEQ ID NO: 276 - DNA, 31 bp, Artificial Sequence
  Synthetic probe
gcaatttcaagtgttttaaatgatatccttg

SEQ ID NO: 277 - DNA, 22 bp, Artificial Sequence
  Synthetic probe
cacgtcttgacaaagttgaggc

SEQ ID NO: 278 - DNA, 23 bp, Artificial Sequence
  Synthetic probe
gcctcaactttgtcaagacgtga

SEQ ID NO: 279 - DNA, 23 bp, Artificial Sequence
  Synthetic probe
gcctcaactttgtcaagacgtgc

SEQ ID NO: 280 - DNA, 31 bp, Artificial Sequence
  Synthetic probe
aaggatatcatttaaaacacttgaaattgca

SEQ ID NO: 281 - DNA, 32 bp, Artificial Sequence
  Synthetic probe
gaatttttatgaaccacaaatcattactacag

SEQ ID NO: 282 - DNA, 32 bp, Artificial Sequence
  Synthetic probe
gaatttttatgaaccacaaatcattactacac

SEQ ID NO: 283 - DNA, 24 bp, Artificial Sequence
  Synthetic probe
acaacacatttgtgtctggtaact

SEQ ID NO: 284 - DNA, 24 bp, Artificial Sequence
  Synthetic probe
gttaccagacacaaatgtgttgtc

SEQ ID NO: 285 - DNA, 24 bp, Artificial Sequence
  Synthetic probe
gttaccagacacaaatgtgttgtg

SEQ ID NO: 286 - DNA, 32 bp, Artificial Sequence
  Synthetic probe
tgtagtaatgatttgtggttcataaaaattcc

SEQ ID NO: 287 - DNA, 24 bp, Artificial Sequence
  Synthetic probe
cgatcatcagcacatctaggtttc

SEQ ID NO: 288 - DNA, 24 bp, Artificial Sequence
  Synthetic probe
cgatcatcagcacatctaggtttt

SEQ ID NO: 289 - DNA, 19 bp, Artificial Sequence
  Synthetic probe
gtccgggtgtgaccgaaag

SEQ ID NO: 290 - DNA, 16 bp, Artificial Sequence
  Synthetic probe
cggtcacacccggacg

SEQ ID NO: 291 - DNA, 16 bp, Artificial Sequence
  Synthetic probe
cggtcacacccggaca

SEQ ID NO: 292 - DNA, 23 bp, Artificial Sequence
  Synthetic probe
aaacctagatgtgctgatgatcg

SEQ ID NO: 293 - DNA, 28 bp, Artificial Sequence
  Synthetic probe
attggcttcacatatgtattgttctttc

SEQ ID NO: 294 - DNA, 28 bp, Artificial Sequence
  Synthetic probe
attggcttcacatatgtattgttctttt

SEQ ID NO: 295 - DNA, 23 bp, Artificial Sequence
  Synthetic probe
taccctccagatgaggatgaaga

SEQ ID NO: 296 - DNA, 23 bp, Artificial Sequence
  Synthetic probe
cttcatcctcatctggagggtag

SEQ ID NO: 297 - DNA, 23 bp, Artificial Sequence
  Synthetic probe
cttcatcctcatctggagggtaa

SEQ ID NO: 298 - DNA, 28 bp, Artificial Sequence
  Synthetic probe
aaagaacaatacatatgtgaagccaatt

SEQ ID NO: 299 - DNA, 25 bp, Artificial Sequence
  Synthetic probe
ttttattctctacagtgttcccacc

SEQ ID NO: 300 - DNA, 25 bp, Artificial Sequence
  Synthetic probe
ttttattctctacagtgttcccact

SEQ ID NO: 301 - DNA, 25 bp, Artificial Sequence
  Synthetic probe
tacaagttttggaccactagtgaga

SEQ ID NO: 302 - DNA, 25 bp, Artificial Sequence
  Synthetic probe
ctcactagtggtccaaaacttgtag

SEQ ID NO: 303 - DNA, 25 bp, Artificial Sequence
  Synthetic probe
ctcactagtggtccaaaacttgtaa

SEQ ID NO: 304 - DNA, 26 bp, Artificial Sequence
  Synthetic probe
gtgggaacactgtagagaataaaact

SEQ ID NO: 305 - DNA, 23 bp, Artificial Sequence
  Synthetic probe
gtccaaattcacacaatcgacgg

SEQ ID NO: 306 - DNA, 23 bp, Artificial Sequence
  Synthetic probe
gtccaaattcacacaatcgacgt

SEQ ID NO: 307 - DNA, 23 bp, Artificial Sequence
  Synthetic probe
ttcatccggagttgttaatccag

SEQ ID NO: 308 - DNA, 24 bp, Artificial Sequence
  Synthetic probe
ctggattaacaactccggatgaac

SEQ ID NO: 309 - DNA, 24 bp, Artificial Sequence
  Synthetic probe
ctggattaacaactccggatgaaa

SEQ ID NO: 310 - DNA, 23 bp, Artificial Sequence
  Synthetic probe
cgtcgattgtgtgaatttggaca

SEQ ID NO: 311 - DNA, 26 bp, Artificial Sequence
  Synthetic probe
aagcatacaatgtaacacaagctttc

SEQ ID NO: 312 - DNA, 26 bp, Artificial Sequence
  Synthetic probe
aagcatacaatgtaacacaagctttt

SEQ ID NO: 313 - DNA, 18 bp, Artificial Sequence
  Synthetic probe
ggcagacgtggtccagaa

SEQ ID NO: 314 - DNA, 17 bp, Artificial Sequence
  Synthetic probe
ctggaccacgtctgccg

SEQ ID NO: 315 - DNA, 17 bp, Artificial Sequence
  Synthetic probe
ctggaccacgtctgcca

SEQ ID NO: 316 - DNA, 27 bp, Artificial Sequence
  Synthetic probe
aaagcttgtgttacattgtatgcttta

SEQ ID NO: 317 - DNA, 27 bp, Artificial Sequence
  Synthetic probe
cagatgattttacaggctgcgttatag

SEQ ID NO: 318 - DNA, 27 bp, Artificial Sequence
  Synthetic probe
cagatgattttacaggctgcgttatat

SEQ ID NO: 319 - DNA, 30 bp, Artificial Sequence
  Synthetic probe
cttggaattctaacaatcttgattctaagg

SEQ ID NO: 320 - DNA, 30 bp, Artificial Sequence
  Synthetic probe
cttagaatcaagattgttagaattccaagc

SEQ ID NO: 321 - DNA, 30 bp, Artificial Sequence
  Synthetic probe
cttagaatcaagattgttagaattccaaga

SEQ ID NO: 322 - DNA, 25 bp, Artificial Sequence
  Synthetic probe
tataacgcagcctgtaaaatcatct

SEQ ID NO: 323 - DNA, 75 bp, Artificial Sequence
  Synthetic oligonucleotide
attaatttagtgcgtgatctccctcagggtttttcggctttagaaccattggtagatttgccaataggtattaac

SEQ ID NO: 324 - DNA, 75 bp, Artificial Sequence
  Synthetic oligonucleotide
attaatttagtgcgtgatctccctcagggtttttcggttttagaaccattggtagatttgccaataggtattaac

SEQ ID NO: 325 - DNA, 75 bp, Artificial Sequence
  Synthetic oligonucleotide
gttaatacctattggcaaatctaccaatggttctaaagccgaaaaaccctgagggagatcacgcactaaattaat

SEQ ID NO: 326 - DNA, 75 bp, Artificial Sequence
  Synthetic oligonucleotide
gttaatacctattggcaaatctaccaatggttctaaaaccgaaaaaccctgagggagatcacgcactaaattaat

SEQ ID NO: 327 - DNA, 75 bp, Artificial Sequence
  Synthetic oligonucleotide
gctagaatggctggcaatggcggtgatgctgctcttgctttgctgctgcttgacagattgaaccagcttgagagc

SEQ ID NO: 328 - DNA, 75 bp, Artificial Sequence
  Synthetic oligonucleotide
gctagaatggctggcaatggcggtgatgctgctcttgttttgctgctgcttgacagattgaaccagcttgagagc

SEQ ID NO: 329 - DNA, 75 bp, Artificial Sequence
  Synthetic oligonucleotide
gctctcaagctggttcaatctgtcaagcagcagcaaagcaagagcagcatcaccgccattgccagccattctagc

SEQ ID NO: 330 - DNA, 75 bp, Artificial Sequence
  Synthetic oligonucleotide
gctctcaagctggttcaatctgtcaagcagcagcaaaacaagagcagcatcaccgccattgccagccattctagc

SEQ ID NO: 331 - DNA, 75 bp, Artificial Sequence
  Synthetic oligonucleotide
cttgtgcagaatgaattctcgtaactacatagcacaagtagatgtagttaactttaatctcacatagcaatcttt

SEQ ID NO: 332 - DNA, 75 bp, Artificial Sequence
  Synthetic oligonucleotide
cttgtgcagaatgaattctcgtaactacatagcacaattagatgtagttaactttaatctcacatagcaatcttt

SEQ ID NO: 333 - DNA, 75 bp, Artificial Sequence
  Synthetic oligonucleotide
aaagattgctatgtgagattaaagttaactacatctacttgtgctatgtagttacgagaattcattctgcacaag

SEQ ID NO: 334 - DNA, 75 bp, Artificial Sequence
  Synthetic oligonucleotide
aaagattgctatgtgagattaaagttaactacatctaattgtgctatgtagttacgagaattcattctgcacaag

SEQ ID NO: 335 - DNA, 75 bp, Artificial Sequence
  Synthetic oligonucleotide
acaccttttgaaattaaattggcaaagaaatttgacaccttcaatggggaatgtccaaattttgtatttccctta

SEQ ID NO: 336 - DNA, 75 bp, Artificial Sequence
  Synthetic oligonucleotide
acaccttttgaaattaaattggcaaagaaatttgacatcttcaatggggaatgtccaaattttgtatttccctta

SEQ ID NO: 337 - DNA, 75 bp, Artificial Sequence
  Synthetic oligonucleotide
taagggaaatacaaaatttggacattccccattgaaggtgtcaaatttctttgccaatttaatttcaaaaggtgt

SEQ ID NO: 338 - DNA, 75 bp, Artificial Sequence
  Synthetic oligonucleotide
taagggaaatacaaaatttggacattccccattgaagatgtcaaatttctttgccaatttaatttcaaaaggtgt

SEQ ID NO: 339 - DNA, 75 bp, Artificial Sequence
  Synthetic oligonucleotide
ggcttattgttggcgttgcacttcttgctgtttttcagagcgcttccaaaatcataaccctcaaaaagagatggc

SEQ ID NO: 340 - DNA, 75 bp, Artificial Sequence
  Synthetic oligonucleotide
ggcttattgttggcgttgcacttcttgctgtttttcatagcgcttccaaaatcataaccctcaaaaagagatggc

SEQ ID NO: 341 - DNA, 75 bp, Artificial Sequence
  Synthetic oligonucleotide
gccatctctttttgagggttatgattttggaagcgctctgaaaaacagcaagaagtgcaacgccaacaataagcc

SEQ ID NO: 342 - DNA, 75 bp, Artificial Sequence
  Synthetic oligonucleotide
gccatctctttttgagggttatgattttggaagcgctatgaaaaacagcaagaagtgcaacgccaacaataagcc

SEQ ID NO: 343 - DNA, 75 bp, Unknown
  WT template strand
tgaatttcaattttgtaatgatccatttttgggtgtttattaccacaaaaacaacaaaagttggatggaaagtga

SEQ ID NO: 344 - DNA, 75 bp, Artificial Sequence
  Synthetic oligonucleotide
gtgaatttcaattttgtaatgatccatttttgggtgtttaccacaaaaacaacaaaagttggatggaaagtgagt

SEQ ID NO: 345 - DNA, 75 bp, Unknown
  WT cDNA strand
tcactttccatccaacttttgttgtttttgtggtaataaacacccaaaaatggatcattacaaaattgaaattca

SEQ ID NO: 346 - DNA, 75 bp, Artificial Sequence
  Synthetic oligonucleotide
actcactttccatccaacttttgttgtttttgtggtaaacacccaaaaatggatcattacaaaattgaaattcac

SEQ ID NO: 347 - DNA, 75 bp, Artificial Sequence
  Synthetic oligonucleotide
aagtttctgcctttccaacaatttggcagagacattgctgacactactgatgctgtccgtgatccacagacactt

SEQ ID NO: 348 - DNA, 75 bp, Artificial Sequence
  Synthetic oligonucleotide
aagtttctgcctttccaacaatttggcagagacattgatgacactactgatgctgtccgtgatccacagacactt

SEQ ID NO: 349 - DNA, 75 bp, Artificial Sequence
  Synthetic oligonucleotide
aagtgtctgtggatcacggacagcatcagtagtgtcagcaatgtctctgccaaattgttggaaaggcagaaactt

SEQ ID NO: 350 - DNA, 75 bp, Artificial Sequence
  Synthetic oligonucleotide
aagtgtctgtggatcacggacagcatcagtagtgtcatcaatgtctctgccaaattgttggaaaggcagaaactt

SEQ ID NO: 351 - DNA, 75 bp, Artificial Sequence
  Synthetic oligonucleotide
tcagttgcttactctaataactctattgccatacccacaaattttactattagtgttaccacagaaattctacca

SEQ ID NO: 352 - DNA, 75 bp, Artificial Sequence
  Synthetic oligonucleotide
tcagttgcttactctaataactctattgccatacccataaattttactattagtgttaccacagaaattctacca

SEQ ID NO: 353 - DNA, 75 bp, Artificial Sequence
  Synthetic oligonucleotide
tggtagaatttctgtggtaacactaatagtaaaatttgtgggtatggcaatagagttattagagtaagcaactga

SEQ ID NO: 354 - DNA, 75 bp, Artificial Sequence
  Synthetic oligonucleotide
tggtagaatttctgtggtaacactaatagtaaaatttatgggtatggcaatagagttattagagtaagcaactga

SEQ ID NO: 355 - DNA, 75 bp, Artificial Sequence
  Synthetic oligonucleotide
ttttggtgcaatttcaagtgttttaaatgatatcctttcacgtcttgacaaagttgaggctgaagtgcaaatatt

SEQ ID NO: 356 - DNA, 75 bp, Artificial Sequence
  Synthetic oligonucleotide
ttttggtgcaatttcaagtgttttaaatgatatccttgcacgtcttgacaaagttgaggctgaagtgcaaatatt

SEQ ID NO: 357 - DNA, 75 bp, Artificial Sequence
  Synthetic oligonucleotide
aatatttgcacttcagcctcaactttgtcaagacgtgaaaggatatcatttaaaacacttgaaattgcaccaaaa

SEQ ID NO: 358 - DNA, 75 bp, Artificial Sequence
  Synthetic oligonucleotide
aatatttgcacttcagcctcaactttgtcaagacgtgcaaggatatcatttaaaacacttgaaattgcaccaaaa

SEQ ID NO: 359 - DNA, 75 bp, Artificial Sequence
  Synthetic oligonucleotide
acaaaggaatttttatgaaccacaaatcattactacagacaacacatttgtgtctggtaactgtgatgttgtaat

SEQ ID NO: 360 - DNA, 75 bp, Artificial Sequence
  Synthetic oligonucleotide
acaaaggaatttttatgaaccacaaatcattactacacacaacacatttgtgtctggtaactgtgatgttgtaat

SEQ ID NO: 361 - DNA, 75 bp, Artificial Sequence
  Synthetic oligonucleotide
attacaacatcacagttaccagacacaaatgtgttgtctgtagtaatgatttgtggttcataaaaattcctttgt

SEQ ID NO: 362 - DNA, 75 bp, Artificial Sequence
  Synthetic oligonucleotide
attacaacatcacagttaccagacacaaatgtgttgtgtgtagtaatgatttgtggttcataaaaattcctttgt

SEQ ID NO: 363 - DNA, 75 bp, Artificial Sequence
  Synthetic oligonucleotide
gtccgtgttgcagccgatcatcagcacatctaggtttcgtccgggtgtgaccgaaaggtaagatggagagccttg

SEQ ID NO: 364 - DNA, 75 bp, Artificial Sequence
  Synthetic oligonucleotide
gtccgtgttgcagccgatcatcagcacatctaggttttgtccgggtgtgaccgaaaggtaagatggagagccttg

SEQ ID NO: 365 - DNA, 75 bp, Artificial Sequence
  Synthetic oligonucleotide
caaggctctccatcttacctttcggtcacacccggacgaaacctagatgtgctgatgatcggctgcaacacggac

SEQ ID NO: 366 - DNA, 75 bp, Artificial Sequence
  Synthetic oligonucleotide
caaggctctccatcttacctttcggtcacacccggacaaaacctagatgtgctgatgatcggctgcaacacggac

SEQ ID NO: 367 - DNA, 75 bp, Artificial Sequence
  Synthetic oligonucleotide
gtgagtttaaattggcttcacatatgtattgttctttctaccctccagatgaggatgaagaagaaggtgattgtg

SEQ ID NO: 368 - DNA, 75 bp, Artificial Sequence
  Synthetic oligonucleotide
gtgagtttaaattggcttcacatatgtattgttctttttaccctccagatgaggatgaagaagaaggtgattgtg

SEQ ID NO: 369 - DNA, 75 bp, Artificial Sequence
  Synthetic oligonucleotide
cacaatcaccttcttcttcatcctcatctggagggtagaaagaacaatacatatgtgaagccaatttaaactcac

SEQ ID NO: 370 - DNA, 75 bp, Artificial Sequence
  Synthetic oligonucleotide
cacaatcaccttcttcttcatcctcatctggagggtaaaaagaacaatacatatgtgaagccaatttaaactcac

SEQ ID NO: 371 - DNA, 75 bp, Artificial Sequence
  Synthetic oligonucleotide
gcaaactttaatgttttattctctacagtgttcccacctacaagttttggaccactagtgagaaaaatatttgtt

SEQ ID NO: 372 - DNA, 75 bp, Artificial Sequence
  Synthetic oligonucleotide
gcaaactttaatgttttattctctacagtgttcccacttacaagttttggaccactagtgagaaaaatatttgtt

SEQ ID NO: 373 - DNA, 75 bp, Artificial Sequence
  Synthetic oligonucleotide
aacaaatatttttctcactagtggtccaaaacttgtaggtgggaacactgtagagaataaaacattaaagtttgc

SEQ ID NO: 374 - DNA, 75 bp, Artificial Sequence
  Synthetic oligonucleotide
aacaaatatttttctcactagtggtccaaaacttgtaagtgggaacactgtagagaataaaacattaaagtttgc

SEQ ID NO: 375 - DNA, 75 bp, Artificial Sequence
  Synthetic oligonucleotide
gagcctgaagaacatgtccaaattcacacaatcgacggttcatccggagttgttaatccagtaatggaaccaatt

SEQ ID NO: 376 - DNA, 75 bp, Artificial Sequence
  Synthetic oligonucleotide
gagcctgaagaacatgtccaaattcacacaatcgacgtttcatccggagttgttaatccagtaatggaaccaatt

SEQ ID NO: 377 - DNA, 75 bp, Artificial Sequence
  Synthetic oligonucleotide
aattggttccattactggattaacaactccggatgaaccgtcgattgtgtgaatttggacatgttcttcaggctc

SEQ ID NO: 378 - DNA, 75 bp, Artificial Sequence
  Synthetic oligonucleotide
aattggttccattactggattaacaactccggatgaaacgtcgattgtgtgaatttggacatgttcttcaggctc

SEQ ID NO: 379 - DNA, 75 bp, Artificial Sequence
  Synthetic oligonucleotide
gtactgccactaaagcatacaatgtaacacaagctttcggcagacgtggtccagaacaaacccaaggaaattttg

SEQ ID NO: 380 - DNA, 75 bp, Artificial Sequence
  Synthetic oligonucleotide
gtactgccactaaagcatacaatgtaacacaagcttttggcagacgtggtccagaacaaacccaaggaaattttg

SEQ ID NO: 381 - DNA, 75 bp, Artificial Sequence
  Synthetic oligonucleotide
caaaatttccttgggtttgttctggaccacgtctgccgaaagcttgtgttacattgtatgctttagtggcagtac

SEQ ID NO: 382 - DNA, 75 bp, Artificial Sequence
  Synthetic oligonucleotide
caaaatttccttgggtttgttctggaccacgtctgccaaaagcttgtgttacattgtatgctttagtggcagtac

SEQ ID NO: 383 - DNA, 75 bp, Artificial Sequence
  Synthetic oligonucleotide
ttataaattaccagatgattttacaggctgcgttatagcttggaattctaacaatcttgattctaaggttggtgg

SEQ ID NO: 384 - DNA, 75 bp, Artificial Sequence
  Synthetic oligonucleotide
ttataaattaccagatgattttacaggctgcgttatatcttggaattctaacaatcttgattctaaggttggtgg

SEQ ID NO: 385 - DNA, 75 bp, Artificial Sequence
  Synthetic oligonucleotide
ccaccaaccttagaatcaagattgttagaattccaagctataacgcagcctgtaaaatcatctggtaatttataa

SEQ ID NO: 386 - DNA, 75 bp, Artificial Sequence
  Synthetic oligonucleotide
ccaccaaccttagaatcaagattgttagaattccaagatataacgcagcctgtaaaatcatctggtaatttataa

SEQ ID NO: 387 - DNA, 19 bp, Artificial Sequence
  Synthetic oligonucleotide
tccctcagggtttttcggy

SEQ ID NO: 388 - DNA, 25 bp, Artificial Sequence
  Synthetic oligonucleotide
tttagaaccattggtagatttgcca

SEQ ID NO: 389 - DNA, 25 bp, Artificial Sequence
  Synthetic oligonucleotide
ggcaaatctaccaatggttctaaar

SEQ ID NO: 390 - DNA, 20 bp, Artificial Sequence
  Synthetic oligonucleotide
ccgaaaaaccctgagggaga

SEQ ID NO: 391 - DNA, 18 bp, Artificial Sequence
  Synthetic oligonucleotide
cggtgatgctgctcttgy

SEQ ID NO: 392 - DNA, 22 bp, Artificial Sequence
  Synthetic oligonucleotide
tttgctgctgcttgacagattg

SEQ ID NO: 393 - DNA, 23 bp, Artificial Sequence
  Synthetic oligonucleotide
caatctgtcaagcagcagcaaar

SEQ ID NO: 394 - DNA, 18 bp, Artificial Sequence
  Synthetic oligonucleotide
caagagcagcatcaccgc

SEQ ID NO: 395 - DNA, 27 bp, Artificial Sequence
  Synthetic oligonucleotide
tgaattctcgtaactacatagcacaak

SEQ ID NO: 396 - DNA, 30 bp, Artificial Sequence; identical to SEQ ID NO: 241
  Synthetic oligonucleotide

SEQ ID NO: 397 - DNA, 31 bp, Artificial Sequence
  Synthetic oligonucleotide
gctatgtgagattaaagttaactacatctam

SEQ ID NO: 398 - DNA, 26 bp, Artificial Sequence
  Synthetic oligonucleotide
ttgtgctatgtagttacgagaattca

SEQ ID NO: 399 - DNA, 31 bp, Artificial Sequence
  Synthetic oligonucleotide
ttgaaattaaattggcaaagaaatttgacay

SEQ ID NO: 400 - DNA, 26 bp, Artificial Sequence
  Synthetic oligonucleotide
cttcaatggggaatgtccaaattttg

SEQ ID NO: 401 - DNA, 26 bp, Artificial Sequence
  Synthetic oligonucleotide
aaaatttggacattccccattgaagr

SEQ ID NO: 402 - DNA, 31 bp, Artificial Sequence; identical to SEQ ID NO: 250
  Synthetic oligonucleotide

SEQ ID NO: 403 - DNA, 24 bp, Artificial Sequence
  Synthetic oligonucleotide
gttgcacttcttgctgtttttcak

SEQ ID NO: 404 - DNA, 23 bp, Artificial Sequence
  Synthetic oligonucleotide
agcgcttccaaaatcataaccct

SEQ ID NO: 405 - DNA, 23 bp, Artificial Sequence
  Synthetic oligonucleotide
gggttatgattttggaagcgctm

SEQ ID NO: 406 - DNA, 23 bp, Artificial Sequence
  Synthetic oligonucleotide
tgaaaaacagcaagaagtgcaac

SEQ ID NO: 407 - DNA, 29 bp, Artificial Sequence
  Synthetic oligonucleotide
ttttgtaatgatccatttttgggtgttta

SEQ ID NO: 408 - DNA, 28 bp, Artificial Sequence
  Synthetic oligonucleotide
aattttgtaatgatccatttttgggtgt

SEQ ID NO: 409 - DNA, 27 bp, Artificial Sequence
  Synthetic oligonucleotide
ttaccacaaaaacaacaaaagttggat

SEQ ID NO: 410 - DNA, 28 bp, Artificial Sequence
  Synthetic oligonucleotide
ccaacttttgttgtttttgtggtaataa

SEQ ID NO: 411 - DNA, 27 bp, Artificial Sequence
  Synthetic oligonucleotide
atccaacttttgttgtttttgtggtaa

SEQ ID NO: 412 - DNA, 28 bp, Artificial Sequence
  Synthetic oligonucleotide
acacccaaaaatggatcattacaaaatt

SEQ ID NO: 413 - DNA, 24 bp, Artificial Sequence
  Synthetic oligonucleotide
ccaacaatttggcagagacattgm

SEQ ID NO: 414 - DNA, 21 bp, Artificial Sequence
  Synthetic oligonucleotide
tgacactactgatgctgtccg

SEQ ID NO: 415 - DNA, 22 bp, Artificial Sequence
  Synthetic oligonucleotide
cggacagcatcagtagtgtcak

SEQ ID NO: 416 - DNA, 24 bp, Artificial Sequence
  Synthetic oligonucleotide
caatgtctctgccaaattgttgga

SEQ ID NO: 417 - DNA, 26 bp, Artificial Sequence
  Synthetic oligonucleotide
tctaataactctattgccatacccay

SEQ ID NO: 418 - DNA, 32 bp, Artificial Sequence; identical to SEQ ID NO: 271
  Synthetic oligonucleotide

SEQ ID NO: 419 - DNA, 33 bp, Artificial Sequence
  Synthetic oligonucleotide
gaatttctgtggtaacactaatagtaaaatttr

SEQ ID NO: 420 - DNA, 29 bp, Artificial Sequence
  Synthetic oligonucleotide
tgggtatggcaatagagttattagagtaa

SEQ ID NO: 421 - DNA, 31 bp, Artificial Sequence
  Synthetic oligonucleotide
gcaatttcaagtgttttaaatgatatccttk

SEQ ID NO: 422 - DNA, 22 bp, Artificial Sequence
  Synthetic oligonucleotide
cacgtcttgacaaagttgaggc

SEQ ID NO: 423 - DNA, 23 bp, Artificial Sequence
  Synthetic oligonucleotide
gcctcaactttgtcaagacgtgm

SEQ ID NO: 424 - DNA, 31 bp, Artificial Sequence; identical to SEQ ID NO: 280
  Synthetic oligonucleotide

SEQ ID NO: 425 - DNA, 32 bp, Artificial Sequence
  Synthetic oligonucleotide
gaatttttatgaaccacaaatcattactacas

SEQ ID NO: 426 - DNA, 24 bp, Artificial Sequence
  Synthetic oligonucleotide
acaacacatttgtgtctggtaact

SEQ ID NO: 427 - DNA, 24 bp, Artificial Sequence
  Synthetic oligonucleotide
gttaccagacacaaatgtgttgts

SEQ ID NO: 428 - DNA, 32 bp, Artificial Sequence; identical to SEQ ID NO: 286
  Synthetic oligonucleotide

SEQ ID NO: 429 - DNA, 24 bp, Artificial Sequence
  Synthetic oligonucleotide
cgatcatcagcacatctaggttty

SEQ ID NO: 430 - DNA, 19 bp, Artificial Sequence
  Synthetic oligonucleotide
gtccgggtgtgaccgaaag

SEQ ID NO: 431 - DNA, 16 bp, Artificial Sequence
  Synthetic oligonucleotide
cggtcacacccggacr

SEQ ID NO: 432 - DNA, 23 bp, Artificial Sequence
  Synthetic oligonucleotide
aaacctagatgtgctgatgatcg

SEQ ID NO: 433 - DNA, 28 bp, Artificial Sequence
  Synthetic oligonucleotide
attggcttcacatatgtattgttcttty

SEQ ID NO: 434 - DNA, 23 bp, Artificial Sequence
  Synthetic oligonucleotide
taccctccagatgaggatgaaga

SEQ ID NO: 435 - DNA, 23 bp, Artificial Sequence
  Synthetic oligonucleotide
cttcatcctcatctggagggtar

SEQ ID NO: 436 - DNA, 28 bp, Artificial Sequence
  Synthetic oligonucleotide
aaagaacaatacatatgtgaagccaatt

SEQ ID NO: 437 - DNA, 25 bp, Artificial Sequence
  Synthetic oligonucleotide
ttttattctctacagtgttcccacy

SEQ ID NO: 438 - DNA, 25 bp, Artificial Sequence
  Synthetic oligonucleotide
tacaagttttggaccactagtgaga

SEQ ID NO: 439 - DNA, 25 bp, Artificial Sequence
  Synthetic oligonucleotide
ctcactagtggtccaaaacttgtar

SEQ ID NO: 440 - DNA, 26 bp, Artificial Sequence
  Synthetic oligonucleotide
gtgggaacactgtagagaataaaact

SEQ ID NO: 441 - DNA, 23 bp, Artificial Sequence
  Synthetic oligonucleotide
gtccaaattcacacaatcgacgk

SEQ ID NO: 442 - DNA, 23 bp, Artificial Sequence
  Synthetic oligonucleotide
ttcatccggagttgttaatccag

SEQ ID NO: 443 - DNA, 24 bp, Artificial Sequence
  Synthetic oligonucleotide
ctggattaacaactccggatgaam

SEQ ID NO: 444 - DNA, 23 bp, Artificial Sequence
  Synthetic oligonucleotide
cgtcgattgtgtgaatttggaca

SEQ ID NO: 445 - DNA, 26 bp, Artificial Sequence
  Synthetic oligonucleotide
aagcatacaatgtaacacaagcttty

SEQ ID NO: 446 - DNA, 18 bp, Artificial Sequence
  Synthetic oligonucleotide
ggcagacgtggtccagaa

SEQ ID NO: 447 - DNA, 17 bp, Artificial Sequence
  Synthetic oligonucleotide
ctggaccacgtctgccr

SEQ ID NO: 448 - DNA, 27 bp, Artificial Sequence
  Synthetic oligonucleotide
aaagcttgtgttacattgtatgcttta

SEQ ID NO: 449 - DNA, 27 bp, Artificial Sequence
  Synthetic oligonucleotide
cagatgattttacaggctgcgttatak

SEQ ID NO: 450 - DNA, 30 bp, Artificial Sequence; identical to SEQ ID NO: 319
  Synthetic oligonucleotide

SEQ ID NO: 451 - DNA, 30 bp, Artificial Sequence
  Synthetic oligonucleotide
cttagaatcaagattgttagaattccaagm

SEQ ID NO: 452 - DNA, 25 bp, Artificial Sequence
  Synthetic oligonucleotide
tataacgcagcctgtaaaatcatct

SEQ ID NO: 453 - DNA, 22 bp, Artificial Sequence
  Synthetic primer
cgtggtgtttattaccctgaca

SEQ ID NO: 454 - DNA, 22 bp, Artificial Sequence
  Synthetic primer
ataagtagggactgggtcttcg

SEQ ID NO: 455 - DNA, 22 bp, Artificial Sequence
  Synthetic primer
ggatggaaagtgagttcagagt

SEQ ID NO: 456 - DNA, 22 bp, Artificial Sequence
  Synthetic primer
gctgtccaacctgaagaagaat

SEQ ID NO: 457 - DNA, 22 bp, Artificial Sequence
  Synthetic primer
caggctgcgttatagcttggaa

SEQ ID NO: 458 - DNA, 22 bp, Artificial Sequence
  Synthetic primer
tgctggtgcatgtagaagttca

SEQ ID NO: 459 - DNA, 22 bp, Artificial Sequence
  Synthetic primer
tgagtgtgacatacccattggt

SEQ ID NO: 460 - DNA, 22 bp, Artificial Sequence
  Synthetic primer
tgctgcattcagttgaatcacc

SEQ ID NO: 461 - DNA, 27 bp, Artificial Sequence
  Synthetic primer
acattcaactcaggacttgttcttacc

SEQ ID NO: 462 - DNA, 30 bp, Artificial Sequence
  Synthetic primer
cagcctcttattatgttagacttctcagtg

SEQ ID NO: 463 - DNA, 27 bp, Artificial Sequence
  Synthetic primer
gttggatggaaagtgagttcagagttt

SEQ ID NO: 464 - DNA, 27 bp, Artificial Sequence
  Synthetic primer
tggcaaatctaccaatggttctaaagc

SEQ ID NO: 465 - DNA, 25 bp, Artificial Sequence
  Synthetic primer
caggaagagaatcagcaactgtgtt

SEQ ID NO: 466 - DNA, 24 bp, Artificial Sequence
  Synthetic primer
tccaagctataacgcagcctgtaa

SEQ ID NO: 467 - DNA, 25 bp, Artificial Sequence
  Synthetic primer
caatcttgattctaaggttggtggt

SEQ ID NO: 468 - DNA, 27 bp, Artificial Sequence
  Synthetic primer
gtactactactctgtatggttggtaac

SEQ ID NO: 469 - DNA, 27 bp, Artificial Sequence
  Synthetic primer
tccacagacacttgagattcttgacat

SEQ ID NO: 470 - DNA, 26 bp, Artificial Sequence
  Synthetic primer
aacacgccaagtaggagtaagttgat

SEQ ID NO: 471 - DNA, 31 bp, Artificial Sequence
  Synthetic primer
gctagttatcagactcagactaattctcctc

SEQ ID NO: 472 - DNA, 26 bp, Artificial Sequence
  Synthetic primer
attgctgcattcagttgaatcaccac

SEQ ID NO: 473 - DNA, 30 bp, Artificial Sequence
  Synthetic primer
gtcagtgtgttaatcttacaaccagaactc

SEQ ID NO: 474 - DNA, 30 bp, Artificial Sequence
  Synthetic primer
gcctcttattatgttagacttctcagtgga

SEQ ID NO: 475 - DNA, 25 bp, Artificial Sequence
  Synthetic primer
tggatggaaagtgagttcagagttt

SEQ ID NO: 476 - DNA, 25 bp, Artificial Sequence
  Synthetic primer
ctgaagaagaatcaccaggagtcaa

SEQ ID NO: 477 - DNA, 24 bp, Artificial Sequence
  Synthetic primer
tacaggctgcgttatagcttggaa

SEQ ID NO: 478 - DNA, 24 bp, Artificial Sequence
  Synthetic primer
aacagttgctggtgcatgtagaag

SEQ ID NO: 479 - DNA, 28 bp, Artificial Sequence
  Synthetic primer
tcatgcagatcaacttactcctacttgg

SEQ ID NO: 480 - DNA, 30 bp, Artificial Sequence
  Synthetic primer
catagacactggtagaatttctgtggtaac

SEQ ID NO: 481 - DNA, 20 bp, Artificial Sequence
  Synthetic primer
aacgccaccagatttgcatc

SEQ ID NO: 482 - DNA, 21 bp, Artificial Sequence
  Synthetic primer
acgcagcctgtaaaatcatct

SEQ ID NO: 483 - DNA, 23 bp, Artificial Sequence
  Synthetic primer
actgtgttgctgattattctgtc

SEQ ID NO: 484 - DNA, 23 bp, Artificial Sequence
  Synthetic primer
ctataacgcagcctgtaaaatca

SEQ ID NO: 485 - DNA, 20 bp, Artificial Sequence
  Synthetic primer
aaacttgtgcccttttggtg

SEQ ID NO: 486 - DNA, 21 bp, Artificial Sequence
  Synthetic primer
aggggtactgctgttatgtct

SEQ ID NO: 487 - DNA, 20 bp, Artificial Sequence
  Synthetic primer
gcagtagcgcgaacaaaatc

SEQ ID NO: 488 - DNA, 25 bp, Artificial Sequence
  Synthetic primer
atttgacatgagtaaatttcccctt

SEQ ID NO: 489 - DNA, 21 bp, Artificial Sequence
  Synthetic primer
gcatccttgatttcaccttgc

SEQ ID NO: 490 - DNA, 26 bp, Artificial Sequence
  Synthetic primer
ttaaattaaggggtactgctgttatg

SEQ ID NO: 491 - DNA, 22 bp, Artificial Sequence
  Synthetic primer
cgcgaacaaaatctgaaggagt

SEQ ID NO: 492 - DNA, 48 bp, Artificial Sequence
  Synthetic probe
actggtaacccagacatgatcggtgctgattttgacacatggtttagt

SEQ ID NO: 493 - DNA, 48 bp, Artificial Sequence
  Synthetic probe
actccctgtgggtgagcttaatgggctgattttgacacatggtttagc

SEQ ID NO: 494 - DNA, 21 bp, Artificial Sequence
  Synthetic probe
cagcgtggtggtagttatact

SEQ ID NO: 495 - DNA, 53 bp, Artificial Sequence
  Synthetic probe
catttggtcattggttcaagacgagatatcggtaattatacagtttcctgttc

SEQ ID NO: 496 - DNA, 53 bp, Artificial Sequence
  Synthetic probe
ctggtccgttgtggtccttctaacgatatcggtaattatacagtttcctgttt

SEQ ID NO: 497 - DNA, 26 bp, Artificial Sequence
  Synthetic probe
accttttacaattaattgccaggaac

SEQ ID NO: 498 - DNA, 47 bp, Artificial Sequence
  Synthetic probe
ctaatagctcctgtgccctcgtatcaggttgctgttctttatcagga

SEQ ID NO: 499 - DNA, 47 bp, Artificial Sequence
  Synthetic probe
aatccgtcgactagcctgagaattcaggttgctgttctttatcaggg

SEQ ID NO: 500 - DNA, 22 bp, Artificial Sequence
  Synthetic probe
tgttaactgcacagaagtccct

SEQ ID NO: 501 - DNA, 54 bp, Artificial Sequence
  Synthetic probe
gtggcaacagaaatcaggtggtgaagtactcaatggtctttgttcttttttttg

SEQ ID NO: 502 - DNA, 54 bp, Artificial Sequence
  Synthetic probe
agaggactgctaaaggtttgtaggagtactcaatggtctttgttcttttttttt

SEQ ID NO: 503 - DNA, 28 bp, Artificial Sequence
  Synthetic probe
tatgaaaatgcctttttaccttttgcta

SEQ ID NO: 504 - DNA, 51 bp, Artificial Sequence
  Synthetic probe
gtggcaacagaaatcaggtggtgagcaaaaggtaaaaaggcattttcatac

SEQ ID NO: 505 - DNA, 51 bp, Artificial Sequence
  Synthetic probe
agaggactgctaaaggtttgtagggcaaaaggtaaaaaggcattttcataa

SEQ ID NO: 506 - DNA, 30 bp, Artificial Sequence
  Synthetic probe
aaaaaaaagaacaaagaccattgagtactc

SEQ ID NO: 507 - DNA, 75 bp, Artificial Sequence
  Synthetic oligonucleotide
ttgctaacaaacatgctgattttgacacatggtttagtcagcgtggtggtagttatactaatgacaaagcttgcc

SEQ ID NO: 508 - DNA, 75 bp, Artificial Sequence
  Synthetic oligonucleotide
ttgctaacaaacatgctgattttgacacatggtttagccagcgtggtggtagttatactaatgacaaagcttgcc

SEQ ID NO: 509 - DNA, 75 bp, Artificial Sequence
  Synthetic oligonucleotide
ggcaagctttgtcattagtataactaccaccacgctgactaaaccatgtgtcaaaatcagcatgtttgttagcaa

SEQ ID NO: 510 - DNA, 75 bp, Artificial Sequence
  Synthetic oligonucleotide
ggcaagctttgtcattagtataactaccaccacgctggctaaaccatgtgtcaaaatcagcatgtttgttagcaa

SEQ ID NO: 511 - DNA, 75 bp, Artificial Sequence
  Synthetic oligonucleotide
cagtacatcgatatcggtaattatacagtttcctgttcaccttttacaattaattgccaggaacctaaattgggt

SEQ ID NO: 512 - DNA, 75 bp, Artificial Sequence
  Synthetic oligonucleotide
cagtacatcgatatcggtaattatacagtttcctgtttaccttttacaattaattgccaggaacctaaattgggt

SEQ ID NO: 513 - DNA, 75 bp, Artificial Sequence
  Synthetic oligonucleotide
acccaatttaggttcctggcaattaattgtaaaaggtgaacaggaaactgtataattaccgatatcgatgtactg

SEQ ID NO: 514 - DNA, 75 bp, Artificial Sequence
  Synthetic oligonucleotide
acccaatttaggttcctggcaattaattgtaaaaggtaaacaggaaactgtataattaccgatatcgatgtactg

SEQ ID NO: 515 - DNA, 75 bp, Artificial Sequence
  Synthetic oligonucleotide
acaaatacttctaaccaggttgctgttctttatcaggatgttaactgcacagaagtccctgttgctattcatgca

SEQ ID NO: 516 - DNA, 75 bp, Artificial Sequence
  Synthetic oligonucleotide
acaaatacttctaaccaggttgctgttctttatcagggtgttaactgcacagaagtccctgttgctattcatgca

SEQ ID NO: 517 - DNA, 75 bp, Artificial Sequence
  Synthetic oligonucleotide
tgcatgaatagcaacagggacttctgtgcagttaacatcctgataaagaacagcaacctggttagaagtatttgt

SEQ ID NO: 518 - DNA, 75 bp, Artificial Sequence
  Synthetic oligonucleotide
tgcatgaatagcaacagggacttctgtgcagttaacaccctgataaagaacagcaacctggttagaagtatttgt

SEQ ID NO: 519 - DNA, 75 bp, Artificial Sequence
  Synthetic oligonucleotide
tactccagagtactcaatggtctttgttcttttttttgtatgaaaatgcctttttaccttttgctatgggtatta

SEQ ID NO: 520 - DNA, 75 bp, Artificial Sequence
  Synthetic oligonucleotide
tactccagagtactcaatggtctttgttcttttttttttatgaaaatgcctttttaccttttgctatgggtatta

SEQ ID NO: 521 - DNA, 75 bp, Artificial Sequence
  Synthetic oligonucleotide
taatacccatagcaaaaggtaaaaaggcattttcatacaaaaaaaagaacaaagaccattgagtactctggacta

SEQ ID NO: 522 - DNA, 75 bp, Artificial Sequence
  Synthetic oligonucleotide
taatacccatagcaaaaggtaaaaaggcattttcataaaaaaaaaagaacaaagaccattgagtactctggacta

SEQ ID NO: 523 - DNA, 24 bp, Artificial Sequence
  Synthetic oligonucleotide
gctgattttgacacatggtttagy

SEQ ID NO: 524 - DNA, 21 bp, Artificial Sequence
  Synthetic oligonucleotide
cagcgtggtggtagttatact

SEQ ID NO: 525 - DNA, 29 bp, Artificial Sequence
  Synthetic oligonucleotide
gatatcggtaattatacagtttcctgtty

SEQ ID NO: 526 - DNA, 26 bp, Artificial Sequence
  Synthetic oligonucleotide
accttttacaattaattgccaggaac

SEQ ID NO: 527 - DNA, 23 bp, Artificial Sequence
  Synthetic oligonucleotide
caggttgctgttctttatcaggr

SEQ ID NO: 528 - DNA, 22 bp, Artificial Sequence
  Synthetic oligonucleotide
tgttaactgcacagaagtccct

SEQ ID NO: 529 - DNA, 30 bp, Artificial Sequence
  Synthetic oligonucleotide
agtactcaatggtctttgttcttttttttk

SEQ ID NO: 530 - DNA, 28 bp, Artificial Sequence
  Synthetic oligonucleotide
tatgaaaatgcctttttaccttttgcta

SEQ ID NO: 531 - DNA, 27 bp, Artificial Sequence
  Synthetic oligonucleotide
gcaaaaggtaaaaaggcattttcatam

SEQ ID NO: 532 - DNA, 30 bp, Artificial Sequence; identical to SEQ ID NO: 506
  Synthetic oligonucleotide

SEQ ID NO: 533 - DNA, 51 bp, Artificial Sequence
  Synthetic probe
cgtaccattgaatctggagaccttacatagaagttatttgactcctggtga

SEQ ID NO: 534 - DNA, 51 bp, Artificial Sequence
  Synthetic probe
tagatgccgctgcaggtatggaaaacatagaagttatttgactcctggtgg

SEQ ID NO: 535 - DNA, 23 bp, Artificial Sequence
  Synthetic probe
ttcttcttcaggttggacagctg

SEQ ID NO: 536 - DNA, 47 bp, Artificial Sequence
  Synthetic probe
cgtaccattgaatctggagaccttagctgtccaacctgaagaagaat

SEQ ID NO: 537 - DNA, 47 bp, Artificial Sequence
  Synthetic probe
tagatgccgctgcaggtatggaaaagctgtccaacctgaagaagaac

SEQ ID NO: 538 - DNA, 29 bp, Artificial Sequence
  Synthetic probe
caccaggagtcaaataacttctatgtaaa

SEQ ID NO: 539 - DNA, 75 bp, Artificial Sequence
  Synthetic oligonucleotide
ttacttgctttacatagaagttatttgactcctggtgattcttcttcaggttggacagctggtgctgcagcttat

SEQ ID NO: 540 - DNA, 75 bp, Artificial Sequence
  Synthetic oligonucleotide
ttacttgctttacatagaagttatttgactcctggtggttcttcttcaggttggacagctggtgctgcagcttat

SEQ ID NO: 541 - DNA, 75 bp, Artificial Sequence
  Synthetic oligonucleotide
ataagctgcagcaccagctgtccaacctgaagaagaatcaccaggagtcaaataacttctatgtaaagcaagtaa

SEQ ID NO: 542 - DNA, 75 bp, Artificial Sequence
  Synthetic oligonucleotide
ataagctgcagcaccagctgtccaacctgaagaagaaccaccaggagtcaaataacttctatgtaaagcaagtaa

SEQ ID NO: 543 - DNA, 27 bp, Artificial Sequence
  Synthetic oligonucleotide
acatagaagttatttgactcctggtgr

SEQ ID NO: 544 - DNA, 23 bp, Artificial Sequence
  Synthetic oligonucleotide
ttcttcttcaggttggacagctg

SEQ ID NO: 545 - DNA, 23 bp, Artificial Sequence
  Synthetic oligonucleotide
agctgtccaacctgaagaagaay

SEQ ID NO: 546 - DNA, 29 bp, Artificial Sequence
  Synthetic oligonucleotide
caccaggagtcaaataacttctatgtaaa

SEQ ID NO: 547 - protein, 6 aa, Artificial Sequence
  Synthetic 6xHis tag
HHHHHH

SEQ ID NO: 548 - protein, 8 aa, Artificial Sequence
  Synthetic peptide
DYKDDDDK

SEQ ID NO: 549 - DNA, 46 bp, Artificial Sequence
  Synthetic
catttggtcattggttcaagacgaagcacaccttgtaatggtgttc

SEQ ID NO: 550 - DNA, 57 bp, Artificial Sequence
  Synthetic
catttggtcattggttcaagacgatgattgtaaaggaaagtaacaattaaaaccttg

SEQ ID NO: 551 - DNA, 54 bp, Artificial Sequence
  Synthetic
ctaatagctcctgtgccctcgtatactagtctctagtcagtgtgttaatcttac

SEQ ID NO: 552 - DNA, 54 bp, Artificial Sequence
  Synthetic
aatccgtcgactagcctgagaattactagtctctagtcagtgtgttaatcttag

SEQ ID NO: 553 - DNA, 22 bp, Artificial Sequence
  Synthetic
aaccagaactcaattaccccct

SEQ ID NO: 554 - DNA, 47 bp, Artificial Sequence
  Synthetic
ctaatagctcctgtgccctcgtatagggggtaattgagttctggttg

SEQ ID NO: 555 - DNA, 47 bp, Artificial Sequence
  Synthetic
aatccgtcgactagcctgagaattagggggtaattgagttctggttc

SEQ ID NO: 556 - DNA, 29 bp, Artificial Sequence
  Synthetic
taagattaacacactgactagagactagt

SEQ ID NO: 557 - DNA, 52 bp, Artificial Sequence
  Synthetic
ctggtccgttgtggtccttctaacctagttatcagactcagactaattctcg

SEQ ID NO: 558 - DNA, 40 bp, Artificial Sequence
  Synthetic
ctggtccgttgtggtccttctaacctacgtgcccgccgac

SEQ ID NO: 559 - DNA, 48 bp, Artificial Sequence
  Synthetic
tagatgccgctgcaggtatggaaacatgtgacttatgtccctgcacaa

SEQ ID NO: 560 - DNA, 48 bp, Artificial Sequence
  Synthetic
cgtaccattgaatctggagaccttcatgtgacttatgtccctgcacat

SEQ ID NO: 561 - DNA, 25 bp, Artificial Sequence
  Synthetic
gaaaagaacttcacaactgctcctg

SEQ ID NO: 562 - DNA, 50 bp, Artificial Sequence
  Synthetic
tagatgccgctgcaggtatggaaacaggagcagttgtgaagttcttttct

SEQ ID NO: 563 - DNA, 50 bp, Artificial Sequence
  Synthetic
cgtaccattgaatctggagaccttcaggagcagttgtgaagttcttttca

SEQ ID NO: 564 - DNA, 22 bp, Artificial Sequence
  Synthetic
tgtgcagggacataagtcacat

SEQ ID NO: 565 - DNA, 46 bp, Artificial Sequence
  Synthetic
actggtaacccagacatgatcgggaaatctatcaggccggtarcac

SEQ ID NO: 566 - DNA, 47 bp, Artificial Sequence
  Synthetic
actccctgtgggtgagcttaatgggaaatctatcaggccggtarcaa

SEQ ID NO: 567 - DNA, 27 bp, Artificial Sequence
  Synthetic
accttgtaatggtgttvaaggttttaa

SEQ ID NO: 568 - DNA, 51 bp, Artificial Sequence
  Synthetic
actggtaacccagacatgatcggttaaaaccttbaacaccattacaaggtg

SEQ ID NO: 569 - DNA, 52 bp, Artificial Sequence
  Synthetic
actccctgtgggtgagcttaatggttaaaaccttbaacaccattacaaggtt

SEQ ID NO: 570 - DNA, 21 bp, Artificial Sequence
  Synthetic
tgytaccggcctgatagattt

SEQ ID NO: 571 - DNA, 46 bp, Artificial Sequence
  Synthetic
catttggtcattggttcaagacgaacaatcacttctggttggacct

SEQ ID NO: 572 - DNA, 46 bp, Artificial Sequence
  Synthetic
ctggtccgttgtggtccttctaacacaatcacttctggttggaccc

SEQ ID NO: 573 - DNA, 17 bp, Artificial Sequence
  Synthetic
ttggtgcaggtgctgca

SEQ ID NO: 574 - DNA, 41 bp, Artificial Sequence
  Synthetic
catttggtcattggttcaagacgagcagcacctgcaccaaa

SEQ ID NO: 575 - DNA, 41 bp, Artificial Sequence
  Synthetic
ctggtccgttgtggtccttctaacgcagcacctgcaccaag

SEQ ID NO: 576 - DNA, 23 bp, Artificial Sequence
  Synthetic
ggtccaaccagaagtgattgtac

SEQ ID NO: 577 - DNA, 48 bp, Artificial Sequence
  Synthetic
ctaatagctcctgtgccctcgtatacatctctggcattaatgcttcag

SEQ ID NO: 578 - DNA, 48 bp, Artificial Sequence
  Synthetic
aatccgtcgactagcctgagaattacatctctggcattaatgcttcat

SEQ ID NO: 579 - DNA, 29 bp, Artificial Sequence
  Synthetic
ttgtaaacattcaaaaagaaattgaccgc

SEQ ID NO: 580 - DNA, 54 bp, Artificial Sequence
  Synthetic
ctaatagctcctgtgccctcgtatgcggtcaatttctttttgaatgtttacaac

SEQ ID NO: 581 - DNA, 54 bp, Artificial Sequence
  Synthetic
aatccgtcgactagcctgagaattgcggtcaatttctttttgaatgtttacaaa

SEQ ID NO: 582 - DNA, 24 bp, Artificial Sequence
  Synthetic
tgaagcattaatgccagagatgtc

SEQ ID NO: 583 - DNA, 49 bp, Artificial Sequence
  Synthetic
gtggcaacagaaatcaggtggtgatatatgcgctagttatcagactcmg

SEQ ID NO: 584 - DNA, 49 bp, Artificial Sequence
  Synthetic
agaggactgctaaaggtttgtaggtatatgcgctagttatcagactcmt

SEQ ID NO: 585 - DNA, 49 bp, Artificial Sequence
  Synthetic
agaggactgctaaaggtttgtaggtatatgcgctagttatcagactcmc

SEQ ID NO: 586 - DNA, 19 bp, Artificial Sequence
  Synthetic
actaattctcvtcggcggg

SEQ ID NO: 587 - DNA, 44 bp, Artificial Sequence
  Synthetic
gtggcaacagaaatcaggtggtgacccgccgabgagaattagtc

SEQ ID NO: 588 - DNA, 44 bp, Artificial Sequence
  Synthetic
agaggactgctaaaggtttgtaggcccgccgabgagaattagta

SEQ ID NO: 589 - DNA, 44 bp, Artificial Sequence
  Synthetic
agaggactgctaaaggtttgtaggcccgccgabgagaattagtg

SEQ ID NO: 590 - DNA, 24 bp, Artificial Sequence
  Synthetic
kgagtctgataactagcgcatata

SEQ ID NO: 591 - DNA, 53 bp, Artificial Sequence
  Synthetic
tagatgccgctgcaggtatggaaattttgtaatgatccatttttgggtgttta

SEQ ID NO: 592 - DNA, 52 bp, Artificial Sequence
  Synthetic
cgtaccattgaatctggagaccttaattttgtaatgatccatttttgggtgt

SEQ ID NO: 593 - DNA, 27 bp, Artificial Sequence
  Synthetic
ttaccacaaaaacaacaaaagttggat

SEQ ID NO: 594 - DNA, 52 bp, Artificial Sequence
  Synthetic
tagatgccgctgcaggtatggaaaccaacttttgttgtttttgtggtaataa

SEQ ID NO: 595 - DNA, 51 bp, Artificial Sequence
  Synthetic
cgtaccattgaatctggagaccttatccaacttttgttgtttttgtggtaa

SEQ ID NO: 596 - DNA, 28 bp, Artificial Sequence
  Synthetic
acacccaaaaatggatcattacaaaatt

SEQ ID NO: 597 - DNA, 76 bp, Artificial Sequence
  Synthetic
aatctatcaggccggtagcacaccttgtaatggtgttcaaggttttaattgttactttcctttacaatcatatggt

SEQ ID NO: 598 - DNA, 75 bp, Artificial Sequence
  Synthetic
ccatatgattgtaaaggaaagtaacaattaaaaccttgaacaccattacaaggtgtgctaccggcctgatagatt

SEQ ID NO: 599 - DNA, 75 bp, Artificial Sequence
  Synthetic
ttattgccactagtctctagtcagtgtgttaatcttacaaccagaactcaattaccccctgcatacactaattct

SEQ ID NO: 600 - DNA, 75 bp, Artificial Sequence
  Synthetic
ttattgccactagtctctagtcagtgtgttaatcttagaaccagaactcaattaccccctgcatacactaattct

SEQ ID NO: 601 - DNA, 75 bp, Artificial Sequence
  Synthetic
agaattagtgtatgcagggggtaattgagttctggttgtaagattaacacactgactagagactagtggcaataa

SEQ ID NO: 602 - DNA, 75 bp, Artificial Sequence
  Synthetic
agaattagtgtatgcagggggtaattgagttctggttctaagattaacacactgactagagactagtggcaataa

SEQ ID NO: 603 - DNA, 75 bp, Artificial Sequence
  Synthetic
aggtatatgcgctagttatcagactcagactaattctcgtcggcgggcacgtagtgtagctagtcaatccatcat

SEQ ID NO: 604 - DNA, 75 bp, Artificial Sequence
  Synthetic
atgatggattgactagctacactacgtgcccgccgacgagaattagtctgagtctgataactagcgcatatacct

SEQ ID NO: 605 - DNA, 75 bp, Artificial Sequence
  Synthetic
gtgtagtcttcttgcatgtgacttatgtccctgcacaagaaaagaacttcacaactgctcctgccatttgtcatg

SEQ ID NO: 606 - DNA, 75 bp, Artificial Sequence
  Synthetic
gtgtagtcttcttgcatgtgacttatgtccctgcacatgaaaagaacttcacaactgctcctgccatttgtcatg

SEQ ID NO: 607 - DNA, 75 bp, Artificial Sequence
  Synthetic
catgacaaatggcaggagcagttgtgaagttcttttcttgtgcagggacataagtcacatgcaagaagactacac

SEQ ID NO: 608 - DNA, 75 bp, Artificial Sequence
  Synthetic
catgacaaatggcaggagcagttgtgaagttcttttcatgtgcagggacataagtcacatgcaagaagactacac

SEQ ID NO: 609 - DNA, 75 bp, Artificial Sequence
  Synthetic
agagatatttcaactgaaatctatcaggccggtarcacaccttgtaatggtgttvaaggttttaattgttacttt

SEQ ID NO: 610 - DNA, 75 bp, Artificial Sequence
  Synthetic
agagatatttcaactgaaatctatcaggccggtarcaaaccttgtaatggtgttvaaggttttaattgttacttt

SEQ ID NO: 611 - DNA, 75 bp, Artificial Sequence
  Synthetic
aaagtaacaattaaaaccttbaacaccattacaaggtgtgytaccggcctgatagatttcagttgaaatatctct

SEQ ID NO: 612 - DNA, 75 bp, Artificial Sequence
  Synthetic
aaagtaacaattaaaaccttbaacaccattacaaggtttgytaccggcctgatagatttcagttgaaatatctct

SEQ ID NO: 613 - DNA, 75 bp, Artificial Sequence
  Synthetic
tgcactgttagcgggtacaatcacttctggttggacctttggtgcaggtgctgcattacaaataccatttgctat

SEQ ID NO: 614 - DNA, 75 bp, Artificial Sequence
  Synthetic
tgcactgttagcgggtacaatcacttctggttggacccttggtgcaggtgctgcattacaaataccatttgctat

SEQ ID NO: 615 - DNA, 75 bp, Artificial Sequence
  Synthetic
atagcaaatggtatttgtaatgcagcacctgcaccaaaggtccaaccagaagtgattgtacccgctaacagtgca

SEQ ID NO: 616 - DNA, 75 bp, Artificial Sequence
  Synthetic
atagcaaatggtatttgtaatgcagcacctgcaccaagggtccaaccagaagtgattgtacccgctaacagtgca

SEQ ID NO: 617 - DNA, 75 bp, Artificial Sequence
  Synthetic
tgttgatttaggtgacatctctggcattaatgcttcagttgtaaacattcaaaaagaaattgaccgcctcaatga

SEQ ID NO: 618 - DNA, 75 bp, Artificial Sequence
  Synthetic
tgttgatttaggtgacatctctggcattaatgcttcatttgtaaacattcaaaaagaaattgaccgcctcaatga

SEQ ID NO: 619 - DNA, 75 bp, Artificial Sequence
  Synthetic
tcattgaggcggtcaatttctttttgaatgtttacaactgaagcattaatgccagagatgtcacctaaatcaaca

SEQ ID NO: 620 - DNA, 75 bp, Artificial Sequence
  Synthetic
tcattgaggcggtcaatttctttttgaatgtttacaaatgaagcattaatgccagagatgtcacctaaatcaaca

SEQ ID NO: 621 - DNA, 75 bp, Artificial Sequence
  Synthetic
ccattggtgcaggtatatgcgctagttatcagactcmgactaattctcvtcggcgggcacgtagtgtagctagtc

SEQ ID NO: 622 - DNA, 75 bp, Artificial Sequence
  Synthetic
ccattggtgcaggtatatgcgctagttatcagactcmtactaattctcvtcggcgggcacgtagtgtagctagtc

SEQ ID NO: 623 - DNA, 75 bp, Artificial Sequence
  Synthetic
ccattggtgcaggtatatgcgctagttatcagactcmcactaattctcvtcggcgggcacgtagtgtagctagtc

SEQ ID NO: 624 - DNA, 75 bp, Artificial Sequence
  Synthetic
gactagctacactacgtgcccgccgabgagaattagtckgagtctgataactagcgcatatacctgcaccaatgg

SEQ ID NO: 625 - DNA, 75 bp, Artificial Sequence
  Synthetic
gactagctacactacgtgcccgccgabgagaattagtakgagtctgataactagcgcatatacctgcaccaatgg

SEQ ID NO: 626 - DNA, 75 bp, Artificial Sequence
  Synthetic
gactagctacactacgtgcccgccgabgagaattagtgkgagtctgataactagcgcatatacctgcaccaatgg

SEQ ID NO: 627 - DNA, 22 bp, Artificial Sequence
  Synthetic
agcacaccttgtaatggtgtts

SEQ ID NO: 628 - DNA, 33 bp, Artificial Sequence
  Synthetic
tgattgtaaaggaaagtaacaattaaaacctts

SEQ ID NO: 629 - DNA, 30 bp, Artificial Sequence
  Synthetic
actagtctctagtcagtgtgttaatcttas

SEQ ID NO: 630 - DNA, 22 bp, Artificial Sequence
  Synthetic
aaccagaactcaattaccccct

SEQ ID NO: 631 - DNA, 23 bp, Artificial Sequence
  Synthetic
agggggtaattgagttctggtts

SEQ ID NO: 632 - DNA, 29 bp, Artificial Sequence
  Synthetic
taagattaacacactgactagagactagt

SEQ ID NO: 633 - DNA, 28 bp, Artificial Sequence
  Synthetic
ctagttatcagactcagactaattctcs

SEQ ID NO: 634 - DNA, 16 bp, Artificial Sequence
  Synthetic
ctacgtgcccgccgas

SEQ ID NO: 635 - DNA, 24 bp, Artificial Sequence
  Synthetic
catgtgacttatgtccctgcacaw

SEQ ID NO: 636 - DNA, 25 bp, Artificial Sequence
  Synthetic
gaaaagaacttcacaactgctcctg

SEQ ID NO: 637 - DNA, 26 bp, Artificial Sequence
  Synthetic
caggagcagttgtgaagttcttttcw

SEQ ID NO: 638 - DNA, 22 bp, Artificial Sequence
  Synthetic
tgtgcagggacataagtcacat

SEQ ID NO: 639 - DNA, 23 bp, Artificial Sequence
  Synthetic
gaaatctatcaggccggtarcam

SEQ ID NO: 640 - DNA, 27 bp, Artificial Sequence
  Synthetic
accttgtaatggtgttvaaggttttaa

SEQ ID NO: 641 - DNA, 28 bp, Artificial Sequence
  Synthetic
ttaaaaccttbaacaccattacaaggtk

SEQ ID NO: 642 - DNA, 21 bp, Artificial Sequence
  Synthetic
tgytaccggcctgatagattt

SEQ ID NO: 643 - DNA, 22 bp, Artificial Sequence
  Synthetic
acaatcacttctggttggaccy

SEQ ID NO: 644 - DNA, 17 bp, Artificial Sequence
  Synthetic
ttggtgcaggtgctgca

SEQ ID NO: 645 - DNA, 17 bp, Artificial Sequence
  Synthetic
gcagcacctgcaccaar

SEQ ID NO: 646 - DNA, 23 bp, Artificial Sequence
  Synthetic
ggtccaaccagaagtgattgtac

SEQ ID NO: 647 - DNA, 24 bp, Artificial Sequence
  Synthetic
acatctctggcattaatgcttcak

SEQ ID NO: 648 - DNA, 29 bp, Artificial Sequence
  Synthetic
ttgtaaacattcaaaaagaaattgaccgc

SEQ ID NO: 649 - DNA, 30 bp, Artificial Sequence
  Synthetic
gcggtcaatttctttttgaatgtttacaam

SEQ ID NO: 650 - DNA, 24 bp, Artificial Sequence
  Synthetic
tgaagcattaatgccagagatgtc

SEQ ID NO: 651 - DNA, 25 bp, Artificial Sequence
  Synthetic
tatatgcgctagttatcagactcmb

SEQ ID NO: 652 - DNA, 19 bp, Artificial Sequence
  Synthetic
actaattctcvtcggcggg

SEQ ID NO: 653 - DNA, 20 bp, Artificial Sequence
  Synthetic
cccgccgabgagaattagtv

SEQ ID NO: 654 - DNA, 24 bp, Artificial Sequence
  Synthetic
kgagtctgataactagcgcatata

SEQ ID NO: 655 - DNA, 24 bp, Artificial Sequence
  Synthetic
tarcatggaaccaagtaacattgg

SEQ ID NO: 656 - DNA, 28 bp, Artificial Sequence
  Synthetic
gagaattagtvkgagtctgataactagc

SEQ ID NO: 657 - DNA, 57 bp, Artificial Sequence; identical to SEQ ID NO: 108
  Synthetic

SEQ ID NO: 658 - DNA, 57 bp, Artificial Sequence; identical to SEQ ID NO: 109
  Synthetic

SEQ ID NO: 659 - DNA, 22 bp, Artificial Sequence
  Synthetic
aacaccattacaaggtktgyta

SEQ ID NO: 660 - DNA, 47 bp, Artificial Sequence
  Synthetic
ctaatagctcctgtgccctcgtatagrgggtaattgagttctgkttg

SEQ ID NO: 661 - DNA, 47 bp, Artificial Sequence
  Synthetic
aatccgtcgactagcctgagaattagrgggtaattgagttctgkttc

SEQ ID NO: 662 - DNA, 29 bp, Artificial Sequence
  Synthetic
taarattaacacactgamtagagactagt

SEQ ID NO: 663 - DNA, 21 bp, Artificial Sequence
  Synthetic
  misc_feature (21)..(21): n is a, c, g, or t
aggggtactgctgttatgtcn

SEQ ID NO: 664 - DNA, 21 bp, Artificial Sequence
  Synthetic
  misc_feature (21)..(21): n is a, c, g, or t
gcatccttgatttcaccttgn

SEQ ID NO: 665 - DNA, 21 bp, Artificial Sequence
  Synthetic
gcatccttgatttcaccttgm

SEQ ID NO: 666 - DNA, 46 bp, Artificial Sequence
  Synthetic
gtggcaacagaaatcaggtggtgaacaatcacttctggttggacct

SEQ ID NO: 667 - DNA, 46 bp, Artificial Sequence
  Synthetic
agaggactgctaaaggtttgtaggacaatcacttctggttggaccc

SEQ ID NO: 668 - DNA, 41 bp, Artificial Sequence
  Synthetic
gtggcaacagaaatcaggtggtgagcagcacctgcaccaaa

SEQ ID NO: 669 - DNA, 41 bp, Artificial Sequence
  Synthetic
agaggactgctaaaggtttgtagggcagcacctgcaccaag

SEQ ID NO: 670 - DNA, 48 bp, Artificial Sequence
  Synthetic
gtggcaacagaaatcaggtggtgatgcacaaaagtttaayggccttac

SEQ ID NO: 671 - DNA, 48 bp, Artificial Sequence
  Synthetic
agaggactgctaaaggtttgtaggtgcacaaaagtttaayggccttaa

SEQ ID NO: 672 - DNA, 21 bp, Artificial Sequence
  Synthetic
tgttttgccacctttgctcac

SEQ ID NO: 673 - DNA, 46 bp, Artificial Sequence
  Synthetic
gtggcaacagaaatcaggtggtgagtgagcaaaggtggcaaaacag

SEQ ID NO: 674 - DNA, 46 bp, Artificial Sequence
  Synthetic
agaggactgctaaaggtttgtagggtgagcaaaggtggcaaaacat

SEQ ID NO: 675 - DNA, 23 bp, Artificial Sequence
  Synthetic
taaggccrttaaacttttgtgca

SEQ ID NO: 676 - DNA, 75 bp, Artificial Sequence
  Synthetic
agagacctcatttgtgcacaaaagtttaayggccttactgttttgccacctttgctcacagatgaaatgattgct

SEQ ID NO: 677 - DNA, 75 bp, Artificial Sequence
  Synthetic
agagacctcatttgtgcacaaaagtttaayggccttaatgttttgccacctttgctcacagatgaaatgattgct

SEQ ID NO: 678 - DNA, 75 bp, Artificial Sequence
  Synthetic
agcaatcatttcatctgtgagcaaaggtggcaaaacagtaaggccrttaaacttttgtgcacaaatgaggtctct

SEQ ID NO: 679 - DNA, 75 bp, Artificial Sequence
  Synthetic
agcaatcatttcatctgtgagcaaaggtggcaaaacattaaggccrttaaacttttgtgcacaaatgaggtctct

SEQ ID NO: 680 - DNA, 24 bp, Artificial Sequence
  Synthetic
tgcacaaaagtttaayggccttam

SEQ ID NO: 681 - DNA, 21 bp, Artificial Sequence
  Synthetic
tgttttgccacctttgctcac

SEQ ID NO: 682 - DNA, 22 bp, Artificial Sequence
  Synthetic
gtgagcaaaggtggcaaaacak

SEQ ID NO: 683 - DNA, 23 bp, Artificial Sequence
  Synthetic
taaggccrttaaacttttgtgca

SEQ ID NO: 684 - DNA, 24 bp, Artificial Sequence
  Synthetic
agtagcatccttgatttcaccttg

SEQ ID NO: 685 - DNA, 21 bp, Artificial Sequence
  Synthetic
aggggtactgctgttatgtct

SEQ ID NO: 686 - DNA, 20 bp, Artificial Sequence
  Synthetic
gatgcaaatctggtggcgtt

SEQ ID NO: 687 - DNA, 23 bp, Artificial Sequence
  Synthetic
ggggtactgctgttatgtcttta

SEQ ID NO: 688 - DNA, 21 bp, Artificial Sequence
  Synthetic
tgcaaatctggtggcgttaaa

SEQ ID NO: 689 - DNA, 20 bp, Artificial Sequence
  Synthetic
ctgagagagggtcaagtgca

SEQ ID NO: 690 - DNA, 23 bp, Artificial Sequence
  Synthetic
actttagagtccaaccaacagaa

SEQ ID NO: 691 - DNA, 20 bp, Artificial Sequence
  Synthetic
gcccctattaaacagcctgc

SEQ ID NO: 692 - DNA, 20 bp, Artificial Sequence
  Synthetic
tttaacgccaccagatttgc

SEQ ID NO: 693 - DNA, 20 bp, Artificial Sequence
  Synthetic
agcctgcacgtgtttgaaaa

SEQ ID NO: 694 - DNA, 23 bp, Artificial Sequence
  Synthetic
actttagagtccaaccaacagaa

SEQ ID NO: 695 - DNA, 21 bp, Artificial Sequence
  Synthetic
acctgtagaataaacacgcca

SEQ ID NO: 696 - DNA, 20 bp, Artificial Sequence
  Synthetic
aacgccaccagatttgcatc

SEQ ID NO: 697 - DNA, 20 bp, Artificial Sequence
  Synthetic
gcaggctgtttaataggggc

SEQ ID NO: 698 - DNA, 20 bp, Artificial Sequence
  Synthetic
agtctgcctgtgatcaacct

SEQ ID NO: 699 - DNA, 20 bp, Artificial Sequence
  Synthetic
ttttcaaacacgtgcaggct

SEQ ID NO: 700 - DNA, 20 bp, Artificial Sequence
  Synthetic
tgcacttcagcctcaacttt

SEQ ID NO: 701 - DNA, 20 bp, Artificial Sequence
  Synthetic
aacacgtgcaggctgtttaa

SEQ ID NO: 702 - DNA, 21 bp, Artificial Sequence
  Synthetic
tcaatttgcacttcagcctca

SEQ ID NO: 703 - DNA, 21 bp, Artificial Sequence
  Synthetic
acgtgcaggctgtttaatagg

SEQ ID NO: 704 - DNA, 22 bp, Artificial Sequence
  Synthetic
ctttgaagtctgcctgtgatca

SEQ ID NO: 705 - DNA, 22 bp, Artificial Sequence
  Synthetic
cacttcagcctcaactttgtca

SEQ ID NO: 706 - DNA, 20 bp, Artificial Sequence
  Synthetic
aggttgatcacaggcagact

SEQ ID NO: 707 - DNA, 20 bp, Artificial Sequence
  Synthetic
gtgcaacgccaacaataagc

SEQ ID NO: 708 - DNA, 21 bp, Artificial Sequence
  Synthetic
acaaagttgaggctgaagtgc

SEQ ID NO: 709 - DNA, 20 bp, Artificial Sequence
  Synthetic
acgccaacaataagccatcc

SEQ ID NO: 710 - DNA, 22 bp, Artificial Sequence
  Synthetic
cttgacaaagttgaggctgaag

SEQ ID NO: 711 - DNA, 20 bp, Artificial Sequence
  Synthetic
agggagtgaggcttgtatcg

SEQ ID NO: 712 - DNA, 22 bp, Artificial Sequence
  Synthetic
gctgaagtgcaaattgataggt

SEQ ID NO: 713 - DNA, 21 bp, Artificial Sequence
  Synthetic
gccaacaataagccatccgaa

SEQ ID NO: 714 - DNA, 54 bp, Artificial Sequence
  Synthetic
catttggtcattggttcaagacgaatttaatgatggtgtttattttgcttccac

SEQ ID NO: 715 - DNA, 54 bp, Artificial Sequence
  Synthetic
ctggtccgttgtggtccttctaacatttaatgatggtgtttattttgcttccat

SEQ ID NO: 716 - DNA, 28 bp, Artificial Sequence
  Synthetic
tgagaagtytaacataataagaggctgg

SEQ ID NO: 717 - DNA, 52 bp, Artificial Sequence
  Synthetic
catttggtcattggttcaagacgacagcctcttattatgttaracttctcag

SEQ ID NO: 718 - DNA, 52 bp, Artificial Sequence
  Synthetic
ctggtccgttgtggtccttctaaccagcctcttattatgttaracttctcaa

SEQ ID NO: 719 - DNA, 30 bp, Artificial Sequence
  Synthetic
tggaagcaaaataaacaccatcattaaatg

SEQ ID NO: 720 - DNA, 52 bp, Artificial Sequence
  Synthetic
ctaatagctcctgtgccctcgtatctaatattacaaacttgtgcccttttgg

SEQ ID NO: 721 - DNA, 52 bp, Artificial Sequence
  Synthetic
aatccgtcgactagcctgagaattctaatattacaaacttgtgcccttttga

SEQ ID NO: 722 - DNA, 26 bp, Artificial Sequence
  Synthetic
tgaagtttttaacgccaccaratttg

SEQ ID NO: 723 - DNA, 50 bp, Artificial Sequence
  Synthetic
ctaatagctcctgtgccctcgtataaatytggtggcgttaaaaacttcac

SEQ ID NO: 724 - DNA, 50 bp, Artificial Sequence
  Synthetic
aatccgtcgactagcctgagaattaaatytggtggcgttaaaaacttcat

SEQ ID NO: 725 - DNA, 28 bp, Artificial Sequence
  Synthetic
caaaagggcacaagtttgtaatattagg

SEQ ID NO: 726 - DNA, 46 bp, Artificial Sequence
  Synthetic
gtggcaacagaaatcaggtggtgaggctgtttaataggggytgaac

SEQ ID NO: 727 - DNA, 46 bp, Artificial Sequence
  Synthetic
agaggactgctaaaggtttgtaggggctgtttaataggggytgaat

SEQ ID NO: 728 - DNA, 27 bp, Artificial Sequence
  Synthetic
atgtyaayaactcatatgagtgtgaca

SEQ ID NO: 729 - DNA, 51 bp, Artificial Sequence
  Synthetic
gtggcaacagaaatcaggtggtgagtcacactcatatgagttrttracatg

SEQ ID NO: 730 - DNA, 51 bp, Artificial Sequence
  Synthetic
agaggactgctaaaggtttgtagggtcacactcatatgagttrttracata

SEQ ID NO: 731 - DNA, 22 bp, Artificial Sequence
  Synthetic
ttcarcccctattaaacagcct

SEQ ID NO: 732 - DNA, 75 bp, Artificial Sequence
  Synthetic
gtcctaccatttaatgatggtgtttattttgcttccactgagaagtctaacataataagaggctggatttttggt

SEQ ID NO: 733 - DNA, 75 bp, Artificial Sequence
  Synthetic
gtcctaccatttaatgatggtgtttattttgcttccattgagaagtctaacataataagaggctggatttttggt

SEQ ID NO: 734 - DNA, 75 bp, Artificial Sequence
  Synthetic
accaaaaatccagcctcttattatgttagacttctcagtggaagcaaaataaacaccatcattaaatggtaggac

SEQ ID NO: 735 - DNA, 75 bp, Artificial Sequence
  Synthetic
accaaaaatccagcctcttattatgttagacttctcaatggaagcaaaataaacaccatcattaaatggtaggac

SEQ ID NO: 736 - DNA, 75 bp, Artificial Sequence
  Synthetic
ttagatttcctaatattacaaacttgtgcccttttggtgaagtttttaacgccaccagatttgcatctgtttatg

SEQ ID NO: 737 - DNA, 75 bp, Artificial Sequence
  Synthetic
ttagatttcctaatattacaaacttgtgcccttttgatgaagtttttaacgccaccagatttgcatctgtttatg

SEQ ID NO: 738 - DNA, 75 bp, Artificial Sequence
  Synthetic
ataaacagatgcaaatctggtggcgttaaaaacttcaccaaaagggcacaagtttgtaatattaggaaatctaac

SEQ ID NO: 739 - DNA, 75 bp, Artificial Sequence
  Synthetic
ataaacagatgcaaatctggtggcgttaaaaacttcatcaaaagggcacaagtttgtaatattaggaaatctaac

SEQ ID NO: 740 - DNA, 75 bp, Artificial Sequence
  Synthetic
ttttcaaacacgtgcaggctgtttaataggggctgaacatgtcaacaactcatatgagtgtgacatacccattgg

SEQ ID NO: 741 - DNA, 75 bp, Artificial Sequence
  Synthetic
ttttcaaacacgtgcaggctgtttaataggggctgaatatgtcaacaactcatatgagtgtgacatacccattgg

SEQ ID NO: 742 - DNA, 75 bp, Artificial Sequence
  Synthetic
ccaatgggtatgtcacactcatatgagttgttgacatgttcagcccctattaaacagcctgcacgtgtttgaaaa

SEQ ID NO: 743 - DNA, 75 bp, Artificial Sequence
  Synthetic
ccaatgggtatgtcacactcatatgagttgttgacatattcagcccctattaaacagcctgcacgtgtttgaaaa

SEQ ID NO: 744 - DNA, 30 bp, Artificial Sequence
  Synthetic
atttaatgatggtgtttattttgcttccay

SEQ ID NO: 745 - DNA, 28 bp, Artificial Sequence
  Synthetic
tgagaagtytaacataataagaggctgg

SEQ ID NO: 746 - DNA, 28 bp, Artificial Sequence
  Synthetic
cagcctcttattatgttaracttctcar

SEQ ID NO: 747 - DNA, 30 bp, Artificial Sequence; identical to SEQ ID NO: 719
  Synthetic

SEQ ID NO: 748 - DNA, 28 bp, Artificial Sequence
  Synthetic
ctaatattacaaacttgtgcccttttgr

SEQ ID NO: 749 - DNA, 26 bp, Artificial Sequence
  Synthetic
tgaagtttttaacgccaccaratttg

SEQ ID NO: 750 - DNA, 26 bp, Artificial Sequence
  Synthetic
aaatytggtggcgttaaaaacttcay

SEQ ID NO: 751 - DNA, 28 bp, Artificial Sequence
  Synthetic
caaaagggcacaagtttgtaatattagg

SEQ ID NO: 752 - DNA, 22 bp, Artificial Sequence
  Synthetic
ggctgtttaataggggytgaay

SEQ ID NO: 753 - DNA, 27 bp, Artificial Sequence
  Synthetic
atgtyaayaactcatatgagtgtgaca

SEQ ID NO: 754 - DNA, 27 bp, Artificial Sequence
  Synthetic
gtcacactcatatgagttrttracatr

SEQ ID NO: 755 - DNA, 22 bp, Artificial Sequence
  Synthetic
ttcarcccctattaaacagcct

SEQ ID NO: 756 - DNA, 21 bp, Artificial Sequence
  Synthetic
aggggtactgctgttatgtct

SEQ ID NO: 757 - DNA, 21 bp, Artificial Sequence
  Synthetic
gcatccttgatttcaccttgc

SEQ ID NO: 758 - DNA, 21 bp, Artificial Sequence
  Synthetic
gcatccttgatttcaccttgc